(19) United States
(12) Patent Application Publication
(10) Pub. No.: US 2019/0352262 A1
(43) Pub. Date: Nov. 21, 2019

(54) New Pleuromutilin Antibiotic Compounds, Compositions and Methods of Use and Synthesis

(71) Applicant: YALE UNIVERSITY, New Haven, CT (US)

(72) Inventors: Seth Herzon, Madison, CT (US); Stephen K. Murphy, Providence, RI (US); Mingshuo Zeng, Berkeley, CA (US)

(21) Appl. No.: 16/475,965

(22) PCT Filed: Feb. 1, 2018
(86) PCT No.: PCT/US2018/016427

§ 371 Date: Jul. 3, 2019

Related U.S. Application Data
(60) Provisional application No. 62/453,330, filed on Feb. 1, 2017.
(60) Provisional application No. 62/483,653, filed on Apr. 10, 2017.

(51) Int. Cl. C07D 209/96; C07C 69/675; C07C 49/573; C07D 317/72

(52) U.S. Cl. CPC C07D 209/96; C07C 69/675; A61K 45/06; C07D 317/72; C07C 49/573

(57) ABSTRACT

The present invention is directed to novel pleuroniutilin antibiotic compounds, intermediates which are useful for making these novel amibioiic compounds and related methods and pharmaceutical compositions for treating pathogens, especially bacterial infections, including gram negative bacteria and synthesizing these compounds.

### RELATED APPLICATIONS

The present application claims the benefit of priority of U.S. provisional application Ser. Nos. U.S. 62/453,330, filed Feb. 1, 2.017 and U.S. 62/483,653, filed Apr. 10, 2017 of identical title, the entire contents of said applications being incorporated by reference herein.

### FIELD OF THE INVENTION

The present invention is directed to novel pleuromutilin antibiotic compounds, intermediates which are useful for making these novel antibiotic compounds and related methods and pharmaceutical compositions for treating pathogens, especially bacterial infections, including gram negative bacteria and synthesizing these compounds.

### BACKGROUND OF THE INVENTION

Although natural products form the basis of nearly 50% of all small molecule drugs, unaltered natural products comprise only 4% of all therapeutics, indicating that natural products are typically not optimized for non-natural uses in humans. (+)-Pleuromutilin is a diterpene fungal metabolite that inhibits the growth of gram-positive pathogens by binding the peptidyl transferase site of the bacterial ribosome. Importantly, resistance to pleuromutilins is slow to develop, and these agents display minimal cross-resistance with existing antibiotics.

(+)-Pleuromutilin (1, FIG. 1) was isolated in 1951 by Kavanagh, Hervey, and Robbins from Pleurotus mutilus and Pleurotus passeckerianus and shown to inhibit the growth of Gram-positive bacteria (FIG. 1). Anchel, Arigoni, and Birch established the structure of 1, which was confirmed by X-ray crystallographic analysis. (+)-Pleuromutilin (1) is comprised of a densely-functionalized eight-membered carbocycle fused to a cis-hydrindanone core and contains eight contiguous stereocenters, three of which are quaternary. The biosynthesis of (+)-pleuromutilin (1), from geranylgeranyl pyrophosphate, has been elucidated.

The antibacterial properties of pleuromutilins derive from the inhibition of bacterial protein synthesis. The tricyclic core and the C14 glycolic acid residue bind the A- and P-sites, respectively, of the peptidyl transferase center. The C14 glycolic acid residue is essential for antibacterial activity; by comparison, the deacylated derivative (+)-mutilin (2) is not active against Gram-positive bacteria. Thousands of C14 analogs have been prepared from natural (+)-pleuromutilin (1). Tiamulin (3) and valnemulin (4) are C14 analogs used in veterinary applications since the 1980s. Retapamulin (5) was approved in 2007 for the treatment of impetigo in humans. Most pleuromutilins tested to date elicit very low mutational frequencies, and as of 2014 clinical resistance to retapamulin (5) therapy has not been recorded. Lefamulin (6) recently passed a Phase III clinical trial for the treatment of community-acquired bacterial pneumonia (IV-to-oral administration).

(+)-Pleuromutilin (compound 1, FIG. 1) itself is available in large quantities by fermentation, and extensive efforts have been devoted toward improving its pharmacological profile by derivatization. The majority of these efforts have focused on modification of the C-14 side chain (the deacylated form of compound 1, (+)-mutilin (compound 2, FIG. 1), is largely inactive), and to date, >3000 C-14 derivatives have been prepared. These efforts culminated in the approval of retapamulin (Compound 4 of FIG. 1) in 2007 for the treatment of topical methicillin-resistant Staphylococcus (MRSA) infections.

The derivatives 3-5, FIG. 1 (and other C14 analogs) are active against primarily Gram-positive pathogens. Functionalization of the cyclooctane ring has the potential to significantly improve the spectrum of activity. For example, epimerization of the C12 position (by an unusual retroallylation-allylation reaction discovered by Berner, vide infra), followed by functionalization of the transposed alkene provides 12-epi-pleuromutilin derivatives, which possess activity against Gram-negative pathogens. This improved activity is due in part to decreased AcrAB-TolC efflux, a common resistance mechanism in Gram-negative strains. Pleuromutilins inhibit the three bacterial strains recently classified as urgent threats by the Centers for Disease Control and Prevention: Clostridium difficile, carbapenem-resistant Enterobacteriaceae (CRE), and drug-resistant Neisseria gonorrhoeae.

As outlined, semisynthesis has primarily enabled modification of C14 and, to a lesser extent, C12. Although a limited number of changes to other positions have been made, much of the chemical space surrounding the carbon skeleton remains unexplored. A fully synthetic route to pleuromutilins would enable access to a greater diversity of antibiotics with potentially expanded activity spectra and improved pharmacological properties. Herein the present inventions describe in full detail their synthetic studies toward (+)-pleuromutilin (1), culminating in the development of a convergent, enantioselective, 16-step route to the (+)-12-epi-mutilin scaffold as well as a 19-step route to (+)-pleuromutilin (1) itself. The inventors believe that the present invention provides a foundation to leverage the wealth of existing target binding and structure-activity data toward the production of improved fully synthetic analogs. Successes in the development of fully synthetic routes to other clinical classes of antibiotics, such as β-lactams, vancomycins, tetracyclines, and macrolides, underscore the potential for antibiotic development through chemical synthesis.

In 1986, Berner reported that the C-12 quaternary stereocenter of pleuromutilin could be epimerized (to an ˜1:1 mixture of C-12 diastereomers) by a zinc-mediated retroallylation-allylation sequence (see 4, FIG. 2A). In 2015, researchers at Nabriva Therapeutics reported that functionalization of the transposed alkene provides 12-epi-mutilin derivatives with broad-spectrum activity, including activity against gram-negative pathogens (GNPs). Given the increasing occurrence of drug-resistant GNPs, we deemed 12-epi-mutilins as valuable targets for synthesis. To maximize the scope of accessible derivatives, the inventors conceived a strategy involving late-stage construction of the macrocycle using a conjunctive reagent that could be easily modified at positions 11-13 (See 5 and 6 of FIG. 2B. Recent successes in the development of fully synthetic routes to tetracycline and macrolide antibiotics under the potential to generate new clinical candidates by total synthesis.

### BRIEF DESCRIPTION OF THE INVENTION

In one embodiment, the present invention is directed to compounds according to the chemical structure:

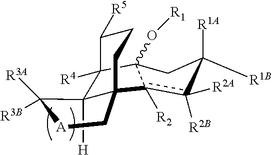

Where A is O, S, —N(R^{N})(C(R_{A})(R_{B}))_{g}— or —(C(R_{A})(R_{B}))ₕ—;
R^{N} is H or a C₁-C₃ alkyl group which is optionally substituted with from 1 to 3 hydroxyl groups or halogen groups (preferably fluoro groups);
R_{A} and R_{B} are each independently H, OH, a halogen group (often F), a C₁-C₃ alkyl which is optionally substituted with from 1-3 halogen groups (often 1-3 fluoro groups) or 1-3 hydroxyl groups (often a single hydroxyl group) or together R_{A} and R_{B} form a cyclopropyl or cyclobutyl group on a single carbon;
R₁ is H, an optionally substituted C₁-C₇ alkyl group (preferably C₁-C₃ alkyl, preferably methyl) which is preferably substituted with from 1-5 halogens (F, Cl, Br or I), often from 1-3 fluoro groups or from 1-3 hydroxyl groups, a Sugar group wherein said sugar group is a monosaccharide or disaccharide sugar as otherwise described herein which forms a glycosidic linkage with the oxygen (preferably at the 1 or 4 carbon position of the sugar moiety bonded to the oxygen), an optionally substituted —(CH₂)ᵢ—C(O)—C₀-C₆ (preferably C₁-C₆) alkyl group (forming an ester) which is preferably substituted with from 1-5 halogens, often 1-3 fluoro groups and from 1-3 hydroxyl groups (preferably, R₁ forms a methyl ester group substituted with a single hydroxyl group) or a —(CH₂)ᵢ—C(O)—(CH₂)ᵢ—O-Sugar group;
R^{1A} and R^{1B} are each independently H, OH, an optionally substituted C₁-C₆ alkyl or C₂-C₆ alkenyl group (preferably vinyl, often R^{1B} is a vinyl group wherein said alkyl group or said alkenyl group is preferably substituted with from 1-5 halogen groups and/or from 1-3 hydroxyl groups), an optionally substituted —(CH₂)NR^{NA}R^{NB} group, OH, an optionally substituted —(CH₂)ᵢO—C₁-C₆ alkyl group, an optionally substituted —(CH₂)ᵢC(O)—C₀-C₆ alkyl (preferably C₁-C₆), an optionally substituted —(CH₂)ᵢC(O)O—C₁-C₆ alkyl or an optionally substituted —(CH₂)ᵢOC(O)—C₁-C₆ alkyl wherein each of the aforementioned alkyl groups is preferably substituted with from 1-5 halogen groups (often 1-3 fluoro groups) or from 1-3 hydroxyl groups, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar, an optionally substituted —(CH₂)ᵢ—C(O)—(CH₂)ᵢ—O-Sugar group, or R^{1A} or R^{1B} together with the carbon atom to which R₂ is attached form an optionally substituted (preferably optionally substituted with from 1 to 4 methyl or 1-3 hydroxyl groups) 5-6 membered carbocyclic ring which links the carbon atoms which are bonded to R^{1A} or R^{1B} and R₂, respectively, wherein the alkylene group extends above or below the plane of the molecule;
R^{NA} and R^{NB} is each independently H, a C₁-C₆ alkyl which is optionally substituted with from 1-3 halo groups (preferably F) or 1-3 hydroxyl groups (often 1 hydroxyl group), an optionally substituted —(CH₂)ᵢO—C₁-C₆ alkyl, an optionally substituted —(CH₂)ᵢC(O)C₀-C₆ alkyl (preferably C₁-C₆), an optionally substituted —(CH₂)ᵢC(O)OC₁-C₆ alkyl, an optionally substituted —(CH₂)ᵢOC(O)C₁-C₆ alkyl, an optionally substituted —(CH₂)ᵢAryl, an optionally substituted —(CH₂)ᵢO-Aryl an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar or an optionally substituted —(CH₂)ᵢ—C(O)—(CH₂)ᵢ—O-Sugar group;
R₂ is H, OH, an optionally substituted C₁-C₈ alkyl group which is preferably substituted with from 1-5 halo groups, often 1-3 fluoro groups or from 1-3 hydroxyl groups, OH, SH, an optionally substituted —(CH₂)ᵢNR^{NA}R^{NB} group, an optionally substituted —(CH₂)ᵢO—C₁-C₆ alkyl group, an optionally substituted —(CH₂)ᵢC(O)—C₁-C₆ an optionally substituted —(CH₂)ᵢC(O)O—C₁-C₆ alkyl or an optionally substituted —(CH₂)ᵢOC(O)—C₁-C₆ alkyl wherein each of the aforementioned alkyl groups is preferably substituted with from 1-5 halogen groups (often 1-3 fluoro groups) or from 1-3 hydroxyl groups, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar or an optionally substituted —(CH₂)ᵢ—C(O)—(CH₂)ᵢ—O-Sugar group, or R₂ together with R^{1A} or R^{1B} forms a C₂-C₅ alkylene group optionally substituted with from 1 to 4 methyl groups which links the carbon atoms which are bonded to R₂ and R^{1A} or R^{1B}, respectively, wherein the alkylene group extends above or below the plane of the molecule;
R^{2A} and R^{2B} are each independently H, OH, an optionally substituted C₁-C₆ alkyl or C₂-C₆ alkenyl group (preferably vinyl) wherein said alkyl group or said alkenyl group is preferably substituted with from 1-5 halogen groups and from 1-3 hydroxyl groups), an optionally substituted —(CH₂)ᵢNR^{NA}R^{NB} group, an optionally substituted —(CH₂)ᵢO—C₁-C₆ alkyl, an optionally substituted —(CH₂)ᵢC(O)—C₀-C₆ alkyl (often C₁-C₆ alkyl), an optionally substituted —(CH₂)ᵢC(O)O—C₁-C₆ alkyl or an optionally substituted —(CH₂)₁OC(O)—C₁-C₆ alkyl wherein each of the aforementioned alkyl groups is preferably substituted with from 1-5 halogen groups (often 1-3 fluoro groups) or from 1-3 hydroxyl groups, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar or an optionally substituted —(CH₂)ᵢ—C(O)—(CH₂)ᵢ—O-Sugar group;
R^{3A} and R^{3B} are each independently H, OH, a C₁-C₆ optionally substituted alkyl group, an optionally substituted —(CH₂)ᵢO—C₁-C₆ alkyl group, or R^{3A} and R^{3B} together with the carbon atom to which they are attached form a C₂-C₆ diether group, often a C₃ or C₄ diether group (each of the two oxygens of the diether group being bonded to the carbon to which R^{3A} and R^{3B} are bonded) or a keto group (═O) with the carbon to which they are bonded;
R⁴ and R⁵ are each independently H or an optionally substituted C₁-C₈ alkyl group (preferably methyl) wherein said substitution is preferably from 1-5 halo groups (often F) or from 1-3 hydroxyl groups (often a single hydroxyl group);
g is 0, 1, 2 or 3;
h is 1, 2, 3 or 4;
i is 0, 1, 2, 3, 4, 5 or 6; and
the carbon atoms to which OR¹ and R₂ are attached optionally are bonded to each other; or
a pharmaceutically acceptable salt, stereoisomer, solvate or polymorph thereof.

In preferred compounds according to the present invention, A is CH₂, —N(R^{N})(C(R_{A})(R_{B}))₈— or —(C(R_{A})(R_{B}))ₕ— where RN is H or a C₁-C₁ alkyl group optionally substituted with from 1-3 fluoro groups or 1-3 hydroxyl groups) and R_{A} and R_{B} are each independently H, halogen (especially fluoro) or a C₁-C₃ alkyl group optionally substituted with from 1-3 fluoro groups (preferably 3 fluoro groups) or 1-3 hydroxyl groups (preferably 1 hydroxyl group);
R₁ is H, an optionally substituted C₁-C₇ alkyl group (preferably C₁-C₃ alkyl, preferably methyl) which is preferably substituted with from 1-5 halogens (F, Cl, Br or I), often from 1-3 fluoro groups or from 1-3 hydroxyl groups, preferably 1 hydroxyl group or a C(O)C₁-C₆ alkyl group optionally substituted with 1-3 fluoro groups or 1-3 hydroxyl groups or a —(CH₂)ᵢ—C(O)—(CH₂)ᵢ—O-Sugar group;
R^{1A} and R^{1B} are each H, a C₁-C₇ alkyl group or a C₂-C₆ alkenyl group, each of which is optionally substituted with 1-3 halogen (preferably fluoro) groups or 1-3 hydroxyl groups, a —(CH₂)ᵢ—O—C₁-C₆ alkyl group, a —(CH₂)ᵢ—C(O)C₁-C₆ alkyl group, a —(CH₂)ᵢ—O—C(O)C₁-C₆ alkyl group or a —(CH₂)ᵢ—C(O)O—C₁-C₆ alkyl group, each of which groups is optionally substituted with from 1-3 halogen (preferably fluoro) or from 1-3 hydroxyl groups, a —(CH₂)ᵢ-Sugar group, a —(CH₂)ᵢ—O-Sugar group, a —(CH₂)ᵢ—C(O)—(CH₂)ᵢ—O-Sugar group or a —(CH.₂)ᵢ—NR^{NA}R^{NB} group, where R^{NA} and R^{NA} are each independently H, a C₁-C₆ alkyl group optionally substituted with 1-3 halogens (preferably fluoro) or 1-3 hydroxyl groups, a —(CH₂)ᵢ—O—C₁-C₆ alkyl group, a —(CH₂)ᵢ—C(O)C₁-C₆ alkyl group, a —(CH₂)ᵢ—O—C(O)C₁-C₆ alkyl group or a —(CH₂)ᵢ—C(O)O—C₁-C₆ alkyl group, each of which groups are optionally substituted with from 1-3 halogen (preferably fluoro) or from 1-3 hydroxyl groups, an optionally substituted —(CH₂)ᵢAryl, an optionally substituted —(CH₂)ᵢO-Aryl, an optionally substituted —(CH₂)ᵢHeteroaryl, an optionally substituted —(CH₂)ᵢO-Heteroaryl, an optionally substituted —(CH₂)ᵢSugar or an optionally substituted —(CH₂)ᵢO-Sugar group, or R^{1A} and the carbon to which R₂ is attached form a 5-6 membered carbocyclic ring, which is optionally substituted;
R₂ is H, a C₁-C₈ alkyl group optionally substituted with 1-3 halogens (preferably fluoro) or 1-3 hydroxyl groups, a —(CH₂)ᵢ—O—C₁-C₆ alkyl group which is optionally substituted with from 1-3 halogens (preferably fluoro) or from 1-3 hydroxyl groups, a —(CH₂)ᵢ—C(O)—(CH₂)ᵢ—O-Sugar group, or a —(CH₂)ᵢ—NR^{NA}R^{NB} group where R^{NA} and R^{NB} are the same as directly described above;
R^{2A} and R^{2B} are each independently H, a C₁-C₆ alkyl group or a C₂-C₆ alkenyl group each of which is optionally substituted with front 1-3 halogens (preferably fluoro) or from 1-3 hydroxyl groups, a —(CH₂)ᵢ—O—C₁-C-₆ alkyl group, a —(CH₂)ᵢ—C(O)C₁-C₆ alkyl group, a —(CH₂)ᵢ—O—C(O)C₁-C₆ alkyl group or a —(CH₂)ᵢ—C(O)O—C-₁-C₆ alkyl group, each of which groups is optionally substituted with from 1-3 halogen (preferably fluoro) or from 1-3 hydroxyl groups, a —(CH₂)ᵢ-Sugar group, a —(CH₂)ᵢ—O-Sugar group, a —(CH₂)ᵢ—C(O)—(CH₂)ᵢ—O-Sugar group, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl;
R^{3A} and R^{3B} are each independently H, OH, a C₁-C-₆ alkyl group which is optionally substituted with from 1-3 halogens or from 1-3 hydroxyls, a keto group (C═O) or together with the carbon to which they are both attached, forth a C₃ or C₄ diether group; and
R⁴ and R⁵ are each independently H or a C₁-C₃ alky group optionally substituted with from 1-3 halogens (preferably fluoro) or from 1-3 hydroxyl groups;
g is 0 or 1;
h is 1, 2 or 3; and
i is 0, 1, 2 or 3, or
a pharmaceutically acceptable salt or stereoisomer thereof.

Preferred compounds according to the present invention are also set forth in FIG. 3 hereof. In the compounds of FIG. 3 hereof, R¹ is preferably H, a C₁-C₇ alkyl group which is optionally substituted with from 1-3 fluoro groups or 1-3 hydroxyl groups, a —C(O)—C₁-C₆ alkyl group which is optionally substituted with from 1-3 fluoro groups and 1-3 hydroxyl groups (more preferably a single hydroxyl group) or an optionally substituted —(CH₂)ᵢ—C(O)—(CH₂)ᵢ—O-Sugar group (i is preferably 0). R² is H, a C₁-C₆ alkyl group which is optionally substituted with from 1-3 halo groups (preferably F) or 1-3 hydroxyl groups (often a single hydroxyl group), —C(O)C₁-C₆ alkyl which is optionally substituted with 1-3 halogens (preferably fluoride) and 1-3 hydroxyl groups (often a single hydroxyl group), —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar or an optionally substituted —(CH₂)ᵢ—C(O)—(CH₂)ᵢ—O-Sugar group.

In a further embodiment, the present invention is directed to pharmaceutical compositions comprising an anti-microbial (preferably, anti-bacterial) effective amount of at least one compound as described above, in combination with a pharmaceutically acceptable carrier, additive or excipient. In a further embodiment, pharmaceutical compositions according to the present invention optionally include an effective amount of an additional bioactive agent, preferably at least one additional antibiotic effective for treating pathogens, especially including bacteria (gram negative or grant positive).

An additional embodiment of the present invention is directed to a method for treating pathogens, often bacterial infections including gram positive and gram negative bacteria, especially gram-negative bacterial infections as well as gram positive Staphylococcus aureus, including MRSA infections, comprising administering to a patient or subject in need an effective amount of at least one compound according to the present invention, optionally in combination with at least one additional bioactive agent, preferably an additional antibiotic.

Still a further embodiment of the present invention is directed to a method of synthesizing compounds according to the present invention, especially 12-epi-pleuromutilin, (+)-pleuromutilin, 11,12-diepi-mutilin and 11,12-diepi-pleuromutilin (the syntheses of 12-epi-mutilin) and other analogs of compounds according to the present invention, following the Schemes 1-17 which are presented in FIGS. 4-20 attached hereto.

Still an additional embodiment of the present invention is directed to a method of synthesizing compound 14 from compound 13 as indicated below by subjecting compound 13 to a Nagata hydrocyanation using an aluminum cyanide reagent (diethylaluminumcyanide or triethylaluminum/HCN) to provide compound 14 below in high yield (greater than 50%, often more than 75% or more than 90% yield from compound 13). This reaction produces two isomers one of which may be recycled to produce further hydrocyanation product 14 (See FIG. 15, Scheme 12, bottom).

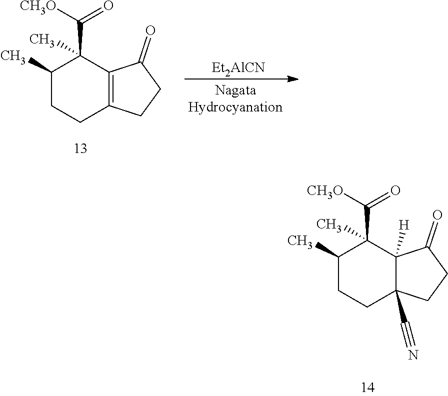

In another embodiment the present invention is directed to a method of synthesizing compound 7 below from compound 16 comprising exposing compound 16 to excess methyl lithium (CH₃Li) followed by exposure of the intermediate to Boc₂O (ditertbutyldicarbonate or Boc anhydride) to provide compound 7 in greater than 70% yield, wherein said synthesis takes place step-wise or in a single pot. This reaction is also depicted in FIG. 17, Scheme 14 hereof.

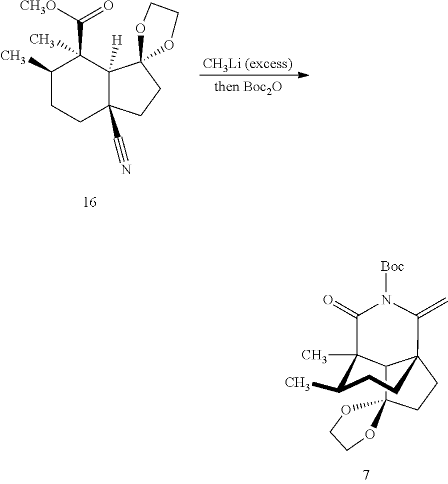

In another embodiment of the present invention, compound 21R, where R is a C₁-C₃ alkyl group or a vinyl group, preferably a methyl or a vinyl group as indicated below is synthesized from compound 8R where R is a C₁-C₃ alkyl group or a vinyl group, preferably a methyl group or a vinyl group as indicated below and compound 7 comprising exposing a mixture of compound 8R and compound 7 to a strong lithium base (e.g. t-BuLi) followed by exposure of the mixture to acidic solution (e.g. HCL, other acidic solution) to provide compound 21R where R is a C₁-C₃ alkyl or a vinyl group, preferably a methyl or vinyl group in high yield (at least 45%, preferably at least 60%). This reaction is also depicted in FIG. 17, Scheme 14, compound 57 being converted to compound 58 in at least 60% yield.

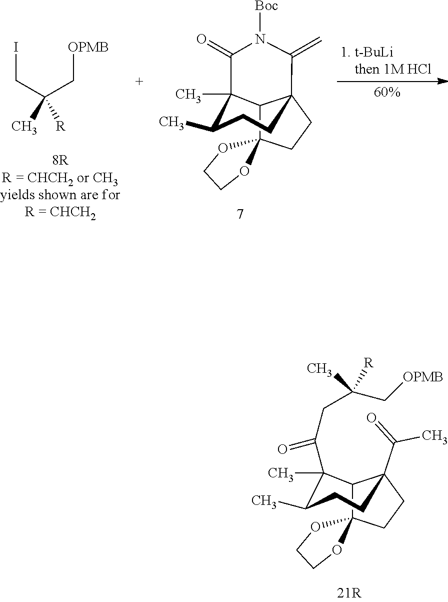

In an additional embodiment, compound 24 is prepared in an exo-selective reductive cyclization by reacting compound 23 in the presence of a nickel metal precatalyst such as Ni(COD)2 (Bis(1,5-cyclooctadiene)nickel), a ligand such as an N-heterocyclic carbine (e.g., IPr or 1,3-Bis(2,6-diisopropylphenyl)imidazol-2-ylidene, alternatively, IPrCl or 1,3-Bis-(2,6-diisopropylphenyl)imidazolinium chloride) and a trialkylhydrosilane (e.g. triethylhydrosilane) to form an allylic silyl ether as an intermediate which is then subjected to cleavage of the silyl ether (e.g. with tetra-n-butyl ammonium fluoride) to provide the allylic alcohol compound 24.

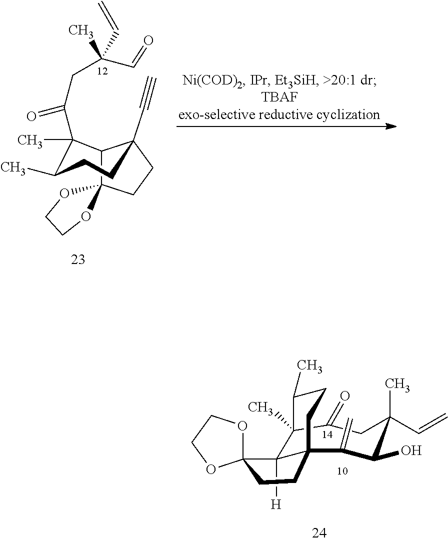

In still another synthetic method embodiment of the present invention, precursor compound 36 undergoes a nickel-catalyzed aldehyde metathesis reaction to form thee eight membered ring-formed compound 37 by exposing compound 36 to a nickel pre-catalyst which may include nickel precatalysts in the 0 or +2 oxidation states such as Ni(COD)₂, a N-heterocyclic carbene such as IPr or IPrCl or a phosphine, further optionally including a silane (such as HSiEt₃ or HSi(iPr)₃) to produce compound 37 which may subsequently be subjected to reduction conditions in sodium borohydride and cesium trichloride (or alternatively, with for example, a borane, an organozinc reagent, alcohol and/or dihydrogen) to provide compound 38 in quantitative yield.

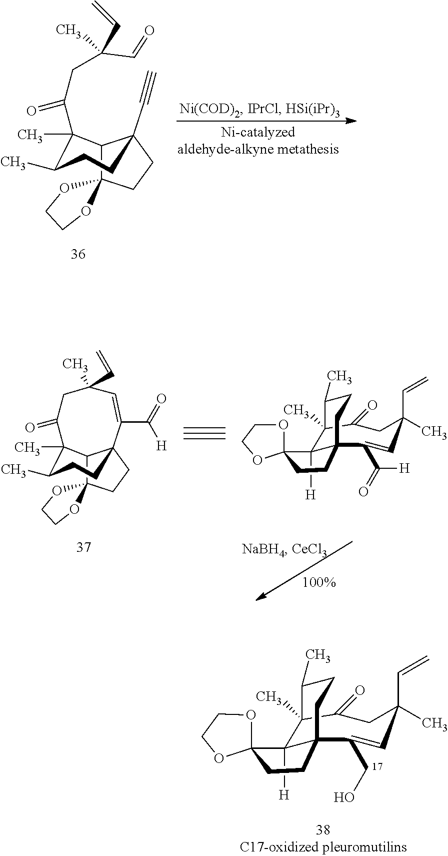

In still a further embodiment of the present invention, compound 36 is objected to nickel catalyzed reductive polycyclization conditions Ni(COD)2, IPrCl and a silane (e.g. HSi(Et)₃) to provide compound 39, which may be exposed to tetra-n-butylammonium fluoride (TBAF) in order to remove the silyl group to provide compound 40, depicted below. These reactions are presented in attached FIG. 9, Scheme 6.

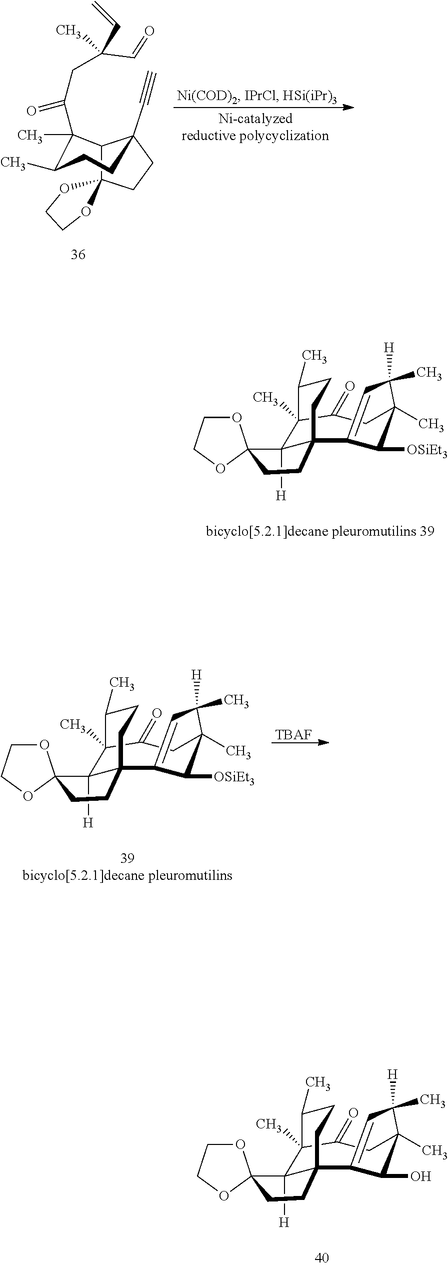

A variation of the reaction presented just above is found in FIG. 18, Scheme 15. In this reaction compound 62 is subjected to Ni catalyzed reductive cyclization under slightly different conditions to produce to compound 63 and 66 respectively using Ni(cod)₂ (preferably 40%) L4 (preferably 40%) and 5 equivalents of a trialkyl silyl group ((iPr)₃—SiH or Et₃SiH), respectively to produce compounds 63 and 66 as depicted below. The proposed mechanism for the reactions are presented in FIG. 18, Scheme 15 below the general reactions.

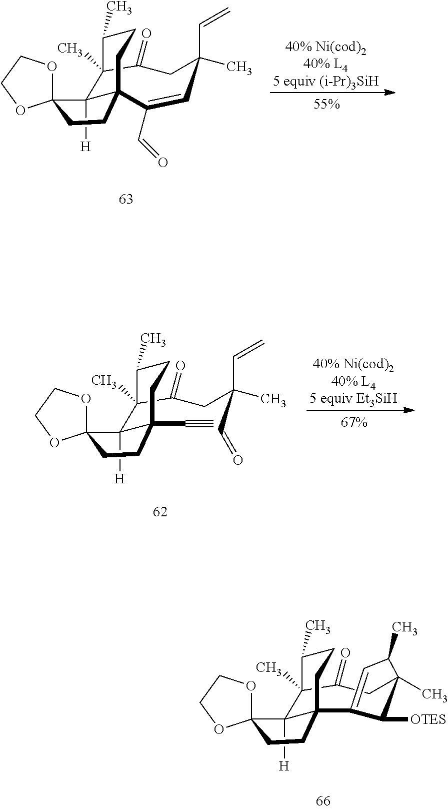

### BRIEF DESCRIPTION OF THE FIGURES

FIG. 1 shows the chemical structure of natural (+)-pleuromutilin (1) and the deacylated derivative (=)-mutilin (2), structures of semisynthetic C14 derivatives tiamulin (3), retapamulin, (4) and lefamulin (5) and the structure of lefamulin (6). Natural (+)-pleuromutilin (1) and the semisynthetic C14 derivatives 3-5 are active primarily against Gram-positive pathogens. 12-epi-mutilin derivatives possess extended spectrum activity against Gram-negative and drug-resistant pathogens.

FIG. 2 shows A. Structures of selected pleuromutilins and 12epi-mutilins. B. The retrosynthetic analysis and the fragments (7,8) employed in the synthesis of 12-epimutilin (4).

FIG. 3 depicts a number of preferred compounds according to the present invention.

FIG. 4, Scheme 1A, shows the chemical synthetic steps of synthesizing the amine-protected and keto-protected compound 7 from an intermediate compound 10 (which may be obtained from cyclohexenone 18 pursuant to scheme FIG. 12, Scheme 9). Scheme 1B shows the chemical synthetic steps of synthesizing intermediate compound 8, from compound 19. Compound 7 and compound 8 are used as reactants to provide complex antibiotic compounds according to FIG. 5, scheme 2.

FIG. 5, Scheme 2, shows the chemical synthetic steps of synthesizing the keto-protected 12-epi-mutilin compound 5A and the keto protected 11,12-diepi-mutilin 26A from intermediates 8 and 7, prepared pursuant to Scheme 1A and 1B, described above, which can be deprotected in acid to produce 12-epimutilin (5) and 11,12-diepi-mutilin (26).

FIG. 6, Scheme 3, shows the chemical synthetic steps of synthesizing (+) pleuromutilin (29) and 12-epi-pleuromutilin from compound 5A and 11,12-epipleuromutilin from compound 26A.

FIG. 7, Scheme 4, shows chemical synthetic steps/reaction conditions for synthesizing 12-epi-mutilin and 11,12-diepi-mutilin.

FIG. 8, Scheme 5, shows chemical synthetic steps for synthesizing 11,12-diepi-pleuromutilin, 12-epi-pleuromutilin and (+)-pleuromutilin.

FIG. 9, Scheme 6, shows the nickel catalyzed aldehyde alkyne metathesis and nickel catalyzed reductive polycyclization reactions of compound 36.

FIG. 10, Scheme 7 shows keys steps in prior art syntheses of Gibbons (A); Boeckman (B) and Procter (C) syntheses of pleuromutilin.

FIG. 11, Scheme 8, shows A. The outlines of the strategy to access (+)-mutilins (2). B. The cyclization substrate 16 targeted. C. Destabilizating syn-pentane and transannular interactions arising from a more flexible and saturated cyclization precursor.

FIG. 12, Scheme 9 shows the stereoselective chemical synthesis of hydrindanone 14 from cyclohexenone 18 through two routes.

FIG. 13, Scheme 10 shows A. An attempted synthesis of diketone 25 via the acid chloride 23 or the lactone 27. B. The synthesis of the alkyl iodiide (S)-30

FIG. 14, Scheme 11 shows A. The synthesis of the C11-C14 aldehyde 37. B. Shows the synthesis of the hydrindanone 42.

FIG. 15, Scheme 12 shows A. 1,4-Addition of lithium divinylcuprate and hydrogen cyanide to the hydrindanone 14. B. An improved procedure for the 1,4-hydrocyanation of 14 involving recycling of the undesired stereoisomer 50.

FIG. 16, Scheme 13 A: shows the synthesis of cyclopentene 53 from enone 42. B: shows proposed mechanism for the synthesis of 53.

FIG. 17, Scheme 14 shows the synthesis of alkynylaldehyde 62 from the hydrocyanation product 49.

FIG. 18, Scheme 15 shows divergent cyclization pathways of alkynylaldehyde 62.

FIG. 19, Scheme 16 shows the synthesis of tetracycle compound 79.

FIG. 20, Scheme 17 shows the synthesis of (+)-pleuromutilin (1), (+)-12-epi-pleuromutilin (97) and (+)-11,12-di-epi-pleuromutilin (93).

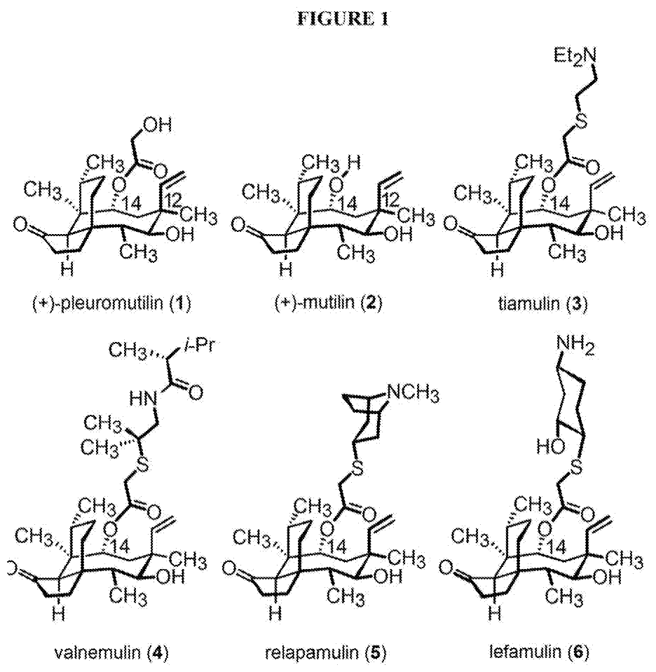

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges that may independently be included in the smaller ranges are also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state, especially a bacterial infection including a MRSA infection within the context of its use or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers individual optical isomers/enantiomers or racemic mixtures and geometric isomers), pharmaceutically acceptable salts and prodrug forms. The term compound herein refers to stable compounds. Within its use in context, the term compound may refer to a single compound or a mixture of compounds as otherwise described herein. It is understood that the choice of substituents or bonds within a Markush or other group of substituents or bonds is provided to form a stable compound from those choices within that Markush or other group. The symbol [symbol] used alone or in the symbol [symbol] in a compound according to the present invention is used to represent an optional bond. Note that no more than one optional bond exists in a compound according to the present invention.

The term “pharmaceutically acceptable” as used herein means that the compound or composition is suitable for administration to a subject to achieve the treatments described herein, without unduly deleterious side effects in light of the severity of the disease and necessity of the treatment.

The term “independently” is used herein to indicate that the variable, which is independently applied, varies independently from application to application.

The term “non-existent” or “absent” refers to the fact that a substituent is absent and the group to which such substituent is attached forms an additional bond with an adjacent atom or group.

The terms “treat”, “treating”, and “treatment”, etc., as used herein within context, also refers to any action providing a benefit to a patient at risk for any of the disease states or conditions (bacterial pathogens, especially MRSA infections) which can be treated pursuant to the present invention (e.g., inhibit, reduce the severity, cure, etc.). Treatment, as used herein, principally encompasses therapeutic treatment, but may also encompass both prophylactic and therapeutic treatment, depending on the context of the treatment. The term “prophylactic” when used in context, means to reduce the likelihood of an occurrence or in some cases, reduce the severity of an occurrence within the context of the treatment of a disease state or condition otherwise described herein.

The term “prevention” is used within context to mean “reducing the likelihood” of a condition or disease state from occurring as a consequence of administration or concurrent administration of one or more compounds or compositions according to the present invention, alone or in combination with another agent. Thus, the term prevention is used within the context of a qualitative measure and it is understood that the use of a compound according to the present invention to reduce the likelihood of an occurrence of a condition or disease state as otherwise described herein will not be absolute, but will reflect the ability of the compound to reduce the likelihood of the occurrence within a population of patients or subjects in need of such prevention.

The term “gram negative bacteria” is used to describe any number of bacteria which are characterized in that they do not retain crystal violet stain used in the gram staining method of bacterial differentiation. These bacteria are further characterized by their cell walls, which are composed of a thing layer of peptidoglycans sandwiched between an outer membrane and an inner cytoplasmic cell membrane. Exemplary gram negative bacteria include, for example, Escherichia sp., (Escherichia coli), as well as a larger number of pathogenic bacteria, including Salmonella sp. Shigella sp., Heliobacter sp. (e.g. H. pylori), Acetic acid bacteria, Legionella sp., Cyanobacteria sp., Neisseria sp. (Neisseria gonorrhaeae), Acinetobacter baumanii, Fusobacterium sp., Haemophilus sp. (Haemophilus influenzae), Klebsiella sp., Leptospiria, Nitrobacter sp., Proteus sp., Rickettsia sp., Serratia sp., Thiobacter sp., Treponema sp., Vibrio sp., and Yersinia sp., among others. Compounds according to the present invention are particularly useful for the treatment of gram negative bacterial infections, especially infections caused by the gram negative bacteria set forth above. In certain embodiments, the infection to be treated is caused by Staphylococcus aureus, especially MRSA, which is a gram positive bacteria.

The term “gram positive bacteria” is used to describe any number of bacteria which are characterized in that they do retain crystal violet stain used in the gram staining method of bacterial differentiation. These bacteria are further characterized by their cell walls, which are composed of a thick layer of peptidoglycans sandwiched underneath an outer membrane. Gram positive bacteria have no inner cytoplasmic cell membrane such as in the case of the gram negative bacteria. Exemplary gram positive bacteria include Actinomyces sp., Bacillus sp., especially Bacillus anthracis (anthrax), Clostridium sp., especially Clostridium tetani, Clostridium perfringens and Clostridium botulinum, Corynebacterium sp., Enterococcus sp., Gardnerella sp., Lactobacillus sp., Listeria sp., Mycobacterium sp., especially Mycobacterium tuberculosis, Nocardia sp., Propionibacterium sp., Staphylococcus sp., especially Staphylococcus aureus, Streptococcus sp., especially Streptococcus pneumonia, and Streptomyces sp., among others.

The term “bacterial infection” or infection is used to describe any disease state and/or condition in a patient or subject which is caused by a bacteria, especially including one or more of the bacteria which are described herein.

The term “additional antibiotic” is used to describe an agent which may be used to treat a bacterial infection which is other than the antibiotic agents pursuant to the present invention and may be used in cotherapy with compounds according to the present invention. Additional antibiotics which may be combined in therapy with antibiotic compounds according to the present invention include:

Aminoglycosides including amikacin, gentamycin kanamycin, neomycin, netilmicin, tobramycin, paromomycin, streptomycin, spectinomycin;

Ansamycins, including geldanamycin, herbimycin and rifazimin; Carbacephems, including, loracarbef, ertapenem, doripenem, imipenem/cilastatin and meropenem;

Cephalosporins, including cefadroxil, cefazolin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxxone, cefepime, ceftaroline fosamil and ceftobiprole;

Glycopeptides, including teicoplanin, vancomycin, telavancin, dalbavancin and orivitavancin;

Lincosamides, including clindamycin and lincomycin;

Lipopeptides, including daptomycin;

Macrolides, including azithromycin, clarithromycin, dirithromycin, erythromycin, roxithromycin, troleandomycin, telithromycin and spiramycin;

Monobactams, including aztreonam;

Nitrofurans, including furazolidone and nitrofurantoin;

Oxazollidinones, including linezolid, posizolid, radezolid and torezolid;

Penicillins, including amoxicillin, ampicillin, azlocillin, carbenicillin, cloxacillin, dicloxacillin, flucloxacillin, mezlicillin, methicillin, oxacillin, penicillin G, penicillin V, piperacillin, temocillin, ticarcillin, amoxicillin/clavulanate, ampcillin/sulbactam, piperacillin/tazobactam and ticarcillin/clavulanate;

Polypeptides, including bacitracin, colistin and polymixin B;

Quinolones/Fluoroquinolines, including ciprofloxacin, enoxacin, gatifloxacin, gemifloxacin, levofloxacin, lomefloxecin, moxifloxacin, naldixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfasalazine, sulfisoxazole, Trimethoprim-sulfamethoxazole and sulfonamidochysoidine;

Tetracyclines, including demeclocycline, doxycycline, minocycline, oxytetracycline and tetracycline;

Anti-Mycobacterial agents, including clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupiocin, platensimycin, quinupristin/dalfopristin, thiamphenicol, tigecycline, tinidazole and trimethoprim.

The term “MRSA” as used herein describes any strain of Staphylococcus aureus that has antibiotic resistance, including resistance to methicillin nafcillin, oxacillin. Staphylococcus aureus (S. aureus) is a gram-positive bacterium that is frequently found in the human respiratory tract and on the human skin. Although S. aureus is not usually pathogenic, it is known to cause skin infections (e.g., boils), respiratory disease (e.g., pneumonia), bloodstream infections, bone infections (osteomyelitis), endocarditis and food poisoning. The bacterial strains that often produce infections generate protein toxins and also express cell-surface proteins that apparently bind and inactivate antibodies. MRSA is responsible for a number of very difficult-to-treat infections in humans. The resistance does render MRSA infections far more difficult to treat. MRSA is often labeled as being community acquired MRSA (“CA-MRSA”) and hospital acquired MRSA (“HA-MRSA”). MSSA (methicillin sensitive Staphylococcus aureus) refers to a strain of Staphylococcus aureus that exhibits sensitivity to methicillin.

The term “additional bioactive agent” including an “additional antibiotic” an “additional anti-Staph aureus agent”, including an “additional anti-MRSA agent” is used to describe a drug or other bioactive agent which itself is useful in the treatment of bacterial infections, including Staphylococcus aureus infections, especially including MRSA and is other than an antibiotic useful in the treatment of bacterial infections, especially gram negative bacterial infections, including Staphylococcus aureus, especially including MRSA infections as described herein.

These additional bioactive agents may be used to treat disease states and conditions which are commonly found in patients who also have Staphylococcus aureus infections, especially MRSA infections. These additional bioactive agents, include additional antibiotics, essential oils and alternative therapies which may be useful for the treatment of bacterial pathogens. In particular, antibiotics and other bioactive agents, including essential oils may be included in compositions and co-administered along with the antibiotics according to the present invention.

Preferred bioactive agents for the treatment of MRSA include, for example, oritavancin (Orbactiv), dalvavancin (Dalvance), tedizolid phosphate, (Sivextro), clindamycin, linezolid (Zyvox), mupirocin (Bactroban), trimethoprim, sulfamethoxazole, trimethoprim-sulfamethoxazole (Septra or Bactrim), tetracyclines (e.g., doxycycline, minocycline), vancomycin, daptomycin, fluoroquinolines (e.g. ciprofloxacin, levofloxacin), macrolides (e.g. erythromycin, clarithromycin, azithromycine) or mixtures thereof. In addition to antibiotics, alternative therapies may be used in combination with the antibiotics pursuant to the present invention and include the use of manuka honey and/or essential oils such as tea tree oil, oregano oil, thyme, clove, cinnamon, cinnamon bark, eucalyptus, rosemary, lemongrass, geranium, lavender, nutmeg and mixtures thereof.

Antibiotics which are useful in the treatment of Staphylococcus aureus infections (both MSSA and MRSA) depend upon the tissue where the infection is found and whether the Staphylococcus aureus infection is MSSA or MRSA. In general, antibiotics which are found useful in the treatment of general MSSA infections include, for example, β-lactams oxacillin, nafcillin and cefazolin, which are often used. For general MRSA infections, vancomycin, daptomycin, linezolid, Quinupristin/dalfopristin, Cotrimoxazole, Ceftaroline and Telavancin are more often used.

For treatment of Staphylococcus aureus infections of the heart or its valves (Endocarditis) oxacillin, cefazolin, nafcillin or gentamycin are used for methicillin sensitive strains (MSSA). For MRSA infections of the heart or its valves, useful antibiotics include ciprofloxacin, rifampin, vancomycin and daptomycin as preferred agents.

For Staphylococcus aureus infections of soft tissues and skin—the primary treatment using antibiotics for MSSA includes Cephalexin, Dicloxacillin, Clindamycin and Amoxicillin/clavulanate. For MRSA infections, the preferred antibiotics include Cotrimoxazole, Clindamycin, tetracyclines, Doxycycline, Minocycline and Linezolide, although others may be used.

For skin infections local application of antibiotics like Mupirocin 2% ointment are generally prescribed.

For lung infections or pneumonia—for MRSA cases Linezolid, Vancomycin and Clindamycin are preferred.

For bone and joint infections—for MSSA oxacillin, cefazolin, nafcillin and gentamycin are often used. For MRSA infections, Linezolid, Vancomycin, Clindamycin, Daptomycin and Cotrimoxazole are often used.

For infections of the brain and meninges infection (meningitis)—for MSSA oxacillin, cefazolin, nafcillin, and gentamycin are preferred. For MRSA infections, Linezolid, Vancomycin, Clindamycin, Daptomycin and Cotrimoxazole may be used.

For Toxic Shock Syndrome—for MSSA oxacillin, nafcillin and clindamycin are often used. For MRSA infections Linezolid, Vancomycin and Clindamycin are often used.

Each of the above antibiotics may be combined in methods of the present invention for treating bacterial pathogens, especially Staphylococcus aureus infections (MSSA or MRSA). In addition, one or more of these antibiotics may be combined with one or GPER modulators in pharmaceutical compositions for the treatment of bacterial pathogens, especially Staphylococcus aureus infections (MSSA or MRSA).

“Hydrocarbon” or “hydrocarbyl” refers to any monovalent (or divalent in the case of alkylene groups) radical containing carbon and hydrogen, which may be straight, branch-chained or cyclic in nature. Hydrocarbons include linear, branched and cyclic hydrocarbons, including alkyl groups, alkylene groups, saturated and unsaturated hydrocarbon groups including aromatic groups both substituted and unsubstituted, alkene groups (containing double bonds between two carbon atoms) and alkyne groups (containing triple bonds between two carbon atoms). In certain instances, the terms substituted alkyl and alkylene are sometimes used synonymously.

“Alkyl” refers to a fully saturated monovalent radical containing carbon and hydrogen, and which may be cyclic, branched or a straight chain containing from 1 to 12 carbon atoms (C1-C12 alkyl) and are optionally substituted. Examples of alkyl groups are methyl, ethyl, n-butyl, n-hexyl, n-heptyl, n-octyl, n-nonyl, n-decyl, isopropyl, 2-methyl-propyl, cyclopropyl, cyclopropylmethyl, cyclobutyl, cyclopentyl, cyclopentylethyl, cyclohexylethyl and cyclohexyl. Preferred alkyl groups are C₁-C₆ alkyl groups. “Alkylene” refers to a fully saturated hydrocarbon which is divalent (may be linear, branched or cyclic) and which is optionally substituted. Preferred alkylene groups are C₁-C₆ alkylene groups. Other terms used to indicate substituent groups in compounds according to the present invention are as conventionally used in the art.

The term “aryl” or “aromatic”, in context, refers to a substituted or unsubstituted monovalent aromatic radical having a single ring (e.g., benzene or phenyl) or fused rings (naphthyl, phenanthryl, anthracenyl). Other examples of aryl groups, in context, may include heterocyclic aromatic ring systems “heteroaryl” groups having one or more nitrogen, oxygen, or sulfur atoms in the ring (5- or 6-membered heterocyclic rings) such as imidazole, furyl, pyrrole, pyridyl, furanyl, thiene, thiazole, pyridine, pyrimidine, pyrazine, triazine, triazole, oxazole, among others, which may be substituted or unsubstituted as otherwise described herein.

The term “Sugar” or “carbohydrate” refers to a monosaccharide, disaccharide or oligosaccharide moiety which may be used as a substituent on compounds according to the present invention. Exemplary sugars useful in the present invention include, for example, monosaccharides, disaccharides and oligosaccharides preferably a monosaccharide, including aldoses and ketoses, and disaccharides, including those disaccharides as otherwise described herein. Monosaccharide aldoses include monosaccharides such as aldotriose (D-glyceraldehdye, among others), aldotetroses (D-erythrose and D-Threose, among others), aldopentoses, (D-ribose, D-arabinose, D-xylose, D-lyxose, among others), aldohexoses (D-allose, D-altrose, D-Glucose, D-Mannose, L-Rhamnose, D-rhamnose, D-gulose, D-idose, D-galactose and D-Talose, among others), and the monosaccharide ketoses include monosaccharides such as ketotriose (dihydroxyacetone, among others), ketotetrose (D-erythrulose, among others), ketopentose (D-ribulose and D-xylulose, among others), ketohexoses (D-Psicone, D-Fructose, D-Sorbose, D-Tagatose, among others), aminosugars, including galactoseamine, sialic acid, N-acetylglucosamine, among others and sulfosugars, including sulfoquinovose, among others. Exemplary disaccharides which find use in the present invention include sucrose (which may have the glucose optionally N-acetylated), lactose (which may have the galactose and/or the glucose optionally N-acetylated), maltose (which may have one or both of the glucose residues optionally N-acetylated), trehalose (which may have one or both of the glucose residues optionally N-acetylated), cellobiose (which may have one or both of the glucose residues optionally N-acetylated), kojibiose (which may have one or both of the glucose residues optionally N-acetylated), nigerose (which may have one or both of the glucose residues optionally N-acetylated), isomaltose (which may have one or both of the glucose residues optionally N-acetylated), β,β-trehalose (which may have one or both of the glucose residues optionally N-acetylated), sophorose (which may have one or both of the glucose residues optionally N-acetylated), laminaribiose (which may have one or both of the glucose residues optionally N-acetylated), gentiobiose (which may have one or both of the glucose residues optionally N-acetylated), turanose (which may have the glucose residue optionally N-acetylated), maltulose (which may have the glucose residue optionally N-acetylated), palatinose (which may have the glucose residue optionally N-acetylated), gentiobiluose (which may have the glucose residue optionally N-acetylated), mannobiose, melibiose (which may have the glucose residue and/or the galactose residue optionally N-acetylated), melibiulose (which may have the galactose residue optionally N-acetylated), rutinose, (which may have the glucose residue optionally N-acetylated), rutinulose and xylobiose, among others. Oligosaccharides for use in the present invention can include any sugar of three or more (up to about 100) individual sugar (saccharide) units as described above (i.e., any one or more saccharide units described above, in any order, especially including glucose and/or galactose units as set forth above), or for example, fructo-oligosaccharides, galactooligosaccharides and mannan-oligosaccharides ranging from three to about ten-fifteen sugar units in size. When sugars are bonded as substituents in the present compounds, preferably they are bonded at 1- or 4-positions of the sugar ring, either directly to a carbon of the sugar ring or through an oxygen group or amine (which is substituted with H or a C₁-C₃ alkyl group preferably H or methyl).

The term “substituted” shall mean substituted at a carbon or nitrogen position within a molecule or moiety within context, a hydroxyl, carboxyl, cyano (C≡N), nitro (NO₂), halogen (preferably, 1, 2 or 3 halogens, especially on an alkyl, especially a methyl group such as a trifluoromethyl), alkyl group (preferably, C₁-C₁₂, more preferably, C₁-C₆), alkoxy group (preferably, C₁-C₆ alkyl or aryl, including phenyl and substituted phenyl), a C₁-C₆ thioether, ester (both oxycarbonyl esters and carboxy ester, preferably, C₁-C₆ alkyl or aryl esters) including alkylene ester (such that attachment is on the alkylene group, rather than at the ester function which is preferably substituted with a C₁-C₆ alkyl or aryl group), thioester (preferably, C₁-C₆ alkyl or aryl), halogen (preferably, F or Cl), nitro or amine (including a five- or six-membered cyclic alkylene amine, further including a C₁-C₆ alkyl amine or C₁-C₆ dialkyl amine which alkyl groups may be substituted with one or two hydroxyl groups), amide, which is preferably substituted with one or two C₁-C₆ alkyl groups (including a carboxamide which is substituted with one or two C₁-C₆ alkyl groups), alkanol (preferably, C₁-C₆ alkyl or aryl), or alkanoic acid (preferably, C₁-C₆ alkyl or aryl) or a thiol (preferably, C₁-C₆ alkyl or aryl), or thioalkanoic acid (preferably, C₁-C₆ alkyl or aryl). Preferably, the term “substituted” shall mean within its context of use alkyl, alkoxy, halogen (preferably F), ester, keto, nitro, cyano and amine (especially including mono- or di-C₁-C₆ alkyl substituted amines which may be optionally substituted with one or two hydroxyl groups). Any substitutable position in a compound according to the present invention may be substituted in the present invention, but often no more than 3, more preferably no more than 2 substituents (in some instances only 1 or no substituents) is present on a ring. Preferably, the term “unsubstituted” shall mean substituted with one or more H atoms.

The term “blocking group” refers to a group which is introduced into a molecule by chemical modification of a function group to obtain chemoselectivity in a subsequent chemical reaction. It plays an important role in providing precursors to chemical components which provide compounds according to the present invention. Blocking groups may be used to protect functional groups on ACM groups, CCT_{E} groups, connector molecules and/or linker molecules in order to assemble compounds according to the present invention. Typical blocking groups are used on alcohol groups, amine groups, carbonyl groups, carboxylic acid groups, phosphate groups and alkyne coups among others.

Exemplary alcohol/hydroxyl protecting groups include acetyl (removed by acid or base), benzoyl (removed by acid or base), benzyl (removed by hydrogenolysis, β-methoxyethoxymethyl ether (MEM, removed by acid), dimethoxytrityl [bis-(4-methoxyphenyl)phenylmethyl] (DMT, removed by weak acid), methoxymethyl ether (MOM, removed by acid), methoxytrityl [(4-methoxyphenyl)diphenylmethyl], (MMT, Removed by acid and hydrogenolysis), p-methoxylbenzyl ether (PMB, removed by acid, hydrogenolysis, or oxidation), methylthiomethyl ether (removed by acid), pivaloyl (Piv, removed by acid, base or reductant agents. More stable than other acyl protecting groups, tetrahydropyranyl (THP, removed by acid), tetrahydrofuran (THF, removed by acid), trityl (triphenyl methyl, (Tr, removed by acid), silyl ether (e.g. trimethylsilyl or TMS, tert-butyldimethylsilyl or TBDMS, tri-iso-propylsilyloxymethyl or TOM, and triisopropylsilyl or TIPS, all removed by acid or fluoride ion such as such as NaF, TBAF (tetra-n-butylammonium fluoride, HF-Py, or HF-NEt₃); methyl ethers (removed by TMS1 in DCM, MeCN or chloroform or by BBr₃ in DCM) or ethoxyethlyl ethers (removed by strong acid).

Exemplary amine-protecting groups include carbobenzyloxy (Cbz group, removed by hydrogenolysis), p-Methoxylbenzyl carbon (Moz or MeOZ group, removed by hydrogenolysis), tert-butyloxycarbonyl (BOC group, removed by concentrated strong acid or by heating at elevated temperatures), 9-Fluorenylmethyloxycarbonyl (FMOC group, removed by weak base, such as piperidine or pyridine), acyl group (acetyl, benzoyl, pivaloyl, by treatment with base), benzyl (Bn groups, removed by hydrogenolysis), carbamate, removed by acid and mild heating, p-methoxybenzyl (PMB, removed by hydrogenolysis), 3,4-dimethoxybenzyl (DMPM, removed by hydrogenolysis), p-methoxyphenyl (PMP group, removed by ammonium cerium IV nitrate or CAN); tosyl (Ts group removed by concentrated acid and reducing agents, other sulfonamides, Mesyl, Nosyl & Nps groups, removed by samarium iodide, tributyl tin hydride.

Exemplary carbonyl protecting groups include acyclical and cyclical acetals and ketals (removed by acid), acylals (removed by Lewis acids) and dithianes (removed by metal salts or oxidizing agents).

Exemplary carboxylic acid protecting groups include methyl esters (removed by acid or base), benzyl esters (removed by hydrogenolysis), tert-butyl esters (removed by acid, base and reductants) esters of 2,6-disubstituted phenols (e.g. 2,6-dimethylphenol, 2,6-diisopropylphenol, 2,6-di-tert-butylphenol, removed at room temperature by DBU-catalyzed methanolis under high-pressure conditions, silyl esters (removed by acid, base and organometallic reagents), orthoesters (removed by mild aqueous acid), oxazoline (removed by strong hot acid (pH<1, T>100° C.) or strong hot alkali (pH>12, T>100° C.)).

Exemplary phosphate group protecting groups including cyanoethyl (removed by weak base) and methyl (removed by strong nucleophiles, e.g. thiophenol/TEA).

Exemplary terminal alkyne protecting groups include propargyl alcohols and silyl groups.

The term “pharmaceutically acceptable salt” or “salt” is used throughout the specification to describe a salt form of one or more of the compositions herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of prostate cancer, including metastatic prostate cancer, the term “salt” shall mean a pharmaceutically acceptable salt, consistent with the use of the compounds as pharmaceutical agents.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. Compounds according to the present invention may be administered with one or more additional bioactive agents, especially including an additional antibiotic for purposes of treating bacterial, especially gram negative bacteria.

Pharmaceutical compositions comprising combinations of an effective amount of at least one compound disclosed herein, often a according to the present invention and one or additional compounds as otherwise described herein, all in effective amounts, in combination with a pharmaceutically effective amount of a carrier, additive or excipient, represents a further aspect of the present invention. These may be used in combination with at least one additional, optional bioactive agents, especially antibiotics as otherwise disclosed herein.

The compositions of the present invention may be formulated in a conventional manner using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl, pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir, among others. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally (including via intubation through the mouth or nose into the stomach), intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially to treat skin bacterial infections or other diseases which occur in or on the skin. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with our without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier materials to produce a single dosage form will vary depending upon the host and disease treated, the particular mode of administration. Preferably, the compositions should be formulated to contain between about 0.05 milligram to about 750 milligrams or more, more preferably about 1 milligram to about 600 milligrams, and even more preferably about 10 milligrams to about 500 milligrams of active ingredient, alone or in combination with at least one additional compound which may be used to treat a pathogen, especially a bacterial (often a gram-negative bacterial) infection or a secondary effect or condition thereof.

Methods of treating patients or subjects in need for a particular disease state or condition as otherwise described herein, especially a pathogen, especially a bacterial infection, in particular, a grant-negative bacterial infection, comprise administration of an effective amount of a pharmaceutical composition comprising therapeutic amounts of one or more of the novel compounds described herein and optionally at least one additional bioactive (e.g. additional antibiotic) agent according to the present invention. The amount of active ingredient(s) used in the methods of treatment of the instant invention that may be combined with the carrier materials to produce a single dosage form will vary depending upon the host treated, the particular mode of administration. For example, the compositions could be formulated so that a therapeutically effective dose of between about 0.01, 0.1, 1, 5, 10, 15, 20, 25, 30, 35, 40, 45, 50, 55, 60, 65, 70, 75, 80, 85, 90 or 100 mg/kg of patient/day or in some embodiments, greater than 100, 110, 120, 130, 140, 150, 160, 170, 180, 190 or 200 mg/kg of the novel compounds can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

A patient or subject (e.g. a human) suffering from a bacterial infection can be treated by administering to the patient (subject) an effective amount of a compound according to the present invention including pharmaceutically acceptable salts, solvates or polymorphs, thereof optionally in a pharmaceutically acceptable carrier or diluent, either alone, or in combination with other known antibiotic or pharmaceutical agents, preferably agents which can assist in treating the bacterial infection or ameliorate the secondary effects and conditions associated with the infection. This treatment can also be administered in conjunction with other conventional therapies known in the art.

The present compounds, alone or in combination with other agents as described herein, can be administered by any appropriate route, for example, orally, parenterally, intravenously, intradermally, subcutaneously, or topically, in liquid, cream, gel, or solid form, or by aerosol form.

The active compound is included in the pharmaceutically acceptable carrier or diluent in an amount sufficient to deliver to a patient a therapeutically effective amount for the desired indication, without causing serious toxic effects in the patient treated. A preferred dose of the active compound for all of the herein-mentioned conditions is in the range from about 10 ng/kg to 300 mg/kg, preferably 0.1 to 100 mg/kg per day, more generally 0.5 to about 25 mg per kilogram body weight of the recipient/patient per day. A typical topical dosage will range from about 0.01-3% wt/wt in a suitable carrier.

The compound is conveniently administered in any suitable unit dosage form, including but not limited to one containing less than 1 mg, 1 mg to 3000 mg, preferably 5 to 500 mg of active ingredient per unit dosage form. An oral dosage of about 25-250 mg is often convenient.

The active ingredient is preferably administered to achieve peak plasma concentrations of the active compound of about 0.00001-30 mM, preferably about 0.1-30 μM. This may be achieved, for example, by the intravenous injection of a solution or formulation of the active ingredient, optionally in saline, or an aqueous medium or administered as a bolus of the active ingredient. Oral administration is also appropriate to generate effective plasma concentrations of active agent.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; excipient such as starch or lactose, a dispersing agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents.

The active compound or pharmaceutically acceptable salt thereof can be administered as a component of an elixir, suspension, syrup, wafer, chewing gum or the like. A syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action, such as other anticancer agents, antibiotics, antifungals, antiinflammatories, or antiviral compounds. In certain preferred aspects of the invention, one or more chimeric antibody-recruiting compound according to the present invention is coadministered with another anticancer agent and/or another bioactive agent, as otherwise described herein.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, preferred carriers are physiological saline or phosphate buffered saline (PBS).

In one embodiment, the active compounds are prepared with carriers that will protect the compound against rapid elimination from the body, such as a controlled and/or sustained release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions or cholestosomes may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin fACM of dried lipid on the surface of the container. An aqueous solution of the active compound are then introduced into the container. The container is then swirled by hand to free lipid material from the sides of the container and to disperse lipid aggregates, thereby forming the liposomal suspension.

### Chemistry

Compounds according to the present invention are synthesized according to the Schemes which are presented in the present application in the attached Figures and Schemes.

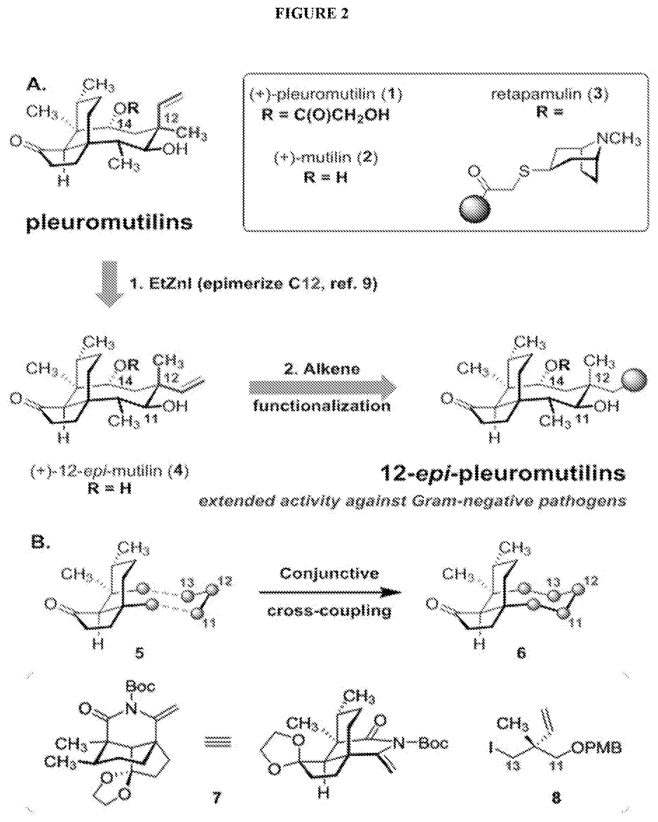

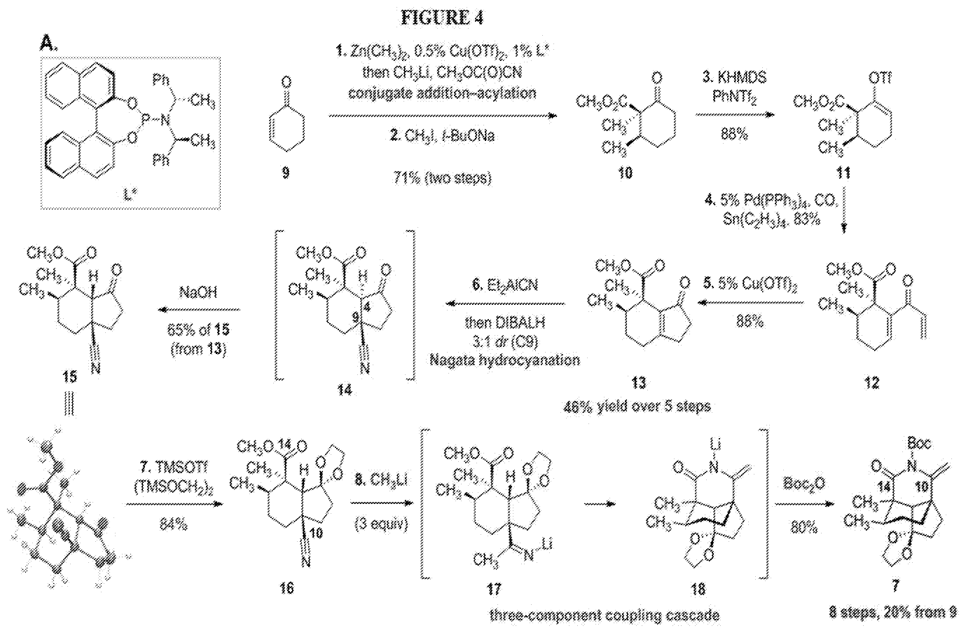

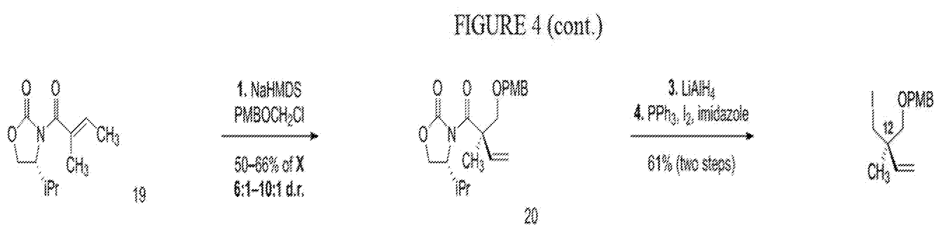

The synthetic chemical approach taken is illustrated through the synthesis of 12-epi-mutilin (4), 11,12-diepi-mutilin (26), 12-epipleuromutilin (29), 11, 12-diepi-pletiromutilin (31), and (+)-pleuromutilin (1) itself. After considerable experimentation, the alkyl iodide 8 and the imide 7 (FIG. 2) were developed as the conjunctive reagent and electrophile, respectively, in the key fragment coupling reaction. The synthetic route to 7 begins with cyclohex-2-ene-1-one which is converted to the β-ketoester 10 by a two-step sequence comprising copper-catalyzed enantioselective 1,4-addition of dimethylzinc, in situ activation of the resulting zinc enolate with methyllithium, C-acylation, and (in a separate flask) diastereoselective methylation (FIG. 4, Scheme 1A; 73% overall, 97:3 er, and >20:1 dr). Deprotonation of 10 (potassium hexamethyldisilazide) and trapping of the resulting enolate with N-phenyltriflimide provided the vinyl triflate 11 (86%). Palladium-mediated carbonylative coupling 13 of 11 with tetravinyltin formed the dienone 12 (93%). Copper-catalyzed Nazarov cyclization 14 formed the dehydrindane 13 as a single double bond regioisomer (88%).

Conditions were developed to productively functionalize the dehydrindane core of 13. 1,4-Addition of diethylaluminum cyanide 15 to 13 proceeded with 3:1 selectivity at C-9 (pleuromutilin numbering), but these were difficult to separate on preparative scales. Fortunately, the inventors found that the minor diastereomer could be reduced selectively in situ with di-iso-butylaluminum hydride (DIBALH). After treatment with aqueous sodium hydroxide (to affect quantitative epimerization of the C-4 position), the desired cis-hydrindane 15 was obtained in 58% yield (from 13). The relative and absolute stereochemistry of 15 was confirmed by X-ray analysis. Protection of the ketone as its glycol acetal 16 provided 16 (84%). Selective functionalization of the ester substituent in 16 was challenging owing to the steric congestion of the system. Ultimately, the inventors found that the addition of excess methyllithium to 16, followed by di-tert-butyldicarbonate, provided the cyclic imide 7 (76%). This cascade sequence may comprise the addition of methyllithium to the nitrile, cyclization of the resulting anion 17, and N-acylation. The alkyl iodide 8 was prepared by site- and stereoselective alkylation of the Evans imide 19 with 1-(chloromethyloxymethyl)-4-methoxybenzene, imide reduction (lithium aluminum hydride), and deoxyiodination (61%, two steps). See FIG. 4, Scheme 16.

Pursuant to Scheme 2, FIG. 5, in a key fragment coupling step, the organolithium derived from 8 was prepared by lithium-halogen exchange (tert-butyllithium) and added to the imide 7. In situ hydrolysis of the resulting acylimine, provided the methyl ketone 21 (47-60%). The methyl ketone 21 was transformed to the alkyne 22 by triflation (potassium hexamethyldisilazide, then N-phenyltriflimide), followed by elimination (tetrabutylammonium fluoride; 71%, two steps). Removal of the para-methoxybenzyl ether (2,3-dichloro-5,6-dicyanobenzoquinone) followed by oxidation (pyridinium chlorochromate) provided the alkynyl aldehyde 23 (90%, two steps).

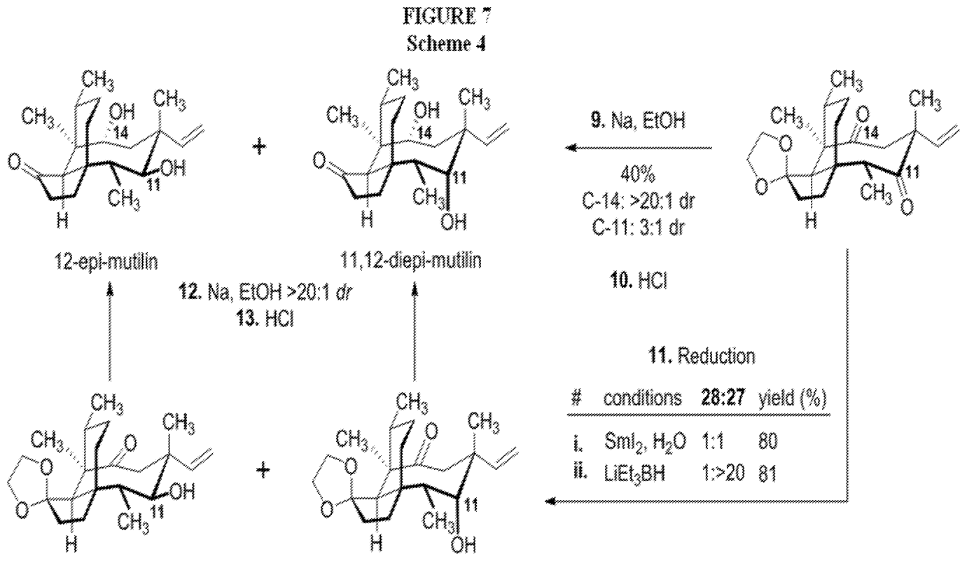

Several methods were investigated to close the 8-membered ring of the target. Ultimately, the inventors found that treatment of 23 with bis(cyclooctadiene) nickel 1,3-bis-(2,6-diisopropylphenyl)imidazolinium chloride and triethylsilane in tetrahydrofuran at ambient temperature resulted in smooth reductive cyclization to form a single allylic silyl ether (not shown). Cleavage of the silyl ether (tetra-nbutylammonium fluoride) provided the allylic alcohol 24. These conditions were based on earlier exaselective alkyne-aldehyde macrocyclizations developed by Montgomery and co-workers. To complete the synthesis, the allylic alcohol 24 was oxidized with the Dess-Martin periodinane (DMP) to provide the unsaturated ketone 25. Single electron reduction of the diketone 25 using an excess of samarium diiodide proceeded with >20:1 selectivity at C-14 and 3:1 selectivity at C-11 to provide, after ketone deprotection in acid, 12-epi-mutilin (5) and 11,12-diepi-mutilin (26) (40%). Alternative methods of reduction were also investigated and found to be successful. Reduction of 25 with an excess of samarium diiodide in H₂O provided a 1:3 mixture of the C-11 alcohols 27 and 28. Treatment of 25 with lithium triethyl borohydride produced the C-11 axial alcohol 27 with >20:1 selectivity. See FIG. 7, Scheme 4. Reduction of the remaining C-14 ketone in 27 or 28 with sodium in ethanol, followed by ketone deprotection, then formed 5 or 26.

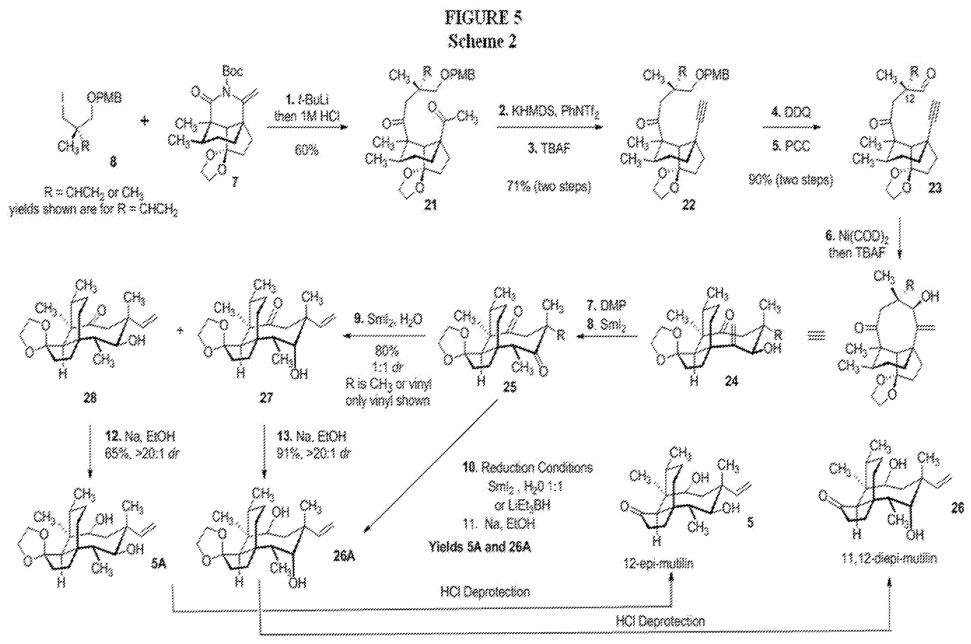

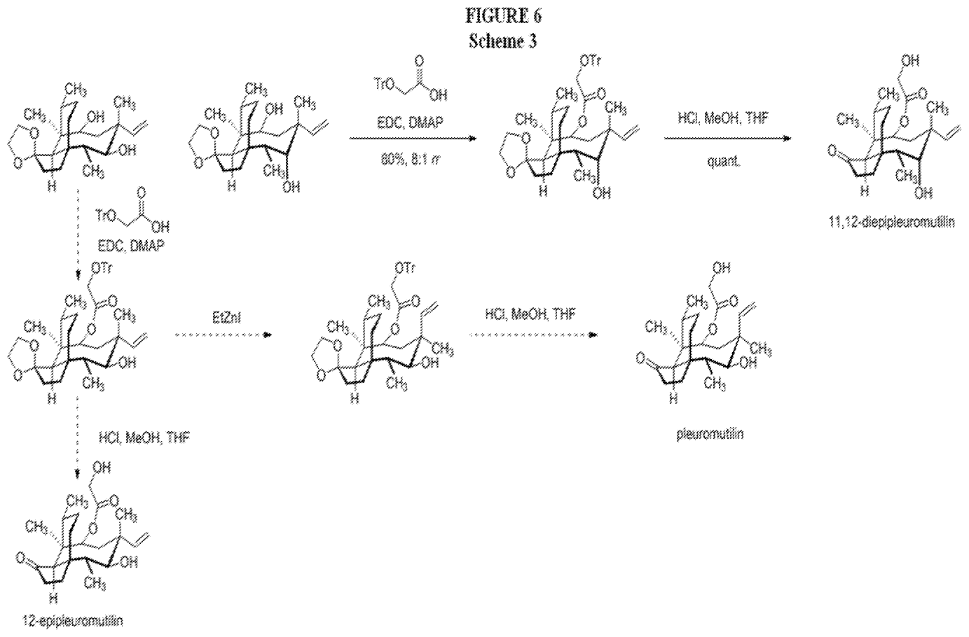

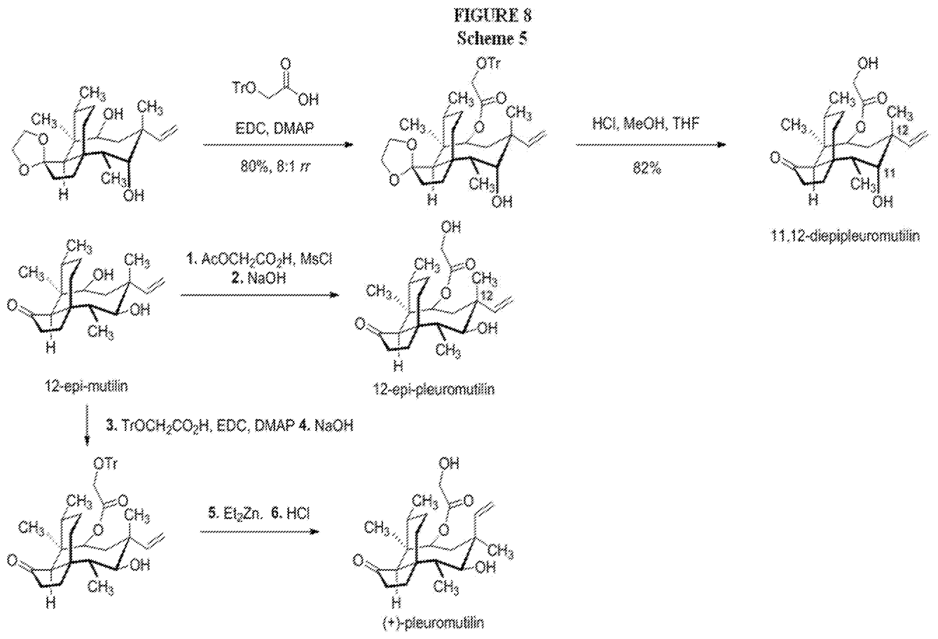

12-Epi-mutilin (5) and 11,12-diepi-mutilin (26) could easily be elaborated to unnatural pleuromutilins and to pleuromutilin (1) itself (FIG. 6, Scheme 3). Double acylation of 5 followed by selective ester saponification (FIG. 8, Scheme 5) provided 12-epi-pleuromutilin (29). Alternatively, as set forth in FIG. 8, Scheme 5, acylation of 5 with O-trityl-glyoxylic acid, EDC, DMAP followed by selective saponification of the C-11 ester, provided the tritylated ester of 12-epi-mutilin (30). Epimerization of that tritylated ester (30), followed by removal of the trityl protecting group (hydrochloric acid) then formed (+)-pleuromutilin. Selective C-14 acylation of 11,12-diepi-mutilin (26, as shown in Schemes 2, 3 and 4) with O-trityl-glyoxylic acid, followed by removal of the trityl protecting group, then generated 11,12-diepi-pleuromutilin 31. (two steps, not shown).

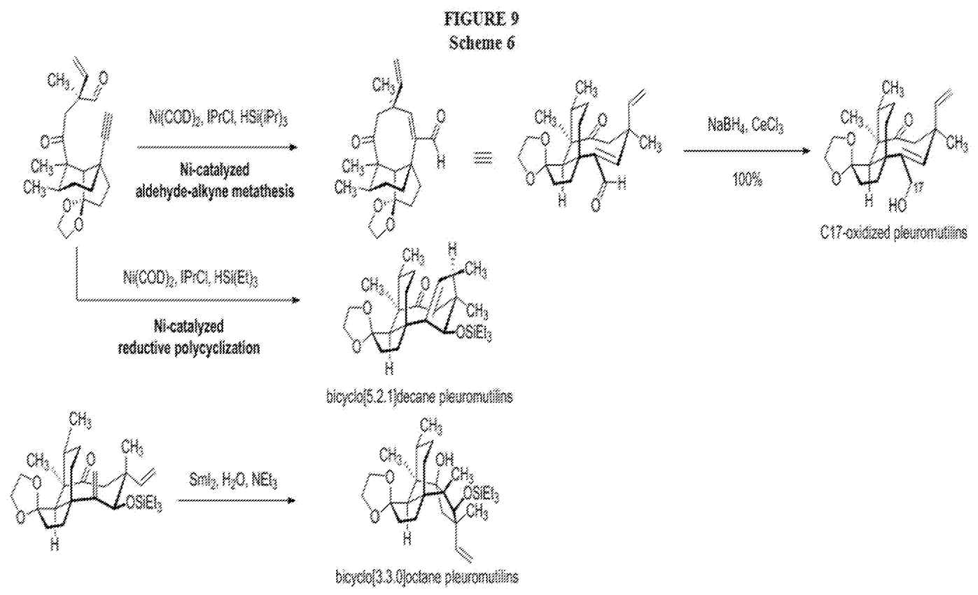

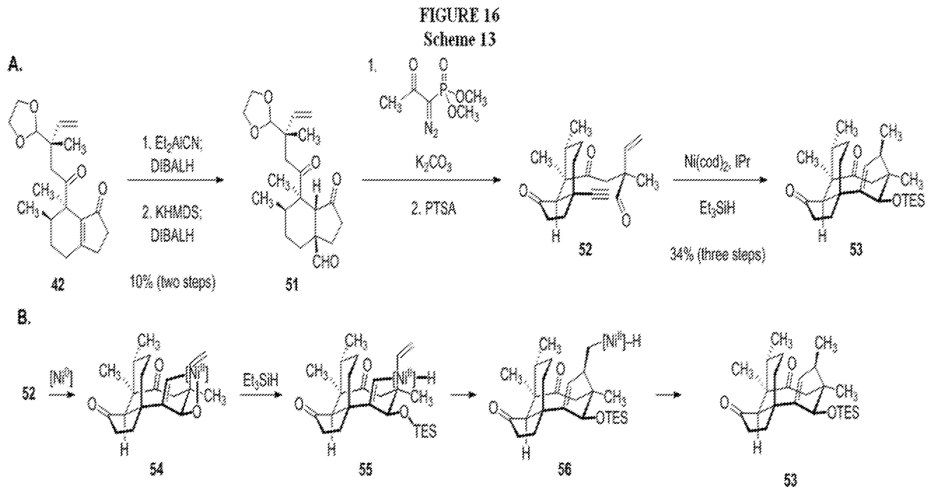

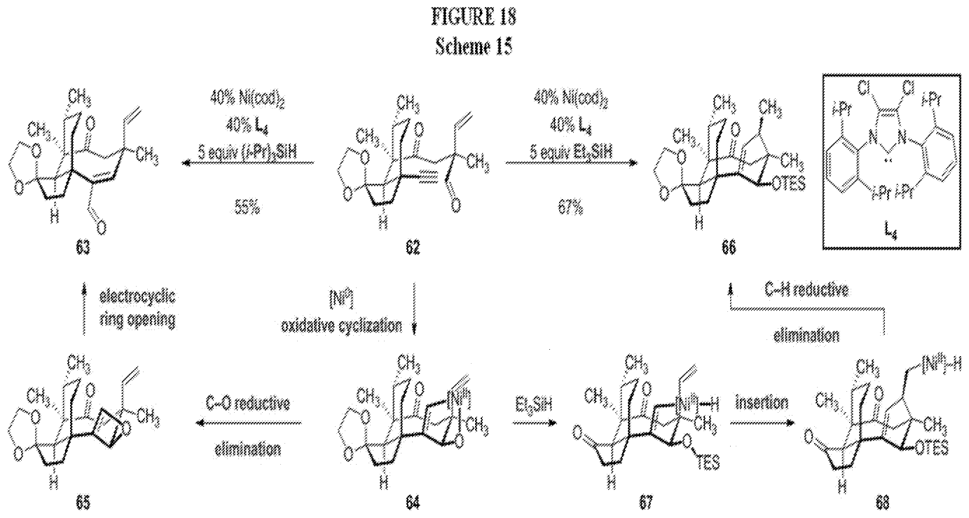

FIG. 9, Scheme 6, shows further detail of a Ni-catalyzed aldehyde-alkyne metathesis to produce the ring-closed compounds set forth therein. Two different reactions are shown, the first wherein compound 23 of FIG. 5, Scheme 2, is subjected to nickel catalyzed ring cyclization (Ni-catalyzed aldehyde-alkyne metathesis to produce the saturated bicyclo[5.2.1]decane pleuromutilins and Ni-catalyzed aldehyde-alkyne metathesis followed by sodium borohydrate reduction in cesium trichloride to produce C-17-oxidized pleuromutilins (FIG. 9. These Ni-catalyzed aldehyde-alkyne metathesis reactions reactions are shown in FIG. 9, Scheme 6, FIG. 16, Scheme 13 and FIG. 18, Scheme 15.

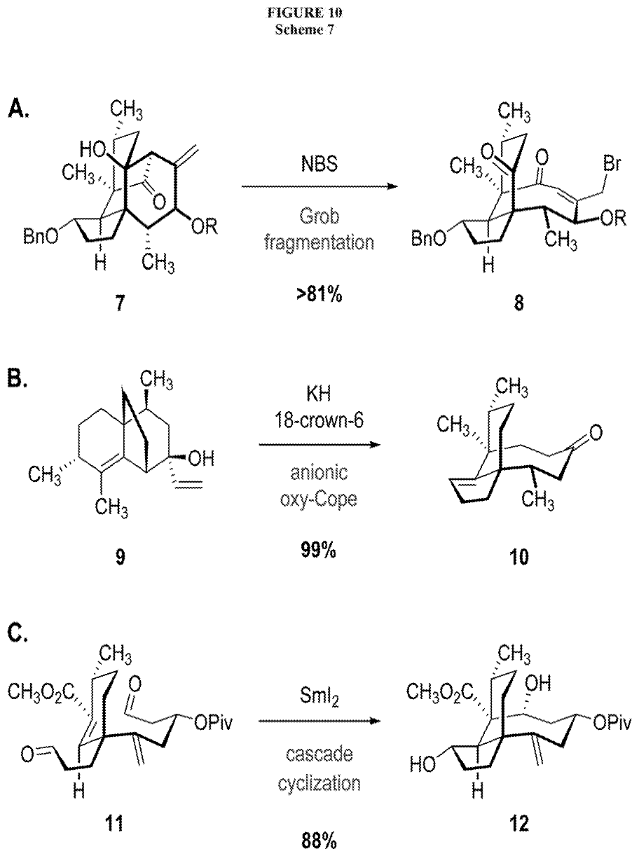

Three syntheses of pleuromutilin (1) have been reported, and the key transformations used to construct the eight-membered ring in each are summarized in FIG. 10, Scheme 7. Gibbons, working in the laboratory of the late R. B. Woodward, introduced this ring by a bromonium ion-induced Grob fragmentation (7→8, Scheme 1A, step 13 of 31 linear steps). This transformation was discovered while attempting to form an epoxide from 7 via a bromohydrin intermediate. Boeckman and co-workers employed an anionic oxy-Cope rearrangement to construct the eight-membered ring (9→10, Scheme 1B, step 7 of 27 linear steps). Finally, Procter and co-workers utilized a samarium diiodide-mediated cyclization cascade to construct the five- and eight-membered rings simultaneously (11→12, Scheme 1C, step 9 of 34 linear steps). This last work constitutes the first enantioselective route to (+)-pleuromutilin (1). These syntheses are timeless achievements in their own right that feature important strategic and methodological advances, and have been reviewed. Several synthetic studies toward (+)-pleuromutilin (1) have also been reported.

The strategy-based design of the present invention was informed by the challenges encountered by Gibbons, Boeckman, and Procter in the steps of their syntheses following formation of the 8-membered carbocycle. Each synthesis constructs the core relatively early (steps 7, 9, or 13) and follows with 18-25 further transformations. The inventors sought to limit the number of reactions after formation of the 8-membered ring to achieve a more step-economic and convergent synthesis. Working within these constraints, we aimed to close the 8-membered ring towards the end of our synthesis using only the innate functionality of the pleuromutilins and with all quaternary centers and functional groups in place. This goal forced the inventors to target powerful transformations for the construction of carbon-carbon bonds in sterically-congested settings.

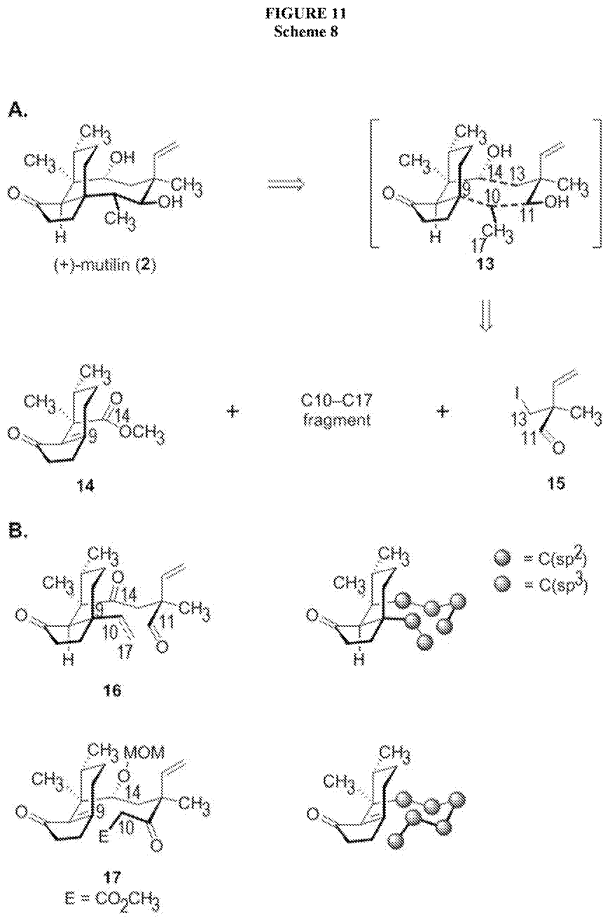

FIG. 11, Scheme 8 depicts the key elements of our retrosynthetic analysis. As with all routes to pleuromutilin, the glycolic acid residue was installed in the final steps of the synthesis. The eight-membered ring was deconstructed via the hypothetical bond disconnections (shown in structure 13) to the hydrindanone 14, a two-carbon (C10-C17) fragment, and the bridging synthon 15. In the forward sense, construction of the C9-C10 and C13-C14 bonds would afford the aldehyde 16 (Scheme 8B). We envisioned many possible modes of C10-C11 bond formation from 16 including a Nozaki-Hiyama-Kishi cyclization through a C10-C17 vinyl triflate or a reductive cyclization of an enal or ynal via a C10-C17 alkene or alkyne, respectively. The design of this cyclization strategy was informed by well-known physical organic properties of medium-sized rings. When using flexible, fully saturated cyclization precursors, entropic and enthalpic penalties arising from substrate reorganization and syn-pentane interactions, respectively, result in a high kinetic barrier to ring formation. For example, C—O bond forming ring closures to make 8-membered cyclic ethers are ˜10⁵ times slower than for 5-membered cyclic ethers. Repulsive non-bonded interactions in the cyclization transition state manifest transannular interactions in the eight-membered ring product. By comparison, the cyclization strategy the inventors designed breaks the 8-membered ring into two shorter fragments (C10-C17 and C11-C14) thereby more effectively exploiting the preorganization afforded by the rigid cis-hydrindanone. This strategy locks 5-out-of-8 atoms (C4, C5, C9, C10, C14) in the developing ring in place. Furthermore, utilizing sp- or sp²-hybridized carbons at C10 and C14 alleviates transannular interactions in the cyclization product 17. Overall, we anticipated that the 8-membered ring formation (C10-C11 bond construction) and the fragment coupling (C13-C14 bond construction) steps, both of which are two-fold neopentylic couplings, would be the most challenging transformations of this synthesis.

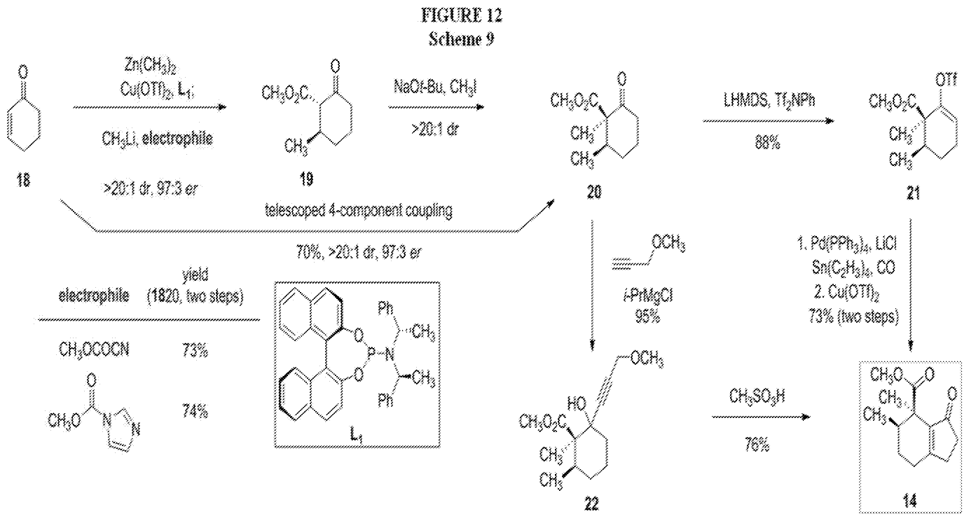

Initially, the inventors prepared the hydrindanone 14 from cyclohex-2-en-1-one (18) by a five-step sequence (FIG. 12, Scheme 9). The route began with a stereoselective conjugate addition-acylation reaction¹ that comprises copper-catalyzed enantioselective 1,4-addition of dimethyzinc to cyclohex-2-en-1-one (18), in situ activation of the resulting alkyl zinc enolate with methyllithium, and C-acylation with methyl cyanoformate (Mander's reagent). Diastereoselective methylation of the resulting β-ketoester 19 provided the α-methyl-β-ketoester 20 in 71% overall yield, >20:1 dr, and 97:3 er. Due to the high cost and safety concerns associated with the use of Mander's reagent, the inventors sought a safe and inexpensive alternative. Methyl 1H-imidazole-1-carboxylate was identified as a superior reagent that afforded the product 20 in comparable yield (75% overall, two steps). Ultimately, the conjugate addition-acylation and alkylation steps were carried out in one flask to access the α-methyl-β-ketoester 20 in one step (70%). Deprotonation of the α-methyl-β-ketoester 20 and trapping of the resulting enolate with N-phenyltriflimide afforded the vinyl triflate 21 (88%). The triflate 21 was subjected to a carbonylative Stille coupling with tetravinyl tin; the resulting dienone (not shown) underwent selective Nazarov cyclization on treatment with copper triflate to provide the hydrindanone 14 in 73% yield from 21 (five steps, 48% overall yield from 18). Although the Nazarav cyclization was in some instances efficient, tin-based impurities carried over from the Stille coupling led to variable yields of 14. To address this and to avoid using toxic alkenylstannane reagents, an alternative cyclopentannulation was developed. 1,2-Addition of the magnesium acetylide derived from methyl propargyl ether provided the alcohol 22 in 97% yield and 10:1 dr (stereoselectivity of addition not determined). Treatment with methanesulfonic acid induced a Rupe rearrangement-Nazarov cyclization cascade to generate the hydrindanone 14 directly in 71% yield (from 22; three steps, 48% overall yield from 18).

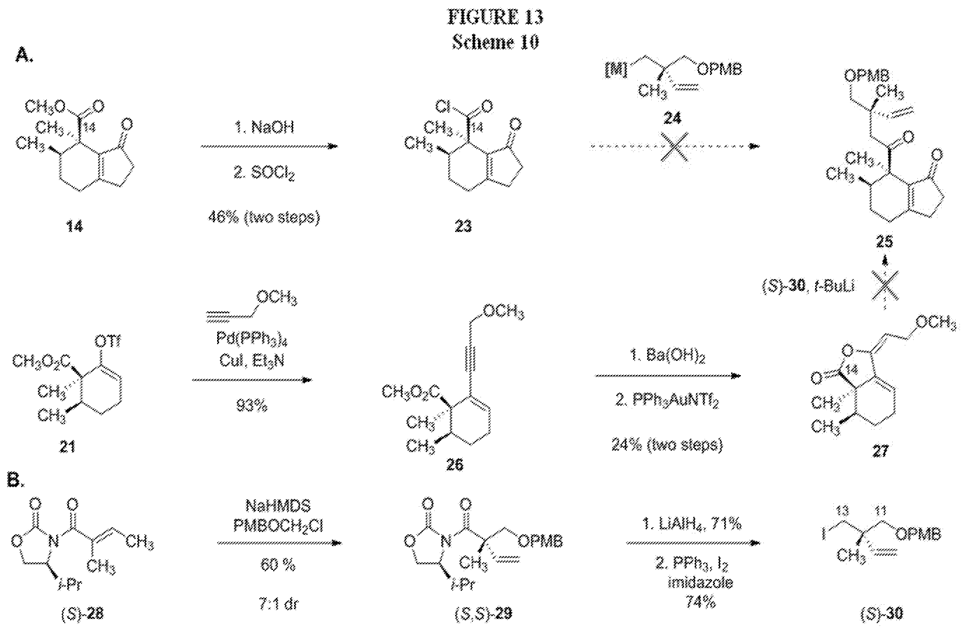

The inventors then focused on developing conditions to functionalize the C14 carbonyl group (FIG. 13, Scheme 10). Saponification of the ester (sodium hydroxide) followed by treatment of the resulting carboxylic acid with thionyl chloride afforded the acid chloride 23 in 46% yield (two steps, Scheme 10A). The acid chloride 23 was surprisingly resistant to hydrolysis and could be purified by flash-column chromatography. This stability may be due to the pseudoaxial disposition of the C14 carbonyl and the presence of an α-quaternary center. These factors, and the observation that the enone function of 23 was readily-enolizable, presaged the difficulties the inventors would encounter in the fragment coupling.

The alkyl iodide fragment (S)-30 contains the C11-C13 atoms of the target and was prepared in three steps from the chiral tigloyl derivative (S)-28 (Scheme 10B). Site- and stereoselective α- alkylation of the imide (S)-28 with para-methoxybenzyl chloromethyl ether afforded the imide (S,S)-29 in 56% yield (6:1 dr). Reduction of the imide and deoxyiodination generated the alkyl iodide (S)-30 in 28% yield (two steps).

The inventors envisioned accessing the diketone 25 by coupling the alkyl iodide (S)-30 with the acid chloride 23. However, despite extensive efforts including cross-coupling with an organozinc reagent 24 ([M]=ZnI) derived from (S)-30 or cross-electrophile coupling with (S)-30 directly, the addition product 25 was never detected. Strongly basic or nucleophilic reagents appeared to enolize or add to the enone, while attempts to activate the acid chloride using many transition metals resulted in rapid decarbonylation, presumably due to the stability of the resulting allylic metal intermediate.

The inventors then targeted the enelactone 27 as a fragment coupling partner (Scheme 10A). This species possesses a fused bicyclic skeleton which was expected to facilitate C14-addition by releasing ring strain on opening, and the cyclopentanone functionality is masked as an acyl enol ether, thereby removing any complications arising from deprotonation or 1,2-addition. The enelactone 27 was obtained in three steps and 22% yield from the vinyl triflate 21. Sonogashira coupling of 21 with methyl propargyl ether afforded the enyne 26 (93%). Saponification of the methyl ester (barium hydroxide) followed by gold-catalyzed 5-exo-dig cyclization⁴⁷ generated the enelactone 27 (24%, two steps). Unfortunately, addition of the alkyllithium reagent derived from (S)-30 (formed by lithium-halogen exchange) did not proceed, and unreacted 27 was recovered. The addition of methyllithium to 27 successfully opened the lactone to afford the desired dienone (not shown), suggesting the combined steric hindrance of the two neopentyl reagents 27 and 30 as the likely cause of failure.

The inventors also pursued an entirely distinct fragment coupling that relied on a Claisen condensation to install the C14 ketone early in the route and a Tsuji-Trost reaction to forge the C12-C13 bond (see examples). Claisen condensation of benzylacetate with the acid chloride derived from the enyne 26 (not shown) provided the β-ketoester 31 in 29% yield (two steps), thereby providing the key C13-C14 bond. Palladium-catalyzed allylic alkylation of the β-ketoester 31 using rac-2-methyl-2-vinyloxirane afforded the lactone 32 (59%). Unfortunately, the inventors were not able to obtain the hydrindanone 33 from the enyne 32. Extensive attempts to hydrate the alkyne within 32 (by inter- or intramolecular addition) were unsuccessful.

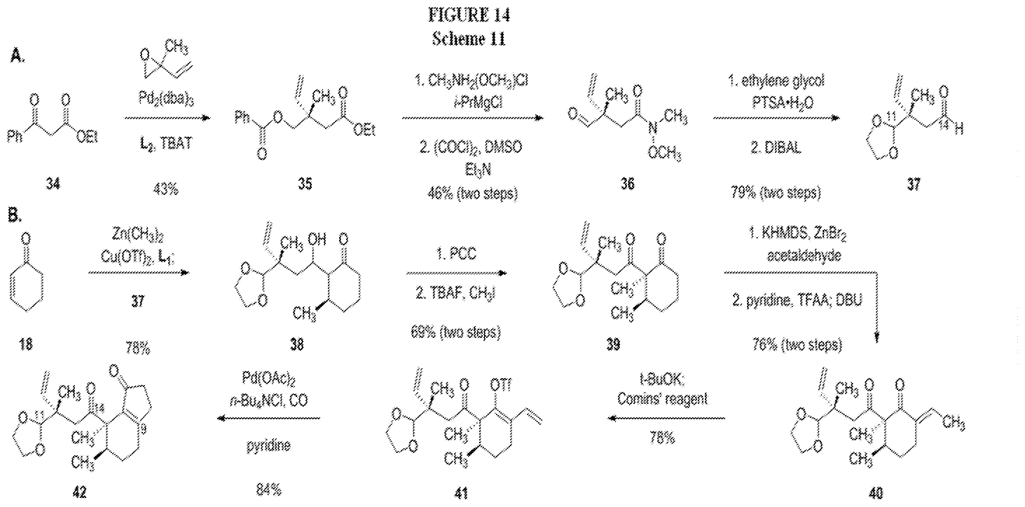

Given the difficulties described above, the inventors temporarily set aside the goal of a convergent synthesis and focused on appending the C11-C14 fragment at the outset. Strategically, this allowed the inventors to advance material to the cyclization reaction and elucidate key aspects of that transformation that would be necessary in the final route. To enable this, the inventors prepared the aldehyde 37, which contains the C11-C14 atoms of the target (FIG. 14, Scheme 11A). Allylic alkylation of ethyl benzoylacetate (34), followed by in situ benzoyl migration, generated the diester 35 (43%, 99:1 er). Cleavage of both esters was effected by treatment with excess N,O-dimethylhydroxylamine hydrogen chloride and iso-propylmagnesium chloride. Swern oxidation of the resulting primary alcohol (not shown) generated the amido aldehyde 36 (93%, two steps). Protection of the aldehyde function and reduction of the Weinreb amide (di-iso-butylaluminum hydride, DIBALH) provided 37 (79%, two steps).

Copper-catalyzed stereoselective 1,4-addition of dimethylzinc to cyclohex-2-en-1-one (18), followed by addition of the aldehyde 37, afforded the β-hydroxyketone 38 in 78% yield and as a mixture of diastereomers (Scheme 11B). The β-hydroxyketone 38 was oxidized with 2-iodoxybenzoic acid (IBX) and the resulting β-diketone (not shown) was treated with iodomethane and tetra-n-butylammonium fluoride (TBAF), to provide the α-methyl-β-diketone 39 (69%, two steps, >20:1 dr). Advancement of this material via the usual route involving Nazarov cyclization was not possible due to the acid-sensitivity of the acetal group. Instead, we implemented a strategy involving site-selective deprotonation of the α-methyl-β-diketone 39 (potassium hexamethyldisilazide, KHMDS) and treatment of the resulting enolate with acetaldehyde to afford a β-hydroxyketone (not shown). Activation of the hydroxyl group with trifluoroacetic anhydride (TFAA) and elimination of the resulting trifluoroacetate ester (1,8-diazabicyclo[5.4.0]undec-7-ene, DBU) provided the enone 40 (76%, two steps). Extended enolate formation and trapping with N-5-chloro-2-pyridyl)triflimide (Comins' reagent) afforded the dienyl triflate 41 in 78% yield. The dienyl triflate 41 was transformed to the hydrindanone 42 in 84% yield by a palladium-catalyzed carbonylative cyclization.

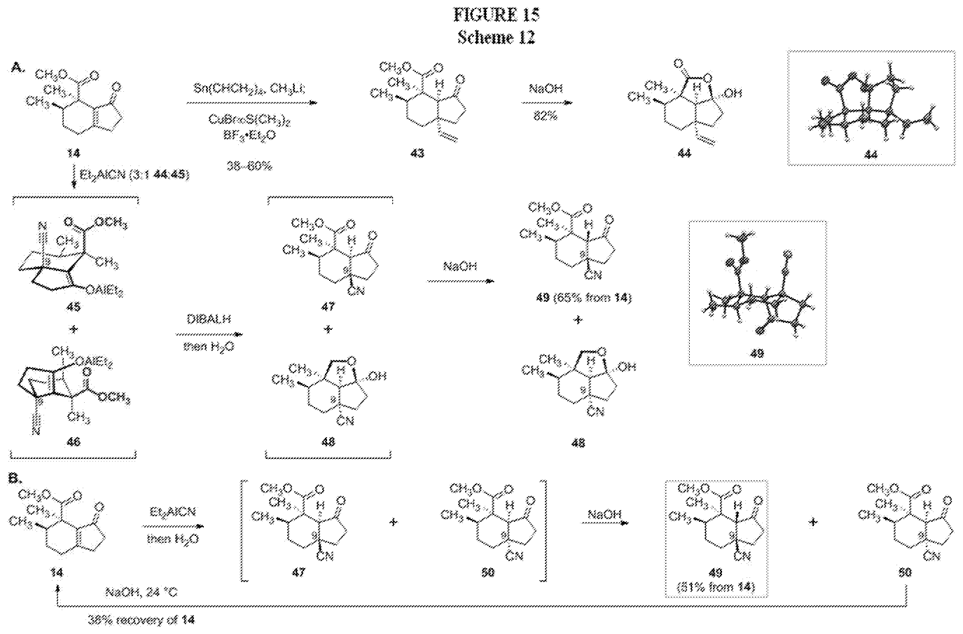

The inventor's attention then turned toward functionalization of the C9 position to install C10-C17 fragment required for eight-membered ring construction. The hydrindanone 14 (FIG. 12, Scheme 9) was employed as a model substrate since it was more accessible than 42. In line with Paquette's attempted intramolecular additions to the C9 position, 1,4-addition to the tetrasubstituted enone functionality within 14 proved challenging (FIG. 15, Scheme 12). Attempted addition of acetylide-⁵⁴ or alkenyl-based⁵⁵ nucleophiles generally resulted in recovery of unreacted 14 or the production of 1,2-addition products. Boron trifluoride-diethyl etherate-promoted addition of lithium divinylcuprate was successful and provided the addition product 43 in variable yields (38-60%) as a single detectable diastereomer (Scheme 12A). Unfortunately, X-ray crystallographic analysis of the hemiketal 44, obtained by saponification of 43, revealed that the addition proceeded with the undesired facial selectivity. Based on NOE analysis, the inventors believe the ester substituent occupies the pseudoaxial orientation. Therefore, the inventors expected nitrile addition syn to the ester substituent, which would correspond to pseudoaxial attack, in accord with the Fürst-Plattner rule. The inventors hypothesize that metal chelation by the 1,4-ketoester may drive the ester into the pseudoequatorial position (as shown in the inset), thereby making addition anti to the ester substituent now the pseudoaxial, and more favorable, mode of approach.

Fortunately, it was found that 1,4-hydrocyanation of 14 proceeded with 3:1 selectivity in favor of the desired C9 diastereomer. Careful analysis of the product mixture revealed that the desired C9-addition product underwent kinetic protonation to the trans diastereomer 47, and that this slowly converted to the desired cis isomer 49 upon concentration and purification. By comparison, the C9 adduct arising from addition to the Si-face was formed exclusively as the cis-diastereomer and was configurationally-stable (see 50, Scheme 12B). Unfortunately, separation of the three diastereomers 47, 49, and 50 was difficult on preparative scales. Accordingly, we investigated methods to resolve them in situ. We found that the undesired C9 addition intermediate 46 could be selectively reduced (at the ester function) by introduction of DIBALH directly. The desired addition intermediate 45 was unreactive, presumably due to the reduced accessibility of the axial ester substituent. The reduction of 46 proceeded to the alcohol oxidation state; upon neutralization this species cyclized to the hemiketal 48, which facilitated its separation from 47. After additional experimentation, we found that 47 could be quantitatively epimerized to 49 by treatment with dilute sodium hydroxide (65% yield of 49 from 14). The relative stereochemistry of 49 was confirmed by X-ray analysis (inset, Scheme 11A). Alternatively, subjecting the mixture of trans-hydrindanone 47 and the undesired addition product 50 to epimerization using aqueous sodium hydroxide provided the cis-diastereomers 49 and 50, which could be separated by flash-column chromatography (Scheme 12B). Although the yield of the desired product 49 is somewhat lower in this approach (53%) the undesired isomer could be efficiently recycled by elimination of hydrogen cyanide using concentrated sodium hydroxide to regenerate 14 (38% recovery based on 14), ultimately allowing higher material throughput (85% yield of 49 based on recovered 14).

1,4-Hydrocyanation of the fully-elaborated hydrindanone 42 and reduction (DIBALH) of the resulting nitrile (with in situ protection of the ketone as its corresponding enolate) provided the aldehyde 51 in 10% yield over two steps (FIG. 16, Scheme 13). Homologation with the Ohira-Bestmann reagent followed by aldehyde deprotection generated the cyclization precursor 52. With the alkynyl aldehyde 52 in hand, conditions to effect the exo-selective reductive cyclization were examined. In the presence of bis(1,5-cyclooctadiene)nickel (Ni(cod)₂), 1,3-bis(2,6-di-iso-propylphenyl)-1,3-dihydro-2H-imidazol-2-ylidene (L₃), and triethylsilane, conditions slightly modified from those developed by Montgomery and co-workers to promote the exo-selective reductive cyclization of ynals, a single product was obtained from the aldehyde 52 (34% over three steps). Although limitations in sample size impeded full characterization of this product at this time, the expected ¹H NMR resonances for the vinyl group in the desired product 54 were conspicuously absent. Subsequently, the structure of this product was identified as the tetracycle 53 by comparison to a related cyclization product (see 66, Scheme 15) and, ultimately, single crystal X-ray analysis (see inset).

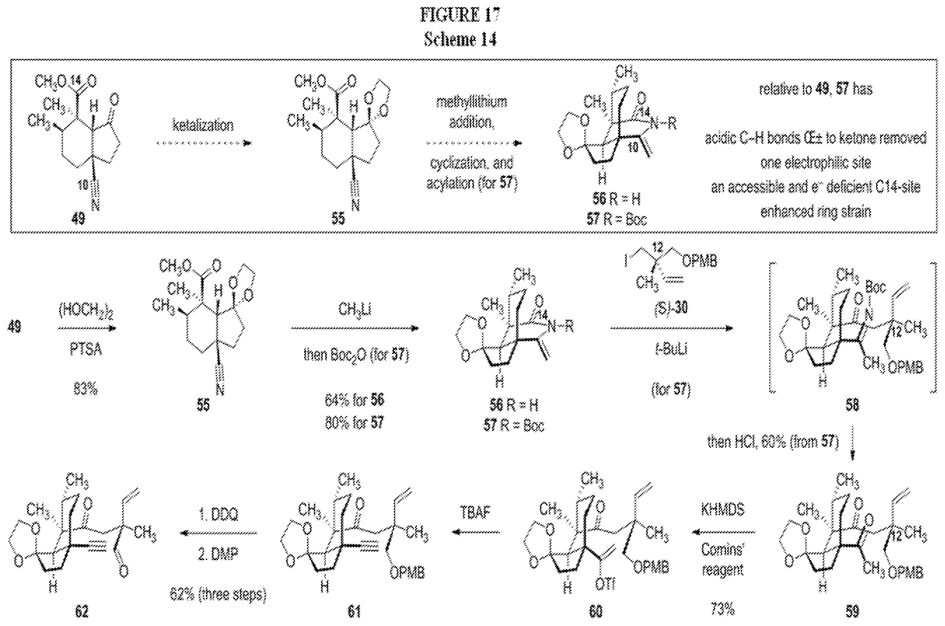

The poor yields and linear nature of the inventors route to the ynal 52 motivated us to explore other pathways to cyclization precursors. Recognizing that access to nitrile 49 provided new avenues for fragment coupling, the inventors applied lessons learned from our prior studies to transform 49 into a viable coupling partner (see top, FIG. 17, Scheme 14). First, the inventors planned to address the acidity of the ketone function of 49 by protection as a ketal (55). Second, they needed strategies to enhance the electrophilicy of the C14 carbonyl group while mitigating the additional steric hindrance imparted by the axial nitrile group. Conversion to an eneamide 56 or eneimide 57 was an attractive strategy as it was expected to alleviate C10-C14 diaxial interactions and make C14 more accessible to nucleophiles. Use of this strategy also introduced ring strain and electronic activation of C14 for fragment coupling. These modifications were envisioned to work together to make the key bis(neopentylic) fragment coupling more favorable. In addition, the inventors anticipated production of a methyl ketone function following eneamine (amide) hydrolysis after ring opening. This would help us to realize our strategy (Scheme 2B, C) to break the 8-membered ring into two short fragments (C10-C17 and C11-C14) and effectively exploit the preorganization afforded by the rigid cis-hydrindanone.

In practice, protection of the ketone [ethylene glycol, p-toluenesulfonic acid (PTSA)] proceeded smoothly to afford the ketal 55 (81%, Scheme 14). Treatment of 55 with methyllithium provided the eneamide 56 (64%), resulting from 1,2-addition to the nitrile and in situ cyclization. In order to protect the acidic amide functionality and enhance the electrophilicity of C14, we synthesized the eneimide 57 by introducing di-tert-butyldicarbonate (Boc₂O) directly to the reaction mixture following the cyclization step (80% yield). Generation of the organolithium reagent derived from (S)-30 (tert-butyllithium) followed by introduction of the eneimide 57 resulted in 1,2-addition to provide an intermediate acylimine (58) that was hydrolyzed (hydrochloric acid) to the methyl ketone 59 (60%). This key fragment coupling served to forge one of the bis(neopentylic) carbon-carbon bonds in the target. The methyl ketone 59 was dehydrated to the alkyne 61 via the vinyl triflate 60. Removal of the p-methoxybenzyl ether (2,3-dichloro-5,6-dicyano-1,4-benzoquinone, DDQ) followed by oxidation (Dess-Martin periodinane, DMP) generated the alkynyl aldehyde 62 (45%, four steps).

With an efficient route to the alkynyl aldehyde 62, the inventors were positioned to fully investigate the key Ni-catalyzed reductive cyclization step. Using tri-iso-propylsilane as a reductant and 4,5-dichloro-1,3-bis(2,6-di-iso-propylphenyl)-1,3-dihydro-2H-imidazol-2-ylidene (L₄) as the ligand, as recommended by Montgomery and co-workers, the enal 63 was obtained unexpectedly (55%, FIG. 18, Scheme 15). The inventors speculate that 63 is formed by oxidative cyclization to the metallacyclopentene 64, C—O bond reductive elimination (64→45), and electrocyclic ring opening (65→43). Although 63 was not the desired product, this result demonstrated the feasibility of the oxidative cyclization step, and suggested that tri-iso-propylsilane was too bulky to engage the metalacycle 64. Consistent with this, when triethylsilane was used as reductant, the pentacycle 66 was obtained in 67% yield. This compound was prepared in sufficient quantities for complete characterization, and provided a basis for elucidating the structure of 53 (FIG. 16, Scheme 13). A logical mechanism for the generation of 66 involves σ-bond metathesis of triethylsilane and the metallacyclopentene 64 to generate 67, 1,2-insertion of the α-olefin into the nickel-carbon bond to generate 68, and carbon-hydrogen bond reductive elimination.

The inventors recognized that the undesired alkene insertion (responsible for the formation of 66) could be avoided by placing the alkene in the pseudoequatorial position; following ring closure, the C12 position could be epimerized. From the standpoint of antibiotic development, this approach could be more useful. As discussed herein above 12-epi-mutilin derivatives bearing polar functionality in the pseudoequatorial C12 position (such as 6, FIG. 2) possess extended spectrum activity, including activity against drug-resistant and Gram-negative pathogens. Synthesis of pleuromutilins with a pseudoequatorial alkene substituent (as in 12-epi-mutilins) would allow for direct functionalization at this position and could capitalize on these known improvements in activity.

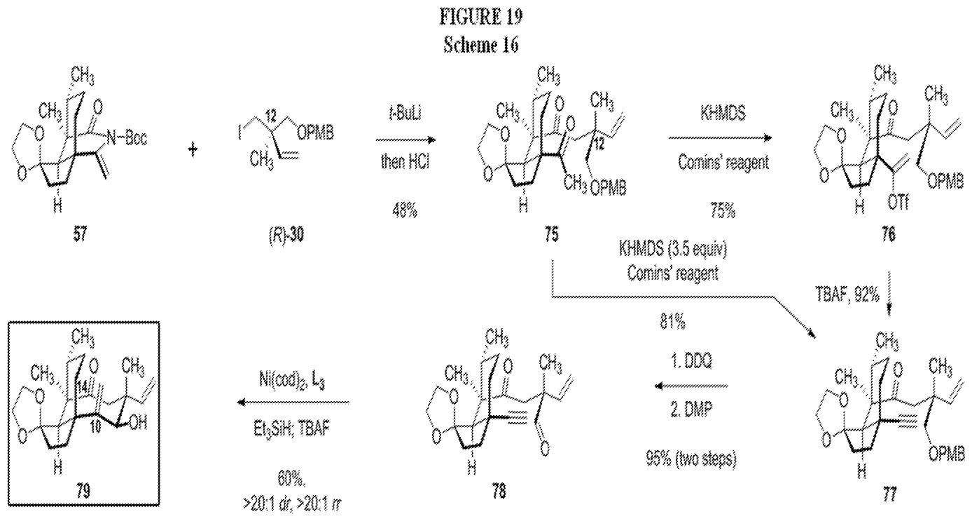

The inventors' approach to the alkyl iodide (S)-30 relied on the stereoselective alkylation of the Evans imide (S)-28 (FIG. 13, Scheme 10B). Because both enantiomers of the Evans auxiliary are commercially-available, this approach to the C11-C13 fragment allowed us to easily obtain the alternate enantiomer (R)-30 by an identical pathway (see Examples section). The eneimide 57 successfully underwent ring opening upon addition of the alkyllithium derived from (R)-30 to provide the diketone 75 in 48% yield after hydrolysis of the acylimine intermediate (FIG. 19, Scheme 16). The methyl ketone 75 was converted to the alkyne 77 by conversion to the vinyl triflate 76, followed by elimination with TBAF (69%, two steps), or more conveniently in one step by vinyl triflate formation in the presence of excess base (81%). Removal of the p-methoxylbenzyl ether with DDQ afforded a primary alcohol (not shown) that was oxidized to the aldehyde 78 (95%, two steps). When the ynal 78 was subjected to the nickel-catalyzed reductive cyclization using L₃ (the chlorinated ligand L₄ was not essential to the success of this transformation), participation of the α-olefin was not observed, and the allylic alcohol 79 was obtained in 60% yield after removal of he silyl ether. As discussed in the introduction and shown in FIG. 11, Scheme 8, preorganization by the cis-hydrindanone skeleton, as well as the presence of sp² centers at C10 and C14 in the product 79 (which alleviate transannular interactions) may facilitate this transformation. Substrate organization by formation of a nickel η²-carbonyl-η²-alkyne complex may be another contributing factor.

The inventors also investigated other ring closure strategies. The vinyl triflate 80, obtained from 76 in two steps (p-methoxybenzylether cleavage and oxidation of the resulting alcohol, 62%, could conceivably undergo a Nozaki-Hiyama-Kishi cyclization, but under a variety of conditions only the reduction product 81 was obtained. The alkene 81 could undergo a titanium(II)-mediated reductive cyclization; however, only the methyl ketone 82 was obtained (24%) when 81 was treated with bis(cyclopentadienyl)-bis(trimethylphosphine)titanium(II). The inventors speculate that 82 is formed by reductive cleavage of the 1,4-dicarbonyl functional group to afford the corresponding enolates. Alternatively, radical cleavage (to generate the α-keto radical corresponding to 82), followed by reduction to a titanium enolate, may be the operative pathway. In a separate strategy, anti-Markovnikov hydration of the terminal alkyne 77 provided the aldehyde 83 (85%). The dialdehyde 84 was obtained after p-methoxybenzylether cleavage and oxidation of the resulting alcohol (68%, two steps). Unfortunately, the dialdehyde 84 did not undergo aldol condensation. The terminal alkene 86, obtained in 52% yield by reduction of the vinyl triflate 76 (see examples), was subjected to ring-closing metathesis using the Grubbs second-generation catalyst, but did not provide the desired product. See examples.

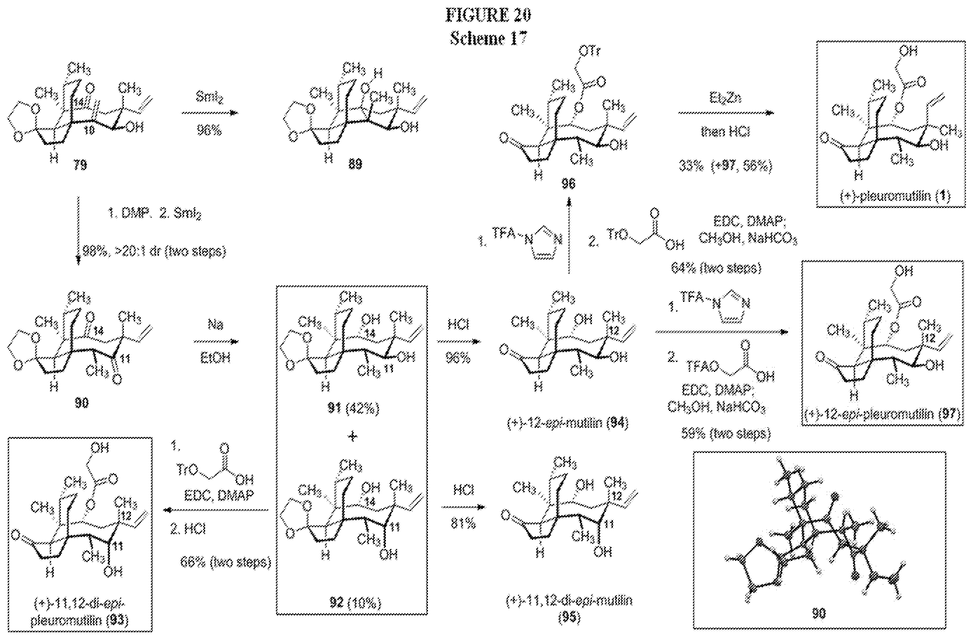

To complete the synthesis, the C14 and C10 positions of the cyclization product 79 needed to be reduced with stereocontrol (FIG. 20, Scheme 17). Given the boat-chair conformation of the substrate, hydridic reagents were expected to approach from the exterior of the 8-membered ring, which would provide the undesired pseudoaxial stereochemical outcome. Accordingly, the inventors focused on single-electron reductions that may proceed by pseudoaxial hydrogen atom abstraction to deliver the desired C10 and C14 pseudoequatorial diastereomers. Attempted samarium diiodide-mediated reduction of the C14 carbonyl of 79 provided the pentacycle 89 (96%), presumably by 5-exo-trig cyclization of a C14 ketyl radical. To prevent this, we investigated reduction of the C10 alkene first in the presence of the C12 olefin. Attempts to effect a net redox-neutral one-step isomerization of the C10-C11 allylic alcohol were unsuccessful. Accordingly, the allylic alcohol within 79 was oxidized to an enone (not shown) that was treated with samarium diiodide to afford the diketone 90 with the expected pseudoequatorial stereochemistry at C10. The relative stereochemistry of 90 was confirmed by X-ray analysis. The diketone 90 was reduced with sodium in ethanol to provide the diol 91 (42%) and the C11 diastereomer 92 (10%). Each product could be separately deprotected (hydrochloric acid) to access 12-epi-mutilin (94, 96%) or 11,12-di-epi-mutilin (95, 81%). (+)-11,12-Di-epi-pleuromutilin (93) was obtained by site-selective acylation of 11,12-di-epi-mutilin (95), followed by deprotection (66%, two steps). To access (+)-pleuromutilin (1), 12-epi-mutilin (94) was treated with trifluoroacetylimidazole and the resulting C11 ester (not shown) was coupled with O-tritylglycolic acid to afford 96 (64%, two steps). Epimerization of the C12 position, followed by acidification, provided (+)-pleuromutilin (1, 33%). Finally, (+)-12-epi-pleuromutilin (97) was obtained by stepwise acylation of the C11 and C14 alcohols with trifluoroacetylimidazole and O-trifluoroacetylglycolic acid, respectively, followed by methanolysis of the trifluoroacetyl esters (59%, two steps).

### EXAMPLES

Examples are presented as follows. These examples provide insight into the chemical syntheses of the various compounds which are presented in the present application, including the various schemes.

### General Experimental Procedures—First Set of Experiments

All reactions were performed in oven-dried (>140° C.) or flame-dried glassware sealed with rubber septa and under a positive pressure of argon. Air and moisture-sensitive reagents were transferred via syringe or stainless steel cannula, or were handled in a nitrogen-filled drybox (working oxygen level <10 ppm). Reactions were monitored using thin-layer chromatography (TLC) on EMD Silica Gel 60 F254 plates. Visualization of the developed plates was performed using UV light (254 nm) and/or by submersion in aqueous p-anisaldehyde solution (PAA) or aqueous potassium permanganate solution (KMnO₄), followed by brief heating with a heat gun. Organic solutions were concentrated under reduced pressure at 20-35° C. Flash-column chromatography was performed as described by Still et al., using silica gel (60 Å, 40-63 μm particle size) purchased from SiliCycle.

### Materials

Dichloromethane, N,N-dimethylformamide, ether, hexanes, pentane, tetrahydrofuran, and toluene were deoxygenated by sparging with nitrogen and then dried according to the method of Pangborn et al. 1,2-Dichloroethane was purchased as anhydrous grade and then deoxygenated by sparging with nitrogen before use. Methanol and ethanol were deoxygenated by sparging with nitrogen and then dried over 3 Å molecular sieves before use. Water employed in the ketone reduction reaction (25→27/28) was deoxygenated by sparging with nitrogen before use. The molarity of organozinc solutions was determined by titration against a standard solution of iodine and lithium chloride in tetrahydrofuran (average of three determinations). The molarity of n-butyllithium solutions was determined by titration against a standard solution of menthol and 1,10-phenanthroline in tetrahydrofuran (average of three determinations). Molecular sieves were activated by heating to 200° C. under vacuum (<1 Torr) for 12 h, and were stored in either an oven at >140° C. or a nitrogen-filled glovebox. Feringa's phosphoramidite ligand, oxazolidinone 19, para-methoxybenzyl chloromethyl ether, trifluoroacetylglycolic acid (S9) and O-tritylglycolic acid (S10) were prepared according to literature procedures (see references first experimental section). All other reagents were purchased and used as received.

### Equipment

Proton nuclear magnetic resonance spectra (¹H NMR) were recorded at 400, 500 or 600 MHz at 24° C. Proton-decoupled carbon nuclear magnetic resonance spectra (¹³C NMR) were recorded at 101, 125 or 151 MHz at 24° C. Fluorine nuclear magnetic resonance (¹⁹F NMR) spectra were recorded at 470 MHz at 24° C. Chemical shifts are expressed in parts per million (ppm, δ scale) downfield from tetramethylsilane and are referenced to the residual solvent signal. Data for ¹H NMR are reported as follows: chemical shift (δ ppm), multiplicity (s=singlet, d=doublet, t=triplet, q=quartet, m=multiplet, br=broad), coupling constant (Hz), integration. Data for ¹³C NMR are reported in terms of chemical shift (δ ppm). High-resolution mass spectrometry (HRMS) data were obtained on a Waters UPLC/HRMS instrument equipped with an ESI high-resolution mass spectrometry detector.

### Synthetic Procedures and Characterization Data

Synthesis of the β-ketoester S1:

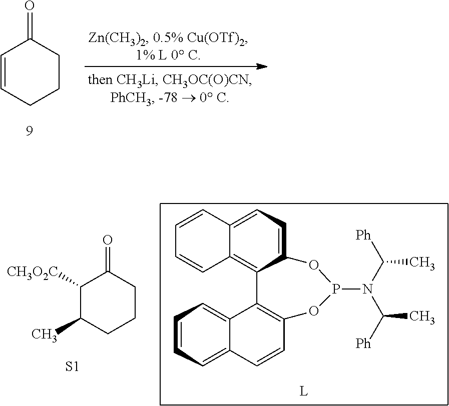

A suspension of copper(II) bis(trifluoromethanesulfonate) (207 mg, 572 μmol, 0.500 mol %) and L (618 mg, 1.14 mmol, 1.00 mol %) in toluene (160 mL) was stirred for 30 min at 20° C. The resulting solution was cooled to 0° C. for 20 min and then cyclohex-2-ene-1-one (9, 11.1 mL, 114 mmol, 1 equiv) was added. A solution of dimethylzinc in toluene (1.2 M, 100 mL, 120 mmol, 1.05 equiv) was then added dropwise over 20 min and the resulting mixture was stirred for an additional 30 min. The resulting mixture was cooled to −78° C. for 20 min and then a solution of methyllithium in ether (1.6 M, 75.1 mL, 120 mmol, 1.05 equiv) was added dropwise over 5 min. After stirring an additional 5 min, methyl cyanoformate (10.9 mL, 137 mmol, 1.20 equiv) was added. The resulting solution was stirred at −78° C. for 2 h and then allowed to warm to 0° C. over a period of 30 min. The warmed mixture was diluted sequentially with saturated aqueous ammonium chloride solution (40 mL) and water (200 mL). The product mixture was warmed to 20° C. over a period of 30 min. The warmed mixture was extracted with ethyl acetate (3×200 mL) and the organic extracts were combined. The combined organic extracts were washed with saturated sodium chloride solution (200 mL). The washed solution was dried over magnesium sulfate. The dried solution was filtered and the filtrate was concentrated. The resulting residue was used directly in the following step.

Synthesis of the α-methyl β-ketoester 10:

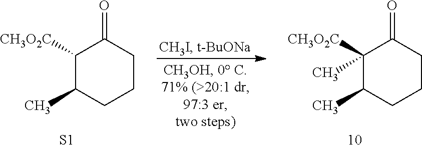

The residue obtained in the preceding step (nominally 114 mmol) was dissolved in methanol (230 mL) and the resulting solution was cooled to 0° C. for 20 min. Iodomethane (35.6 mL, 572 mmol, 5.00 equiv) and sodium tert-butoxide (22.0 g, 229 mmol, 2.00 equiv) were then added in sequence. The resulting solution was allowed to warm to 20° C. over a period of 12 h. The product mixture was concentrated. The residue obtained was treated with saturated aqueous ammonium chloride solution (200 mL), and the resulting mixture was extracted with ether (3×200 mL). The organic layers were combined and the combined layers were dried over magnesium sulfate. The dried solution was filtered and the filtrate was concentrated. The residue obtained was purified by flash-column chromatography (eluting with 5% ether-hexanes) to provide the α-methyl β-ketoester 10 as a colorless oil (14.9 g, 71%). Spectroscopic data were in agreement with those previously reported. (24) For determination of the enantiomeric excess of the product, see ref 24.

R_{f}=0.30 (5% ethyl acetate-hexanes; KMnO₄).

¹H NMR (400 MHz, CDCl₃) δ 3.69 (s, 3H), 2.72 (td, J=14, 6.8 Hz, 1H), 2.47-2.39 (m, 1H), 2.08-1.97 (m, 1H), 1.97-1.83 (m, 1H), 1.72-1.58 (m, 3H), 1.34 (s, 3H), 1.14 (d, J=6.4 Hz, 3H).

¹³C NMR (126 MHz, CDCl₃) δ 208.18, 171.67, 60.75, 51.82, 43.72, 39.72, 30.10, 25.34, 18.68, 16.92.

### Synthesis of the Vinyl Triflate 11:

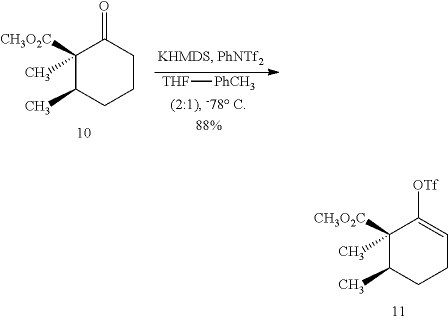

A solution of the α-methyl β-ketoester 10 (12.2 g, 66.2 mmol, 1 equiv) and N-phenyl-bis(trifluoromethanesulfonimide) (28.4 g, 49.5 mmol, 1.20 equiv) in tetrahydrofuran (400 mL) was stirred at −78° C. for 5 min. A solution of potassium bis(trimethylsilyl)amide in toluene (0.5 M, 200 mL, 1.50 equiv) was added dropwise over 10 min via cannula. The resulting solution was stirred for 50 min at −78° C. The cold product mixture was diluted with saturated aqueous ammonium chloride solution (200 mL) and the diluted solution was allowed to warm to 20° C. over 20 min. The warmed product mixture was extracted with ethyl acetate (3×200 mL). The organic layers were combined and the combined layers were dried over magnesium sulfate. The dried solution was filtered and the filtrate was concentrated. The residue obtained was purified by flash-column chromatography (eluting with 25% dichloromethane-hexanes initially, grading to 50% dichloromethane-hexanes, four steps) to provide the vinyl triflate 11 as a colorless oil (18.4 g, 88%).

R_{f}=0.40 (5% ethyl acetate-hexanes; KMnO₄).

¹H NMR (500 MHz, CDCl₃) δ 5.91 (dd, J=5.3, 3.0 Hz, 1H), 3.71 (s, 3H), 2.36-2.16 (m, 2H), 1.85-1.67 (m, 2H), 1.65-1.53 (m, 1H), 1.42 (s, 3H), 0.96 (d, J=6.4 Hz, 3H).

¹³C NMR (126 MHz, CDCl₃) δ 172.08, 150.07, 119.30, 118.45 (q, J=319.4 Hz), 52.29, 50.79, 40.27, 26.24, 23.40, 20.78, 16.92.

¹⁹F NMR (470 MHz, CDCl₃) δ−74.90.

HRMS-ESI (m/z): calculated for [C₁₁H₁₅F₃O₅SNa]⁺ 339.0490, found 339.0493.

### Synthesis of the Dienone 12:

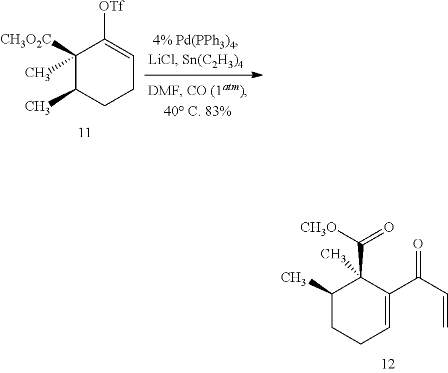

### Caution: This Reaction to be Performed in a Well-Ventilated Fume Hood

A solution of the vinyl triflate 11 (5.90 g, 18.7 mmol, 1 equiv), tetrakis(triphenylphosphine) palladium (862 mg, 746 μmol, 4.00 mol %) and lithium chloride (3.95 g, 93.3 mmol, 5.00 equiv) in N,N-dimethylformamide (190 mL) was sparged with carbon monoxide for 30 mM at 20° C. A balloon of carbon monoxide was attached to the reaction vessel and then tetravinyltin (4.42 mL, 24.3 mmol, 1.30 equiv) was added. The reaction mixture was stirred and heated at 40° C. for 6 h and then cooled to 20° C. The cooled product mixture was diluted with water (500 mL) and extracted with hexanes-ethyl acetate (35% v/v, 3×200 mL). The organic layers were combined and the combined organic layers were washed with aqueous ammonium hydroxide solution (10%, 200 mL). The washed organic layer was dried over magnesium sulfate. The dried solution was filtered and the filtrate was concentrated. The residue obtained was purified by flash-column chromatography (eluting with 10% ether-hexanes initially, linearly grading to 35% ether-hexanes) to provide the dienone 12 as a white solid (3.44 g, 83%).

R_{f}=0.44 (30% ether-hexanes; UV).

¹H NMR (600 MHz, CDCl₃) δ 6.93 (dd, J=5.2, 3.0 Hz, 1H), 6.80 (dd, J=17.1, 10.6 Hz, 1H), 6.21 (dd, J=17.1, 1.8 Hz, 1H), 5.73 (dd, J=10.6, 1.8 Hz, 1H), 3.66 (s, 3H), 2.41-2.32 (m, 1H), 2.32-2.24 (m, 1H), 1.74-1.60 (m, 3H), 1.37 (s, 3H), 0.93 (d, J=6.6 Hz, 3H).

¹³C NMR (151 MHz, CDCl₃) δ 191.63, 174.94, 143.47, 141.07, 132.77, 128.61, 51.73, 47.56, 38.82, 25.88, 25.11, 23.23, 16.75.

HRMS-ESI (m/z): calculated for [C₁₃H₁₈O₃Na]⁺ 245.1154, found 245.1150.

### Synthesis of the Cyclopentenone 13:

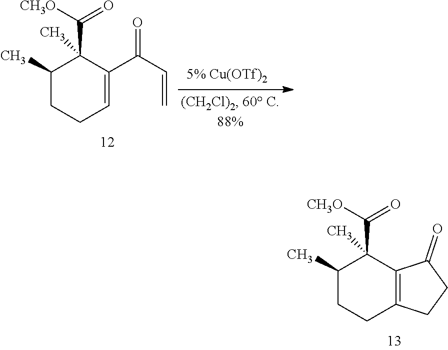

A solution of the dienone 12 (3.00 g, 13.5 mmol, 1 equiv) and copper(II) bis(trifluoromethanesulfonate) (244 mg, 675 μmol, 5.00 mol %) in 1,2-dichloroethane (140 mL) was stirred and heated at 60° C. for 16 h. The product mixture was cooled to 20° C. for 1 h. The cooled product mixture was then concentrated. The residue obtained was dissolved in ethyl acetate (100 mL) and the resulting solution was washed with saturated sodium bicarbonate solution (100 mL). The washed organic layer was dried over magnesium sulfate. The dried solution was filtered and the filtrate was concentrated. The residue obtained was purified by flash-column chromatography (eluting with 10% ethyl acetate-hexanes initially, linearly grading to 35% ethyl acetate-hexanes) to provide the cyclopentenone 13 as a light yellow solid (2.60 g, 88%).

R_{f}=0.38 (30% ethyl acetate-hexanes; UV).

¹H NMR (600 MHz, CDCl₃) δ 3.61 (s, 3H), 2.51 (t, J=4.7 Hz, 2H), 2.42-2.27 (m 4H), 1.72-1.62 (m, 3H), 1.41 (s, 3H), 0.90 (d, J=6.4 Hz, 3H).

¹³C NMR (151 MHz, CDCl₃) δ 207.31, 174.28, 173.46, 140.93, 51.75, 45.49, 39.15, 34.83, 29.85, 27.83, 27.12, 21.48, 16.22.

HRMS-ESI (m/z): calculated for [C₁₃H₁₉O₃]⁺ 223.1334, found 223.1332.

α_{D}²⁰=+17.90° (c=1.0, CH₂Cl₂)

HPLC: Chiralpak IA, hexane:EtOH 95:5, 1.0 mL/min, T_{4R,5R}=7.0 min, T_{4S,5S}=10.6 min, 97:3 er.

Synthesis of the β-cyano Ketone 15:

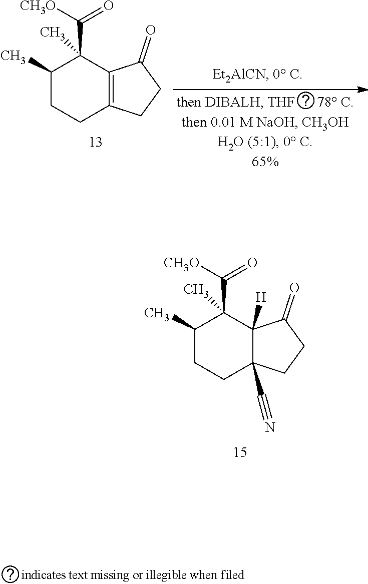

A solution of the cyclopentenone 1.3 (3.60 g, 16.2 mmol, 1 equiv) in tetrahydrofuran (160 mL) was cooled to 0° C. for 20 min. A solution of diethylaluminum cyanide in toluene (1.0 M, 48.6 mL, 48.6 mmol, 3.00 equiv) was added dropwise over 10 min via syringe. The reaction mixture was stirred at 0° C. for 2 h and then cooled to −78° C. for 10 min. A solution of di-iso-butylaluminum hydride in toluene (1.0 M, 48.6 mL, 48.6 mmol, 3.00 equiv) was added dropwise via syringe over 10 mm. After stirring for an additional 30 min at −78° C., aqueous potassium sodium tartrate solution (1.0%, 40 mL) was added via syringe over 30 min. The product mixture was diluted with ether (200 mL) and then warmed to 0° C. for 30 min. The warmed mixture was further diluted sequentially with aqueous potassium sodium tartrate solution (10%, 200 mL) and ether (200 mL). The resulting mixture was warmed to 20° C. and was stirred vigorously at this temperature for 1 h. The organic layer was separated and the aqueous layer was extracted with ether (2×200 mL). The organic layers were combined and the combined organic layers were dried over magnesium sulfate. The dried solution was filtered and the filtrate was concentrated. The residue obtained was dissolved in methanol (100 mL) and the resulting solution was cooled to 0° C. for 30 min. Aqueous sodium hydroxide solution (100 mM, 20 mL) was added to the cooled solution. After stirring the resulting mixture at 0° C. for 1 h, saturated aqueous ammonium chloride solution (200 mL) was added and the resulting mixture was warmed to 20° C. for 10 min. The product mixture was extracted with ethyl acetate (3×200 mL). The organic layers were combined and the combined organic layers were dried over magnesium sulfate. The dried solution was filtered and the filtrate was concentrated. The residue obtained was purified by flash-column chromatography (eluting with 10% ethyl acetate-hexanes initially, linearly grading to 30% ethyl acetate-hexanes) to provide the β-cyano ketone 15 as white solid (2.64 g, 65%).

R_{f}=0.33 (25% ethyl acetate-hexanes; KMnO₄).

¹H NMR (600 MHz, CDCl₃) δ 3.68 (s, 3H), 3.12 (s, 1H), 2.49-2.34 (m, 1H), 2.33-2.17 (m, 2H), 2.16-2.07 (m, 1H), 2.06-1.91 (m, 2H), 1.56 (s 3H), 1.54-1.48 (m, 1H), 1.48-1.40 (m, 1H), 1.36 (td, J=13.7, 4.1 Hz, 1H), 1.16 (d, J=7.0 Hz, 3H).

¹³C NMR (151 MHz, CDCl₃) δ 211.12, 173.97, 122.50, 58.76, 51.74, 46.12, 38.67, 36.63, 33.96, 32.30, 30.54, 27.67, 22.05, 15.88.

HRMS-ESI (m/z): calculated for [C₁₄H₁₉NO₃Na]⁺ 272.1263, found 272.1266.

### Synthesis of the Cyano Ketal 16:

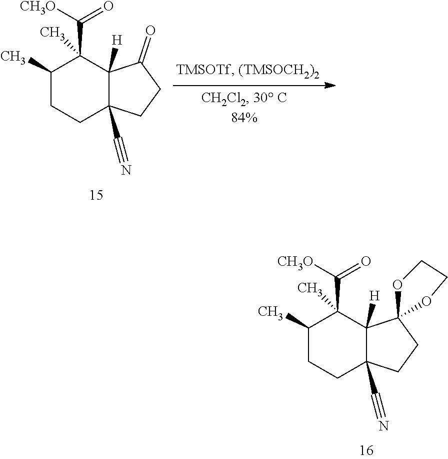

Bis(trimethylsilyl)ethylene glycol (8.26 mL, 33.7 mmol, 7.00 equiv) and trimethylsilyl trifluoromethanesulfonate (1.74 mL, 9.63 mmol, 2.00 equiv) were added it sequence to a solution of the β-cyano ketone 15 (1.20 g, 4.81 mmol, 1 equiv) in dichloromethane (60 mL) at 20° C. The resulting mixture was heated and stirred at 30° C. An additional portion of trimethylsilyl trifluoromethanesulfonate (1.74 mL, 9.63 mmol, 2.00 equiv) was added every two days thereafter. After stirring at 30° C. for 7 days total, the product mixture was cooled to 0° C. for 20 min. The cooled product mixture was slowly diluted with saturated aqueous sodium bicarbonate solution (60 mL). The resulting mixture was diluted with water (60 mL) and then the organic layer was separated. The aqueous layer was extracted with dichloromethane (2×60 mL). The organic layers were combined and the combined organic layers were dried over magnesium sulfate. The dried solution was filtered and the filtrate was concentrated. The residue obtained was purified by flash-column chromatography (eluting with 15% ethyl acetate-hexanes initially, linearly grading to 30% ethyl acetate-hexanes) to provide the cyano ketal 16 as a white solid (1.18 g, 84%).

R_{f}=0.36 (20% ethyl acetate-hexanes; PAA stains brawn).

¹H NMR (500 MHz, CDCl₃) δ 4.03-3.96 (m, 1H), 3.94-3.85 (m, 2H), 3.84-3.77 (m, 1H), 3.69 (s, 3H), 3.08 (s, 1H), 2.18-1.72 (m, 8H), 1.58-1.50(m, 1H), 1.32 (s, 3H), 1.13 (d, J=6.9 Hz, 3H).

¹³C NMR (126 MHz, CDCl₃) δ 175.46, 124.14, 117.50, 64.38, 62.53, 53.35, 51.63, 46.46, 40.28, 36.18, 35.37, 33.79, 31.61, 28.07, 21.37, 16.17.

HRMS-ESI (m/z): calculated for [C₁₆H₂₃NO₄Na]⁺ 316.1525, found 316.1530.

### Synthesis of the Enimide 7:

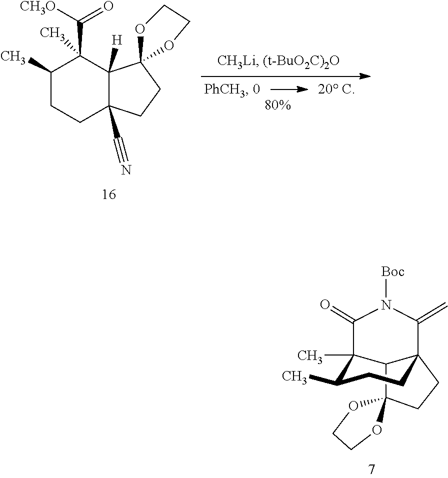

A solution of methyllithium in hexane (1.6 M, 2.56 mL, 4.09 mmol, 3.00 equiv) was added dropwise via syringe over 2 min to a solution of the cyano ketal 16 (400 mg, 1.36 mmol, 1 equiv) in toluene (20 mL) at 0° C. The resulting solution was stirred for 1.5 min at 0° C. Di-tert-butyl-dicarbonate (1.25 mL, 5.46 mmol, 4.00 equiv) was added dropwise via syringe over 2 min and the resulting solution was stirred for 1 h at 0° C. The solution was then warmed to 20° C. over 15 min. The warmed product mixture was diluted with saturated aqueous sodium hydrogen carbonate solution (50 mL), and the diluted mixture was extracted with ethyl acetate (3×50 mL). The organic layers were combined and the combined organic layers were dried over magnesium sulfate. The dried solution was filtered and the filtrate was concentrated. The residue obtained was purified by flash-column chromatography (eluting with 5% ethyl acetate-hexanes initially, linearly grading to 20% ethyl acetate-hexanes) to provide the enimide 7 as a colorless oil (413 mg, 80%).

R_{f}=0.42 (20% ethyl acetate-hexanes; UV, PAA stains orange).

¹H NMR (600 MHz, C₆D₆) δ 4.51 (d, J=1.9 Hz, 1H), 4.01 (d, J=1.9 Hz, 1H), 3.39-3.33 (m, 1H), 3.33-3.25 (m, 2H), 3.20-3.15 (m, 1H), 2.49 (dqd, J=13.5, 6.9, 4.7 Hz, 1H), 2.31 (d, J=1.1 Hz, 1H), 2.03-1.95 (m, 1H), 1.81-1.35 (m, 7H), 1.49 (s, 3H), 1.44 (s, 9H), 1.11 (d, J=6.9 Hz, 3H).

¹³C NMR (151 MHz, C₆D₆) δ 171.59, 152.84, 150.87, 117.32, 87.26, 84.03, 64.43, 62.58, 52.60, 46.42, 44.15, 38.35, 34.68, 33/82, 33.63, 29.71, 27.49, 19.26, 16.77.

HRMS-ESI (m/z): calculated for [C₂₁H₃₁NO₅Na]⁺ 400.2100, found 400.2096.

Synthesis of the α-alkylated Imide 20:

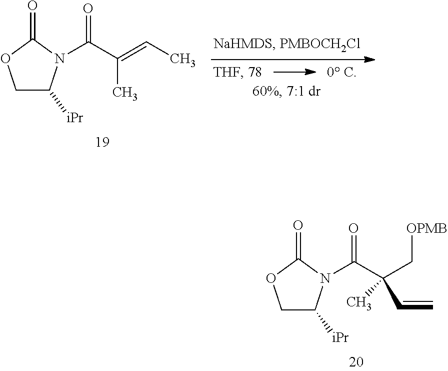

A solution of the oxazolidinone 19 (2.66 g, 12.6 mmol, 1 equiv) in tetrahydrofuran (100 mL) was cooled to −78° C. for 10 min and then a solution of sodium bis(trimethylsilyl)amide in tetrahydrofuran (1.6 M, 15.7 mL, 25.2 mmol, 2.00 equiv) was added via cannula. The resulting solution was stirred at −78° C. for 30 min. Para-methoxybenzyl chloromethyl ether (4.70 g, 25.2 mmol, 2.00 equiv) was then added dropwise via syringe and the resulting solution was stirred at −78° C. for an additional 1 h. The resulting solution was allowed to warm to 0° C. over a period of 1 h and then to 20° C. over a period of 30 min. The warmed product mixture was diluted sequentially with saturated aqueous ammonium chloride solution (100 mL) and water (100 mL). The diluted product mixture was extracted with ether (3×200 mL). The organic layers were combined and the combined organic layers were dried over sodium sulfate. The dried solution was filtered and the filtrate was concentrated. The α-alkylated imide 20 was formed as a 7:1 mixture of diastereomers based on ¹H NMR analysis of the unpurified product mixture. In general, the diastereoselectivity of this transformation varied from 5:1 to 10:1. The residue obtained was purified by flash-column chromatography (eluting with 5% ether-pentane initially, grading to 50% ether-pentane, five steps) to provide the α-alkylated imide 20 as a pale yellow oil (2.88 g, 60%, 7:1 dr).

R_{f}=0.19 (25% ether-pentane, UV, KMnO₄).

¹H NMR (600 MHz, CDCl₃) δ7.21 (d, J=8.5 Hz, 2H), 6.85 (d, J=8.6 Hz, 2H), 6.17 (dd, J=17.7, 10.7 Hz, 1H), 5.08 (d, J=10.8 Hz, 1H), 4.97 (d, J=17.7 Hz, 1H), 4.53-4.37 (m, 3H), 4.26-4.14 (m, 3H), 3.80 (s, 3H), 3.51 (d, J=8.9 Hz, 1H), 2.36-2.27 (m, 1H), 1.48 (s, 3H), 0.88 (d, J=7.1 Hz, 3H), 0.81 (d, J=6.9 Hz, 3H).

¹H NMR (151 MHz, CDCl₃) δ174.04, 159.19, 152.73, 139.53, 130.47, 129.19, 113.94, 113.77, 75.13, 73.13, 63.32, 60.12, 55.41, 52.29, 28.47, 22.77, 18.17, 14.78.

HRMS-ESI (m/z): calculated for [C₂₀H₂₇NO₅Na]⁺ 384.1787, found 384.1782.

### Synthesis of the Alcohol S2:

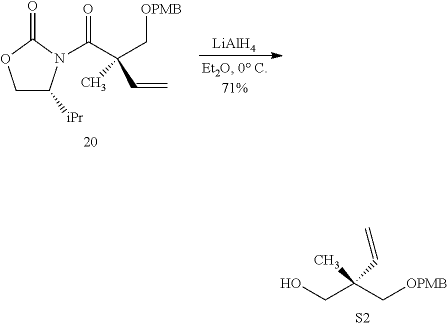

A solution of the α-alkylated imide 20 (6.67 g, 18.5 mmol, 1 equiv) in ether (30 mL) was added over 5 min via cannula to a stifling suspension of lithium aluminum hydride (1.40 g, 36.9 mmol, 2.00 equiv) in ether (150 mL) that had been preceded to 0° C. for 10 min. The resulting solution was stirred for 10 min at 0° C. Water (1.0 mL) and aqueous sodium hydroxide solution (3 M, 1.0 mL) were added sequentially to the product mixture at 0° C. and the resulting mixture was gradually warmed to 20° C. over a period of 15 min. Sodium sulfate (˜2 g) was added and the resulting mixture was filtered through a pad of celite. The filtrate was collected and concentrated. The residue obtained was purified by flash-column chromatography (eluting with 10% ethyl acetate-pentane initially, grading to 30% ethyl acetate-pentane, four steps) to provide the alcohol S2 as a colorless oil (3.10 g, 71%).

R_{f}=0.30 (25% ethyl acetate-pentane, PAA stains blue).

¹H NMR (600 MHz, CDCl₃) δ7.24 (d, J=8.5 Hz, 2H), 6.88 (d, J=8.6 Hz, 2H), 5.84 (dd, J=17.7, 11.0 Hz, 1H), 5.14 (d, J=12.1 Hz, 1H), 5.11 (d, J=18.4 Hz, 1H), 4.47 (d, J=11.8 Hz, 1H), 4.44 (d, J=11.8 Hz, 1H), 3.81 (s, 3H), 3.57 (dd, J=10.7, 5.3 Hz, 1H), 3.52 (dd, J=10.9, 5.3 Hz 1H), 3.45 (d, J=8.8 Hz, 1H), 3.36 (d, J=8.8 Hz, 1H), 2.40 (t, J=6.0 Hz, 1H), 1.04 (s, 3H).

¹³C NMR (151 MHz, CDCl₃), δ 159.34, 141.59, 130.21, 129.29, 114.56, 113.94, 76.82, 73.34, 69.69, 55.39, 42.84, 19.16.

HRMS-ESI (m/z): calculated for [C₁₄H₂₆O₃Na]⁺ 259.1310, found 259.1319.

### Synthesis of Neopentyl Iodide 8:

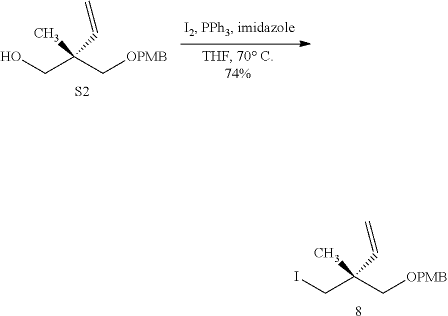

Iodine (2.48 g, 9.78 mmol 1.10 equiv) was added in one portion to a stirring solution of the alcohol S2 (2.10 g, 8.89 mmol, 1 equiv), triphenylphosphine (2.56 g, 9.78 mmol, 1.10 equiv), and imidazole (1.21 g, 17.8 mmol, 2.00 equiv) in tetrahydrofuran (44 mL) at 20° C. The resulting mixture was stirred and heated at 70° C. for 3 h and then cooled to 20° C. over a period of 30 min. The cooled product mixture was concentrated. The residue obtained was treated with saturated aqueous ammonium chloride solution (50 mL) and the resulting mixture was extracted with ethyl acetate (3×50 mL). The organic layers were combined and the combined organic layers were washed with aqueous sodium thiosulfate solution (20% w/v, 50 mL). The washed organic layers were dried over sodium sulfate. The dried solution was filtered and the filtrate was concentrated. The residue obtained was purified by flash-column chromatography (eluting with 1% ethyl acetate-hexane initially, linearly grading to 5% ethyl acetate-hexane) to provide the neopentyl iodide 8 as a pale yellow oil (2.30 g, 74%).

R_{f}=0.50 (4% ethyl acetate-pentane, UV; PAA stains blue).

¹H NMR (600 MHz, CDCl₃) δ 7.26 (d, J=8.6 Hz, 1H), 6.88 (d, J=8.6 Hz, 2H), 5.85 (dd, J=17.6, 10.9 Hz, 1H), 5.10 (dd, J=22.7, 14.2 Hz, 2H), 4.46 (s, 2H), 3.81 (s, 3H), 3.37-3.23 (m, 4H), 1.15 (s, 3H).

¹³C NMR (151 MHz, CDCl₃) δ 159.27, 141.53, 130.56, 129.31, 114.65, 113.85, 75.97, 73.23, 55.42, 41.01, 21.88, 18.28.

HRMS-ESI (m/z): calculated for [C₁₄H₁₉IO₂Na]⁺ 369.0327, found 369.0325.

### Synthesis of Diketone 21:

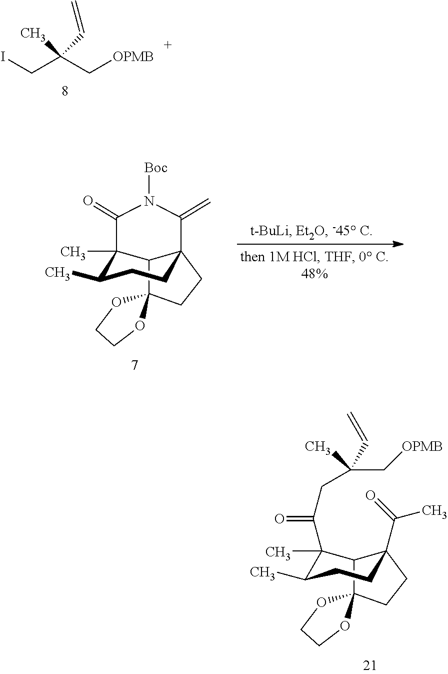

A solution of pentane-ether (8:1, 3.6 mL) was cooled to −45° C. for 10 min. A solution of tert-butyllithium in pentane (1.7 M, 680 μL, 1.16 mmol, 4.40 equiv) was added followed by the neopentyl iodide 8 (218 mg, 630 μmol, 2.40 equiv) over 5 min. The resulting mixture was stirred at −45° C. for 40 min. A solution of the enimide 7 in ether (140 mM, 1.9 mL, 263 μmol, 1 equiv) was added dropwise over 5 min and the resulting mixture was stirred for an additional 2 h at −45° C. Aqueous sodium thiosulfate solution (20% w/v, 2.0 mL) was then added and the resulting mixture was warmed to 20° C. over 10 min. The warmed mixture was further diluted with aqueous sodium thiosulfate solution (20% w/v, 30 mL). The diluted mixture was extracted with ethyl acetate (3×20 mL). The organic layers were combined and the combined organic layers were dried over magnesium sulfate. The dried solution was filtered and the filtrate was concentrated. The residue obtained was dissolved in tetrahydrofuran (10 mL) and cooled to 0° C. for 10 min. Aqueous hydrochloric acid solution (1 M, 10 mL) was added dropwise via syringe. The resulting mixture was stirred for 3 h at 0° C. The product mixture was diluted with aqueous sodium hydroxide solution (10 M, 4.5 mL) and the diluted mixture was warmed to 20° C. The warmed mixture was extracted with ethyl acetate (3×50 mL). The organic layers were combined and the combined layers were dried over magnesium sulfate. The dried solution was filtered and the filtrate was concentrated. The residue obtained was purified by flash-column chromatography (eluting with 10% ethyl acetate-hexanes initially, linearly grading to 30% ethyl acetate-hexanes) to provide the diketone 21 as a colorless oil. The purity of the diketone 21 was determined by NMR analysis against an internal standard (84.0 mg, 73% w/w purity, 48%).

R_{f}=0.36 (40% v/v ether-pentane; UV, PAA stains pink).

¹H NMR (500 MHz, CDCl₃): δ7.23 (d, J=8.5 Hz, 2H), 6.87 (d, J=8.5 Hz, 2H), 5.98 (dd, J=11, 18 Hz, 1H), 5.04-4.98 (m, 2H), 4.42 (dd, J=12, 17 Hz, 2H), 3.92-3.82 (m, 2H), 3.80 (s, 3H), 3.62-3.50 (m, 2H), 3.46 (d, J=8.5 Hz, 1H), 3.29 (d, J=8.5 Hz, 1H), 3.23 (s, 1H), 2.70 (d, J=18 Hz, 1H), 2.64 (d, J=18 Hz, 1H), 2.52-2.53 (m, 1H), 2.22 (s, 3H), 1.95-1.77 (m, 4H), 1.71-1.60 (m, 1H), 1.59-1.41 (m, 2H), 1.50 (s, 3H), 1.27-1.18 (m, 1H), 1.16 (s, 3H), 0.80 (d, J=7.1 Hz, 3H).

¹³C NMR (126 Hz, CDCl₃): δ 212.07, 211.70, 159.15, 144.50, 130.87, 129.15, 119.60, 113.78, 112.41, 76.94, 72.99, 65.01, 63.90, 57.44, 55.39, 49.82, 45.50, 43.71, 40.04, 37.14, 35.25, 27.93, 24.92, 24.83, 24.32, 22.54, 21.39, 15.62.

HRMS-ESI (m/z): calculated for [C₃₀H₄₃O₆]⁺ 499.3060, found 499.3065.

### Synthesis of the Alkyne 22:

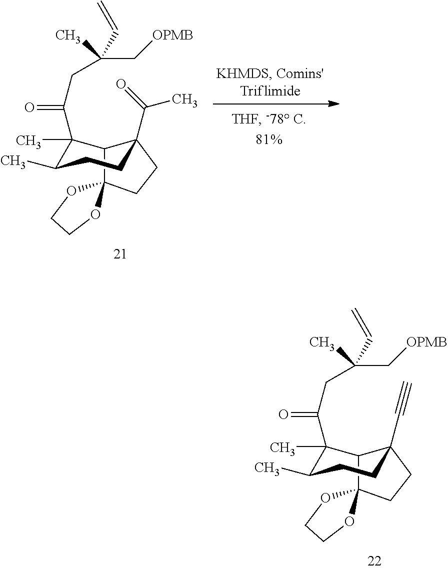

A solution of potassium bis(trimethylsilyl)amide in tetrahydrofuran (0.5 M, 860 μL, 430 μmol, 3.50 equiv) was added dropwise via syringe over 10 min to a solution of the diketone 21 (84.0 mg, 73% w/w purity, 123 μmol, 1 equiv) and N-(5-chloro-2-pyridyl)triflimide (Comins' reagent, 62.8 mg, 160 μmol, 1.30 equiv) in tetrahydrofuran (2.4 mL) at −78° C. The resulting solution was stirred for 30 min at −78° C. and then methanol (1.2 mL) was added. The resulting mixture was warmed to 20° C. over 10 min. The warmed mixture was diluted with aqueous sodium hydroxide solution (1 M, 4.0 mL) and the diluted mixture was extracted with ether (3×4.0 mL). The organic layers were combined and the combined organic layers were dried over magnesium sulfate. The dried solution was filtered and the filtrate was concentrated. The residue obtained was purified by flash-column chromatography (eluting with 5% ethyl acetate-hexanes initially, linearly grading to 15% ethyl acetate-hexanes) to provide the alkyne 22 as a colorless oil (59.1 mg, 81%). In some instances, then trimethylsilyl-protected alkyne was formed in approximately 0-30% yield depending on the purity of diketone 21. In cases where this side product was formed, the aqueous sodium hydroxide solution was replaced with aqueous lithium hydroxide solution (4 M) and the resulting mixture was stirred at 20° C. for 0.5-4 h to quantitatively desilylate the alkyne.

R_{f}=0.59 (40% v/v ether-hexane; PAA stains blue).

¹H NMR (500 MHz, CDCl₃) δ7.23 (d, J=8.6 Hz, 2H), 6.85 (d, J=8.7 Hz, 2H), 6.03 (dd, J=17.6, 10.9 Hz, 1H), 5.04-4.94 (m, 2H), 4.43 (d, J=12.0 Hz, 1H), 4.40 (d, J=11.8 Hz, 1H), 3.93-3.72 (m, 4H), 3.79 (s, 3H), 3.44 (d, J=8.5 Hz, 1H), 3.36 (d, J=8.6 Hz, 1H), 2.81 (d, J=17.3 Hz, 1H), 2.71 (s, 1H), 2.70 (d, J=17.2 Hz, 1H), 2.01 (s, 1H), 2.14-1.74 (m, 6H),1.70-1.56 (m, 2H), 1.49-1.41 (m, 1H), 1.25 (s, 3H), 1.14 (s, 3H), 1.03 (d, J=6.9 Hz, 3H).

¹³C NMR (151 MHz, CDCl₃) δ210.75, 159.03, 144.95, 131.06, 129.05, 119.03, 113.72, 112.01, 90.56, 76.77, 72.90, 70.66, 64.19, 62.46, 55.38, 52.53, 51.17, 44.00, 40.72, 40.31, 36.80, 36.50, 33.41, 30.44, 27.74, 22.27, 21.34, 15.99.

HRMS-ESI (m/z): calculated for [C₃₀H₄₁O₅]⁺ 481.2954, found 481.2956.

### Synthesis of the Alkynyl Alcohol 83:

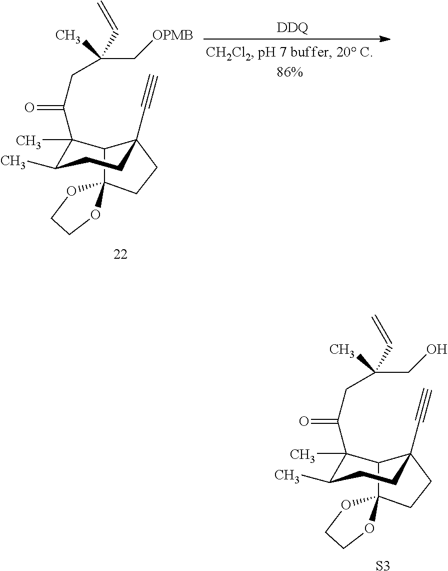

Aqueous potassium phosphate buffer (10 mM, pH 7, 1.0 mL.) was added to a solution of the alkyne 22 (146 mg, 303 μmol, 1 equiv) in dichloromethane (3 mL) at 20° C. 2,3-Dichloro-5,6-dicyano-p-benzoquinone (275 mg, 1.21 mmol, 4.00 equiv) was then added in one portion and the resulting solution was stirred at 20° C. open to air for 30 min. The product mixture was diluted with saturated aqueous sodium bicarbonate solution (40 mL). The diluted product mixture was extracted with dichloromethane (3×30 mL). The organic layers were combined and the combined organic layers were dried over sodium sulfate. The dried solution was filtered and the filtrate was concentrated. The residue obtained was purified by flash-column chromatography (eluting with 10% ethyl acetate-pentane initially, grading to 30% ethyl acetate-pentane, four steps) to provide the alkynyl alcohol S3 as a colorless oil (94.0 mg, 86%).

R_{f}=0.19 (40% etherpentane; PAA stains brown).

¹H NMR (500 MHz, CDCl₃) δ5.84 (dd, J=18.0, 10.6 Hz, 1H), 5.03 (d, J=11.4 Hz, 1H), 5.02 (d, J=17.0 Hz, 1H), 3.99-3.76 (m, 4H), 3.55 (d, J=11.0 Hz, 1H), 3.49 (d, J=10.9 Hz, 1H), 3.09 (s, br, 1H), 2.82 (d, J=17.0 Hz, 1H), 2.75 (d, J=17.0 Hz, 1H), 2.67 (s, 1H), 2.13 (s, 1H), 2.17-1.72 (m, 6H), 1.72-1.54 (m, 2H), 1.49-1.38 (m, 1H), 1.27 (s, 3H), 1.06 (s, 3H), 1.05 (d, J=7.0 Hz, 3H).

¹³C NMR (101 Hz, CDCl₃), δ212.60, 144.81, 118.72, 112.40, 90.04, 70.63, 69.85, 64.02, 62.22, 52.45, 51.19, 45.16, 41.79, 40.25, 36.46, 36.44, 36.24, 33.21, 27.70, 20.85, 20.55, 15.74.

HRMS-ESI (m/z): calculated for [C₂₂H₃₂O₄Na]⁺ 383.2198, found 383.2207.

### Synthesis of the Alkynyl Aldehyde 23:

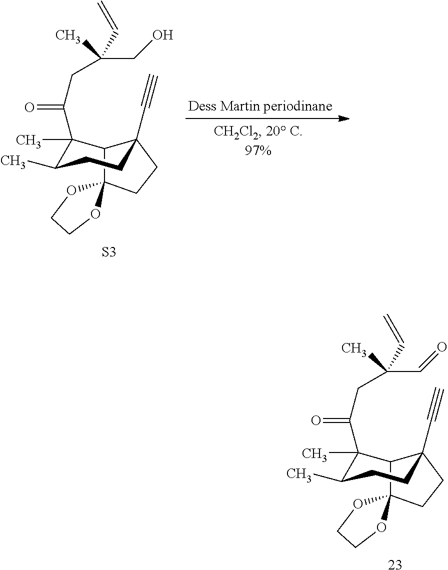

The Dess-Martin periodinane (419 mg, 988 μmol, 4.00 equiv) was added in one portion to a solution of the alkynyl alcohol S3 (89.2 mg, 247 μmmol, 1 equiv) in dichloromethane (2.5 mL) at 20° C. The resulting mixture was stirred open to air for 1 h at 20° C. The product mixture was diluted sequentially with ether (2.5 mL), aqueous sodium thiosulfate solution (20% w/v, 2.0 mL), and saturated aqueous sodium bicarbonate solution (2.0 mL). The resulting mixture was stirred until it became clear (approximately 15 min) and then extracted with ether (3×3.0 mL). The organic layers were combined and the combined organic layers were dried over magnesium sulfate. The dried solution was filtered and the filtrate was concentrated to provide the alkynyl aldehyde 23 as a colorless oil (88.5 mg, 97%). The product so obtained was judged to be of >95% purity (¹H NMR analysis) and was used without further purification.

R_{f}=0.54 (40% ether-pentane, PAA stains purple).

¹H NMR (400 MHz, CDCl₃) δ 9.50 (s, 1H), 5.75 (dd, J=17.5, 10.7 Hz, 1H), 5.18 (d, J=10.7 Hz, 1H), 5.12 (d, J=17.5 Hz, 1H), 4.01-3.77 (m, 4H), 3.35 (d, J=17.1 Hz, 1H), 2.95 (d, J=17.1 Hz, 1H), 2.70 (s, 1H), 2.16 (s, 1H), 2.11-1.76 (m, 6H), 1.72-1.55 (m, 2H), 1.48-1.39 (m, 1H), 1.27 (s, 3H), 1.18 (s, 3H), 1.05 (d, J=6.5 Hz, 3H).

¹³C NMR (101 Hz, CDCl₃) δ 209.77, 201.22, 138.93, 118.82, 116.06, 90.23, 71.17, 64.25, 62.40, 53.06, 51.21, 50.84, 46.22, 40.53, 36.58, 36.56, 36.50, 33.46, 27.82, 20.97, 18.58, 15.83.

HRMS-ESI (m/z): calculated for [C₂₂H₃₁O₄]⁺ 359.2222, found 359.2217.

### Synthesis of the Allylic Alcohol 24:

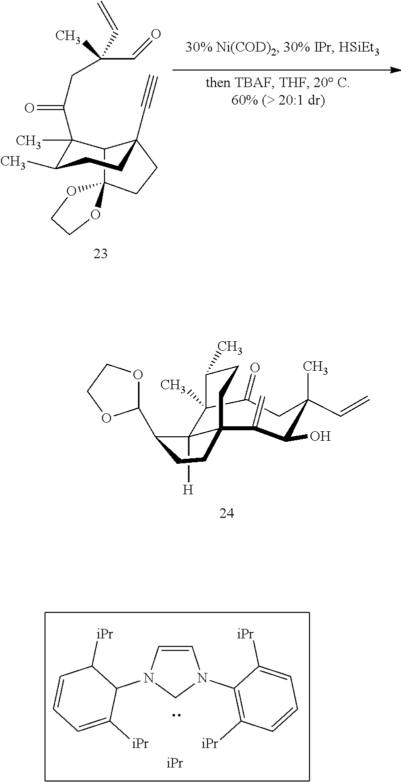

A stock solution of the catalyst was prepared by stirring a solution of bis(1,5-cyclooctadiene)nickel(0) (46.0 mg, 167 μmol, 1.00 equiv) and 1,3-bis(2,6-diisopropylphenyl)imidazol-2-ylidene (IPr, 65.4 mg, 167 μmol, 1.00 equiv) in tetrahydrofuran (1.0 mL) at 20° C. for 30 min. A portion of the catalyst stock solution (250 μL, 25 mol %) was added to a stirring solution of the alkynyl aldehyde 23 (60.0 mg, 167 μmol, 1 equiv) and triethylsilane (80.0 μL, 502 μmol, 3.00 equiv) in tetrahydrofuran (3.0 mL) at 20° C. The resulting solution was stirred for 4 h at 20° C. Another portion of the catalyst stock solution (100 μL, 10 mol %) was added to the reaction mixture and the resulting solution was stirred for an additional 2 h. The resulting mixture was filtered through a short pad of silica gel (eluting with 50% ethyl acetate-hexanes). The filtrate was concentrated and the residue obtained was dissolved in tetrahydrofuran (840 μL). Tetra n-butylammonium fluoride in tetrahydrofuran (1.0M, 837 μL, 837 μmol, 5.00 equiv) was added and the resulting solution was stirred at 20° C. for 15 min under air. The resulting mixture was diluted sequentially with saturated aqueous ammonium chloride solution (3.0 mL) and water (2.0 mL). The diluted solution was extracted with ether (3×4.0 mL). The organic layers were combined and the combined organic layers were dried over magnesium sulfate. The dried solution was filtered and the filtrate was concentrated. The residue obtained was purified by flash-column chromatography (eluting with 20% ethyl acetate-hexanes initially, linearly grading to 45% ethyl acetate-hexanes) to provide the allylic alcohol 24 as a colorless oil (36.0 mg, 60%).

R_{f}=0.28 (40% v/v ethyl acetate-hexanes; PAA stains pink).

¹H NMR (600 MHz, C₆D₆) δ 5.57-5.50 (m, 2H), 5.32 (s, 1H), 4.97 (d, J=17.4 Hz, 1H), 4.87 (d, J=10.7 Hz, 1H), 4.15 (s, 1H), 3.48-3.42 (m, 1H), 3.42-3.34 (m, 2H), 3.31-3.24 (m, 1H), 2.95 (s, 1H), 2.87 (d, J=12.2 Hz, 1H), 2.58-2.50 (m, 1H), 2.15-2.03 (m, 1H), 1.86-1.73 (m, 5H), 1.65 (d, J=12.2 Hz, 1H), 1.49 (d, J=7.1 Hz, 3H), 1.45-1.42 (m, 1H), 1.40 (s, 3H), 1.36 (s, 1H), 1.20 (s, 3H), 1.17-1.14 (m, 1H).

¹³C NMR (151 MHz, C₆D₆) δ 210.06, 149.00, 146.75, 119.27, 115.81, 113.16, 72.06, 63.87, 61.54, 51.40, 51.33, 48.81, 46.98, 44.95, 36.71, 35.37, 35.02, 28.98, 26.75, 19.81, 16.47, 14.55.

HRMS-ESI (m/z): calculated for [C₂₂H₃₃O₄]⁺ 361.2379, found 361.2383.

### Synthesis of the Enone S4:

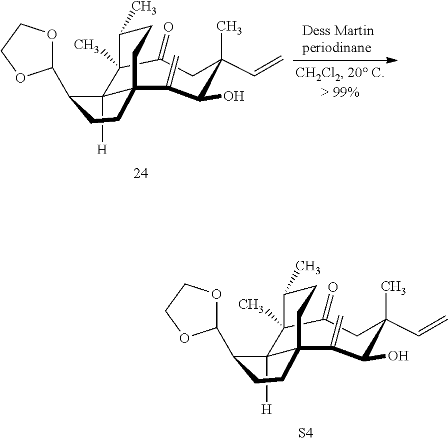

The Dess-Martin periodinane (61.2 mg, 144 μmol, 4.00 equiv) was added to a solution of the allylic alcohol 24 (13.0 mg, 36.1 μmol, 1 equiv) in dichloromethane (500 μL) at 20° C. The resulting mixture was stirred open to air and for 6 h at 20° C. The product mixture was diluted sequentially with ether (1.0 mL), aqueous sodium thiosulfate solution (20% w/v, 1.0 mL), and saturated aqueous sodium bicarbonate solution (1.0 mL). The resulting mixture was stirred until it became clear (approximately 15 min) and was then extracted with ether (3×2.0 mL). The organic layers were combined and the combined organic layers were dried over magnesium sulfate. The dried solution was filtered and the filtrate was concentrated to provide the enone S4 as a white solid (13.0 mg, >99%). The product so obtained was judged to be of >95% purity (¹H NMR analysis) and was used without further purification.

R_{f}=0.49 (15% v/v ethyl acetate-hexanes; UV; PAA stains yellow).

¹H NMR (400 MHz, C₆D₆) δ 6.33 (dd, J=17.5, 10.9 Hz, 1H), 5.06 (s, 1H), 4.99 (dd, J=10.9, 0.5 Hz, 1H), 4.84 (dd, J=17.5, 0.5 Hz, 1H), 4.76 (s, 1H), 3.45-3.24 (m, 3H), 3.23-3.15 (m, 1H), 3.04 (d, J=11.9 Hz, 1H), 2.72-2.59 (m, 2H), 2.52 (dqd, J=14.2, 7.1, 3.8 Hz, 1H), 2.08 (qd, J=12.9, 4.2 Hz, 1H), 1.79-1.59 (m, 5H), 1.47 (s, 3H), 1.46 (d, J=7.1 Hz, 3H), 1.37 (ddd, J=13.2, 7.0, 3.6 Hz, 1H), 1.27-1.22 (m, 1H), 1.21 (s, 3H).

¹³C NMR (101 MHz, C₆D₆) δ 210.54, 210.17, 152.28, 142.23, 119.27, 115.47, 112.27, 64.13, 61.97, 55.24, 53.42, 52.34, 49.16, 44.55, 36.84, 35.75, 35.57, 28.99, 26.93, 21.96, 20.09, 16.74.

HRMS-ESI (m/z): calculated for [C₂₂H₃₁O₄]⁺ 359.2222, found 359.2227.

### Synthesis of the Diketone 25:

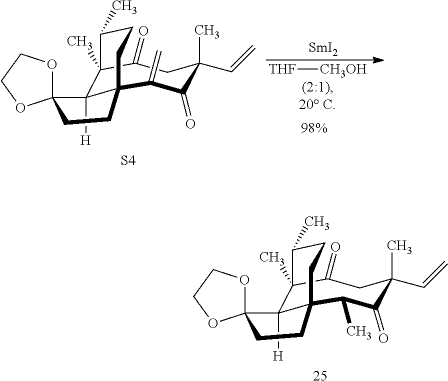

Methanol (1.0 mL, 250 mmol, 500 equiv) was added to a solution of samarium(II) iodide in tetrahydrofuran (0.1 M, 2.00 mL, 200 μmol, 4.00 equiv) at 20° C., resulting in a green solution. A solution of the enone S4 (17.7 mg, 49.4 μmol, 1 equiv) in tetrahydrofuran (1.0 mL) was then added. The resulting mixture was stirred for 5 min at 20° C. The product mixture was diluted sequentially with saturated aqueous ammonium chloride solution (2.0 mL) and water (2.0 mL). The diluted product mixture was extracted with ethyl acetate (3×2.0 mL). The organic layers were combined and the combined organic layers were washed with aqueous sodium thiosulfate solution (20% w/v, 2.0 mL). The washed organic layer was dried over magnesium sulfate. The dried solution was filtered and the filtrate was concentrated to provide the diketone 25 as white solid (17.4 mg, 98%). The product so obtained was judged to be of >95% purity (¹H NMR analysis) and was used without further purification.

R_{f}=0.50 (15% v/v ethyl acetate-hexanes; PAA stains pink).

¹H NMR (500 MHz, CDCl₃) δ 6.20 (dd, J=17.6, 10.9 Hz, 1H), 5.13 (d, J=10.9 Hz, 1H), 5.00 (d, J=17.6 Hz, 1H), 3.98 (q, J=6.9 Hz, 1H), 3.89 (dd, J=13.9, 7.1 Hz, 1H), 3.84 (dd, J=13.5, 6.9 Hz, 1H), 3.74 (dd, J=13.8, 7.2 Hz, 1H), 3.09 (d, J=11.6 Hz, 1H), 3.07 (q, J=7.0 Hz, 1H), 2.36-2.25 (m, 1H), 2.17 (s, 1H), 2.11 (dd, J=23.3, 10.9 Hz, 1H), 1.93 (d, J=11.7 Hz, 1H), 1.81 (d, J=10.5 Hz, 2H), 1.79-1.69 (m, 1H), 1.54-1.48 (m, 2H), 1.42 (s, 3H), 1.41-1.34 (m, 1H), 1.31-1.27 (m, 1H), 1.22 (s, 3H), 1.21 (d, J=7.1 Hz, 3H), 0.95 (d, J=6.7 Hz, 3H).

¹³C NMR (126 MHz, CDCl₃) δ 216.30, 214.62, 141.91, 119.56, 112.54, 64.21, 62.03, 55.46, 53.58, 51.83, 46.85, 44.53, 41.35, 35.90, 35.06, 29.58, 27.41, 26.61, 20.59, 20.30, 16.39, 13.47.

HRMS-ESI (m/z): calculated for [C₂₂H₃₃O₄]⁺ 361.2379, found 361.2375.

### Synthesis of the Ketoalcohol 27:

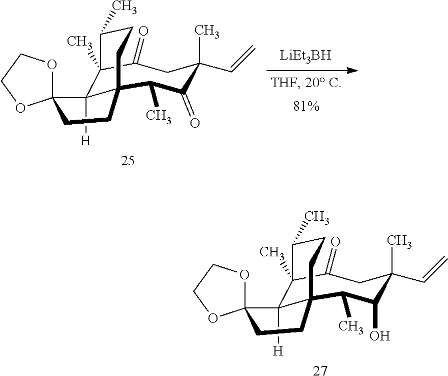

Lithium triethylborohydride (78.4 μL, 78.4 μmol, 2.50 equiv) was added dropwise via syringe to a solution of the diketone 25 (11.3 mg, 31.4 μmol, 1 equiv) in tetrahydrofuran (150 μL) at 20° C. The resulting mixture was stirred for 2 h at 20° C. and then was diluted sequentially with ethyl acetate (2.0 mL), saturated aqueous ammonium chloride solution (2.0 mL), and water (2.0 mL). The resulting mixture was extracted with ethyl acetate (3×3.0 mL). The organic layers were combined and the combined organic layers were dried over magnesium sulfate. The dried solution was filtered and the filtrate was concentrated. The residue obtained was purified by preparative thin-layer chromatography (eluting with 20% ethyl acetate-hexanes) to provide the ketoalcohol 27 as a white solid (9.2 mg, 81%).

R_{f}=0.31 (15% v/v ethyl acetate-hexanes; PAA stains pink).

¹H NMR (400 MHz, C₆D₆) δ 5.18 (dd, J=17.5, 10.8 Hz, 1H), 4.69 (d, J=10.8 Hz, 1H), 4.60 (d, J=17.5 Hz, 1H), 3.83 (s, 1H), 3.50-3.29 (m, 4H), 3.26 (d, J=11.4 Hz, 1H), 3.11 (s, 1H), 2.69-2.57 (m, 1H), 2.54 (ddd, J=12.3, 10.9, 9.1 Hz, 1H), 2.00-1.85 (m, 3H), 1.85-1.74 (m, 1H), 1.68 (td, J=13.8, 4.2 Hz, 1H), 1.62 (d, J=11.3 Hz, 1H), 1.55 (t, J=5.6 Hz, 3H), 1.55-1.48 (m, 1H), 1.44 (ddd, J=13.6, 6.9, 3.6 Hz, 1H), 1.36 (s, 3H), 1.27 (d, J=1.8 Hz, 1H), 1.21-1.12 (m, 1H), 1.10 (s, 3H), 0.92 (d, J=7.1 Hz, 3H).

¹³C NMR (151 MHz, C₆D₆) δ 214.74, 146.37, 121.03, 114.01, 84.69, 63.83, 61.87, 51.89, 50.46, 47.33, 46.75, 40.02, 35.89, 35.67, 33.73, 32.24, 30.72, 28.02, 23.33, 20.94, 20.27, 17.23.

HRMS-ESI (m/z): calculated for [C₂₂H₃₅O₄]⁺ 363.2535, found 363.2538.

### Synthesis of the Ketoalcohols 28 and 27:

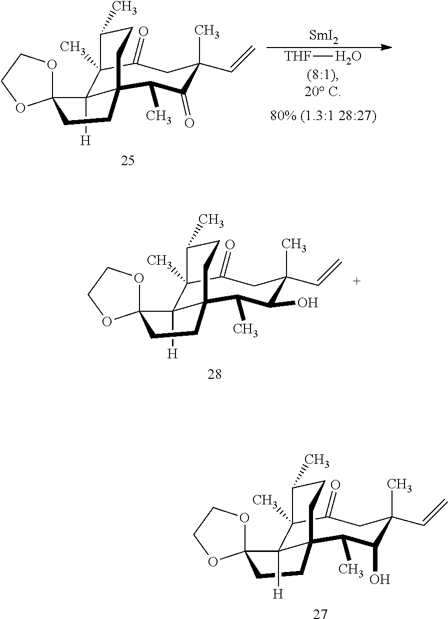

Water (400 μL, 22.2 mmol, 800 equiv) was added to a solution of samarium(II) iodide in tetrahydrofuran (0.1 M, 2.22 mL, 222 μmol, 8.00 equiv) at 20° C. resulting in a red solution. A solution of the diketone 25 (10.0 mg, 27.7 μmol, 1 equiv) in tetrahydrofuran (1.0 mL) was then added. The resulting mixture was stirred for 10 min at 20° C. The product mixture was diluted sequentially with saturated aqueous ammonium chloride solution (1.0 mL) and water (5.0 mL). The diluted product mixture was extracted with ether (3×3.0 mL). The organic layers were combined and the combined organic layers were washed with aqueous sodium thiosulfate solution (20% w/v, 3.0 mL). The washed organic layers were dried over magnesium sulfate. The dried solution was filtered and the filtrate was concentrated. The ketoalcohols 28 and 27 were formed in a 1.3:1 ratio based on ¹H NMR analysis of the unpurified product mixture. The residue obtained was purified by preparative thin-layer chromatography (eluting with 25% ethyl acetate-hexanes) to provide the ketoalcohol 28 as a white solid (4.0 mg, 40%) and the ketoalcohol 27 as a white solid (4.0 mg, 40%).

27:

The spectroscopic data were in agreement with those reported above.

28:

R_{f}=0.30 (30% v/v ethyl acetate-hexanes; UV; PAA stains pink). (equatorial keto-alcohol)

¹H NMR (500 MHz, C₆D₆) δ 5.25 (dd, J=17.4, 10.7 Hz, 1H), 4.89 (dd, J=17.4, 0.9 Hz, 1H), 4.80 (dd, J=10.7, 0.9 Hz, 1H), 3.53-3.49 (m, 1H), 3.49-3.44 (m, 1H), 3.42-3.35 (m, 2H), 3.32-3.25 (m, 1H), 2.79 (d, J=11.9 Hz, 1H), 2.72 (s, 1H), 2.53 (dqd, J=14.3, 7.2, 4.0 Hz, 1H), 2.05 (dt, J=13.8, 7.1 Hz, 1H), 1.91 (ddd, J=26.4, 13.5, 3.7 Hz, 1H), 1.78 (d, J=5.8 Hz, 1H), 1.76 (d, J=5.8 Hz, 1H), 1.74-1.67 (m, 2H), 1.64-1.58 (m, 1H), 1.55 (d, J=7.1 Hz, 3H), 1.58-1.50 (m, 1H), 1.44 (ddd, J=13.3, 6.9, 3.6 Hz, 1H), 1.27 (s, 3H), 1.24 (s, 3H), 1.26-1.19 (m, 1H), 1.15 (s, 1H), 0.99 (d, J=7.1 Hz, 3H).

¹³C NMR (151 MHz, C₆D₆) δ 212.93, 146.71, 120.03, 114.39, 72.00, 64.21, 61.83, 51.56, 51.45, 49.08, 46.64, 46.35, 36.55, 35.53, 33.76, 30.14, 28.21, 27.02, 20.58, 17.03, 13.88, 10.94.

HRMS-ESI (m/z): calculated for [C₂₂H₃₅O₄]⁺ 363.2535, found 363.2530.

### Synthesis of the Diol S5:

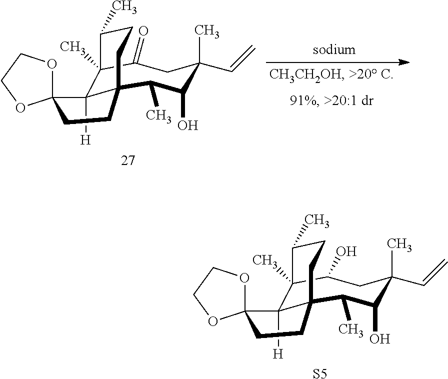

Freshly cut sodium metal (˜50 mg, excess) was added to a solution of the ketoalcohol 27 (9.0 mg, 24.8 μmol, 1 equiv) in ethanol (1.5 mL) at 20° C. CAUTION: THE ADDITION IS EXOTHERMIC. Additional freshly cut sodium metal (˜150 mg total) and ethanol (approx. 3 mL) were added as needed until no further conversion of the substrate was observed by thin-layer chromatography (which occurred at approximately 70% conversion and in 20 min). The product mixture was diluted sequentially with aqueous saturated aqueous ammonium chloride solution (2.0 mL) and water (2.0 mL). The diluted product mixture was extracted with ethyl acetate (3×5.0 mL). The organic layers were combined and the combined organic layers were dried over magnesium sulfate. The dried solution was filtered and the filtrate was concentrated. The residue obtained was dissolved in ethanol (1.5 mL) and resubjected to the above reaction conditions to achieve full conversion of the substrate. Purification of the mixture via preparatory thin-layer chromatography (eluting with 25% v/v ethyl acetate-hexanes) provided the diol S5 as a white solid (8.2 mg, 91%).

R_{f}=0.49 (30% v/v ethyl acetate-hexanes; PAA stains purple).

¹H NMR (500 MHz, C₆D₆) δ 5.31 (dd, J=17.7, 11.0 Hz, 1H), 4.83-4.77 (m, 2H), 4.35 (d, J=6.0 Hz, 1H), 3.61 (dd, J=12.7, 6.9 Hz, 1H), 3.53 (dd, J=13.1, 6.8 Hz, 1H), 3.51-3.43 (m, 1H), 3.40-3.34 (m, 1H), 3.18 (s, 2H), 2.77-2.67 (m,1H), 2.68-2.54 (m, 2H), 2.09 (q, J=7.0 Hz, 1H), 1.98-1.89 (m, 1H), 1.86-1.79 (m, 1H), 1.77-1.69 (m, 1H), 1.58-1.49 (m, 2H), 1.48-1.41 (m, 1H), 1.38 (s, 3H), 1.31 (s, 1H), 1.20 (ddd, J=12.7, 9.5, 3.4 Hz, 1H), 1.15 (d, J=7.3 Hz, 3H), 1.11 (d, J=15.0 Hz, 1H), 1.05 (d, J=7.0 Hz, 3H), 1.00 (s, 3H).

¹³C NMR (151 MHz, C₆D₆) δ 147.28, 121.76, 114.39, 84.43, 68.12, 63.58, 61.66, 51.26, 46.01, 44.41, 42.73, 40.28, 36.29, 35.65, 33.71, 33.18, 31.24, 28.82, 22.86, 20.70, 19.23, 14.21.

HRMS-ESI (m/z) calculated for [C₂₂H₃₇O₄]⁺ 365.2694, 365.2698.

### Synthesis of the Diol S6:

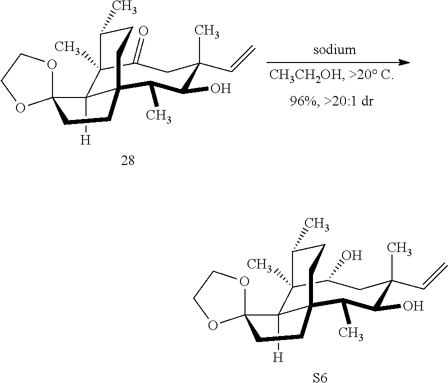

Freshly cut sodium metal (˜50 mg, excess) was added to a solution of the ketoalcohol 28 (5.6 mg, 15.5 μmol, 1 equiv) in ethanol (750 μL) at 20° C. CAUTION: THE ADDITION IS EXOTHERMIC. Additional freshly cut sodium metal (˜150 mg total) and ethanol (approx. 1.5 mL) were added as needed until no further conversion of the substrate was observed by thin-layer chromatography (which occurred at approximately 70% conversion and in 20 min). The reaction mixture was diluted sequentially with aqueous saturated aqueous ammonium chloride solution (2.0 mL) and water (2.0 mL). The diluted mixture was extracted with ethyl acetate (3×5.0 mL). The organic layers were combined and the combined organic layers were dried over magnesium sulfate. The dried solution was filtered and the filtrate was concentrated. The residue obtained was dissolved in ethanol (750 μL) and resubjected to the above reaction conditions to achieve full conversion of the substrate. Purification of the mixture via preparatory thin-layer chromatography (eluting with 30% v/v ethyl acetate-hexanes) provided the diol S6 as a white solid (5.4 mg, 96%).

R_{f}=0.30 (30% ethyl acetate-hexanes PAA stains blue).

¹H NMR (500 MHz, C₆D₆) δ 5.31 (dd, J=17.5, 10.7 Hz, 1H), 4.94 (dd, J=17.5, 1.3 Hz, 1H), 4.83 (dd, J=10.7, 1.3 Hz, 1H), 4.31 (d, J=8.1 Hz, 1H), 3.62-3.53 (m, 1H), 3.50-3.45 (m, 1H), 3.44-3.39 (m, 1H), 3.35 (d, J=6.4 Hz, 1H), 3.33-3.27 (m, 1H), 2.61-2.48 (m, 1H), 2.26-2.11 (m, 3H), 1.81-1.66 (m, 3H), 1.65-1.42 (m, 3H), 1.41 (tt, J=14.2, 4.1 Hz, 1H), 1.25 (s, 3H), 1.29-1.21 (m, 3H), 1.18 (d, J=15.4 Hz, 1H), 1.12 (s, 3H), 1.08 (d, J=7.2 Hz, 3H), 1.07 (d, J=7.2 Hz, 3H)

¹³C NMR (126 MHz, C₄D₆) δ 148.38, 120.86, 113.63, 71.90, 67.39, 63.85, 61.57, 52.20, 47.17, 46.38, 45.75, 43.12, 36.35, 36.12, 34.42, 29.58, 28.86, 28.05, 18.95, 14.03, 13.97, 11.80.

HRMS-ESI (m/z): calculated for [C₂₂H₃₇O₄]⁺ 365.2694, found 365.2700.

### Synthesis of the Diols S6 and S5:

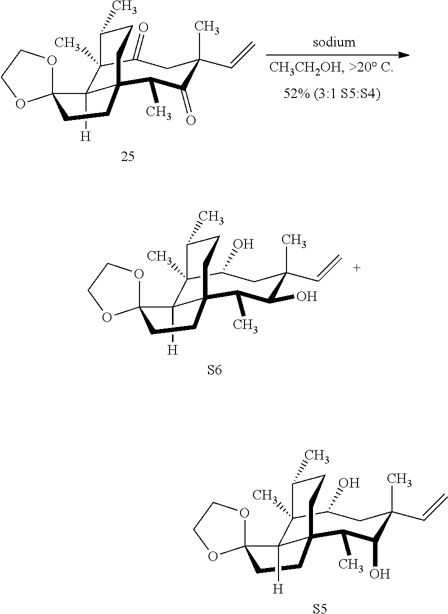

Freshly cut sodium metal (˜50 mg, excess) was added to a solution of the diketone 25 (5.0 mg, 13.9 μmol, 1 equiv) in ethanol (750 μL) at 20° C. CAUTION: THE ADDITION IS EXOTHERMIC. Additional freshly cut sodium metal (˜150 mg total) and ethanol (approx. 1.5 mL, total) were added as needed until no further conversion of the substrate was observed by thin-layer chromatography (which occurred at approximately 50% conversion and in 20 min). The reaction mixture was diluted sequentially with aqueous saturated ammonium chloride solution (2.0 mL) and water (2.0 mL). The diluted mixture was extracted with ethyl acetate (3×5.0 mL). The organic layers were combined and the combined organic layers were dried over magnesium sulfate. The dried solution was filtered and the filtrate was concentrated. The residue obtained was dissolved in ethanol (750 μL) and resubjected to the above reaction conditions to achieve full conversion of the substrate. The diols S6 and S5 were formed in a 3:1 ratio based on ¹H NMR analysis of the unpurified product mixture. Purification of the product mixture via preparatory thin-layer chromatography (eluting with 30% ethyl acetate-hexanes) afforded separately the diol S6 as a white solid (2.1 mg, 42%) and the diol S5 as a white solid (0.5 mg, 10%). The spectroscopic data for S5 and S6 were in agreement with those reported above.

Syntheses of (+)-12-epi-mutilin 4:

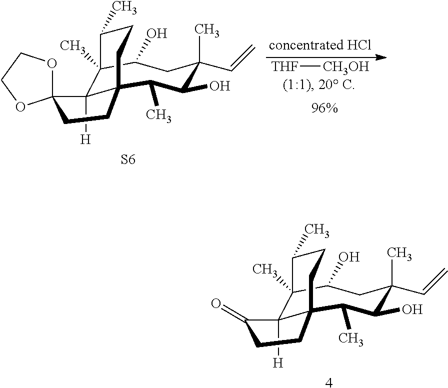

Concentrated aqueous hydrochloric acid solution (approximately 1.2 M, 50 μL) was added to a solution of 12-epi-mutilin-ketal S6 (2.5 mg, 6.86 μmol, 1 equiv) in tetrahydrofuran-methanol (1:1 v/v, 1.0 mL) at 20° C. The resulting mixture was stirred for 20 min open to air and then diluted with water (3.0 mL). The product mixture was extracted with ethyl acetate (3×3.0 mL). The organic layers were combined and the combined organic layers were dried over magnesium sulfate. The dried solution was filtered and the filtrate was concentrated to provide 12-epi-mutilin 4 as white solid (2.1 mg, 96%). The product so obtained was judged to be of >95% purity (¹H NMR analysis) and was used without further purification.

R_{f}=0.30 (30% ethyl acetate-hexanes; PAA stains blue).

¹H NMR (600 MHz, CDCl₃) δ 5.76 (dd, J=17.7, 10.6 Hz, 1H), 5.21 (m, 2H), 4.37 (d, J=7.7 Hz, 1H), 3.41 (d, J=6.7 Hz, 1H), 2.31-2.13 (m, 3H), 2.05 (s, 1H), 1.99 (dd, J=15.6, 7.8 Hz, 1H), 1.79 (dq, 15.4, 3.2 Hz, 1H), 1.73-1.38 (m, 5H), 1.35 (s, 3H), 1.20 (s, 3H), 1.23-1.10 (m, 2H), 0.98 (d, J=7.5 Hz, 3H), 0.95 (d, J=7.5 Hz, 3H).

¹³C NMR (151 Hz, CDCl₃) δ 217.83, 147.48, 114.99, 72.19, 66.66, 59.23, 46.19, 45.54, 45.33, 42.65, 37.04, 34.65, 34.61, 30.46, 27.36, 25.24, 18.36, 13.81, 13.56, 11.11.

HRMS-ESI (m/z): calculated for [C₂₀H₃₁O₂]⁺ 303.2319, found 303.2317.

α_{D}²⁰=+20+ (c=0.12, CHCl₃)

α_{D}²⁰=+34° (c=0.15, CHCl₃) (for 4 prepared by degradation of natural (+)-pleuromutilin)

Synthesis of (+)-11,12-di-epi-mutilin 26:

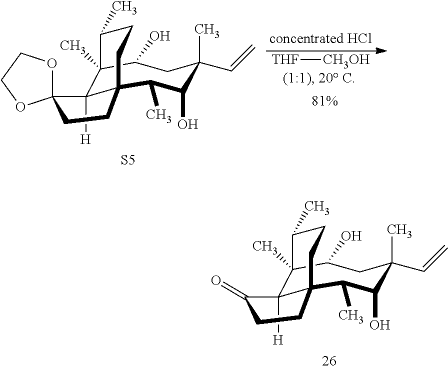

Concentrated aqueous hydrochloric acid solution (approximately 12 M, 50 μL) was added to a solution of 12-epi-mutilin-ketal S6 (2.1 mg, 5.76 μmol, 1 equiv) in tetrahydrofuran-methanol (1:1 v/v, 1.0 mL) at 20° C. The resulting mixture was stirred for 5 min open to air and then was diluted with water (3.0 mL). The diluted product mixture was extracted with ethyl acetate (3×3.0 mL). The organic layers were combined and the combined organic layers were dried over magnesium sulfate. The dried solution was filtered and then concentrated. The residue obtained was purified by preparative thin-layer chromatography (eluting with 25% ethyl acetate-hexanes) to provide 11,12-di-epi-mutilin 26 as a white solid (1.5 mg, 81%).

R_{f}=0.48 (25% ethyl acetate-hexanes; PAA stains purple).

¹H NMR (600 MHz, CDCl₃) δ 5.71 (dd, J=17.7, 11.0 Hz, 1H), 5.20 (d, J=11.0 Hz, 1H), 5.13 (d, J=17.7 Hz, 1H), 4.39 (d, J=5.3 Hz, 1H), 3.45 (s, 1H), 2.98 (s, 1H), 2.44 (dd, J=22.5, 10.4 Hz, 1H), 2.36 (dd, J=15.2, 6.4 Hz, 1H), 2.25-2.08 (m, 4H), 2.06-1.97 (m, 1H), 1.71-1.55 (m, 3H), 1.42 (s, 3H), 1.39-1.24 (m, 2H), 1.20 (s, 3H), 1.13 (d, J=7.1 Hz, 3H), 1.13-1.06 (m, 2H), 1.00 (d, J=7.1 Hz, 3H).

¹³C NMR (151 MHz, CDCl₃) δ 220.31, 146.99, 115.18, 84.17, 67.50, 59.34, 45.15, 44.10, 42.54, 39.07, 37.47, 35.12, 33.69, 32.94, 27.90, 27.59, 22.53, 19.97, 18.56, 13.94.

HRMS-ESI (m/z): calculated for [C₂₀H₃₃O₃]⁺ 321.2430, found 321.2422.

α_{D}²⁰=+1.4° (c=0.03, CHCl₃)

### Synthesis of Ester S8:

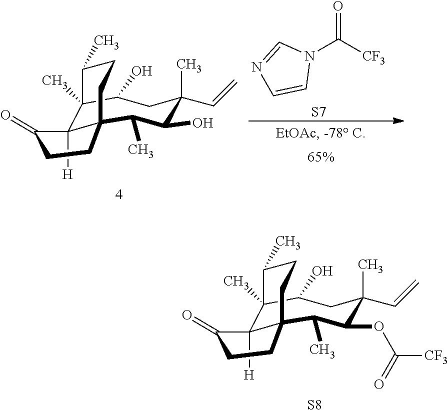

A solution of 12-epi-mutilin 4 (17.7 mg, 55.2 μmol, 1 equiv) in ethyl acetate (1.0 mL) was cooled at −78° C. for 5 min. 1-(Trifluoroacetyl)imidazole S7 (37.7 μL, 331 μmol, 6.00 equiv) was added dropwise and the resulting mixture was stirred at −78° C. for 50 min. The resulting mixture was diluted with aqueous hydrochloric acid solution (1 M, 200 μL) and then was warmed to 20° C. for 1 h. The product mixture was diluted with aqueous hydrochloric acid solution (1 M, 1 mL) and then extracted with ethyl acetate (3×5 mL). The organic layers were combined and the combined organic layers were dried over sodium sulfate. The dried solution was filtered and the filtrate was concentrated. The residue obtained was purified by preparative thin-layer chromatography (eluting with 40% ether-pentane) to provide the ester SS as a white solid (15.0 mg, 65%).

R_{f}=0.65 (40% ether-pentane, PAA stains purple)

¹H NMR (500 MHz, CDCl₃) δ 5.62 (dd, J=17.4, 10.8 Hz, 1H), 5.12-4.98 (m, 3H), 4.39-4.33 (m, 1H), 2.53 (p, J=7.2 Hz, 1H), 2.38-2.03 (m, 4H), 1.82-1.66 (m, 3H), 1.60-1.40 (m, 3H), 1.38 (s, 3H), 1.33 (s, 3H), 1.29-1.13 (m, 2H), 0.99 (d, J=7.1 Hz, 3H), 0.83 (d, J=7.1 Hz, 3H).

¹⁹F NMR (470 MHz, CDCl₃) δ 75.09 (s, 3F).

¹³C NMR (151 MHz, CDCl₃) δ 216.75, 156.81 (q, J=42.0 Hz), 144.96, 114.78 (q, J=286.1 Hz), 114.05, 80.10, 66.36, 59.17, 59.17, 45.97, 43.85, 42.70, 36.95, 34.95, 34.46, 30.39, 27.26, 25.30, 18.31, 14.99, 13.49, 11.60.

HRMS-ESI (m/z): calculated for [C₂₂H₃₁F₃O₄Na]⁺ 439.2067, found 439.2046.

Synthesis of (+)-12-epi-pleuromutilin (29):

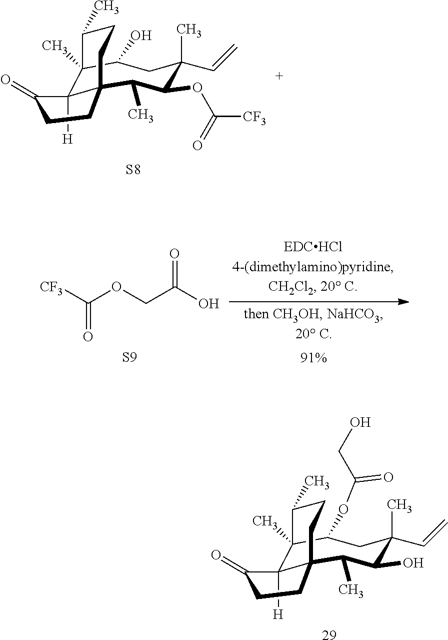

Trifluoroacetylglycolic acid (5.2 mg, 30.1 μmol, 3.30 equiv) was added dropwise via syringe to a stirring solution of the ester S8 (3.8 mg, 9.12 μmol, 1 equiv), N-(3-dimethylaminopropyl)-N′-ethylcarbodiimide hydrochloride (EDC.HCl, 4.7 mg, 30.1 μmol, 3.30 equiv) and 4-(dimethylamino)pyridine (3.7 mg, 30.1 μmol, 3.30 equiv) in dichloromethane (500 μL) at 20° C. under air. The resulting mixture was stirred at 20° C. for 30 min and then methanol (500 μL) and sodium bicarbonate (20.0 mg, 238 μmol, 26.1 equiv) were added in sequence. The resulting mixture was stirred at 20° C. for 22 h. The product mixture was diluted with saturated aqueous ammonium chloride solution (1.5 mL) and the diluted mixture was extracted with ethyl acetate (3×1.5 mL). The organic layers were combined and the combined organic layers were dried over sodium sulfate. The dried solution was filtered and the filtrate was concentrated. The residue obtained was dissolved in methanol (200 μL) and then sodium bicarbonate (12.0 mg, 143 μmol, 15.6 equiv) was added at 20° C. The resulting solution was stirred for 21 h at 20° C. The product mixture was diluted with saturated aqueous ammonium chloride solution (1.5 mL) and the diluted solution was extracted with ethyl acetate (3×1.5 mL). The organic layers were combined and the combined organic layers were dried over sodium sulfate. The dried solution was filtered and the filtrate was concentrated. The residue obtained was purified by preparative thin-layer chromatography (eluting with 40% ethyl acetate-pentane) to provide 12-epi-pleuromutilin 29 as a white solid (3.2 mg, 91%).

R_{f}=0.22 (40% ethyl acetate in pentane, PAA stains purplish-blue)

¹H NMR, (600 MHz, CDCl₃) δ 5.73 (m, 2H), 5.22 (m, 2H), 4.04 (dq, J=16.9, 5.3 Hz, 2H), 3.45 (d, J=6.4 Hz, 1H), 2.42-2.03 (m, 5H), 1.85-1.36 (m, 6H), 1.44 (s, 3H), 1.25 (s, 3H), 1.17-1.10 (m, 2H), 0.98 (d, J=7.1 Hz, 3H), 0.70 (d, J=7.0 Hz, 3H).

¹³C NMR (151 MHz, CDCl₃) δ 217.07, 172.29, 146.93, 115.52, 72.06, 70.24, 61.44, 58.34, 45.54, 45.41, 43.75, 42.00, 36.75, 34.62, 34.54, 30.26, 27.07, 25.14, 16.87, 14.98, 14.30, 10.99.

HRMS-ESI (m/z): calculated for [C₂₂H₃₄NaO₅]⁺ 401.2298, found 401.2297.

α_{D}²⁰=+36 (c=0.36, CHCl₃)

α_{D}²⁰=+37 (c=0.15, CHCl₃) (for 29 prepared by degradation of natural (+)-pleuromutilin)

Synthesis O-trityl-12-epi-pleuromutilin (30):

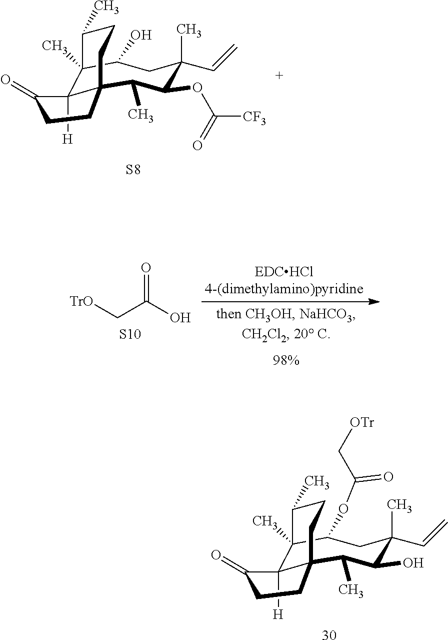

O-tritylglycolic acid (10.3 mg, 32.5 μmol, 3.30 equiv) was added to a stirring solution of the ester S8 (4.1 mg, 9.84 μmol, 1 equiv), N-(3-dimethylaminopropyl)-N′-ethylcarbodiimide hydrochloride (EDC.HCl, 5.0 mg, 32.5 μmol, 3.30 equiv) and 4-(dimethylamino)pyridine (4.0 mg, 32.5 μmol, 3.30 equiv) in dichloromethane (500 μL) at 20° C. under air. The resulting mixture was stirred at 20° C. for 30 min and then methanol (500 μL) and sodium bicarbonate (20.0 mg, 238 μmol, 24.2 equiv) were added in sequence. The resulting mixture was stirred at 20° C. for 46 h. The product mixture was diluted with saturated aqueous ammonium chloride solution (1.5 mL) and the diluted product mixture was extracted with ethyl acetate (3×1.5 mL). The organic layers were combined and the combined organic layers were dried over sodium sulfate. The dried solution was filtered and the filtrate was concentrated. The residue obtained was purified by preparative thin-layer chromatography (eluting with 30% ethyl acetate-pentane) to provide O-trityl-12-epi-pleuromutilin 30 as a white solid (6.0 mg, 98%).

R_{f}=0.43 (40% ethyl acetate in pentane, PAA stains green)

¹H NMR (600 MHz, CDCl₃) δ 7.49-7.43 (m, 6H), 7.33-7.21 (m, 9H), 5.72 (dd, J=17.4, 10.8 Hz, 1H), 5.67 (d, J=8.4 Hz, 1H), 5.25-5.17 (m, 2H), 3.75 (d, J=15.8 Hz, 1H), 3.65 (d, J=15.9 Hz, 1H), 3.42 (d, J=6.3 Hz, 1H), 2.40 (p, J=7.0 Hz, 1H), 2.29-2.14 (m, 2H), 2.09 (s, 1H), 1.98 (dd, J=15.9, 8.4 Hz, 1H), 1.80 (dd, J=14.6, 3.1 Hz, 1H), 1.68-1.35 (m, 5H), 1.42 (s, 3H), 1.23 (s, 3H), 1.12 (td, J=13.9, 4.8 Hz, 1H), 1.00-0.93 (m, 4H), 0.69 (d, J=6.8 Hz, 3H).

¹³C NMR (151 MHz, CDCl₃) δ 217.31, 169.02, 147.13, 143.41, 128.72, 128.11, 127.40, 115.30, 87.51, 72.11, 68.93, 63.35, 58.45, 45.55, 45.39, 43.72, 41.98, 36.91, 34.66, 34.46, 30.32, 27.09, 25.12, 16.99, 15.11, 14.31, 10.89.

HRMS-ESI (m/z): calculated for [C₄₁H₄₈NaO₅]⁺ 643.3394, found 643.3395.

Synthesis of (+)-12-epi-pleuromutilin (29) and (+)-pleuromutilin (1):

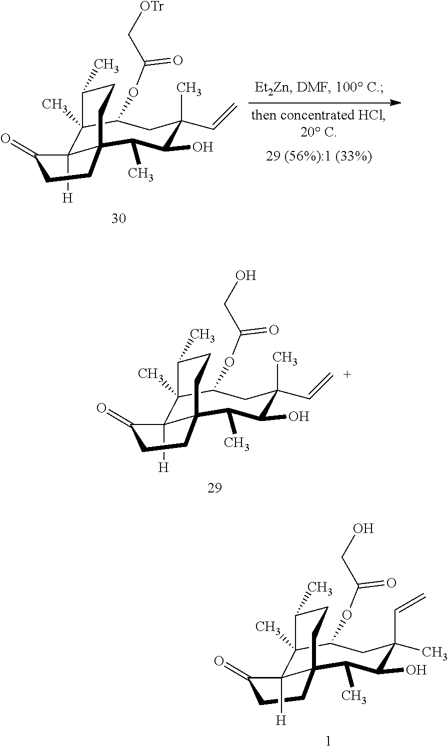

A solution of diethyl zinc in hexanes (1.0 M, 15.0 μL, 15.0 μmol, 1.03 equiv) was added to a solution of O-trityl-12-epi-pleuromutilin 30 (9.0 mg, 14.5 μmol, 1 equiv) in N,N-dimethylformamide (150 μL) at 20° C. The resulting mixture was heated at 100° C. for 2 h and then was cooled to 20° C. over 5 min. Concentrated aqueous hydrochloric acid solution (approximately 12 M, 50 μL) was added and the resulting mixture was stirred for 18 h at 20° C. The product mixture was diluted with saturated aqueous ammonium chloride solution (1.5 mL) and the diluted mixture was extracted with ethyl acetate (3×1.5 mL). The organic layers were combined and the combined organic layers were dried over sodium sulfate. The dried solution was filtered and the filtrate was concentrated. The residue obtained was purified by preparative thin-layer chromatography (eluting with 25% ethyl acetate-dichloromethane, two elutions) to provide separately (+)-pleuromutilin 1 (1.8 mg, 33%) and 12-epi-pleuromutilin 29 (3.1 mg, 56%) as white solids. The spectroscopic data for 1 were agreement with those obtained for a commercial sample.

1:

R_{f}=0.28 (25% ethyl acetate-dichloromethane, PAA stains greenish brown.)

¹H NMR (400 MHz, CDCl₃) δ 6.50 (dd, J=17.4, 11.0 Hz, 1H), 5.85 (d, J=8.6 Hz, 1H), 5.37 (dd, J=11.0, 1.5 Hz, 1H), 5.22 (dd, J=17.4, 1.5 Hz, 1H), 4.04 (qd, J=17.1, 5.4 Hz, 2H), 3.37 (d, J=6.5 Hz, 1H), 2.29-2.41 (m, 1H), 2.17-2.19 (m, 2H), 2.11 (s, 1H), 2.06-2.16 (m, 1H), 1.78 (dd, J=14.4, 2.9 Hz, 1H), 1.63-1.74 (m, 2H), 1.51-1.61 (m, 1H), 1.45-1.55 (m, 1H), 1.44 (s, 3H), 1.35-1.43 (m, 1H), 1.32 (d, J=16.2 Hz, 1H), 1.18 (s, 3H), 1.08-1.18 (m, 1H), 0.90 (d, J=7.0 Hz, 3H), 0.71 (d, J=7.1 Hz, 3H).

¹³C NMR (151 MHz, CDCl₃) δ 217.00, 172.33, 138.95, 117.59, 74.73, 69.98, 61.48, 58.24, 45.59, 44.88, 44.17, 41.99, 36.76, 36.20, 34.59, 30.55, 26.99, 26.46, 25.00, 16.80, 14.94, 11.71.

α_{D}²⁰=+32° (c=0.25, CHCl₃)

lit. α_{D}²³=+33° (c=0.2, CDCl₃) (15)

29:

The spectroscopic data were in agreement with those reported above.

Comparison of ¹H NMR Data of Natural and Synthetic (+)-pleuromutilin (1)*

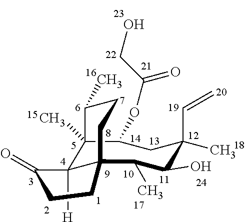

| | Partial ¹H NMR | Complete ¹H NMR | ¹H NMR |
|---|---|---|---|
| | Natural (+)-1 | Natural (+)-1 | Synthetic (+)-1 |
| Position | (CDCl₃) | (CDCl₃) | (CDCl₃) |
| 1α | —^{†} | 1.41-1.53 (m) | 1.45-1.55 (m) |
| 1β | —^{†} | 1.61-1.73 (m) | 1.63-1.74 (m) |
| 2α | —^{†} | 2.16-2.30 (m) | 2.17-2.29 (m) |
| 2β | —^{†} | 2.16-2.30 (m) | 2.17-2.29 (m) |
| 3 | —^{†} | | |
| 4 | —^{†} | 2.11 (s) | 2.11 (s) |
| 5 | | | |
| 6 | —^{†} | 1.61-1.73 (m) | 1.63-1.74 (m) |
| 7α | —^{†} | 1.51-1.61 (m) | 1.51-1.61 (m) |
| 7β | —^{†} | 1.35-1.43 (m) | 1.35-1.43 (m) |
| 8α | —^{†} | 1.79 (dd, 14.5, 2.9) | 1.78 (dd, 14.4, 2.9) |
| 8β | —^{†} | 1.09-1.22 (m) | 1.08-1.18 (m |
| 9 | | | |
| 10 | —^{†} | 2.29-2.40 (m) | 2.29-2.41 (m) |
| 11 | 3.38 (dd, 7, 6) | 3.34 (br) | 3.37 (d, 6.5) |
| 12 | | | |
| 13α | —^{†} | 2.05-2.16 (m) | 2.06-2.16 (m) |
| 13β | —^{†} | 1.33 (d, 16.1) | 1.32 (d, 16.2) |
| 14 | 5.84 (d, 8) | 5.85 (d, 8.6) | 5.85 (d, 8.6) |
| 15 | 1.45 (s) | 1.44 (s) | 1.44 (s) |
| 16 | 0.72 (d, 6) | 0.71 (d, 7.1) | 0.71 (d, 7.1) |
| 17 | 0.91 (d, 7) | 0.90 (d, 7.0) | 0.90 (d, 7.0) |
| 18 | 1.18 (s) | 1.18 (s) | 1.18 (s) |
| 19 | 6.50 (dd, 17, 11) | 6.50 (dd, 17.4, 11.0) | 6.50 (dd, 17.4, 11.0) |
| 20α | 5.36 (dd, 11.0, 1.5) | 5.37 (dd, 11.0, 1.0) | 5.37 (dd, 11.0, 1.5) |
| 20β | 5.21 (dd, 15, 1.5) | 5.22 (dd, 17.4, 1.0) | 5.22 (dd, 17.4, 1.5) |
| 21 | | | |
| 22 | 4.05 (d, 5) | 4.04 (qd, 17.1, 5.4) | 4.04 (qd, 17.1, 5.4) |
| 23 | 2.60 (t, 6) | 2.52 (br) | —^{†} |
| 24 | —^{†} | 1.44-1.55 (br) | —^{†} |

*Partial data for natural (+)-pleuromutilin obtained from ref. Complete data for
natural (+)-pleuromutilin obtained based on analysis of a commerical sample
by heteronuclear single quantum coherence spectroscopy (HSQC ¹H—¹³C)
and comparison with ¹³C NMR data reported for natural (+)-pleuromutilin in
ref. (¹¹). ^{†} not reported

Comparison of ¹³C NMR Data of Natural and Synthetic (+)-pleuromutilin (1)*

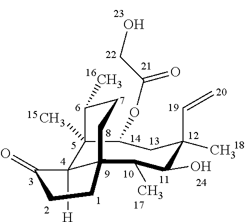

| | ¹³C NMR Natural (+)-1 | ¹³C NMR Synthetic (+)-1 |
|---|---|---|
| Position | (CDCl₃) | (CDCl₃) |
| 1 | 24.9 | 25.0 |
| 2 | 34.5 | 34.6 |
| 3 | 216.8 | 217.0 |
| 4 | 58.2 | 58.2 |
| 5 | 41.9 | 42.0 |
| 6 | 36.7 | 36.8 |
| 7 | 26.9 | 27.0 |
| 8 | 30.4 | 30.5 |
| 9 | 45.5 | 45.6 |
| 10 | 36.1 | 36.2 |
| 11 | 74.7 | 74.7 |
| 12 | 44.1 | 44.2 |
| 13 | 44.9 | 44.9 |
| 14 | 69.9 | 70.0 |
| 15 | 14.8 | 14.9 |
| 16 | 16.6 | 16.8 |
| 17 | 11.5 | 11.7 |
| 18 | 26.5 | 26.5 |
| 19 | 138.9 | 139.0 |
| 20 | 117.3 | 117.6 |
| 21 | 172.2 | 172.3 |
| 22 | 61.3 | 61.5 |

*Data for natural (+)-pleuromutilin obtained from ref. (11).

Synthesis of O-trityl-11,12-di-epi-pleuromutilin (S11):

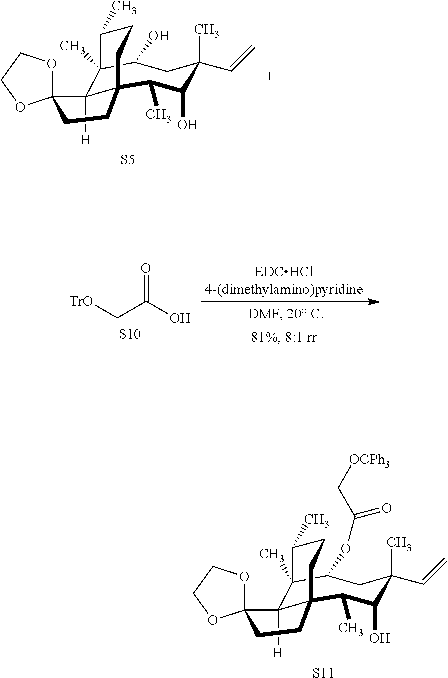

To a solid mixture of the diol S5 (5.4 mg, 14.8 μmol, 1 equiv), O-tritylglycolic acid S10 (28.3 mg, 88.9 μmol, 6.00 equiv), 4-(dimethylamino)pyridine (25.3 mg, 207.4 μmol, 16.0 equiv), and N-(3-dimethylaminopropyl)-N′-ethylcarbodiimide hydrochloride (EDC.HCl, 17.0 mg, 88.9 μmol, 6.00 equiv) was added N,N-dimethylformamide (450 μL) at 20° C. The resulting mixture was stirred for 1.8 h at 20° C. The product mixture was diluted with saturated aqueous sodium chloride solution (5.0 mL) and the diluted mixture was extracted with ethyl acetate (3×3.0 mL). The organic layers were combined and the combined layers were dried over magnesium sulfate. The dried solution was filtered and the filtrate was concentrated. The residue obtained was purified by preparative thin-layer chromatography (eluting with 25% ethyl acetate-hexanes) to provide O-trityl-11,12-di-epi-pleuromutilin (S11) as a white solid (7.9 mg, 81%, 8:1 rr, inseparable regioisomers). The mixture was used directly in the next step.

R_{f}=0.48 (25% ethyl acetate-hexanes; PAA stains green).

¹H NMR (400 MHz, C₆D₆) δ 7.58 (d, J=7.6 Hz, 6H), 7.07 (t, J=7.6 Hz, 6H), 6.98 (t, J=7.3 Hz, 3H), 5.98 (d, J=7.7 Hz, 1H), 5.25 (dd, J=17.6, 11.1 Hz, 1H), 4.74 (d, J=11.1, 1H), 4.72 (d, J=17.6, 1H), 4.00 (d, J=15.4 Hz, 1H), 3.90 (d, J=15.4 Hz, 1H), 3.61-3.34 (m, 4H), 3.32 (s, 1H), 3.19 (s, 1H), 2.79-2.54 (m, 3H), 2.30 (q, J=7.1 Hz, 1H), 2.17-2.03 (m, 1H), 2.00-1.88 (m, 1H), 1.55 (s, 3H), 1.27 (s, 3H), 1.61-0.85 (m, 7H), 1.02 (d, J=6.8 Hz, 3H), 1.00 (d, J=6.8 Hz, 3H).

Synthesis of (+)-11,12-di-epi-pleuromutilin (31):

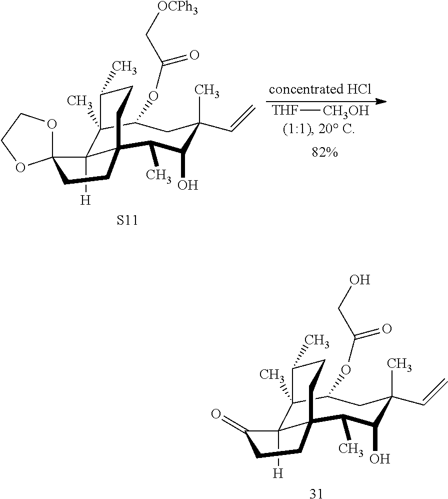

Concentrated hydrochloric acid (approximately 12 M, 50 μL) was added to a solution of O-trityl-11,12-di-epi-pleuromutilin (S11, 1.7 mg, 4.19 μmol, 1 equiv) tetrahydrofuran-methanol (1:1 mixture, 1.0 mL) at 20° C. The resulting mixture was stirred for 1 h open to air and then diluted with water (3.0 mL). The dilated product mixture was extracted with ethyl acetate (3×3.0 mL). The organic layers were combined and the combined organic layers were dried over magnesium sulfate. The dried solution was filtered and the filtrate was concentrated. The residue obtained was purified via preparative thin-layer chromatography (eluting with 30% ethyl acetate-hexanes) to provide 11,12-di-epi-pleuromutilin (31) as a white solid (1.3 mg, 82%).

R_{f}=0.25 (30% v/v ethyl acetate-hexanes; UV; PAA stains black).

¹H NMR (600 MHz, CDCl₃) δ 5.80 (d, J=7.3 Hz, 1H), 5.69 (dd, J=17.7, 11.0 Hz, 1H), 5.19 (d, J=11.0 Hz, 1H), 5.09 (d, J=17.7 Hz, 1H), 4.09 (dd, J=17.0, 5.4 Hz, 1H), 4.02 (dd, J=17.0, 5.4 Hz, 1H), 3.47 (s, 1H), 3.10 (s, 1H), 2.53-2.41 (m, 2H), 2.34 (t, J=5.4 Hz, 1H), 2.31 (q, J=7.1 Hz, 1H), 2.25-2.08 (m, 3H), 1.70-1.57 (m, 3H), 1.50 (s, 3H), 1.39-1.32 (m, 2H), 1.22 (s, 3H), 1.15 (d, J=7.1 Hz, 1H), 1.12-1.07 (m, 1H), 1.08 (d, J=15.1 Hz, 2H), 0.70 (d, J=6.6 Hz, 3H).

¹³C NMR (151 MHz, CDCl₃) δ 219.63, 172.26, 146.69, 115.11, 84.19, 71.05, 61.57, 58.54, 45.12, 44.05, 42.29, 37.18, 37.11, 35.19, 33.42, 32.85, 27.49, 27.45, 23.62, 19.85, 16.80, 15.32.

HRMS-ESI (m/z): calculated for [C₂₂H₃₅O₅]⁺ 379.2485, found 379.2495.

α_{D}²⁰=+114° (c=0.04, CHCl₃)

### General Experimental Procedures—Second Set of Experiments

All reactions were performed in single-neck, oven- (>140° C.) or flame-dried, round-bottomed flasks fitted with rubber septa under a positive pressure of argon, unless otherwise noted. Air- and moisture-sensitive liquids were transferred via syringe or stainless steel cannula, or were handled in a nitrogen-filled drybox (working oxygen level <5 ppm). Nitrogen-sensitive titanium complexes were stored and handled in an argon-filled drybox (working oxygen level <5 ppm). Flash-column chromatography was performed as described by Still et al.,¹ using silica gel (60 Å, 40-63 μm particle size) purchased from SiliCycle. Analytical thin-layered chromatography (TLC) was performed using glass plates pre-coated with silica gel (0.25 mm, 60 Å pore size) impregnated with a fluorescent indicator (254 nm). TLC plates were visualized by exposure to ultraviolet light (UV) and/or submersion in aqueous p-anisaldehyde solution (PAA) or aqueous potassium permanganate solution (KMnO₄), followed by brief heating with a heat gun.
Materials. Dichloromethane, N,N-dimethylformamide, ether, hexanes, pentane, tetrahydrofuran, and toluene were purified according to the method of Pangborn et al.² Methanol and ethanol were deoxygenated by sparging with nitrogen and then dried over 3 Å molecular sieves before use. Water and N-methyl-2-pyrrolidinone were deoxygenated by sparging with nitrogen before use. The molarity of organozinc solutions was determined by titration against a standard solution of iodine and lithium chloride in tetrahydrofuran (average of three determinations).³ The molarity of t-butyllithium solutions was determined by titration against a standard solution of menthol and 1,10-phenanthroline in tetrahydrofuran (average of three determinations).⁴ Molecular sieves were activated by heating to 200° C. under vacuum (<1 Torr) for 12 h, and were stored in either an oven at >140° C. or a nitrogen-filled glovebox. Feringa's phosphoramidite ligand (L₁),⁵ the oxazolidinone 28,⁶p-methoxybenzyl chloromethyl ether,⁷ dimethyl-1-diazo-2-oxopropylphosphonate (Ohira-Bestmann reagent),⁸ 2-iodoxybenzoic acid (IBX),⁹ Dess-Martin periodinane (DMP),¹⁰ 4,5-dichloro-1,3-bis(2,6-diisopropylphenyl)imidazol-2-ylidene (IPr^{Cl}),¹¹ bis(cyclopentadienyl)bis(trimethyl-phosphine)titanium,¹² trifluoroacetyl-glycolic acid (S17),¹³ and O-tritylglycolic acid (S18)¹⁴ were prepared according to literature procedures. Thionyl chloride was purified by fractional distillation. All other commercial reagents were used as received.
Equipment. Proton nuclear Magnetic resonance spectra (¹H NMR) were recorded at 400, 500 or 600 MHz at 24° C. Chemical shifts are expressed in parts per million (ppm, δ scale) downfield from tetramethylsilane and are referenced to residual protium in the NMR solvent (CHCl₃, δ 7.26; C₆D₅H, δ 7.15). Data are represented as follows: chemical shift, multiplicity (s=singlet, d=doublet, t=triplet, q=quartet, m=multiplet and/or multiple resonances, br=broad), integration, coupling constant in Hertz, and assignment. Proton-decoupled carbon nuclear magnetic resonance spectra (¹³C NMR) were recorded at 100, 125, or 151 MHz at 24° C., unless otherwise noted. Chemical shifts are expressed in parts per million (ppm, δ scale) downfield from tetramethylsilane and are referenced to the carbon resonances of the solvent (CDCl₃, δ 77.0; C₆D₆, 128.0). ¹³C NMR data are represented as follows: chemical shift. Proton-decoupled fluorine nuclear magnetic resonance (¹⁹F NMR) spectra were recorded at 376 or 470 MHz at 24° C. ¹⁹F NMR data are represented as follows: chemical shift. High-resolution mass spectra (HRMS) were obtained on a Waters UPLC/HRMS instrument equipped with a dual API/ESI high-resolution mass spectrometry detector and photodiode array detector. Unless otherwise noted, samples were eluted over a reverse-phase BEH C18 column (1.7 μm particle size, 2.1×50 mm) with a linear gradient of 5% acetonitrile-water containing 0.1% formic acid→95% acetonitrile-water containing 0.1% formic acid over 1.6 min, followed by 100% acetonitrile containing 0.1% formic acid for 1 min, at a flow rate of 600 μL/min. Optical rotations were measured on a Perkin Elmer polarimeter equipped with a sodium (589 nm, D) lamp. Optical rotation data are represented as follows: specific rotation (α₂₀^{D}), concentration (g/mL), and solvent. HPLC data were obtained on an Agilent 1100 Series HPLC system equipped with a photodiode array detector.

### Synthetic Procedures.

A transformation is considered a single step if the reaction mixture remains in the reaction flask and is not subjected to rotary evaporation, aqueous workup, or any level of purification.

### Synthesis of β-ketoester 19:

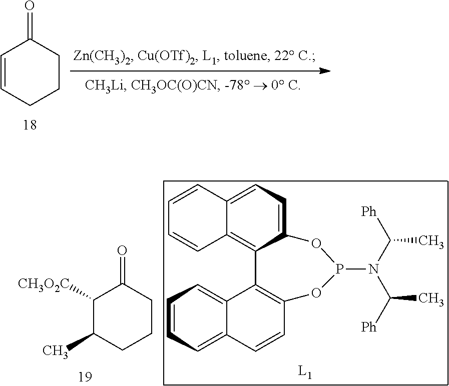

A suspension of copper(II) bis(trifluoromethansulfonate) (207 mg, 572 μmol, 0.500 mol %) and L₁ (618 mg, 1.14 mmol, 1.00 mol %) in toluene (160 mL) was stirred for 30 min at 22° C. The resulting solution was cooled to 0° C. for 20 min and then cyclohex-2-en-1-one (18, 11.1 mL, 114 mmol, 1 equiv) was added. A solution of dimethylzinc in toluene (1.2 M, 100 mL, 120 mmol, 1.05 equiv) was then added dropwise over 20 min and the resulting mixture was stirred for an additional 30 min at 22° C. The resulting mixture was cooled to −78° C. for 20 min and then a solution of methyllithium in ether (1.6 M, 75.1 mL, 120 mmol, 1.05 equiv) was added dropwise over 5 min. After stirring for an additional 5 min, methyl cyanoformate (10.9 mL, 137 mmol, 1.20 equiv) was added. The resulting solution was stirred at −78° C. for 2 h and then was allowed to warm to 0° C. over 30 min. The warmed product mixture was diluted sequentially with saturated aqueous ammonium chloride solution (40 mL) and water (200 mL). The diluted product mixture was further warmed to 22° C. over 30 min. The warmed mixture was extracted with ethyl acetate (3×200 mL) and the organic extracts were combined. The combined organic extracts were washed with saturated aqueous sodium chloride solution (200 mL). The washed solution was dried over magnesium sulfate. The dried solution was filtered and the filtrate was concentrated. ¹H spectroscopic data for the product 19 were in agreement with those previously reported.¹⁵ The unpurified residue was used directly in the following step.

Synthesis of the α-methyl β-ketoester 20:

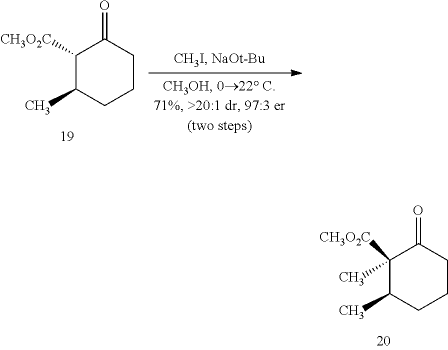

Iodomethane (35.6 mL, 572 mmol, 5.00 equiv) and sodium t-butoxide (22.0 g, 229 mmol, 2.00 equiv) were added in sequence to a solution of the residue obtained in the preceding step (nominally 114 mmol) in methanol (230 mL) at 0° C. The resulting solution was allowed to warm to 22° C. over a period of 12 h. The product mixture was concentrated. The residue obtained was treated with saturated aqueous ammonium chloride solution (200 mL), and the resulting mixture was extracted with ether (3×200 mL). The organic layers were combined and the combined layers were dried over magnesium sulfate. The dried solution was filtered and the filtrate was concentrated. The residue obtained was purified by flash-column chromatography (eluting with 5% ether-hexanes) to provide the α-methyl β-ketoester 20 as a colorless oil (14.9 g, 71%).

The enantiomeric ratio of the α-methyl β-ketoester 20 was determined to be 97:3.¹⁵

R_{f}=0.30 (5% ethyl acetate-hexanes; KMnO₄).

¹H NMR (400 MHz, CDCl₃) δ 3.69 (s, 3H), 2.72 (td, J=14, 6.8 Hz, 1H), 2.47-2.39 (m, 1H), 2.08-1.97 (m, 1H), 1.97-1.83 (m, 1H), 1.72-1.58 (m, 3H), 1.34 (s, 3H), 1.14 (d, J=6.4 Hz, 3H).

¹³C NMR (126 MHz, CDCl₃) δ 208.3, 171.8, 60.9, 51.9, 43.8, 39.8, 30.2, 25.5, 18.8, 17.0.

Synthesis of the α-methyl-β-ketoester 20 (One-Step Procedure):

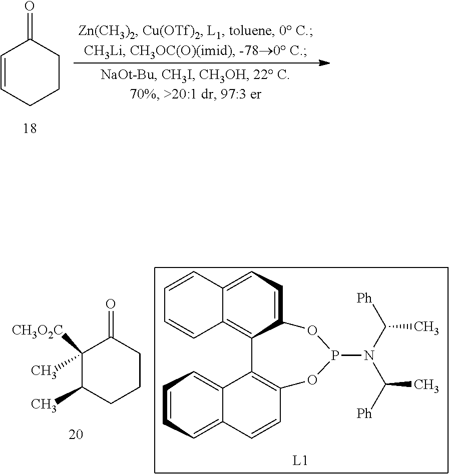

A suspension of copper(II) bis(trifluoromethansulfonate) (94.0 mg, 260 μmol, 0.500 mol %) and L₁ (281 mg, 520 μmol, 1.00 mol %) in toluene (70 mL) was stirred for 30 min at 22° C. The resulting solution was cooled to 0° C. for 20 min and then cyclohex-2-ene4-one (18, 5.04 mL, 52.0 mmol, 1 equiv) was added. A solution of dimethylzinc in toluene (1.2 M, 46.8 mL, 56.2 mmol, 1.08 equiv) was then added dropwise over 20 min and the resulting mixture was stirred for an additional 20 min at 0° C. The resulting mixture was cooled to −78° C. for 20 min and then a solution of methyllithium in ether (1.6 M, 35.1 mL, 56.2 mmol, 1.08 equiv) was added dropwise over 10 min. The resulting mixture was stirred for 5 min at −78° C. A solution N-carbomethoxyimidazole in toluene (4.33 M, 15.0 mL, 65.0 mmol, 1.25 equiv) was then added dropwise over 10 min. The resulting solution was stirred at −78° C. for 10 min and then allowed to warm to −30° C. over 2 h. The mixture was then further warmed to 0° C. over 2 h. The warmed mixture was slowly diluted with methanol (100 mL) and then cooled to 0° C. for 20 min. Iodomethane (16.2 mL, 260 mmol, 5.00 equiv) and sodium t-butoxide (9.97 g, 104 mmol, 2.00 equiv) were then added in sequence. The resulting solution was allowed to warm to 22° C. over 14 h. The product mixture was diluted with aqueous citric acid solution (10% w/v, 400 mL) and the resulting mixture was extracted with ether (3×150 mL). The organic layers were combined and the combined organic layers were dried over magnesium sulfate. The dried solution was filtered and the filtrate was concentrated. The residue obtained was purified by flash-column chromatography (eluting with 5% ether-hexanes) to provide the α-methyl β-ketoester 20 as a colorless oil (6.71 g, 70%). The purity of the material was determined to be >95% by quantitative ¹H NMR analysis. Spectroscopic data for the α-methyl β-ketoester 20 obtained in this way were in agreement with those previously reported.¹⁵

### Synthesis of the Vinyl Triflate 21:

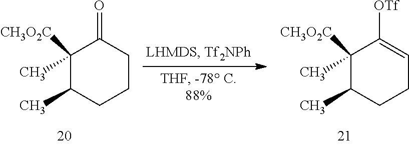

A solution of potassium bis(trimethylsilyl)amide in toluene (0.5 M, 200 mL, 1.50 equiv) was added dropwise over 10 min to a solution of the α-methyl β-ketoester 20 (12.2 g, 66.2 mmol, 1 equiv) and N-phenyl bis(trifluoromethanesulfonimide) (28.4 g, 49.5 mmol, 1.20 equiv) in tetrahydrofuran (400 mL) at −78° C. The resulting solution was stirred for 50 min at −78° C. The cold product mixture was diluted with saturated aqueous ammonium chloride solution (200 mL) and the diluted mixture was allowed to warm to 22° C. over 20 min. The warmed product mixture was extracted with ethyl acetate (3×200 mL). The organic layers were combined and the combined organic layers were dried over magnesium sulfate. The dried solution was filtered and the filtrate was concentrated. The residue obtained was purified by flash-column chromatography (eluting with 25% dichloromethane-hexanes initially, grading to 50% dichloromethane-hexanes, four steps) to provide the vinyl triflate 21 as a colorless oil (18.4 g, 88%).

R_{f}=0.40 (5% ethyl acetate-hexanes; KMnO₄).

¹H NMR (500 MHz, CDCl₃) δ 5.91 (dd, J=5.3, 3.0 Hz, 1H), 3.71 (s, 3H), 2.36-2.16 (m, 2H), 1.85-1.67 (m, 2H), 1.65-1.53 (m, 1H), 1.42 (s, 3H), 0.96 (d, J=6.4 Hz, 3H).

¹³C NMR (126 MHz, CDCl₃) δ 172.1, 150.1, 119.7, 118.5 (q, J=319.4 Hz), 52.3, 50.8, 40.3, 26.2, 23.4, 20.8, 16.9.

¹⁹F NMR (470 MHz, CDCl₃) δ−74.90.

HRMS-ESI (m/z): calculated for [C₁₁H₁₄F₃O₅SNa]⁺ 339.0490, found 339.0493.

### Synthesis of the Dienone S1:

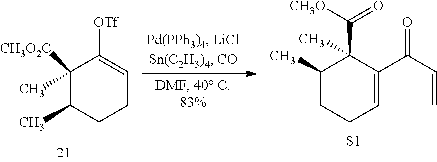

A solution of the vinyl triflate 21 (5.90 g, 18.7 mmol, 1 equiv), tetrakis(triphenylphosphine) palladium (862 mg, 746 μmol, 4.00 mol %) and lithium chloride (3.95 g, 93.3 mmol, 5.00 equiv) in N,N-dimethylformamide (190 mL) was sparged with carbon monoxide for 30 min at 22° C. A balloon of carbon monoxide was attached to the reaction vessel and then tetravinyltin (4.42 mL, 24.3 mmol, 1.30 equiv) was added. The reaction mixture was stirred and heated for 6 h at 40° C., and then was cooled to 22° C. The cooled product mixture was diluted with water (500 mL) and extracted with a mixture of hexanes-ethyl acetate (35% v/v, 3×200 mL). The organic layers were combined and the combined organic layers were washed with aqueous ammonium hydroxide solution (10%, 200 mL). The washed organic layer was dried over magnesium sulfate. The dried solution was filtered and the filtrate was concentrated. The residue obtained was purified by flash-column chromatography (eluting with 10% ether-hexanes initially, grading to 35% ether-hexanes, linear gradient) to provide the dienone S1 as a white solid (3.44 g, 83%).

R_{f}=0.44 (30% ether-hexanes; UV).

¹H NMR (600 MHz, CDCl₃) δ 6.93 (dd, J=5.2, 3.0 Hz, 1H), 6.80 (dd, J=17.1, 10.6 Hz, 1H), 6.21 (dd, J=17.1, 1.8 Hz, 1H), 5.73 (dd, J=10.6, 1.8 Hz, 1H), 3.66 (s, 3H), 2.41-2.32 (m, 1H), 2.32-2.24 (m, 1H), 1.74-1.60 (m, 3H), 1.37 (s, 3H), 0.93 (d, J=6.6 Hz, 3H).

¹³C NMR (151 MHz, CDCl₃) δ 191.6, 174.9, 143.5, 141.1, 132.8, 128.6, 51.7, 47.6, 38.8, 25.9, 25.1, 23.2, 16.7.

HRMS-ESI (m/z): calculated for [C₁₃H₁₈O₃Na]⁺ 245.1154, found 245.1150.

### Synthesis of the Cyclopentenone 14:

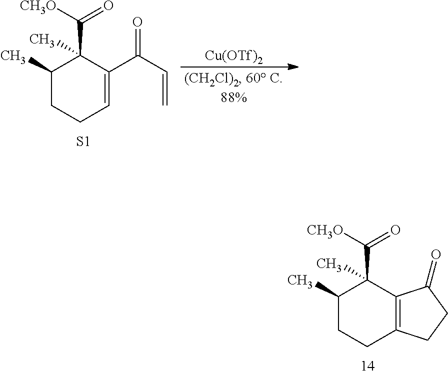

A solution of the dienone S1 (3.00 g, 13.5 mmol, 1 equiv) and copper(II) bis(trifluoromethanesulfonate) (244 mg, 675 μmol, 5.00 mol %) in 1,2-dichioroethane (140 mL) was stirred and heated for 16 h at 60° C. The product mixture was allowed to cool to 22° C. over 1 h. The cooled product mixture was then concentrated. The residue obtained was dissolved in ethyl acetate (100 mL) and the resulting solution was washed with saturated aqueous sodium bicarbonate solution (100 mL). The washed organic layer was dried over magnesium sulfate. The dried solution was filtered and the filtrate was concentrated. The residue obtained was purified by flash-column chromatography (eluting with 10% ethyl acetate-hexanes initially, grading to 35% ethyl acetate-hexanes, linear gradient) to provide the cyclopentenone 14 as a pale yellow solid (2.60 g, 88%). The enantiomeric ratio of the cyclopentenone 14 was determined to be 97:3 by chiral stationary phase HPLC analysis.

R_{f}=0.38 (30% ethyl acetate-hexanes; UV).

¹H NMR (600 MHz, CDCl₃) δ 3.61 (s, 3H), 2.51 (t, J=4.7 Hz, 2H), 2.42-2.27 (m, 4H), 1.72-1.62 (m, 3H), 1.41 (s, 3H), 0.90 (d, J=6.4 Hz, 3H).

¹³C NMR (151 MHz, CDCl₃) δ 207.3, 174.3, 173.5, 140.9, 51.8, 45.5, 39.2, 34.8, 29.9, 27.8, 27.1, 21.5, 16.2.

HRMS-ESI (m/z): calculated for [C₁₃H₁₉O₃]⁺ 223.1334, found 223.1332.

α_{D}²⁰=+17.90° (c=1.0, CH₂Cl₂)

HPLC: Chiralpak IA, hexane:EtOH 95:5, 1.0 mL/min, T_{4R,5R}=7.0 min, T_{4S,5S}=10.6 min, 97:3 er.

### Synthesis of Propargylic Alcohol 22:

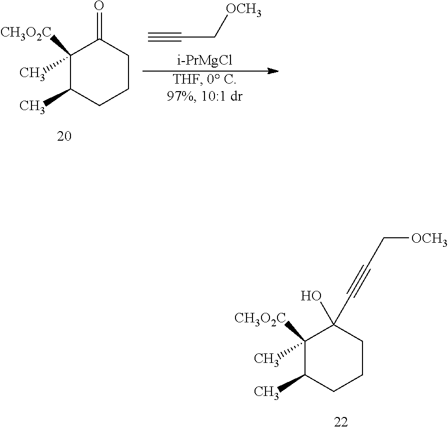

A solution of i-propylmagnesium chloride in tetrahydrofuran (2.0 M, 24.7 mL, 49.4 mmol, 1.30 equiv) was added dropwise over 10 min to a solution of methylpropargyl ether (4.17 mL, 49.4 mmol, 1.30 equiv) in tetrahydrofuran (25 mL) at 0° C. The resulting solution was stirred for 20 min at 0° C. The solution of the resulting Grignard reagent was added dropwise over 10 min to a solution of the α-methyl β-ketoester 20 (7.00 g, 38.0 mmol, 1 equiv) in tetrahydrofuran (190 mL) at 0° C. The resulting mixture was stirred for 20 min at 0° C. The cold product mixture was diluted sequentially with saturated aqueous ammonium chloride solution (20 mL) and water (80 mL). The diluted mixture was warmed to 22° C. over 10 min. The warmed mixture was extracted with ether (3×100 mL) and then the organic layers were combined. The combined organic layers were dried over magnesium sulfate. The dried solution was filtered and the filtrate was concentrated to provide the propargylic alcohol 22 as a colorless oil. (9.37 g, 97%, 10:1 dr). The purity of the propargylic alcohol 22 was determined to be >95% by quantitative ¹H NMR analysis. An analytically-pure sample of the propargylic alcohol 22 was obtained by preparative thin-layered chromatography (eluting with 35% ethyl acetate-hexanes).

### Major Diastereomer:

R_{f}=0.48 (35% ethyl acetate-hexanes; PAA, stains green).

¹H NMR (400 MHz, CDCl₃) δ 4.15 (s, 1H), 3.70 (s, 2H), 3.61-141 (br s, 1H), 3.37 (s, 3H), 2.19 (ddd, J=12.8, 11.4, 4.2 Hz, 1H), 1.94-1.43 (m, 6H), 1.46 (s, 3H), 0.98 (d, J=7.0 Hz, 3H).

¹³C NMR (101 MHz, CDCl₃) δ 175.7, 88.3, 81.5, 74.4, 60.0, 57.6, 54.4, 51.7, 38.9, 36.7, 29.7, 21.7, 20.5, 17.4.

HRMS: calculated for [C₁₄H₂₂O₄Na]⁺ 277.1416, found 277.1411

### Synthesis of the Cyclopentenone 14:

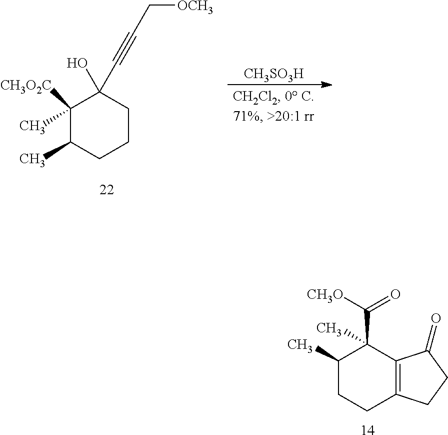

Methanesulfonic acid (10.9 mL, 167 mmol, 5 equiv) was added dropwise over 20 min to a solution of the propargylic alcohol 22 (8.50 g, 33.4 mmol, 1 equiv) in dichloromethane (30 mL) at 0° C. The resulting mixture was stirred for 1 h at 0° C. and then was allowed to warm to 22° C. over 2 h. The warmed product mixture was diluted sequentially with ether (100 mL), water (100 mL), and aqueous sodium hydroxide solution (3 M, 60 mL). The diluted mixture was extracted with ether (3×100 mL) and t the organic layers were combined. The combined organic layers were dried over magnesium sulfate. The dried solution was filtered and the filtrate was concentrated. The residue obtained was purified by flash-column chromatography (eluting with 10% ethyl acetate-hexanes initially, grading to 35% ethyl acetate-hexanes, linear gradient) to provide the cyclopentenone 14 as a yellow solid (5.26 g, 71%). The purity of the cyclopentenone 14 was determined to be >95% by quantitative ¹H NMR analysis. Spectroscopic data for the cyclopentenone 14 obtained in this way were in agreement with those reported above (see S1→14).

### Synthesis of Carboxylic Acid S2:

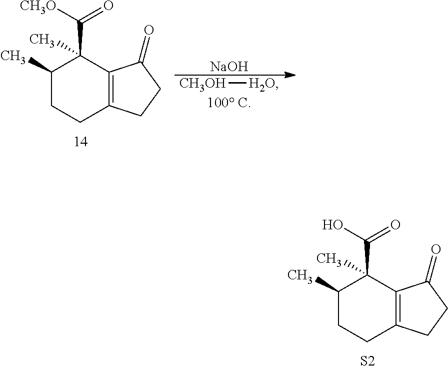

Aqueous sodium hydroxide solution (3 N, 100 μL) was added to a solution of the methyl ester 14 (20.0 mg, 90.0 μmol, 1 equiv) in methanol (100 μL) at 22° C. The resulting mixture was stirred and heated for 5 h at 100° C. The product mixture was cooled to 22° C. and the cooled product mixture was diluted with water (1.0 mL). The diluted mixture was washed with ether (4×1.5 mL). The aqueous phase was isolated and the pH was adjusted to 4 using aqueous hydrochloric acid solution (1 N). The acidified aqueous phase was extracted with ether (5×1.5 mL). The organic layers were combined and the combined organic layers were dried over sodium sulfate. The dried solution was filtered and the filtrate was concentrated. The residue obtained was used directly in the following step.

R_{f}=0.19 (1% acetic acid-50% ethyl acetate-pentane; UV).

¹H NMR (600 MHz, CDCl₃) δ 2.55-2.48 (m, 2H), 2.44-2.29 (m, 4H), 1.81-1.66 (m, 3H), 1.44 (s, 3H), 1.02 (d, J=6.6 Hz, 3H).

^{1.3}C NMR (151 MHz, CDCl₃) δ 207.9, 179.0, 174.7, 140.5, 45.5, 39.1, 34.9, 30.0, 28.0, 27.0, 21.2, 16.2.

HRMS-ESI (m/z): calculated for [C₁₂H₁₆O₃Na]⁺ 231.0992, found 231.1001.

### Synthesis of Acid Chloride 23:

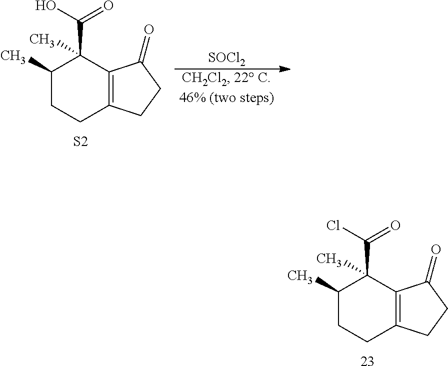

Thionyl chloride (16.0 μL, 225 μmol, 2.50 equiv) was added to a solution of the carboxylic acid S2 obtained in the preceding step (nominally 90.0 μmol, 1 equiv) in dichloromethane (900 μL) 22° C. The resulting mixture was stirred for 9 h at 22° C. The product mixture was concentrated to dryness. The residue obtained was purified by flash-column chromatography (eluting with 20% ethyl acetate-pentane) to provide the acid chloride 23 as an orange oil (9.4 mg, 46% two steps).

R_{f}=0.21 (25% ethyl acetate-pentane; UV).

¹H NMR (400 MHz, CDCl₃) δ2.62-2.55 (m, 2H), 2.51-2.32 (m, 4H), 1.96-1.75 (m, 3H), 1.55 (s, 3H), 1.08 (d, J=6.5 Hz, 3H).

¹³C NMR (101 MHz, CDCl₃) δ206.5, 176.1, 174.7, 139.9, 55.0, 38.8, 34.7, 30.2, 27.4, 26.7, 23.1, 16.0.

HRMS-ESI (m/z): calculated for [C₁₂H₁₅ClO₂Na]⁺ 249.0653, found 249.0655.

Synthesis of the α-alkylated Imide (S,S)-29:

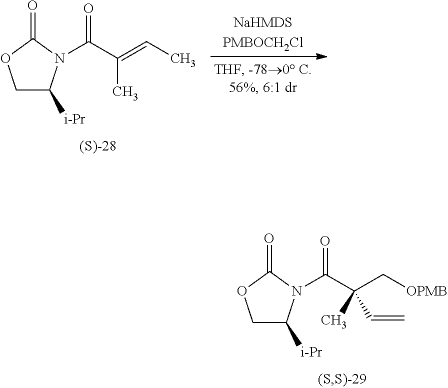

A solution of sodium bis(trimethylsilyl)amide (15.6 g, 85.2 mmol, 2.00 equiv) in tetrahydrofuran (50 mL) was added dropwise to a solution of the imide (S)-28 (9.00 g, 42.6 mmol, 1 equiv) in tetrahydrofuran (300 mL) at −78° C. The resulting solution was stirred for 30 min at −78° C. p-Methoxybenzyl Chloromethyl ether (15.9 g, 85.2 mmol, 2.00 equiv) was then added dropwise and the resulting solution was stirred for 1 h at −78° C. The resulting solution was allowed to warm to 0° C. over 1 h and then to 22° C. over 30 min. The warmed product mixture was diluted sequentially with saturated aqueous ammonium chloride solution (150 and water (150 mL). The diluted product mixture was extracted with ether (3×200 mL). The organic layers were combined and the combined organic layers were dried over sodium sulfate. The dried solution was filtered and the filtrate was concentrated. The α-alkylated imide (S,S)-29 was formed as a 6:1 mixture of diastereomers based on ¹H NMR analysis of the unpurified product mixture. Over several experiments, the diastereoselectivity of this transformation varied from 5:1 to 10:1. The residue obtained was purified by flash-column chromatography (eluting with 5% ether-pentane initially, grading to 50% ether-pentane, five steps) to provide the α-alkylated imide (S,S)-29 as a pale yellow oil (7.92 g, 56%, 6:1 dr).

R_{f}=0.19 (25% ether-pentane; UV; KMnO₄).

¹H NMR (600 MHz, CDCl₃) δ 7.21 (d, J=8.5 Hz, 2H), 6.85 (d, J=8.6 Hz, 2H), 6.17 (dd, J=17.7, 10.7 Hz, 1H), 5.08 (d, J=10.8 Hz, 1H), 4.97 (d, J=17.7 Hz, 1H), 4.53-4.37 (m, 3H), 4.26-1.14 (m, 3H), 3.80 (s, 3H), 3.51 (d, J=8.9 Hz, 1H), 2.36-2.27 (m, 1H), 1.48 (s, 3H), 0.88 (d, J=7.1 Hz, 3H), 0.81 (d, J=6.9 Hz, 3H).

¹³C NMR (151 MHz, CDCl₃) δ 174.0, 159.2, 152.7, 139.5, 130.5, 129.2, 113.9, 113.8, 75.1, 73.1, 63.3, 60.1, 55.4, 52.3, 28.5, 22.7, 18.2, 14.8.

HRMS-ESI (m/z) calculated for [C₂₀H₂₇NO₅Na]⁺ 384.1787, found 384.1782.

### Synthesis of the Alcohol S3:

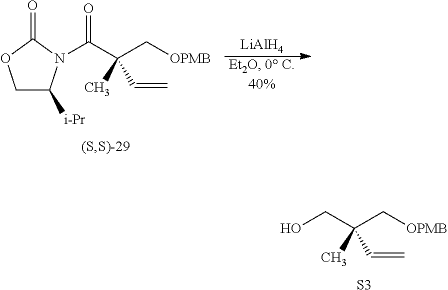

A solution of the α-alkylated imide (S,S)-29 (5.20 g, 15.7 mmol, 1 equiv) in ether (50 mL) was added over 5 min to a stirring suspension of lithium aluminum hydride (1.20 g, 31.4 mmol, 2.00 equiv) in ether (100 mL) at 0° C. The resulting solution was stirred for 10 mm at 0° C. Water (4.0 mL) and aqueous sodium hydroxide solution (3 M, 1.0 mL) were added sequentially to the product mixture at 0° C. and the resulting mixture was gradually warmed to 22° C. over 15 min. Sodium sulfate (˜2 g) was then added to the warmed product mixture. The resulting heterogeneous mixture was filtered through a pad of elite. The filtrate was collected and concentrated. The residue obtained was purified by flash-column chromatography (eluting, with 10% ethyl acetate-pentane initially, grading to 30% ethyl acetate-pentane, four steps) to provide the alcohol S3 as a colorless oil (1.49 g, 40%).

R_{f}=0.30 (25% ethyl acetate-pentane; PAA, stains blue).

¹H NMR (600 MHz, CDCl₃) δ 7.24 (d, J=8.5 Hz, 2H), 6.88 (d, J=8.6 Hz, 2H), 5.84 (dd, J=17.7, 11.0 Hz, 1H), 5.14 (d, J=12.1 Hz, 1H), 5.11 (d, J=18.4 Hz, 1H), 4.47 (d, J=11.8 Hz, 1H), 4.44 (d, J=11.8 Hz, 1H), 3.81 (s, 3H), 3.57 (dd, J=10.7, 5.3 Hz, 1H), 3.52 (dd, J=10.9, 5.3 Hz, 1H), 3.45 (d, J=8.8 Hz, 1H), 3.36 (d, J=8.8 Hz, 1H), 2.40 (t, J=6.0 Hz, 1H), 1.04 (s, 3H).

¹³C NMR (151 MHz, CDCl₃) δ 159.3, 141.6, 130.2, 129.3, 114.5, 113.9, 76.8, 73.3, 69.6, 55.4, 42.8, 19.1.

HRMS-ESI (m/z): calculated for [C₁₄H₂₀O₃Na]⁺ 259.1310, found 259.1319.

### Synthesis of the Neopentyl Iodide (S)-30:

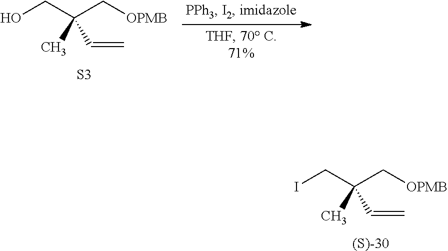

Iodine (1.77 g, 6.98 mmol, 1.10 equiv) was added in one portion to a stirring solution of the alcohol S3 (1.50 g, 6.35 mmol, 1 equiv), triphenylphosphine (1.83 g, 6.98 mmol, 1.10 equiv), and imidazole (864 mg, 12.7 mmol, 2.00 equiv) in tetrahydrofuran (40 mL) at 22° C. The resulting mixture was stirred and heated at 70° C. for 3 h and then cooled to 22° C. over 30 min. The cooled product mixture was concentrated. The residue obtained was treated with saturated aqueous ammonium chloride solution (50 mL) and the resulting mixture was extracted with ethyl acetate (3×50 mL). The organic layers were combined and the combined organic layers were washed with aqueous sodium thiosulfate solution (20% w/v, 50 mL). The washed organic layers were dried over sodium sulfate. The dried solution was filtered and the filtrate was concentrated. The residue obtained was purified by flash-column chromatography (eluting with 1% ethyl acetate-hexanes initially, grading to 5% ethyl acetate-hexanes, linear gradient) to provide the neopentyl iodide (S)-30 as a pale yellow oil (1.56 g, 71%).

R_{f}=0.50 (4% ethyl acetate-pentane; UV; PAA, stains blue).

¹H NMR (600 MHz, CDCl₃) δ 7.26 (d, J=8.6 Hz, 2H), 6.88 (d, J=8.6 Hz, 2H), 5.85 (dd, J=17.6, 10.9 Hz, 1H), 5.10 (dd, J=22.7, 14.2 Hz, 2H), 4.46 (s, 2H), 3.81 (s, 3H), 3.37-3.23 (m, 4H), 1.15 (s, 3H).

¹³C NMR (151 MHz, CDCl₃) δ 159.3, 141.5, 130.6, 129.3, 114.6, 113.9, 76.0, 73.2, 55.4, 41.0, 21.9, 18.3.

HRMS-ESI (m/z): calculated for [C₁₄H₁₉IO₂Na]⁺ 369.0327, found 369.0325.

### Synthesis of the Enyne 26:

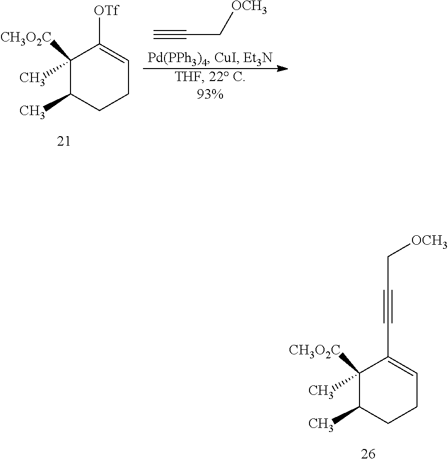

Triethylamine (2.76 mL, 19.8 mmol, 10.0 equiv) was added to a solution of the vinyl triflate 21 (627 mg, 1.98 mmol, 1 equiv) tetrakis(triphenylphosphine)palladium(0) (114 mg, 99.0 μmol, 0.0500 equiv), copper(I) iodide (37.7 mg, 19.8 μmol, 0.100 equiv) and methyl propargyl ether (208 mg, 2.97 mmol, 1.50 equiv) in tetrahydrofuran (10 mL) at 22° C. The resulting black solution was stirred for 50 min at 22° C. The product mixture was diluted with saturated aqueous ammonium chloride solution (30 mL) and the diluted mixture was extracted with ether (3×20 mL). The organic layers were combined and the combined organic layers were dried over sodium sulfate. The dried solution was filtered and the filtrate was concentrated. The residue obtained was purified by flash-column chromatography (eluting with 15% ether-pentane initially, grading to 20% ether-pentane, linear gradient) to provide the enyne 26 as colorless oil (437 mg, 93%).

R_{f}=0.71 (5% ether-dichloromethane; UV).

¹H NMR (400 MHz, CDCl₃) δ 6.29 (dd, J=6.5, 3.0 Hz, 1H), 4.18 (s, 2H), 3.69 (s, 3H), 3.35 (s, 3H), 2.67-2.04 (m, 2H), 1.77-1.48 (m, 3H), 1.43 (s, 3H), 0.93 (d, J=6.7 Hz, 3H).

¹³C NMR (101 MHz, CDCl₃) δ174.5, 137.2, 124.0, 85.7, 84.1, 60.4, 57.4, 51.9, 49.9, 38.5, 26.5, 25.4, 23.4, 17.5.

HRMS-ESI (m/z): calculated for [C₁₄H₂₀NaO₃]⁺ 259.1305, found 259.1314.

### Synthesis of the Carboxylic Acid S4:

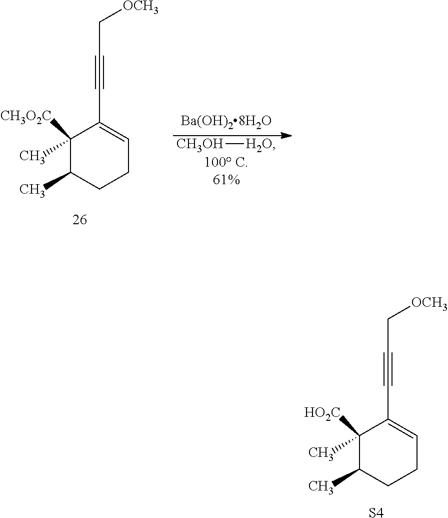

Barium hydroxide octahydrate (133 mg, 423 μmol, 5.00 equiv) was added to a solution of the methyl ester 26 (20.0 mg, 84.6 μmol, 1 equiv) in a mixture of methanol (400 μL) and water (100 μL) at 22° C. The reaction mixture was stirred and heated for 26 h at 100° C. The product mixture was cooled to 22° C. and the cooled product mixture was diluted with water (1.5 mL). The diluted mixture was washed with ether (3×1.5 mL). The pH of the aqueous layer was adjusted to 3 using aqueous hydrochloric acid solution (1 N). The acidified aqueous layer was extracted with ether (3×1.5 mL). The organic layers were combined and the combined organic layers were dried over magnesium sulfate. The dried solution was filtered and the filtrate was concentrated to provide the carboxylic acid S4 as a colorless oil (11.5 mg, 61%). The residue obtained was used directly in the following step.

### Synthesis of the Lactone 27:

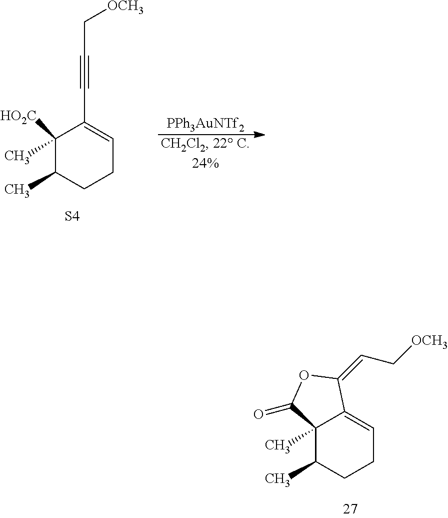

Bis(trifluoromethanesulfonyl)imidate (triphenylphosphine)gold(I) (3.6 mg, 4.90 μmol, 0.100 equiv) was added to a solution of the carboxylic acid S4 obtained in the preceding step (nominally 490 μmol, 1 equiv) in methylene chloride at 22° C. The reaction mixture was stirred for 30 min at 22° C. The product mixture was concentrated to dryness and the residue obtained was purified by preparative thin-layered chromatography (eluting with 25% ether-pentane) to provide the lactone 27 as a colorless oil (4.5 mg, 24%).

R_{f}=0.39 (25% ether-pentane; UV).

¹H NMR (400 MHz, CDCl₃) δ 6.08 (t, J=3.8 Hz, 1H), 5.25 (t, J=7.2 Hz, 1H), 4.13 (dt, J=8.0, 4.1 Hz, 2H),3.35 (s, 3H), 2.25-2.13 (m, 3H), 1.96 (dtd, J=13.6, 9.0, 4.1 Hz, 1H), 1.60-1.51 (m, 1H), 1.38 (s, 3H), 0.86 (d, J=6.9 Hz, 3H).

¹³C NMR (151 MHz, CDCl₃) δ 176.8, 149.7, 132.0, 123.0, 97.8, 66.2, 58.1, 46.3, 31.0, 26.0, 23.3, 21.0, 14.6.

HRMS-ESI (m/z): calculated for [C₁₃H₁₈NaO₃]⁺ 245.1148, found 245.1157.

Synthesis of the β-ketoester 31:

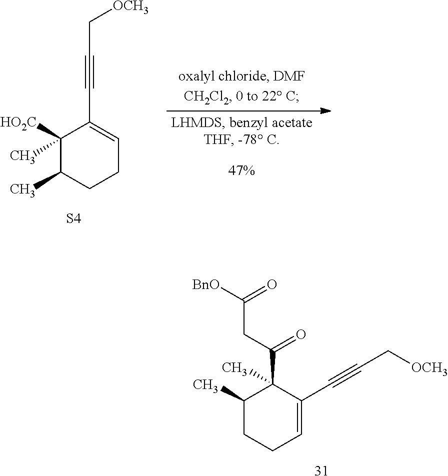

Oxalyl chloride (35.0 μL, 414 μmol, 8.00 equiv) and one drop of N,N-dimethylformamide (˜5 μL) was added to a solution of the carboxylic acid S4 (11.5 mg, 51.7 μmol, 1 equiv) in dichloromethane (500 μL) at 0° C. The resulting mixture was warmed to 22° C. over 5 min. The mixture was stirred for 1 h at 22° C. and then was concentrated to dryness. The residue obtained was dried by azeotropic distillation with toluene (3×500 μL) to provide an acid chloride (not shown) that was used immediately in the following step.

Benzyl acetate (37.0 μL, 259 μmol, 5.00 equiv) was added to a solution of lithium bis(trimethylsilyl)amide (51.9 mg, 310 μmol, 6.00 equiv) in tetrahydrofuran (1.7 mL) at −78° C. The resulting mixture was stirred at for 1 h at −78° C. A solution of the acid chloride obtained in the preceding step (nominally 51.7 μmol, 1 equiv) in tetrahydrofuran (500 μL) was then added dropwise. The reaction mixture was stirred for 14 h at −78° C. The cold product mixture was diluted with saturated aqueous ammonium chloride solution (1.5 mL). The diluted mixture was warmed to 22° C. over 10 min. The warmed solution was extracted with ethyl acetate (3×1.5 mL). The organic layers were combined and the combined organic layers were dried over sodium sulfate. The dried solution was filtered and the filtrate was concentrated. The residue obtained was purified by preparative thin-layered chromatography (eluting with 3% ether-dichloromethane) to provide the β-ketoester 31 as a colorless oil (8.6 mg, 47%).

R_{f}=0.31 (dichloromethane; UV).

Product exists as keto-enol tautomers (keto:enol=13:7).

### Keto-Tautomer

¹H NMR (500 MHz, CDCl₃) δ7.40-7.28 (m, 5H), 6.38 (dd, J=5.2, 3.2 Hz, 1H), 5.17 (s, 2H), 4.12 (s, 2H), 3.72 (d, J=16.6 Hz, 1H), 3.67 (d, J=16.6 Hz, 1H), 3.30 (s, 3H), 2.33-2.07 (m, 2H), 1.71-1.45 (m, 3H), 1.40 (s, 3H), 0.96 (d, J=6.5 Hz, 3H).

¹³C NMR (126 MHz, CDCl₃) δ 205.1, 167.5, 138.6, 135.8, 128.7 (2C), 128.3 (3C), 124.1, 85.6, 85.4, 67.0, 60.3, 57.6, 55.1, 49.0, 38.7, 26.9, 25.2, 23.3, 17.3.

### Enol-Tautomer

¹H NMR (500 MHz, CDCl₃) δ 12.35 (s, 1H), 7.40-7.28 (m, 5H), 6.31 (dd, J=5.2, 3.1 Hz, 1H), 5.17 (s, 2H), 5.15 (s, 1H), 4.17 (s, 2H), 3.31 (s, 3H), 2.33-2.07 (m, 2H), 1.71-1.45 (m, 3H), 1.40 (s, 3H), 0.96 (d, J=6.5 Hz, 3H).

¹³C NMR (126 MHz) CDCl₃) δ 180.6, 172.8, 138.1, 135.9, 128.7 (2C), 128.5 (3C), 125.1, 91.2, 86.0, 84.0, 66.0, 60.5, 57.4, 47.2, 39.4, 26.4, 25.8, 22.7, 17.8.

HRMS-ESI (m/z): calculated for [C₂₂H₂₆O₄Na]⁺ 377.1723, found 377.1723.

### Synthesis of the Lactone 32:

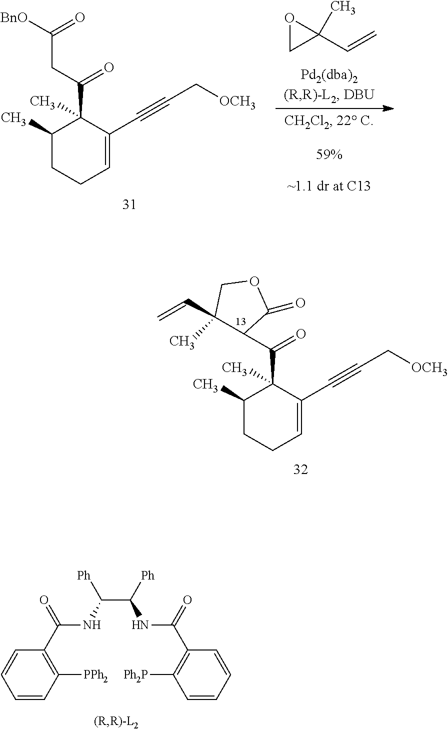

A solution of tris(dibenzylideneacetone)dipalladium(0) (5.6 mg, 6.08 μmol, 25.0 mol %) and the ligand L₂ (12.6 mg, 18.2 μmol, 75.0 mol %) in dichloromethane (500 μL) was stirred at 22° C. for 10 min. The resulting catalyst solution was addedto the β-ketoester 31 (8.6 mg, 24.3 μmol, 1 equiv) at 22° C. Isoprene oxide (29.0 μL, 291 μmol, 12.0 equiv) and 1,8-diazabicyclo[5.4.0]undec-7-ene (3.6 μL, 24.3 μmol, 1.00 equiv) were then added in one portion. The resulting solution was stirred for 1 h at 22° C. The product mixture was filtered through short pad of silica gel. The silica was washed with ether (10 mL). The filtrates were collected, combined, and concentrated. The residue obtained was purified by preparative thin-layered chromatography (eluting with 1.5% acetone-hexanes) to provide separately the C13 lactone diastereomers 32 as colorless oils (2.1 mg, 26%; 2.6 mg, 33%, stereoehemistry not assigned).

### Less Polar Minor Diastereomer:

R_{f}=0.47 (20% ethyl acetate-pentane; UV; PAA, stains blue).

¹H NMR (400 MHz, CDCl₃) δ6.49 (t, J=4.1 Hz, 1H), 5.98 (dd, J=17.6, 10.9 Hz, 1H), 5.18 (d, J=10.9 Hz, 1H), 5.11 (d, J=17.5 Hz, 1H), 4.62 (d, J=8.4 Hz, 1H), 4.16 (s, 2H), 4.11 (s, 1H), 3.89 (d, J=8.4 Hz, 1H), 3.37 (s, 3H), 2.43-2.18 (m, 2H), 2.06-1.91 (m, 1H), 1.65-1.51 (m, 2H), 1.37 (s, 3H), 1.33 (s, 3H), 0.98 (d, J=6.8 Hz, 3H).

¹³C NMR (101 MHz, CDCl₃) δ208.0, 172.8, 140.9, 137.8, 124.2, 116.4, 86.4, 85.7, 76.7, 61.5, 60.5, 57.8, 56.2, 47.6, 40.5, 26.5, 26.2, 24.9, 22.7, 17.3.

HRMS-ESI (m/z): calculated for [C₂₀H₂₇O₄]⁺ 331.1904, found 331.1904.

### More Polar Major Diastereomer:

R_{f}=0.37 (20% ethyl acetate pentane; UV; PAA, stains blue).

¹H NMR (400 MHz, CDCl₃) δ6.37 (t, J=4.1 Hz, 1H), 5.93 (dd, J=17.4, 10.7 Hz, 1H), 5.19 (d, J=17.0 Hz, 1H), 5.18 (d, J=11.1 Hz, 1H), 4.28 (d, J=8.6 Hz, 1H), 4.14 (d, J=9.3 Hz, 1H), 4.17 (s, 2H), 4.16 (s, 1H), 3.37 (s, 3H), 2.24-2.10 (m, 2H), 1.84-1.49 (m, 3H), 1.62 (s, 3H), 1.22 (s, 3H), 1.13 (d, J=6.9 Hz, 3H).

¹³C NMR (101 Hz, CDCl₃) δ206.0, 173.3, 141.7, 140.8, 122.4, 114.3, 86.5, 85.1, 76.5, 60.5, 58.5, 57.8, 55.0, 47.5, 39.2, 25.8, 25.5, 22.6, 18.4, 16.7.

HRMS-ESI (m/z): calculated for [C₂₀H₂₇O₄]⁺ 331.1904, found 331.1896.

### Synthesis of the Diester 35:

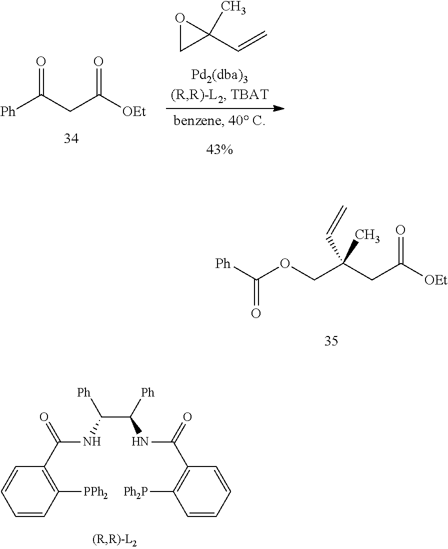

This compound was prepared by a modification of the literature procedure.¹⁶ Tetrabutylammonium difluorotriphenylsilicate (TBAT, 324 mg, 600 μmol, 1 mol %), tris(dibenzylideneacetone)dipalladium (385 mg, 420 μmol, 0.700 mol %), and (R,R)-L₂ (994 mg, 1.26 mmol, 2.1 mol %) were added to a 500-mL round-bottomed flask. Benzene (300 mL) was added at 22° C. The resulting dark purple solution was stirred for 15 mm at 22° C., over which time it became orange. Ethylbenzoyl acetate (10.39 mL, 60.0 mmol, 1 equiv) and isoprene oxide (6.45 mL, 66.0 mmol, 1.10 equiv) were then added in sequence. The reaction mixture was stirred and heated for 18 h at 40° C. The product mixture was cooled to 22° C. The cooled product mixture was concentrated to dryness and the residue obtained was dissolved in ether (300 mL). The resulting solution was washed with aqueous sodium hydroxide solution (1 M, 200 mL). The organic layer was isolated and dried over magnesium. The dried solution was filtered and the filtrate was concentrated. The residue obtained was purified by flash-column chromatography (eluting with hexanes initially, grading to 15% ethyl acetate-hexanes, linear gradient) to provide the diester 35 as a yellow oil (7.15 g, 43% yield). Spectroscopic data for the diester 35 prepared in this way were in agreement with those previously reported.¹⁶

R_{f}=0.58 (15% ethyl acetate-hexanes; UV).

¹H NMR (400 MHz, CDCl₃) δ 8.07-8.01 (m, 2H), 7.57 (t, J=7.4 Hz, 1H), 7.45 (t, J=7.7 Hz, 2H), 5.97 (dd, J=17.7, 10.8 Hz, 1H), 5.15 (d, J=17.7, 1H), 5.15 (d, J=10.8, 1H), 4.27 (s, 2H), 4.10 (q, J=7.1 Hz, 2H), 2.52 (s, 2H), 1.29 (s, 3H), 1.22 (t, J=7.1 Hz, 3H).

### Synthesis of the Alcohol S5:

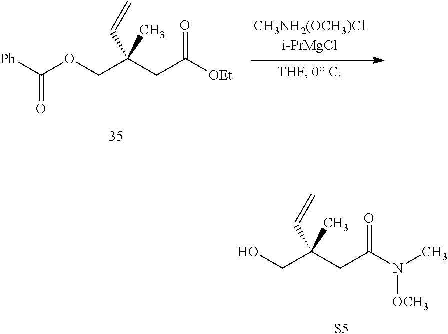 [SMILES: C=C[C@@](C)(COC(=O)c1ccccc1)CC(=O)OCC ; C=C[C@@](C)(CO)CC(=O)N(C)OC ; "S5"]

A solution of i-propylmagnesium chloride in tetrahydrofuran (2.0 M, 152 mL, 304 mmol, 6.00 equiv) was added dropwise over 15 min to a stirring suspension of N,O-dimethylhydroxylamine hydrogen chloride (14.8 g, 152 mmol, 3.00 equiv) and the diester 35 (14.0 g, 50.7 mmol, 1 equiv) in tetrahydrofuran (255 mL) at 0° C. The resulting mixture was stirred for 30 min at 0° C. and then was diluted sequentially with saturated aqueous ammonium chloride solution (200 mL) and water (300 mL). The resulting mixture was extracted with ether (3×500 mL). The organic layers were combined and the combined organic layers were dried over sodium sulfate. The dried solution was filtered and the filtrate was concentrated to provide a mixture of N-methoxy-N-methylbenzamide and the alcohol S5. This material was used directly in the following step.

### Synthesis of the Aldehyde 36:

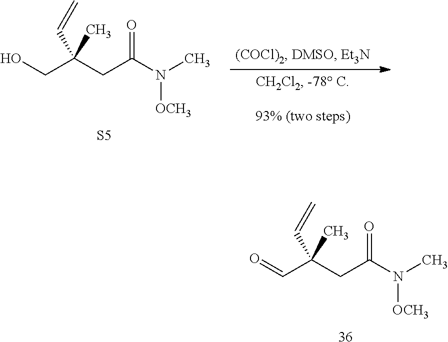

Dimethyl sulfoxide (18.0 mL, 254 mmol, 5.00 equiv) was added dropwise over 10 min to a solution of oxalyl chloride (8.70 mL, 101 mmol, 2.00 equiv) in dichloromethane (200 mL) at −78° C. The resulting mixture was stirred for 10 min at −78° C., and then a solution of the unpurified alcohol obtained in the preceding step (nominally 50.7 mmol, 1 equiv) in dichloromethane (30 mL) was added dropwise over 20 min. The resulting mixture was stirred for 30 min at −78° C. Triethylamine (70.7 mL, 507 mmol, 10.0 equiv) was then added dropwise over 20 min. The resulting mixture was stirred for 1 h at −78° C. The mixture was then allowed to warm to 22° C. over 30 min. The warmed product mixture was diluted with saturated aqueous ammonium chloride solution (500 mL) and the organic layer was separated. The aqueous layer was extracted with dichloromethane (2×500 mL) and organic layers were combined. The combined organic layers were dried over sodium sulfate. The dried solution was filtered and the filtrate was concentrated. The residue obtained was purified by flash-column chromatography (eluting with 12% ether-hexanes initially, grading to 70% ether-hexanes, linear gradient) to provide the aldehyde 36 as a colorless oil (8.70 g, 93%, two steps). The isolated material contained small amounts of impurities. The yield is based on this material.

R_{f}=0.48 (50% ethyl acetate-hexanes; KMnO₄).

¹H NMR (600 MHz, CDCl₃) δ 9.56 (s, 1H), 5.93 (dd, J=17.6, 10.8 Hz, 1H), 5.26 (d, J=10.8 Hz, 1H), 5.17 (d, J=17.6 Hz, 1H), 3.69 (s, 3H), 3.15 (s, 3H), 2.92 (d J=16.5 Hz, 1H), 2.84 (d, J=16.5 Hz, 1H), 1.30 (s, 3H).

¹³C NMR (151 MHz, CDCl₃) δ 201.8, 171.5, 138.9, 116.4, 61.4, 50.5, 39.4, 32.1, 19.6.

HRMS-ESI (m/z): calculated for [C₉H₁₆NO₃]⁺ 186.1125, found 186.1124.

### Synthesis of the Acetal S6:

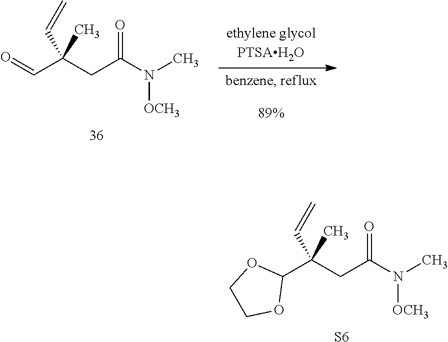

A solution of the aldehyde 36 (2.13 g, 11.5 mmol, 1 equiv) in benzene (5.0 mL) was added to a solution of p-toluenesulfonic acid (43.8 mg, 230 μmol, 2.00 mol %) and ethylene glycol (1.29 mL, 23.0 mmol, 2.00 equiv) in benzene (23 mL) at 22° C. The reaction vessel was fitted with a Dean-Stark trap and the reaction mixture was heated and stirred for 4 h at reflux. The product mixture was cooled to 22° C. The cooled product mixture was diluted with ethyl acetate (300 mL) and then washed with saturated sodium bicarbonate solution (2×300 mL). The organic layer was isolated and dried over magnesium sulfate. The dried solution was filtered and the filtrate was concentrated to afford the acetal S6 as an orange oil (2.34 g, 89%).

R_{f}=0.37 (50% ethyl acetate-hexanes; KMnO₄).

¹³C NMR (400 MHz, CDCl₃) δ 6.04 (dd, J=17.6, 11.0 Hz, 1H), 5.14 (m, 2H), 4.87 (s, 1H), 4.03-3.79 (m, 4H), 3.67 (s, 3H), 3.15 (s, 3H), 2.69 (d, J=14.9 Hz, 1H), 2.56 (d, J=14.9 Hz, 1H), 1.23 (s, 3H).

¹³C NMR (101 MHz, CDCl₃) δ172.6, 141.5, 114.4, 108.3, 65.53, 65.50, 61.2, 43.7, 36.86, 32.1, 18.3.

HRMS-ESI (m/z): calculated for [C₁₁H₂₀NO₄]⁺ 230.1387, found 230.1380.

### Synthesis of the Aldehyde 37:

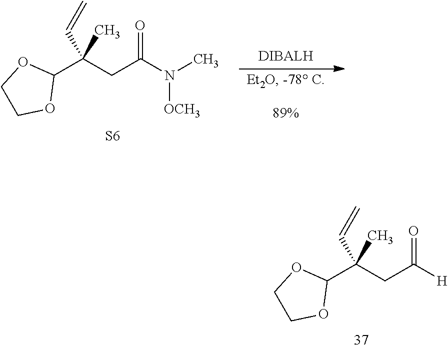

A solution of di-iso-butylaluminum hydride (DBALH) in pentane (1.0 M, 83.0 mL, 83.0 mmol, 2.00 equiv) was added dropwise over 10 min to a solution of the Weinreb amide S6 (9.50 g, 41.4 mmol, 1 equiv) in ether (400 mL) at −78° C. The reaction mixture was stirred for 1 h at −78° C. and then methanol (10 mL) was added dropwise over 5 min. The product mixture was warmed to 22° C. over 10 min. The warmed product mixture was diluted sequentially with aqueous hydrochloric acid solution (0.5 M, 300 mL) and ether (300 mL). The organic layer was separated and the aqueous layer was extracted with ether (2×300 mL). The organic layers were combined and the combined organic layers were dried over sodium sulfate. The dried solution was filtered and the filtrate was concentrated. The residue obtained was purified by flash-column chromatography (eluting with 20% ether-hexanes initially, grading to 40% ether-hexanes, linear gradient) to provide the aldehyde 37 as colorless oil (5.76 g, 89%).

R_{f}=0.40 (25% ether-pentane; PAA, stains blue).

¹³C NMR (400 MHz, CDCl₃) δ 9.74 (t, J=3.1 Hz, 1H), 6.00 (dd, J=17.6, 11.0 Hz, 1H), 5.26-5.15 (m, 2H), 4.67 (s, 1H), 3.99-3.77 (m, 4H), 2.50-2.31 (m, 2H), 1.23 (s, 3H).

¹³C NMR (500 MHz, CDCl₃) δ 202.1, 140.8, 115.3, 108.0, 65.4, 65.2, 48.2, 43.7, 19.9.

HRMS-ESI (m/z): calculated for [C₉H₁₅O₃]⁺ 171.1016, found 171.1018.

Synthesis of the β-hydroxyketone 38:

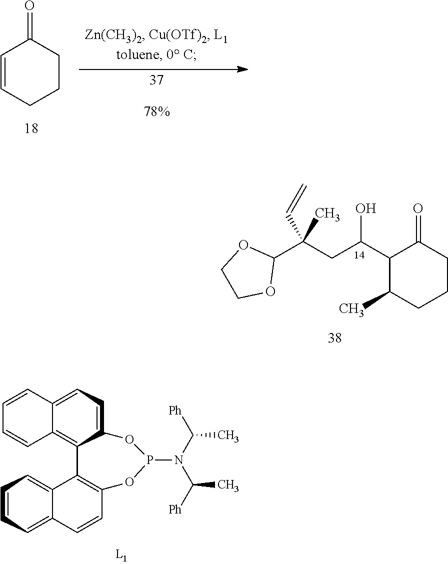

A suspension of copper (II) bis(trifluoromethanesulfonate) (1.93 mg, 608 μmol, 2.00 mol %) and L₁ (656 mg, 1.22 mmol 4.00 mol %) in toluene (50 mL) was stirred for 30 min at 22° C. The resulting solution was cooled to 0° C. for 5 min and then cyclohex-2-ene-1-one (18, 3.71 mL, 39.5 mmol, 1.30 equiv) was added dropwise. A solution of dimethylzinc in toluene (1.2 M, 38.0 mL, 45.6 mmol, 1.50 equiv) was then added dropwise over 10 min. The resulting mixture was stirred for 1 h at 0° C. A solution of the aldehyde 37 (5.17 g, 30.4 mmol, 1 equiv) in toluene (15 mL) was added dropwise over 10 min. The resulting solution was stirred for 30 min at 0° C. The cold product mixture was diluted with saturated aqueous ammonium chloride solution (30 mL). The diluted product mixture was warmed to 22° C. over 5 min. The warmed mixture was extracted with ethyl acetate (3×30 mL). The organic layers were combined and the combined organic layers were dried over sodium sulfate. The dried solution was filtered and the filtrate was concentrated. The residue obtained was purified by flash-column chromatography (eluting with 15% ethyl acetate-hexanes initially, grading to 55% ethyl acetate-hexanes, linear gradient) to provide the β-hydroxyketone 38 as a colorless oil (6.71 g, 78%). The β-hydroxyketone 38 was obtained as an approximately 1:2 mixture of C14 diastereomers (stereochemistry not assigned).

R_{f}=0.46 (40% ethyl acetate-pentane; PAA, stains blue).

### Minor Diastereomer:

¹H NMR (600 MHz, CDCl₃) δ 6.01 (dd, J=17.7, 11.0 Hz, 1H), 5.18-5.05 (m, 2H), 4.72 (s, 1H), 4.02-3.75 (m, 5H), 3.16 (d, J=7.8 Hz, 1H), 2.36-1.35 (m, 10H), 1.08 (s, 3H), 0.99 (d, J=6.7 Hz, 3H).

¹³C NMR (600 MHz, CDCl₃) δ 214.3, 142.4, 114.3, 108.6, 67.2, 65.35, 65.29, 63.24, 43.5, 41.9, 40.5, 34.4, 31.9, 24.2, 20.0, 18.8.

### Major Diastereomer:

¹H NMR (600 MHz, CDCl₃) δ5.81 (dd, J=17.7, 10.9 Hz, 1H), 5.18-5.05 (m, 2H), 4.59 (s, 1H), 4.02-3.75 (m, 5H), 2.97 (d, J=10.1 Hz, 1H), 2.36-1.35 (m, 10H), 1.14 (s, 3H), 1.04 (d, J=6.2 Hz, 3H).

¹³C NMR (600 MHz, CDCl₃) δ 215.4, 142.0, 114.9, 109.1, 66.7, 65.33, 65.29, 63.24, 44.2, 43.0, 42.4, 37.1, 33.6, 26.4, 20.3, 17.6.

HRMS-ESI (m/z): calculated for [C₁₆H₂₆O₄Na]⁺ 305.1723, found 305.1731.

Synthesis of the β-diketone S7:

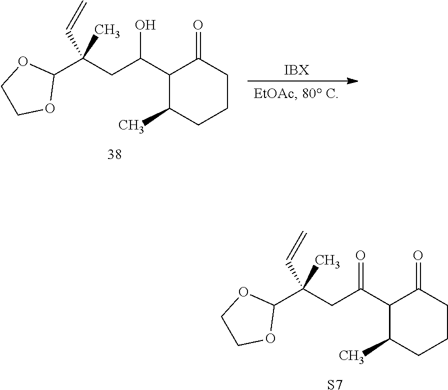

2-Iodoxybenzoic acid (4.50 g, 16.1 mmol, 3.00 equiv) was added to a solution of the β-hydroxyketone 38 (1.52 g, 5.38 mmol, 1 equiv) in ethyl acetate (35 mL) at 22° C. The resulting mixture was stirred and heated for 5 h at 80° C. The mixture was cooled to 22° C., and an additional portion of 2-iodoxybenzoic acid (3.00 g, 10.7 mmol, 1.99 equiv) was added. The resulting mixture was stirred and heated for 17 h at 80° C. The product mixture was cooled to 22° C. over 10 min. The cooled product mixture was filtered through pad of silica gel. The silica pad was washed with ethyl acetate (100 mL). The filtrates were combined and concentrated. The residue obtained was used directly in the following step.

Synthesis of the α-methyl-β-diketone 39:

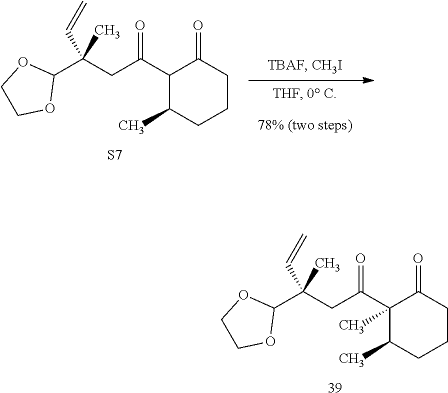

Iodomethane (1.70 mL, 27.3 mmol, 5.08 equiv) and a solution of tetra-n-butylammonium fluoride in tetrahydrofuran (TBAF, 1 M, 21.0 mL, 21.0 mmol, 3.90 equiv) were added in sequence to a solution of the p-diketone obtained in the preceding step (nominally 5.38 mmol, 1 equiv) in tetrahydrofuran (70 mL) at 0° C. The resulting solution was stirred for 4 h at 0° C. The product mixture was concentrated. The residue obtained was purified by flash-column chromatography (eluting with 2% ethyl acetate-hexanes grading to 20% ethyl acetate-hexanes, linear gradient) to provide the α-methyl-β-diketone 39 as a colorless oil (1.49 g, 78%).

R_{f}=0.36 (15% ethyl acetate-pentane; PAA, stains purple).

¹H NMR (400 MHz, CDCl₃), δ5.88 (dd, J=17.7, 10.9 Hz, 1H), 5.09 (m, 2H), 4.74 (s, 1H), 3.95-3.77 (m, 4H), 2.54 (s, 2H), 2.53-2.44 (m, 1H), 2.42-2.33 (m, 1H), 2.22-2.08 (m, 1H), 2.05-1.96 (m, 1H), 1.68-1.52 (m, 3H), 1.39 (s, 3H), 1.15 (s, 3H), 1.11 (d, J=6.8 Hz, 3H).

¹³C NMR (101 MHz, CDCl₃) δ 209.5, 207.1, 141.3, 114.4, 108.0, 68.2, 65.4 (2C), 45.3, 45.1, 43.0, 40.3, 30.0, 26.0, 19.3, 18.2, 16.7.

HRMS-ESI (m/z): calculated for [CH₁₇H₂₆NaO₄]⁺ 317.1727 found 317.1722.

Synthesis of the β-hydroxyketone S8:

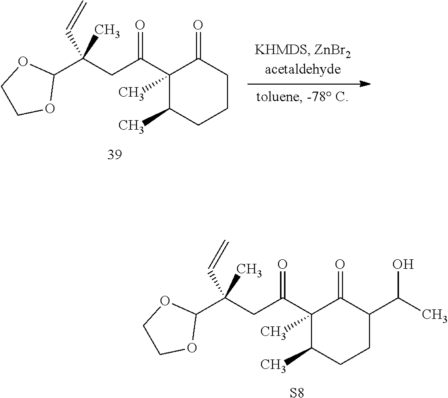

A solution of potassium bis(trimethylsilyl)amide (960 mg, 4.18 mmol, 1.20 equiv) in toluene (20 mL) was added dropwise over 10 min to a solution of the α-methyl-β-diketone 39 (1.18 g, 4.01 mmol, 1 equiv) in toluene (20 mL) at −78° C. The resulting solution was stirred for 30 min at −78° C. A solution of zinc bromide (1.08 g, 4.18 mmol, 1.20 equiv) in tetrahydrofuran (10 mL) was added dropwise over 5 min at −78° C. and the resulting mixture was warmed to 0° C. and stirred for 1 h. Acetaldehyde (450 μL, 8.02 mmol, 2.00 equiv) was then added. The resulting solution was stirred for 1 h at 0° C. The product mixture was diluted with saturated aqueous potassium sodium tartrate solution (100 mL). The diluted solution was allowed to warm to 22° C. over 10 min. The warmed product mixture was extracted with dichloromethane (3×100 mL). The organic layers were combined and the combined organic layers were dried over sodium sulfate. The dried solution was filtered and the filtrate was concentrated. The residue obtained was used directly in the following step.

### Synthesis of Enone 40:

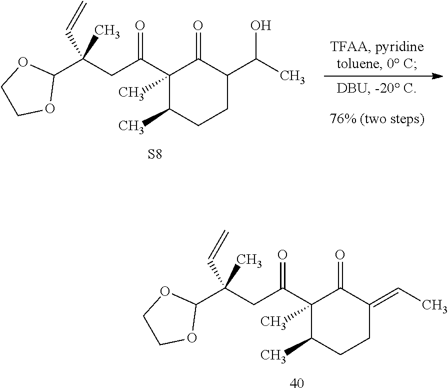

Pyridine (650 μL, 8.04 mmol, 2.00 equiv) and trifluoroacetic anhydride (1.10 mL, 8.02 mmol, 2.00 equiv) were added in sequence to a solution of the residue obtained in the preceding step (nominally 4.01 mmol, 1 equiv) in toluene (30 mL) at 0° C. The resulting solution was stirred for 10 min at 0° C. for 10 min. The creation mixture was cooled to −20° C. and then 1.8-diazabicyclo(5.4.0)undec-7-ene (2.40 mL, 16.0 mmol, 3.99 equiv) was added dropwise. The resulting mixture was stirred for 30 min at −20° C. The cold product mixture was diluted with saturated aqueous sodium bicarbonate solution (50 mL) and then was allowed to warm to 22° C. over 10 min. The warmed product mixture was extracted with ether (3×50 mL). The organic layers were combined and the combined organic layers were dried over sodium sulfate. The dried solution was filtered and the filtrate was concentrated. The residue obtained was purified by flash-column chromatography (eluting with 5% ether-hexanes initially, grading to 40% ether-hexanes, linear gradient) to provide the enone 40 as colorless oil (965 mg, 76%, stereochemistry not assigned).

R_{f}=0.21 (20% ether-pentane; UV).

¹H NMR (500 MHz, CDCl₃) δ 6.72 (tdd, J=7.2, 5.0, 2.2 Hz, 1H), 5.90 (dd, J=17.6, 11.0 Hz, 1H), 5.14-4.99 (m, 2H), 4.78 (s, 1H), 3.94-3.77 (m, 4H), 2.64 (s, 2H), 2.68-2.60 (m, 1H), 2.38-2.27 (m, 1H), 2.00-1.87 (m, 1H), 1.82-1.68 (m, 2H), 1.75 (d, J=7.2 Hz, 3H), 1.39 (s, 3H), 1.13 (s, 3H), 1.06 (d, J=6.8 Hz, 3H).

¹³C NMR (126 MHz, CDCl₃) δ 207.9, 201.3, 141.5, 136.4, 136.2, 114.2, 108.1, 65.5 (2C), 65.1, 46.6, 43.3, 40.1, 27.7, 24.7, 21.4, 18.3, 17.0, 14.0.

HRMS-ESI (m/z): calculated for [C₁₉H₂₈NaO₄]⁺ 343.1880, found 343.1886.

### Synthesis of the Dienyl Triflate 41:

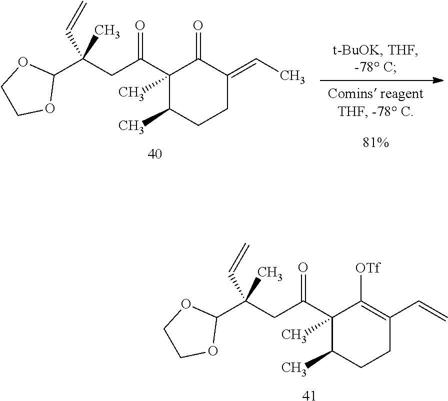

A solution of potassium tort-butoxide (386 mg, 3.44 mmol, 1.50 equiv) in tetrahydrofuran (5.0 mL) was added dropwise over 5 min to a solution of the enone 40 (735 mg, 2.29 mmol, 1 equiv) in tetrahydrofuran (1.0 mL) at −78° C. The resulting solution was stirred for 40 min at −78° C. A solution of N-(5-chloro-2-pyridyl)bis(trifluoromethanesulfonimide) (2.70 g, 6.88 mmol, 3.00 equiv) in tetrahydrofuran (5.0 mL) was then added dropwise over 5 min. The resulting solution was stirred for 30 min at −78° C. The cold product mixture was diluted with saturated aqueous ammonium chloride solution (10 mL) and the diluted solution was allowed to warm to 22° C. over 10 min. The warmed product mixture was diluted with saturated aqueous ammonium chloride solution (50 mL) and extracted with dichloromethane (3×50 mL). The organic layers were combined and the combined organic layers were dried over sodium sulfate. The dried solution was filtered and the filtrate was concentrated. The residue obtained was purified by flash-column chromatography (eluting with 20% dichloromethane-pentane initially, grading to 100% dichloromethane-pentane, linear gradient) to provide the dienyl triflate 41 as a colorless oil (835 mg, 81%).

R_{f}=0.29 (80% dichloromethane-pentane; UV).

¹H NMR (400 MHz, CDCl₃) δ 6.77 (dd, J=17.4, 11.0 Hz, 1H), 5.96 (dd, J=17.6, 11.0 Hz, 1H), 5.42 (d, J=17.4 Hz, 1H), 5.32 (d, J=11.0 Hz, 1H), 5.16-5.08 (m, 2H), 4.84 (s, 1H), 3.95-3.79 (m, 4H), 2.80 (d, J=19.2 Hz, 1H), 2.73 (d, J=19.1 Hz, 1H), 2.57-2.47 (m, 1H), 2.39-2.27 (m, 1H), 1.84-1.60 (m, 3H), 1.52 (s, 3H), 1.19 (s, 3H), 0.99 (d, J=6.4 Hz, 3H).

¹³C NMR (151 MHz, CDCl₃) δ 207.4, 146.7, 141.4, 130.4, 129.8, 118.7 (q, J=319.9 Hz, 1C), 118.1, 114.2, 108.0, 65.5, 65.4, 56.7, 46.8, 43.1, 41.3, 25.3, 24.0, 20.9, 17.9, 17.0.

¹⁹F NMR (376 MHz, CDCl₃) δ−73.66.

HRMS-ESI (m/z): calculated for [C₂₀H₂₇NaO₆S]⁺ 475.1373, found 475.1375.

### Synthesis of the Hydrindenone 42:

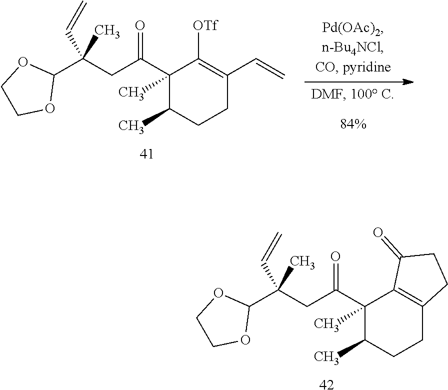

A solution of the dienyl triflate 41 (255 mg, 564 μmol, 1 equiv), palladium(II) acetate (19.0 mg, 84.5 μmol, 0.150 equiv) and tetra-n-butylammonium chloride (157 mg, 564 μmol, 1.00 equiv) in N,N-dimethylformamide (10 mL) was sparged with carbon monoxide for 20 min at 22° C. A balloon of carbon monoxide was attached to the reaction vessel and the reaction mixture was stirred and heated for 1 h at 100° C. The product mixture was cooled to 22° C. The cooled product mixture was diluted with saturated aqueous ammonium chloride solution (50 mL). The diluted mixture was extracted with ethyl acetate (3×35 mL). The organic layers were combined and the combined organic layers were washed with saturated aqueous sodium chloride solution (100 mL). The washed solution was dried over sodium sulfate and the dried solution was filtered. The filtrate was concentrated and the residue obtained was purified by flash-column chromatography (eluting with 8% ethyl acetate-hexanes initially, grading to 40% ethyl acetate-hexanes, linear gradient) to provide the hydrindenone 42 as a colorless oil (157 mg, 84%).

R_{f}=0.35 (40% ethyl acetate-pentane; UV).

¹H NMR (600 MHz, CDCl₃) δ 5.92 (dd, J=17.6, 10.9 Hz, 1H), 5.10 (dd, J=11.0, 1.3 Hz, 1H), 5.08 (dd, J=17.7, 1.2 Hz, 1H), 4.79 (s, 1H), 3.94-3.80 (m, 4H), 2.95 (d, J=191, 1H), 2.64 (d, J=18.9 Hz, 1H), 2.52-2.48 (m, 2H), 2.42-2.25 (m, 4H), 1.83-1.56 (m, 3H), 1.48 (s, 3H), 1.16 (s, 3H), 0.92 (d, J=7.0 Hz, 3H).

¹³C NMR (151 MHz, CDCl₃) δ 210.5, 207.5, 174.0, 142.1, 141.6, 114.1, 108.3, 65.44, 65.43, 50.6, 46.5, 43.1, 40.1, 34.8, 30.0, 28.0, 27.2, 21.5, 17.9, 16.0.

HRMS-ESI (m/z): calculated for [C₂₀H₂₈NaO₄]⁺ 355.1880, found 355.1886.

### Synthesis of the Ketone 43:

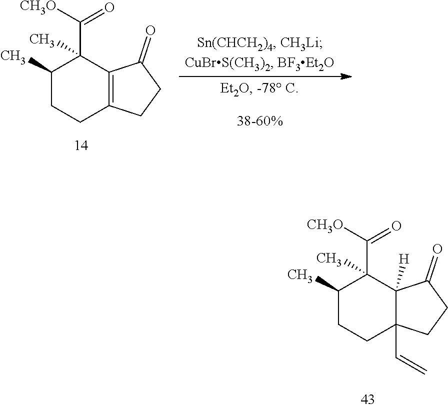

A solution of methyllithium in ether (1.6 M, 1.01 mL, 1.61 mmol, 2.99 equiv) was added dropwise to a solution of tetravinyltin (74.0 μL, 405 μmol, 0.750 equiv) in ether (3.0 mL) at 0° C. The resulting solution was stirred for 30 min at 0° C. The resulting solution of vinyllithium in ether was added dropwise to a solution of copper bromide dimethyl sulfide complex (166 mg, 810 μmol, 1.50 equiv) in ether (1.0 mL) at −40° C. The dark grey or black solution that formed was stirred vigorously for 20 min at −40° C. The mixture was cooled to −78° C. and boron trifluoride diethyl etherate (100 μL, 810 μmol, 1.50 equiv) was added. The resulting dark purple or green solution was stirred for 10 min at −78° C. A solution of the hydrindenone 14 (120 mg, 540 μmol, 1 equiv) in ether (1.0 mL) was then added dropwise. The resulting mixture was stirred for 2 h at −78° C. The product mixture was warmed to 0° C. over 5 min and then was diluted sequentially with saturated aqueous ammonium chloride solution (10 mL) and ether (5.0 mL). The diluted mixture was warmed to 22° C. and the organic layer was separated. The aqueous layer was extracted with ether (3×10 mL). The organic layers were combined and the combined organic layers were dried over magnesium sulfate. The dried solution was filtered and the filtrate was concentrated. The residue obtained was purified by flash-column chromatography (eluting with 100% hexanes initially, grading to 20% ethyl acetate-hexanes, linear gradient) to provide the ketone 43 as colorless oil (51.2 mg, 38%). The yields of this transformation were highly variable (38%-60%).

R_{f}=0.64 (20% ethyl acetate-pentane; KMnO₄).

¹H NMR (400 MHz, CDCl₃) δ 5.87 (dd, J=17.5, 10.8 Hz, 1H), 5.07 (d, J=17.2 Hz, 1H), 5.03 (d, J=10.5 Hz, 1H), 3.65 (s, 3H), 2.35-2.22 (m, 3H), 1.99-1.89 (m, 1H), 1.78-1.63 (m, 5H), 1.49-1.41 (m, 1H), 1.41 (s, 3H), 0.94 (d, J=6.9 Hz, 3H).

¹³C NMR (101 MHz, CDCl₃) δ 218.8, 176.2, 147.7, 111.2, 60.8, 51.5, 48.3, 45.2, 36.6, 36.0, 33.0, 27.3, 27.2, 25.6, 16.8.

HRMS-ESI (m/z): calculated for [C₁₅H₂₂NaO₃]⁺ 273.1461, found 273.1463.

### Synthesis of the Hemiketal 44:

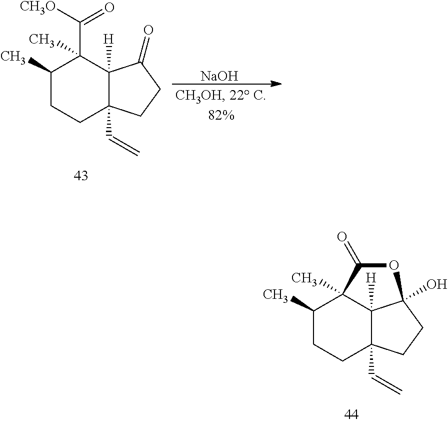

Aqueous sodium hydroxide solution (2 N, 200 μL) was added to a solution of the ketone 43 (4.9 mg, 19.7 μmol, 1 equiv) in methanol (200 μL) at 22° C. The resulting solution was stirred for 2 h at 22° C. The product solution was diluted sequentially with saturated aqueous ammonium chloride solution (1.5 mL) and ethyl acetate (1.5 mL). The layers that formed were separated and the aqueous layer was extracted with ethyl acetate (2×1.5 mL). The organic layers were combined and the combined organic layers were dried over sodium sulfate. The dried solution was filtered and the filtrate was concentrated to provide the hemiketal 44 as white solid (3.8 mg, 82%). The isolated material contained small amounts of impurities. The yield is based on this material.

R_{f}=0.65 (50% ethyl acetate-pentane; KMnO₄).

¹H NMR (400 MHz, CDCl₃) δ 5.83 (dd, J=17.5, 10.7 Hz, 1H), 5.04 (d, J=17.5 Hz, 1H), 5.04 (d, J=10.9 Hz, 1H), 3.32 (s, br, 1H), 2.24 (s, 1H), 2.14-1.94 (m, 2H), 1.82-1.70 (m, 1H), 1.69-1.21 (m, 6H), 1.49 (s, 3H), 1.27 (d, J=6.8 Hz, 3H).

¹³C NMR (101 MHz, CDCl₃) δ 179.5, 147.7, 114.2, 111.0, 58.5, 46.7, 46.6, 38.4, 36.8, 34.1, 33.9, 27.7, 27.1, 15.5.

HRMS-ESI (m/z): calculated for [C₁₄H₁₉O₂]⁺ (—OH) 219.1380, found 219.1386.

### Synthesis of the Nitrile 49:

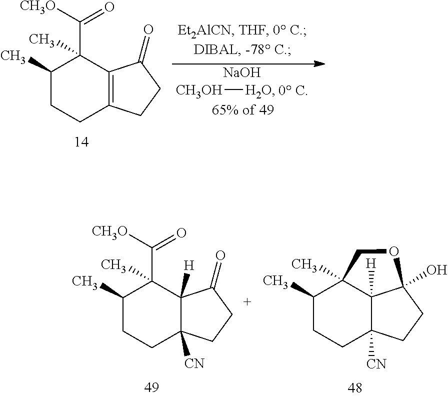

A solution of diethylaluminum cyanide in toluene (1.0 M, 48.6 mL, 48.6 mmol, 3.00 equiv) was added dropwise over 10 min to a solution of the hydrindenone 14 (3.60 g, 16.2 mmol, 1 equiv) in tetrahydrofuran (160 mL) at 0° C. The reaction mixture was stirred for 2 h at 0° C. and then was cooled to −78° C. A solution of di-iso-butylaluminum hydride in toluene (1.0 M, 48.6 mL, 48.6 mmol, 3.00 equiv) was added dropwise over 10 min. The resulting mixture was stirred for 30 min at −78° C. and then aqueous potassium sodium tartrate solution (10% w/v, 40 mL) was added dropwise over 30 min. The product mixture was diluted with ether (200 mL) and then warmed to 0° C. for 30 min. The warmed mixture was further diluted sequentially with aqueous potassium sodium tartrate solution (10% w/v, 200 mL) and ether (200 mL). The resulting mixture was warmed to 22° C. and was stirred vigorously at this temperature for 1 h. The organic layer was separated and the aqueous layer was extracted with ether (2×200 mL). The organic layers were combined and the combined organic layers were dried over magnesium sulfate. The dried solution was filtered and the filtrate was concentrated. The residue obtained was dissolved in methanol (100 mL) and the resulting solution was cooled to 0° C. for 30 min. Aqueous sodium hydroxide solution (100 mM, 20 mL) was added to the cooled solution. The resulting mixture was stirred for 1 h at 0° C. Saturated aqueous ammonium chloride solution (200 mL) was added, and the resulting mixture was warmed to 22° C. over 10 min. The warmed product mixture was extracted with ethyl acetate (3×200 mL). The organic layers were combined and the combined organic layers were dried over magnesium sulfate. The dried solution was filtered and the filtrate was concentrated. The residue obtained was purified by flash-column chromatography (eluting with 10% ethyl acetate-hexanes initially, linearly grading to 30% ethyl acetate-hexanes) to provide separately the nitrile 49 (2.64 g, 65%, white solid. The isolated material contained small amounts of impurities. The yield is based on this material.) and the hemiketal 48 (white solid).

### Nitrite 49:

R_{f}=0.33 (25% ethyl acetate-hexanes; KMnO₄).

¹H NMR (600 MHz, CDCl₃) δ 3.68 (s, 3H), 3.12 (s, 1H), 2.49-2.34 (m, 1H), 2.33-2.17 (m, 2H), 2.16-2.07 (m, 1H), 2.06-1.91 (m, 2H), 1.56 (s, 3H), 1.54-1.48 (m, (1H), 1.48-1.40 (m, 1H), 1.36 (td, J=13.7, 4.1 Hz, 1H), 1.16 (d, J=7.0 Hz, 3H).

¹³C NMR (151 MHz, CDCl₃) δ 211.1, 174.0, 122.5, 58.8, 51.7, 46.1, 38.7, 36.6, 34.0, 32.3, 30.5, 27.7, 22.0, 15.9.

HRMS-ESI (m/z): calculated for [C₁₄H₁₉NO₃Na]⁺ 272.1263, found 272.1266.

### Hemiketal 48:

R_{f}=0.31 (40% ethyl acetate-pentane; KMnO₄).

¹H NMR (600 MHz, CDCl₃) δ 3.82 (d, J=8.8 Hz, 1H), 3.68 (d, J=8.9 Hz, 1H), 2.80 (s, 1H), 2.37 (s, 1H), 2.32-2.23 (m, 1H), 2.22-2.15 (m, 1H), 2.06-1.86 (m, 4H), 1.58-1.50 (m, 1H), 1.50-1.43 (m, 1H), 1.33 (s, 3H), 1.32-1.21 (m, 1H), 0.87 (d, J=6.9 Hz, 3H).

¹³C NMR (1.51 MHz, CDCl₃) δ126.3, 117.5, 76.2, 61.7, 44.6, 40.8, 39.2, 37.0, 34.34, 31.8, 28.9, 25.0, 16.8.

HRMS-ESI (m/z): calculated for [C₁₃H₁₈NO+](—OH) 204.1383, found 204.1393.

### Improved Synthesis of the Nitrile 49:

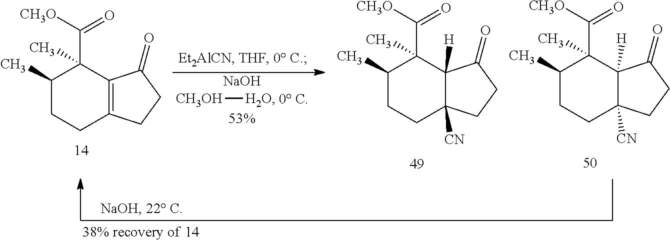

A solution of diethylaluminum cyanide in toluene (1.0 M, 16.6 mL, 16.6 mmol, 3.00 equiv) was added dropwise over 10 min to a solution of the hydrindenone 14 (1.23 g, 5.53 mmol, 1 equiv) in tetrahydrofuran (37 mL) at 0° C. The resulting mixture was stirred for 1 h at 0° C. The product mixture was diluted sequentially with saturated aqueous sodium bicarbonate solution (50 mL) and ether (50 mL). The resulting mixture was warmed to 22° C. The warmed mixture was diluted with water (30 mL) and the mixture formed was stirred vigorously for 30 min at 22° C. The organic layer was separated and the aqueous layer was extracted with ether (3×100 mL). The organic layers were combined and the combined organic layers were dried over sodium sulfate. The dried solution was filtered and the filtrate was concentrated. The residue obtained was dissolved in methanol (30 mL) and the resulting solution was cooled to 0° C. for 5 min. Aqueous sodium hydroxide solution (100 mM, 9.0 mL) was added to the cooled solution. The resulting mixture was stirred for 1 h at 0° C. Saturated aqueous ammonium chloride solution (50 mL) was then added, and the resulting solution was extracted with ether (3×50 mL). The organic layers were combined and the combined organic layers were dried over sodium sulfate. The dried solution was filtered and the filtrate was concentrated. The residue obtained was purified by flash-column chromatography (eluting with 100% toluene initially, grading to 15% ethyl acetate-toluene, four steps) to provide the nitrite 49 as white solid (727 mg, 53%).

The fractions containing the nitrile 50 and the hydrindenone 14 were isolated. separately, combined, and concentrated. The residue obtained was dissolved in methanol (40 mL). Aqueous sodium hydroxide solution (1 N, 30 mL) was then added. The resulting mixture was stirred for 16 h at 22° C. Methanol was removed from the product mixture by rotary evaporation, and the concentrated mixture was diluted with saturated aqueous ammonium chloride solution (50 mL). The diluted solution was extracted with ether (3×50 mL). The organic layers were combined and the combined organic layers were dried over sodium sulfate. The dried solution was filtered and the filtrate was concentrated to provide the hydrindenone 14 (467 mg, 38%). The purity of the hydrindenone 14 obtained in this way was judged to be >95% by ¹H NMR analysis.

### Synthesis of the Nitrile S9:

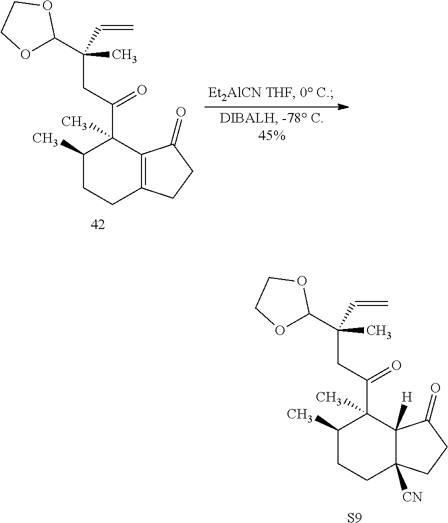 [SMILES: C=C[C@](C)(CC(=O)[C@@]1(C)C2=C(CCC2=O)CC[C@H]1C)C1OCCO1 ; C=C[C@](C)(CC(=O)[C@]1(C)[C@H](C)CC[C@@]2(C#N)CCC(=O)[C@H]21)C1OCCO1 ; "S9"]

A solution of diethylaluminum cyanide in toluene (1.0 M, 540 μL, 540 μmol, 2.98 equiv) was added dropwise to a solution of the hydrindenone 42 (60.1 mg, 181 μmol, 1 equiv) in tetrahydrofuran (2.4 mL) at 0° C. The resulting mixture was stirred for 3 h at 0° C. and then was cooled to −78° C. A solution of di-iso-butylaluminum hydride in toluene (1.0 M, 150 μL, 150 μmol, 0.829 equiv) was added dropwise. After stirring for an additional 30 min at −78° C., aqueous potassium sodium tartrate solution (10% w/v, 1.0 mL) was added. The mixture was then warmed to 22° C. over 30 min. The warmed product mixture was diluted sequentially with aqueous potassium sodium tartrate solution (10% w/v, 5.0 mL) and ether (3.0 mL). The organic layer was separated and the aqueous layer was extracted with ether (3×3 mL). The organic layers were combined and the combined organic layers were dried over sodium sulfate. The dried solution was filtered and the filtrate was concentrated. The residue obtained was purified with flash-column chromatography (eluting with 1% ether-dichloromethane initially, grading to 10% ether-dichloromethane, four steps) to provide the nitrile S9 as colorless oil (29.0 mg, 45%).

R_{f}=0.45 (10% ether-dichloromethane; PAA, stains purple).

¹H NMR (500 MHz, CDCl₃) δ 6.03 (dd, J=17.6, 11.0 Hz, 1H), 5.19-5.10 (m, 2H), 4.81 (s, 1H), 3.97-3.78 (m, 4H), 3.18 (s, 1H), 2.78 (d, J=17.6 Hz, 1H), 2.76 (d, J=17.6 Hz, 1H), 2.43-2.41 (m, 1H), 2.35-2.25 (m, 2H), 2.19-2.11 (m, 1H), 2.08-1.91 (m, 2H), 1.65-1.48 (m, 3H), 1.46 (s, 3H), 1.20 (s, 3H), 1.05 (d, J=7.0 Hz, 3H).

¹³C NMR (126 MHz, CDCl₃) δ 212.8, 207.8, 141.9, 123.4, 114.2, 108.2, 65.5, 65.4, 54.6, 51.7, 43.34, 43.27, 38.8, 37.5, 35.1, 31.5, 31.0, 26.5, 22.1, 18.2, 15.9.

HRMS-ESI (m/z): calculated for [C₂₁H₂₉NNaO₄]⁺ 382.1989, found 382.1991.

### Synthesis of the Aldehyde 51:

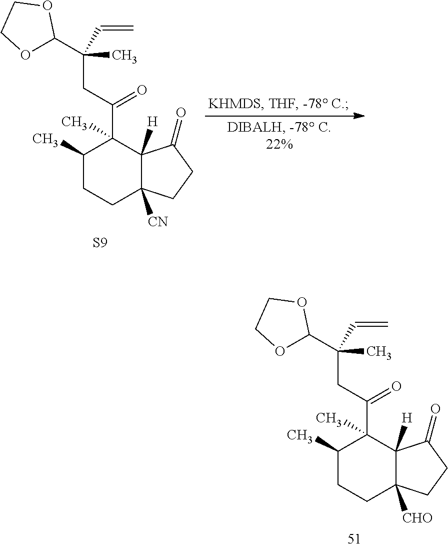

A solution of potassium bis(trimethylsilyl)amide (6.50 mg, 32.7 μmol, 1.20 equiv) in toluene (500 μL) was added dropwise via syringe to a solution of the nitrile S9 (9.8 mg, 27.3 μmol, 1 equiv) in toluene (500 μL) at −78° C. The resulting solution was stirred for 30 min at −78° C. A solution of di-iso-butylaluminum hydride in hexane (1.0 M, 82.0 μL, 82.0 μmol, 3.00 equiv) was added dropwise. The resulting solution was stirred for 20 min at −78° C. The cold product mixture was diluted with aqueous potassium sodium tartrate solution (10% w/v, 300 μL) and the diluted solution was warmed to 22° C. over 30 min. The warmed product mixture was diluted sequentially with aqueous potassium sodium tartrate solution (10% w/v, 700 μL) and ether (3×1.0 mL). The organic layer was isolated and the aqueous layer was extracted with ether (3×1.0 mL). The organic layers were combined and the combined organic layers were dried over sodium sulfate. The dried solution was filtered and the filtrate was concentrated. The residue obtained was purified via preparative thin-layered chromatography (eluting with 40% ethyl acetate-pentane) to provide the aldehyde 51 as a colorless oil. (2.2 mg, 22%).

R_{f}=0.52 (40% ethyl acetate-pentane; PAA, stains blue).

¹H NMR (600 MHz, CDCl₃) δ 9.42 (s, 1H), 5.93 (dd, J=17.7, 10.9 Hz, 1H), 5.15-5.05 (m, 2H), 4.78 (s, 1H), 3.99-3.81 (m, 4H), 3.02 (s, 1H), 2.83 (d, J=18.1 Hz, 1H), 2.51 (d, J=18.1 Hz, 1H), 2.39-2.21 (m, 2H), 2.17-2.09 (m, 1H), 2.05-1.95 (m, 1H), 1.70-1.42 (m, 4H), 1.50 (s, 3H), 1.28-1.19 (m, 1H), 1.17 (s, 3H), 0.99 (d, J=7.1 Hz, 3H).

¹³C NMR (151 MHz, CDCl₄) δ 216.3, 211.7, 200.6, 141.6, 114.4, 108.2, 65.5, 65.4, 53.82, 53.81, 53.0, 44.4, 43.2, 37.4, 35.6, 27.0, 26.9, 25.5, 21.3, 18.5, 16.8.

HRMS-ESI (m/z): calculated for [C₂₁H₃₀NaO₅]⁺ 385.1985, found 385.1991.

### Synthesis of the Alkyne S10:

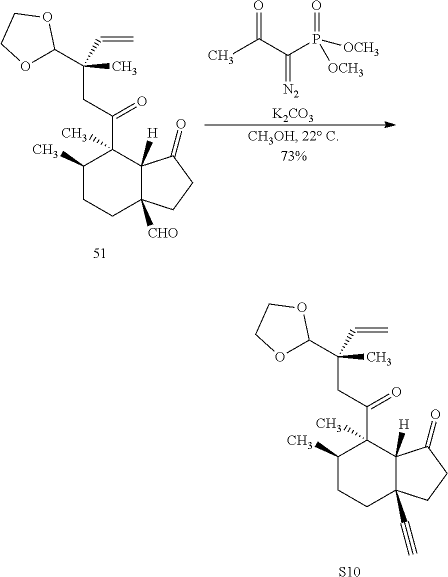

Dimethyl (1-diazo-2-oxopropyl)phosphonate (4.6 mg, 23.9 μmol, 5.78 equiv) was added to a solution of the aldehyde 51 (1.5 mg, 4.14 μmol, 1 equiv) and potassium carbonate (3.6 mg, 26.0 μmol, 6.29 equiv) in methanol (250 μL) at 22° C. under air. The resulting mixture was stirred for 90 min at 22° C. The product mixture was diluted with saturated aqueous ammonium chloride solution (1.5 mL) and the diluted mixture was extracted with ethyl acetate (3×1.5 mL). The organic layers were combined and the combined organic layers were dried over sodium sulfate. The dried solution was filtered and the filtrate was concentrated. The residue obtained was purified by preparative thin-layered chromatography (eluting with 40% ethyl acetate-pentane) to provide the alkyne S10 as a colorless oil (1.1 mg, 73%).

R_{f}=0.31 (20% ethyl acetate-pentane; PAA, stains purple).

¹H NMR (400 MHz, CDCl₃) δ 6.01 (dd, J=17.5, 11.0 Hz, 1H), 5.20-5.08 (m, 2H), 4.84 (s, 1H), 4.02-3.79 (m, 4H), 2.90 (s, 1H), 2.87 (d, J=17.6 Hz, 1H), 2.80 (d, J=17.5 Hz, 1H), 2.45-1.96 (m, 5H), 2.25 (s, 1H), 1.88-1.77 (m, 1H), 1.52-1.36 (m, 3H), 1.46 (s, 3H), 1.15 (s, 3H), 1.06 (d, J=6.8 Hz, 3H).

¹³C NMR (101 MHz, CDCl₃) δ 215.8, 208.4, 142.3, 113.8, 108.3, 88.9, 72.1, 65.4 (2C), 57.3, 51.3, 43.4, 43.2, 39.0, 38.1, 35.4, 33.7, 33.5, 26.9, 22.6, 18.0, 15.9.

HRMS-ESI (m/z): calculated for [C₂₂H₃₁O₄]⁺ 359.2217, found 359.2225.

### Synthesis of the Aldehyde 52:

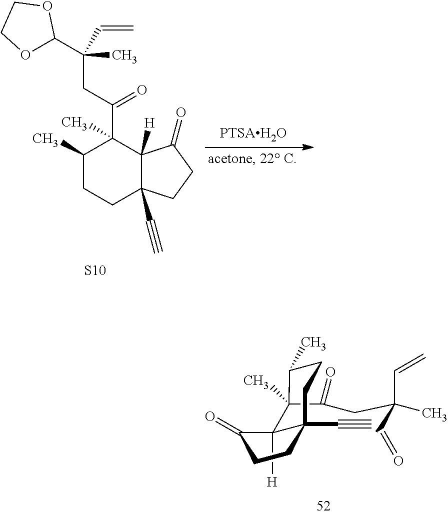

p-Toluenesulfonic acid (4.7 mg, 24.5 μmol, 4.00 equiv) was added to a solution of the acetal S10 (2.2 mg, 6.14 μmol, 1 equiv) in acetone (500 μL) at 22° C. under air. The resulting mixture was stirred for 3 h at 22° C. The product mixture was diluted sequentially with saturated aqueous sodium bicarbonate solution (1.0 mL) and saturated aqueous potassium carbonate solution (500 μL). The diluted mixture was extracted with ether (4×1.5 mL). The organic layers were combined and the combined organic layers were dried over sodium sulfate. The dried solution was filtered and the filtrate was concentrated. The residue obtained was used directly in the following step.

R_{f}=0.38 (20% ethyl acetate-pentane; PAA, stains purple).

¹H NMR (500 MHz, CDCl₃) δ 9.59 (s, 1H), 5.96 (dd, J=17.7, 10.9 Hz, 1H), 5.25 (d, J=10.9 Hz, 1H), 5.16 (d, J=17.7 Hz, 1H), 3.17 (d, J=17.5 Hz, 1H), 3.04 (d, J=17.6 Hz, 1H), 2.85 (s, 1H), 2.25-1.94 (m, 5H), 2.28 (s, 1H), 1.86-1.78 (m, 1H), 1.52-1.35 (m, 3H), 1.48 (s, 3H), 1.26 (s, 3H), 1.07 (d, J=6.9 Hz, 3H).

¹³C NMR (126 MHz, CDCl₃) δ 215.1, 207.9, 202.6, 139.2, 116.3, 88.7, 72.6, 57.7, 50.7, 50.3, 47.6, 39.1, 38.1, 35.2, 33.9, 33.5, 27.1, 22.7, 20.2, 15.8.

HRMS-ESI (m/z): calculated for [C₂₀H₂₇O₃]⁺ 315.1955, found 315.1953.

### Synthesis of the Cyclopentene 53:

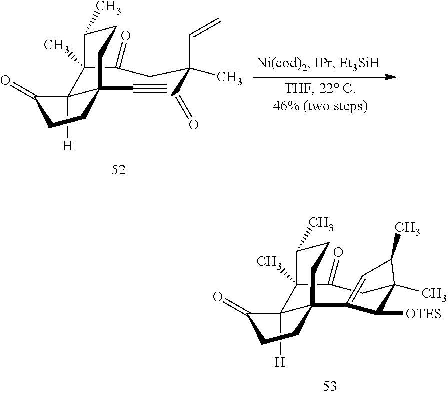

A mixture of bis(1,5-cyclooctadiene)nickel(0) (2.0 mg, 7.31 μmol, 1.19 equiv) and 1,3-bis(2,6-di-iso-propylphenyl)imidazol-2-ylidene (IPr, 2.8 mg, 7.31 μmol, 1.19 equiv) in tetrahydrofuran (500 μL) was stirred for 30 min at 22° C. resulting in a dark green solution. An aliquot of this solution (250 μL, 60 mol % of nickel and ligand) was added to a solution of the alkynyl aldehyde 52 (1.9 mg, 6.14 μmol, 1 equiv) and triethylsilane (3.5 μL, 21.9 μmol, 3.57 equiv) in tetrahydrofuran (480 μL) at 22° C. The resulting pale yellow solution was stirred for 2 h at 22° C. The product mixture was filtered through a pad of silica gel (eluting with 40% dichloromethane-hexanes). The filtrates were combined and concentrated to provide the cyclopentene 53 as a white solid (1.2 mg, 46%, two steps).

R_{f}=0.20 (60% dichloromethane-pentane; PAA, stains purple).

¹H NMR (600 MHz, CDCl₃) δ 5.83 (d, J=3.7 Hz, 1H), 4.17 (s, 1H), 2.63-2.55 (m, 1H), 2.58 (s, 1H), 2.43-2.31 (m, 2H), 2.28 (d, J=11.8 Hz, 1H), 2.05-1.94 (m, 2H), 1.88 (qd, J=13.3, 5.1 Hz, 1H), 1.78 (d, J=11.9 Hz, 1H), 1.78-1.72 (m, 1H), 1.62-1.34 (m, 3H), 1.35 (s, 3H), 1.20 (d, J=7.4 Hz, 3H), 1.14 (d, J=7.0 Hz, 3H), 1.06 (s, 3H), 0.96 (t, J=8.0 Hz, 9H), 0.63 (q, J=8.0 Hz, 6H).

¹³C NMR (151 MHz, CDCl₃) δ 215.9, 211.5, 150.6, 144.2, 82.8, 61.9, 54.5, 51.2, 49.7, 46.8, 45.7, 36.2, 36.0, 29.7, 28.7, 28.0, 21.2, 19.2, 16.8, 15.6, 7.3(3C), 5.9(3C).

HRMS-ESI (m/z): calculated for [C₂₆H₄₃O₃Si]⁺ 431.2976, found 431.2982.

### Synthesis of the Ketal 55:

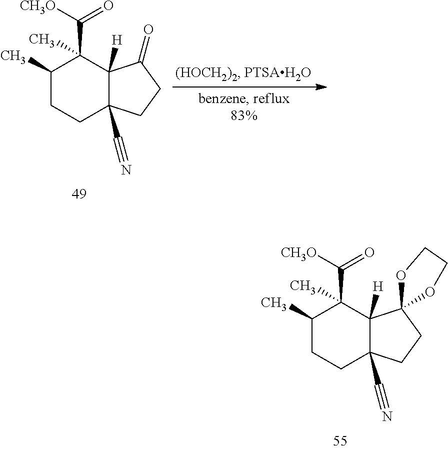

Ethylene glycol (674 μL, 12.1 mmol, 5.00 equiv) and p-toluenesulfonic acid (PTSA) monohydrate (9.2 mg, 48.1 μmol, 2.00 mol %) were added in sequence to the ketone 49 (600 mg, 2.41 mmol, 1 equiv) in benzene (6.0 mL) at 22° C. The reaction vessel was fitted with a Dean-Stark trap. The reaction mixture was stirred for 72 h at reflux. The product mixture was cooled to 22° C. and the cooled product mixture was concentrated. The residue obtained was purified by flash-column chromatography (eluting with 5% ethyl acetate-hexanes initially, grading to 40% ethyl acetate-hexanes, linear gradient) to provide the ketal 55 as a white solid (589 mg, 63%).

R_{f}=0.36 (20% ethyl acetate-hexanes; PAA, stains brown).

¹H NMR (500 MHz, CDCl₃) δ 4.03-3.96 (m, 1H), 3.94-3.85 (m, 2H), 3.84-3.77 (m, 1H), 3.69 (s 3H), 3.08 (s, 1H), 2.18-1.72 (m, 8H), 1.58-1.50 (m, 1H), 1.32 (s, 3H), 1.13 (d, J=6.9 Hz, 3H).

¹³C NMR (126 MHz, CDCl₃) δ 175.5, 124.1, 117.5, 64.4, 62.5, 53.3, 51.6, 46.5, 40.3, 36.2, 35.4, 33.8, 31.6, 28.1, 21.4, 16.2.

HRMS-ESI (m/z): calculated for [C₁₆H₂₃NO₄Na]⁺ 316.1525, found 316.1530.

### Synthesis of the Lactam 56b:

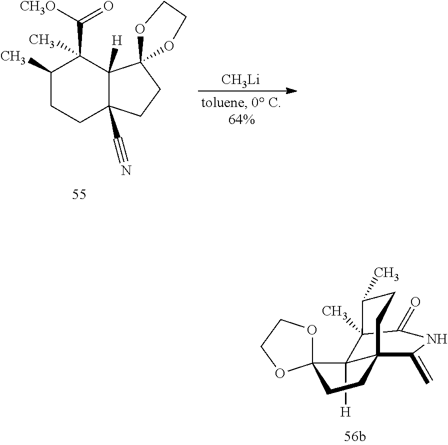

A solution of methyllithium in ether (1.6 M, 63 μL, 102 μmol, 3.00 equiv) was added dropwise to a solution of the ketal 55 (10.0 mg, 34.1 μmol, 1 equiv) in toluene (340 μL) at 0° C. The resulting mixture was stirred for 30 min at 0° C. The cold product mixture was diluted with saturated aqueous ammonium chloride solution (1.5 mL). The diluted product mixture was warmed to 22° C. over 5 min. The warmed product mixture was extracted with ethyl acetate (3×1.5 mL). The organic layers were combined and the combined organic layers were dried over sodium sulfate. The dried solution was filtered and the filtrate was concentrated. The residue obtained was purified by preparative thin-layered chromatography (eluting with 40% ethyl acetate-pentane) to provide the lactam 56 as a white solid (6.1 mg, 64%).

R_{f}=0.51 (40% ethyl acetate-pentane; UV).

¹H NMR (600 MHz, C₆D₆) δ 8.41 (s, 1H), 4.08 (s, 1H), 3.74 (s, 1H), 3.44-3.17 (m, 4H), 2.49 (dp, J=12.3, 6.6 Hz, 1H), 2.27 (s, 1H), 1.97 (td, J=13.1, 4.8 Hz, 1H), 1.86-1.71 (m, 2H), 1.66-1.36 (m, 4H), 1.56 (s, 3H), 1.34-1.27 (m, 1H), 1.12 (d, J=6.9 Hz, 3H).

¹³C NMR (151 MHz, C₆D₆) δ 173.7, 151.4, 117.4, 86.5, 64.5, 62.6, 53.7, 46.2, 44.2, 38.7, 34.2, 33.4, 33.2, 30.1, 19.3, 16.9.

HRMS-ESI (m/z): calculated for [C₁₆H₂₄NO₃]⁺ 278.1751, found 278.1754.

### Synthesis of the Eneimide 57:

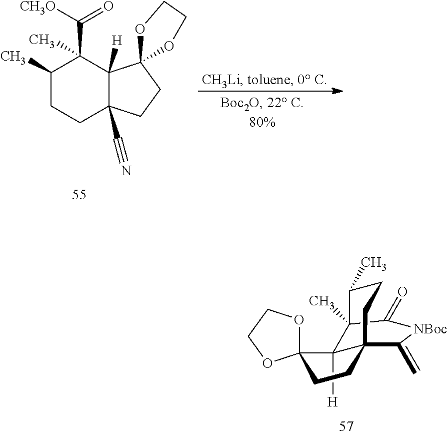

A solution of methyllithium in hexane (1.6 M, 2.56 mL, 4.09 mmol, 3.00 equiv) was added dropwise over 2 min to a solution of the cyano ketal 55 (400 mg, 1.36 mmol, 1 equiv) in toluene (20 mL) at 0° C. The resulting solution was stirred for 15 min at 0° C. Di-tert-butyl-dicarbonate (1.25 mL, 5.46 mmol, 4.00 equiv) was added dropwise over 2 min and the resulting solution was stirred for 1 h at 0° C. The solution was then warmed to 22° C. over 15 min. The warmed product mixture was diluted with saturated aqueous sodium hydrogen carbonate solution (50 mL), and the diluted mixture was extracted with ethyl acetate (3×50 mL). The organic layers were combined and the combined organic layers were dried over magnesium sulfate. The dried solution was filtered and the filtrate was concentrated. The residue obtained was purified by flash-column chromatography (eluting with 5% ethyl acetate-hexanes initially, grading to 20% ethyl acetate-hexanes, linear gradient) to provide the eneimide 57 as a viscous colorless oil (41.3 mg, 80%).

R_{f}=0.42 (20% ethyl acetate-hexanes; UV; PAA, stains orange).

¹H NMR (600 MHz, C₆D₆) δ 4.51 (d, J=1.9 Hz, 1H), 4.01 (d, J=1.9 Hz, 1H), 3.39-3.33 (m, 1H), 3.33-3.25 (m, 2H), 3.20-3.15 (m, 1H), 2.49 (dqd, J=13.5, 6.9, 4.7 Hz, 1H), 2.31 (d, J=1.1 Hz, 1H), 2.03-1.95 (m, 1H), 1.81-1.35 (m, 7H), 1.49 (s, 3H), 1.44 (s, 9H), 1.11 (d, J=6.9 Hz, 3H).

¹³C NMR (151 MHz, C₆D₆) δ 171.6, 152.8, 150.9, 117.3, 87.3, 84.0, 64.4, 62.6, 52.6, 46.4, 44.1, 38.3, 34.7, 33.8, 33.6, 29.7, 27.5 (3C), 19.3, 16.8.

HRMS-ESI (m/z): calculated for [C₂₁H₃₁NO₅Na]⁺ 400.2100, found 400.2096.

### Synthesis of the Diketone 59:

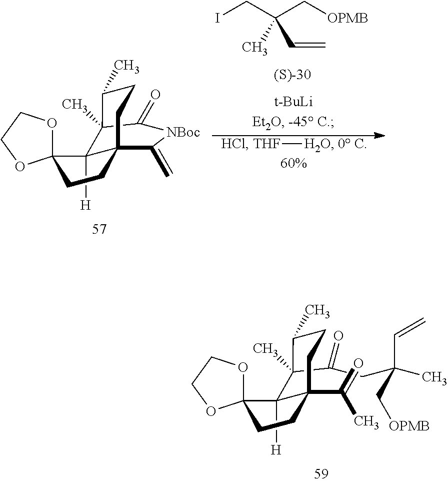

A solution of t-butyllithium in pentane (1.63 M, 1.05 mL, 1.72 mmol, 6.00 equiv) and the neopentyl iodide (S)-30 (297 mg, 858 μmol, 3.00 equiv) were added dropwise in sequence over 5 min to ether (3.0 mL) at −45° C. The resulting mixture was stirred for 40 min at −45° C. A solution of the eneimide 57 (108 mg, 286 μmol, 1 equiv) in ether (2 mL) was then added dropwise over 5 min. The resulting mixture was stirred for 1 h at −45° C. Aqueous sodium thiosulfate solution (20% w/v, 2.0 mL) was then added and the resulting mixture was warmed to 22° C. over 10 min. The warmed mixture was further diluted with aqueous sodium thiosulfate solution (20% w/v, 30 mL). The diluted mixture was extracted with ethyl acetate (3×25 mL). The organic layers were combined and the combined organic layers were dried over magnesium sulfate. The dried solution was filtered and the filtrate was concentrated. The residue obtained was dissolved in tetrahydrofuran (20 mL) and the resulting solution was cooled to 0° C. Aqueous hydrochloric acid solution (1 M, 20 mL) was added dropwise. The resulting mixture was stirred for 3 h at 0° C. The product mixture was diluted with aqueous sodium hydroxide solution (10 M, 2.0 mL) and the diluted mixture was warmed to 22° C. The warmed mixture was extracted with ethyl acetate (3×30 mL). The organic layers were combined and the combined organic layers were dried over magnesium sulfate. The dried solution was filtered and the filtrate was concentrated. The residue obtained was purified by flash-column chromatography (eluting with 10% ethyl acetate-hexanes initially, grading to 30% ethyl acetate-hexanes, linear gradient) to provide the diketone 59 as a colorless oil. (85.7 mg, 60%).

R_{f}=0.36 (25% ethyl acetate-pentane; UV; PAA, stains pink).

¹H NMR (500 MHz, C₆D₆, 65° C.): δ 7.22 (d, J=8.6 Hz, 2H), 6.80 (d, J=8.6 Hz, 2H), 6.16 (dd, J=17.7, 10.9 Hz, 1H), 5.08 (dd, J=17.7, 1.2 Hz, 1H), 5.04 (dd, J=10.9, 1.2 Hz, 1H), 4.37 (s, 2H), 3.69 (q, J=8.5, 7.6 Hz, 1H), 3.57 (d, J=8.5 Hz, 1H), 3.49 (d, J=8.6 Hz, 1H), 3.48-3.31 (m, 3H), 3.39 (s, 1H), 3.36 (s, 3H), 2.79 (d, J=18.0 Hz, 1H), 2.66 (d, J=18.0 Hz, 1H), 2.51-2.41 (m, 1H), 2.13 (s, 3H), 1.96-1.86 (m, 1H), 1.84-1.74 (m, 1H), 1.67-1.52 (m, 3H), 1.38 (s, 3H), 1.38-1.26 (m, 2H), 1.30 (s, 3H), 1.24-1.14 (m, 1H), 0.81 (d, J=6.9 Hz, 3H).

¹³C NMR (126 Hz, C₆D₆, 65° C.): δ211.2, 209.2, 159.9, 145.7, 131.6, 129.4 (2C), 120.1, 114.3 (2C), 112.0, 77.3, 73.5, 65.0, 63.7, 57.5, 54.9, 50.5, 46.4, 43.7, 40.6, 37.5, 35.9, 29.7, 26.4, 25.3, 24.7, 22.3, 21.5, 15.7.

HRMS-ESI (m/z): calculated for [C₃₀H₄₂NaO₆]⁺ 521.2874, found 521.2872.

### Synthesis of the Vinyl Triflate 60:

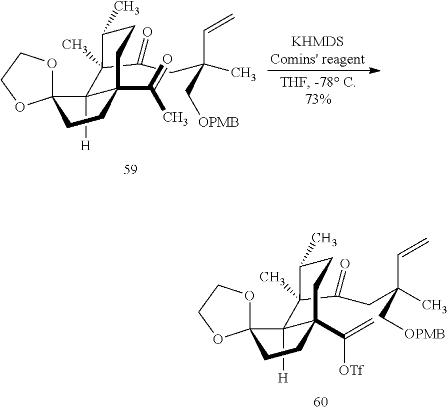

A solution of potassium bis(trimethylsilyl)amide in toluene (0.5 M, 1.00 mL, 500 μmol, 2.91 equiv) was added dropwise to a solution of the diketone 59 (85.6 mg, 172 μmol, 1 equiv) and N-(5-chloro-2-pyridyl) bis(trifluoromethanesulfonimide) (135 mg, 344 μmol, 2.00 equiv) in tetrahydrofuran (3.0 mL) at −78° C. The resulting solution was stirred for 10 min at −78° C. The cold product mixture was diluted with saturated aqueous sodium bicarbonate solution (3.0 mL) and then was allowed to warm to 22° C. over 5 min. The warmed mixture was extracted with ether (3×5 mL). The organic layers were combined and the combined organic layers were dried over sodium sulfate. The dried solution was filtered and the filtrate was concentrated. The residue obtained was purified by flash-column chromatography (eluting with 5% ethyl acetate-hexanes initially, grading to 40% ethyl acetate-hexanes, linear gradient) to provide the vinyl triflate 60 as a colorless oil (79.6 mg, 73%).

R_{f}=0.27 (20% ether-hexanes; PAA, stains dark green).

¹H NMR (500 MHz, C₆D₆, 65° C.) δ 7.20 (d, J=7.9 Hz, 2H), 6.80 (d, J=7.5 Hz, 2H), 6.12 (dd, J=17.7, 10.9 Hz, 1H), 5.19 (d, J=4.4 Hz, 1H), 5.06 (dd, J=17.9, 1.9 Hz, 1H), 5.02 (dd, J=10.9, 1.8 Hz, 1H), 4.77 (d, J=5.0 Hz, 1H), 4.34 (s, 2H), 3.81 (q, J=7.7 Hz, 1H), 3.57-3.34 (m, 2H), 3.52 (d, J=8.5 Hz, 1H), 3.46 (d, J=8.4 Hz, 1H), 3.37 (s, 3H), 3.30 (q, J=7.2 Hz, 1H), 3.22 (s, 1H), 2.66 (s, 2H), 1.95-1.85 (m, 1H), 1.84-1.73 (m, 2H), 1.70-1.59 (m, 2H), 1.58-1.48 (m, 2H), 1.37 (s, 3H), 138-1.22 (m, 2H), 1.27 (s, 3H), 0.85 (d, J=7.0 Hz, 3H).

¹³C NMR (151 MHz, C₆D₆) δ 210.1, 162.4, 159.7, 145.5, 131.2, 129.4 (2C), 119.3, 119.1 (q, J=320 Hz), 114.1 (2C), 112.1, 100.2, 76.7, 73.2, 64.7, 64.1, 54.8, 50.5, 49.6, 46.0, 43.0, 40.4, 37.0, 35.7, 31.0, 27.8, 24.4, 22.0, 21.7, 15.0.

¹⁹F NMR (470 MHz, C₆D₆, 65° C.) δ−75.23.

HRMS-ESI (m/z): calculated for [C₃₁H₄₁F₃O₈SNa]⁺ 653.2366, found 653.2374.

### Synthesis the Alkyne 61:

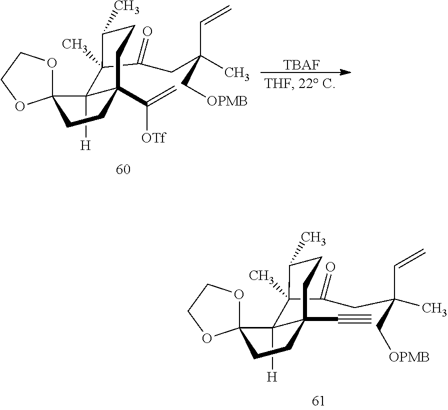

A solution of tetra-n-butylammonium fluoride in tetrahydrofuran (1.0M, 124 μL, 124 μmol, 4.00 equiv) was added to a solution of the triflate 60 (19.5 mg, 30.9 μmol, 1 equiv) in tetrahydrofuran (300 μL) at 22° C. The resulting mixture was stirred for 30 min at 22° C. The product mixture was diluted with saturated aqueous ammonium chloride solution (3.0 mL). The diluted product mixture was extracted with ether (3×3 mL). The organic layers were combined and the combined organic layers were washed with saturated aqueous ammonium chloride solution (5.0 mL). The organic layer was isolated and dried over sodium sulfate. The dried solution was filtered and the filtrate was concentrated to afford the alkyne 61 as a colorless oil. This unpurified alkyne 61 (containing small amount of TBAF) was used directly in the following step.

R_{f}=0.43 (20% ethyl acetate-hexanes; PAA, stains blue).

¹H NMR (500 MHz, C₆D₆, 65° C.) δ 7.21 (d, J=8.6 Hz, 2H), 6.79 (d, J=8.6 Hz, 2H), 6.18 (dd, J=17.7, 10.9 Hz, 1H), 5.09 (dd, J=17.7, 1.3 Hz, 1H), 5.01 (dd, J=10.9, 1.3 Hz, 1H), 4.36 (s, 2H), 3.63 (d, J=8.5 Hz, 1H), 3.59 (d, J=8.5 Hz, 1H), 3.53-3.41 (m, 2H), 3.39-3.28 (m, 2H), 3.36 (s, 3H), 3.01 (d, J=17.0, 1H), 2.95 (s, 1H), 2.95 (d, J=17.1 Hz, 1H), 2.53-237 (m, 1H), 2.17-2.07 (m, 1H), 1.97 (s, 1H), 1.95-1.86 (m, 1H), 1.78-1.59 (m, 5H), 1.55-1.45 (m, 1H), 1.39 (s, 3H), 1.31 (d, J=7.3 Hz, 3H), 1.30 (s, 3H).

¹³C NMR (126 MHz, C₆D₆, 65° C.) δ 209.5, 159.8, 145.9, 131.7, 129.3 (2C), 119.4, 114.3 (2C), 111.9, 90.8, 77.2, 73.3, 71.1, 64.1, 62.4, 54.9, 53.2, 51.8, 44.3, 41.3, 40.8, 37.4, 37.2, 36.5, 34.2, 28.5, 22.7, 21.6, 16.5.

HRMS-ESI (m/z) calculated for [C₃₀H₄₀NaO₅]⁺ 503.2768, found 503.2770.

### Synthesis of the Alcohol S11:

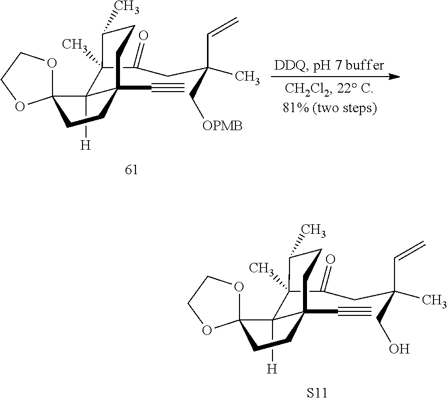

Aqueous potassium phosphate buffer (1.0 mM, pH 7, 130 μL) was added to a solution of the alkyne 61 (30.9 μmol, 1 equiv) in dichloromethane (600 μL) at 22° C. 2,3-Dichloro-5,6-dicyano-p-benzoquinone (DDQ, 28.1 mg, 124 μmol, 4.00 equiv) was then added in one portion and the resulting solution was stirred for 1 h at 22° C. open to air. The product mixture was diluted with saturated aqueous sodium bicarbonate solution (1.5 mL). The diluted product mixture was extracted with dichloromethane (3×1.5 mL). The organic layers were combined and the combined organic layers were dried over sodium sulfate. The dried solution was filtered and the filtrate was concentrated. The residue obtained was purified by flash-column chromatography (eluting with 20% ethyl acetate-hexanes initially, grading to 30% ethyl acetate-hexanes, two steps) to provide the alkynyl alcohol S11 as a colorless oil (9.0 mg, 81%, two steps). The isolated sample contains minor amounts of the C12 diastereomer, which was inseparable.

R_{f}=0.15 (25% ethyl acetate-hexanes; PAA, stains brown).

¹H NMR (600 MHz, CDCl₃) δ 5.91 (dd, J=17.6, 10.9 Hz, 1H), 5.08 (d, J=10.1 Hz, 1H), 5.06 (d, J=16.8 Hz, 1H), 4.02-3.74 (m, 4H), 3.60 (d, J=10.9 Hz, 1H), 3.45 (d, J=10.9 Hz, 1H), 2.81 (d, J=17.2 Hz, 1H), 2.76 (d, J=16.9 Hz, 1H), 2.67 (s, 1H), 2.17-2.05 (m, 1H), 2.15 (s, 1H), 2.03-1.77 (m, 5H), 1.71-1.57 (m, 2H), 1.49-1.39 (m, 1H), 1.27 (s, 3H), 1.08 (s, 3H), 1.05 (d, J=6.9 Hz, 3H).

¹³C NMR (151 Hz, CDCl₃), δ 212.4, 143.5, 118.9, 113.4, 90.4, 70.7, 70.1, 64.2, 62.4, 52.6, 51.2, 46.3, 42.1, 40.4, 36.7, 36.6, 36.5, 33.4, 27.9, 23.1, 21.0, 15.9.

HRMS-ESI (m/z): calculated for [C₂₂H₃₂O₄Na]⁺ 383.2198, found 383.1294.

### Synthesis of the Aldehyde 62:

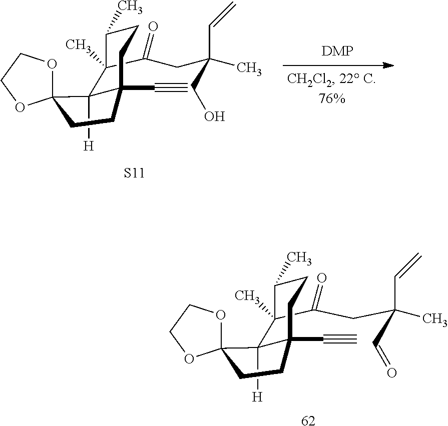

The Dess-Martin periodinane (42.4 mg, 99.9 μmol, 4.00 equiv) was added in one portion to a solution of the alkynyl alcohol S11 (9.0 mg, 25.0 μmol, 1 equiv) in dichloromethane (500 μL) at 22° C. The resulting mixture was stirred for 30 min at 22° C. open to air. The product mixture was diluted sequentially with ether (1.5 mL), aqueous sodium thiosulfate solution (20% w/v, 1.0 mL), saturated aqueous sodium bicarbonate solution (1.0 and water (1.0 mL). The resulting mixture was stirred at 22° C. until it became clear (approximately 1 h) and then extracted with ether (3×3.0 mL). The organic layers were combined and the combined organic layers were dried over magnesium sulfate. The dried solution was filtered and the filtrate was concentrated. The residue obtained was purified by flash-column chromatography (eluting with 25% ethyl acetate-hexanes) to provide the alkynyl aldehyde 62 as a colorless oil (6.8 mg, 76%). The isolated sample contains minor amounts of the C12 diastereomer, which was inseparable.

R_{f}=0.32 (25% ethyl acetate-hexanes; PAA, stains purple).

¹H NMR (600 MHz, CDCl₃) δ 9.60 (s, 1H), 5.98 (dd, J=17.6, 10.8 Hz, 1H), 5.22 (d, J=10.9 Hz, 1H), 5.14 (d, J=17.7 Hz, 1H), 4.00-3.77 (m, 4H), 3.23 (d, J=17.4 Hz, 1H), 3.03 (d, J=17.4 Hz, 1H), 2.69 (s, 1H), 2.15 (s, 1H), 2.08-1.76 (m, 6H), 1.70-1.55 (m, 2H), 1.49-1.38 (m, 1H), 1.27 (s, 3H), 1.23 (s, 3H), 1.03 (d, J=6.8 Hz, 3H).

¹³C NMR (151 Hz, CDCl₃) δ 209.5, 202.9, 139.4, 118.9, 116.0, 90.2, 71.1, 64.3, 62.5, 53.0, 50.7, 50.3, 47.7, 40.4, 36.7, 36.6, 36.5, 33.4, 27.8, 21.2, 20.3, 15.9.

HRMS-ESI (m/z): calculated for [C₂₂H₃₀NaO₄]⁺ 381.2036, found 381.2034.

### Synthesis of Enal 63:

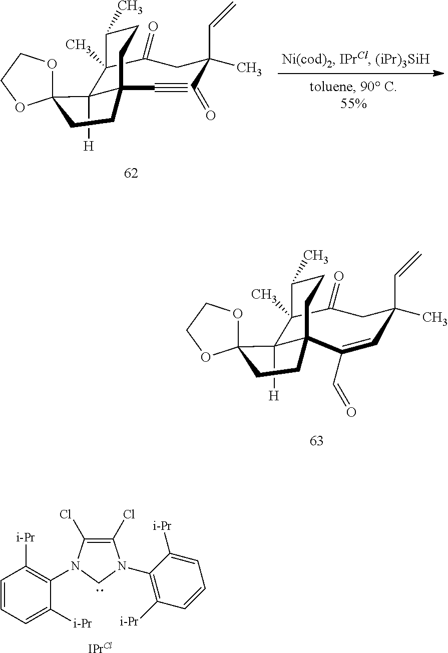

A solution of bis(1,5-cyclooctadiene)nickel(0) (2.0 mg, 7.36 μmol, 40 mol %) and 4,5-dichloro-1,3-bis(2,6-diisopropylphenyl)imidazol-2-ylidene (IPr^{Cl}, 3.4 mg, 7.36 μmol, 40 mol %) in toluene (300 μL) was stirred at 22° C. for 20 min. The resulting orange solution was added the aldehyde 6.2 (6.6 mg, 18.4 μmol, 1 equiv) in toluene (1.0 mL) and tri-i-propylsilane (18.9 μL, 92.1 μmol, 5.00 equiv) in sequence. The resulting pale yellow solution was heated and stirred for 2 h at 90° C. The product mixture was cooled to 22° C. The cooled product mixture was concentrated and the residue obtained was purified by flash-column chromatography (eluting with 20% ethyl acetate-hexanes) to provide the enal 63 as a white solid (3.6 mg, 55%).

R_{f}=0.28 (20% ethyl acetate-hexanes, UV; PAA, stains green).

¹H NMR (500 MHz, CDCl₃) δ 9.21 (s, 1H), 6.31 (dd, J=17.3, 10.6 Hz, 1H), 6.14 (s, 1H), 5.12 (d, J=17.4 Hz, 1H), 5.08 (d, J=10.5 Hz, 1H), 4.08-4.00 (m, 1H), 3.99-3.89 (m, 2H), 3.86-3.78 (m, 1H), 3.33 (d, J=12.8 Hz, 1H), 3.06 (s, 1H), 2.46 (ddd, J=13.0, 8.6, 2.3 Hz, 1H), 2.36-2.33 (m, 1H), 2.32 (d, J=12.8 Hz, 1H), 1.97-1.83 (m, 2H), 1.62-1.52 (m, 1H), 1.48-1.24 (m, 4H), 1.38 (s, 3H), 1.26 (s, 3H), 1.15 (d, J=7.1 Hz, 3H).

¹³C NMR (126 MHz, CDCl₃) δ 213.3, 198.0, 160.1, 147.9, 146.3, 119.5, 110.4, 64.3, 62.1, 52.6, 51.3, 49.0, 45.0, 43.4, 36.2, 36.0, 35.8, 28.8, 28.2, 27.6, 19.5, 16.2.

HRMS-ESI (m/z): calculated for [C₂₂H₃₀NaO₄]⁺ 381.2036, found 381.2037.

### Synthesizing of the Cyclopentene 66:

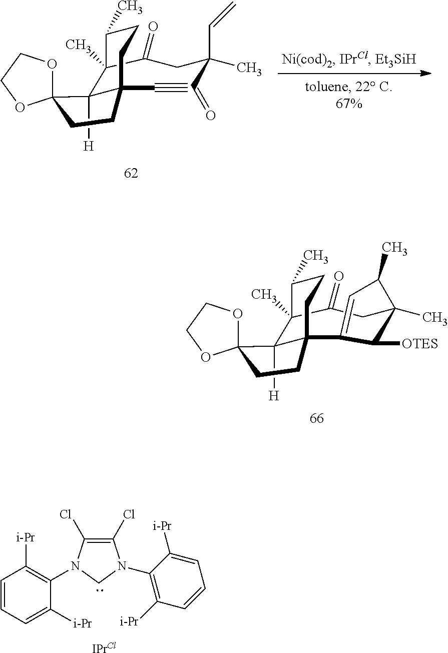

A solution of bis(1,5-cyclooctadiene)nickel(0) (9.8 mg, 35.6 μmol, 1.00 equiv) and 4,5-dichloro-1,3-bis(2,6-diisopropylphenyl)imidazol-2-ene (IPr^{Cl}, 16.3 mg, 35.6 μmol, 1.00 equiv) in toluene (3.6 mL) was stirred for 20 min at 22° C. A portion of the orange catalyst stock solution (230 μL, 40 mol % of nickel and ligand) was added to a solution of the alkynyl aldehyde 62 (2.0 mg, 5.60 μmol, 1 equiv) and triethylsilane (4.5 μL, 27.9 μmol, 5.00 equiv) in toluene (1.0 mL) at 22° C. The resulting solution was stirred for 3 h at 22° C. The product mixture was filtered through a pad of silica gel (eluting with 40% dichloromethane-hexanes, grading to 80% dichloromethane-hexanes, three steps) to provide the cyclopentene 66 as a white solid (1.8 mg, 67%).

R_{f}=0.23 (50% dichloromethane-pentane; PAA, stains purple).

¹H NMR (600 MHz, CDCl₃) δ 5.79 (dd, J=4.2, 1.5 Hz, 1H), 4.23 (s, 1H), 4.01-3.86 (m, 3H), 3.82-3.44 (m, 1H), 2.61 (s, 1H), 2.59-2.51 (m, 1H), 2.43 (d, J=11.8 Hz, 1H), 2.41-2.33 (m, 1H), 2.02-1.97 (m, 2H), 1.92-1.71 (m, 4H), 1.71 (d, J=11.7 Hz, 1H), 1.49 (dt, J=12.9, 5.3 Hz, 1H), 1.44-1.38 (m, 1H), 1.20 (d, J=7.4 Hz, 3H), 1.14 (d, J=7.1 Hz, 3H), 1.06 (s, 3H), 1.05 (s, 3H), 0.96 (t, J=8.0 Hz, 9H), 0.62 (q, J=7.8 Hz, 6H).

¹³C NMR (151 MHz, CDCl₃) δ 213.6, 152.2, 143.1, 118.4, 83.0, 64.7, 62.3, 55.6, 54.1, 51.7, 49.4, 46.9, 45.8, 37.8, 33.8, 31.5, 29.4, 28.3, 21.1, 19.3, 17.0, 15.7, 7.3 (6C), 5.9 (6C).

HRMS-ESI (m/z): calculated for [C₂₂H₃₁O₃]⁺(—OSiEt₃) 343.2268, found 343.2275.

### Synthesis of the Diketone 75:

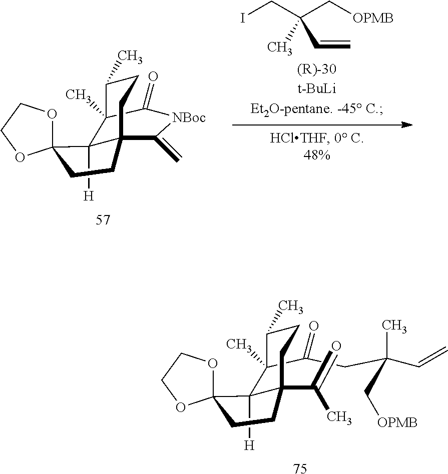

A solution of r-butyllithium in pentane (1.7 M, 680 μL, 1.16 mmol, 4.40 equiv) and the neopentyl iodide (R)-30 (218 mg, 630 μmol, 2.40 equiv) were added in sequence over 5 min to a solution of pentane-ether (8:1 v/v, 3.6 mL) at −45° C. The resulting mixture was stirred for 40 min at −45° C. A solution of the enimide 57 in ether (140 mM, 1.9 mL, 263 μmol, 1 equiv) was added dropwise over 5 min. Upon completion of the addition, the reaction mixture was stirred for 2 h at −45° C. Aqueous sodium thiosulfate solution (20% w/v, 2.0 mL) was then added and the resulting mixture was warmed to 22° C. over 10 min. The warmed mixture was further diluted with aqueous sodium thiosulfate solution (20% w/v, 30 mL). The diluted mixture was extracted with ethyl acetate (3×20 mL). The organic layers were combined and the combined organic layers were dried over magnesium sulfate. The dried solution was filtered and the filtrate was concentrated. The residue obtained was dissolved in tetrahydrofuran (10 mL) and cooled to 0° C. for 10 min. Aqueous hydrochloric acid solution (1 M, 10 mL) was added dropwise via syringe. The resulting mixture was stirred for 3 h at 0° C. The product mixture was diluted with aqueous sodium hydroxide solution (10 M, 4.5 mL) and the diluted mixture was warmed to 22° C. The warmed mixture was extracted with ethyl acetate (3×50 mL). The organic layers were combined and the combined organic layers were dried over magnesium sulfate. The dried solution was filtered and the filtrate was concentrated. The residue obtained was purified by flash-column chromatography (eluting with 10% ethyl acetate-hexanes initially, grading to 30% ethyl acetate-hexanes, linear gradient) to provide the diketone 75 as a colorless oil. The purity of the diketone 75 was determined by NMR analysis against an internal standard (84.0 mg, 73% w/w purity, 48%).

R_{f}=0.36 (40% ether-pentane; UV, PAA, stains pink).

¹H NMR (500 MHz, CDCl₃): δ 7.23 (d, J=8.5 Hz, 2H), 6.87 (d, J=8.5 Hz, 2H), 5.98 (dd, J=11, 18 Hz, 1H), 5.04-4.98 (m, 2H), 4.42 (dd, J=12, 17 Hz, 2H), 3.92-3.82 (m, 2H), 3.80 (s, 3.62-3.50 (m, 2H), 3.46 (d, J=8.5 Hz, 1H), 3.29 (d, J=8.5 Hz, 1H), 3.23 (s, 1H), 2.70 (d, J=18 Hz, 1H), 2.64 (d, J=18 Hz, 1H), 2.52-2.53 (m, 1H), 2.22 (s, 3H), 1.95-1.77 (m, 4H), 1.71-1.60 (m, 1H), 1.59-1.41 (m, 2H), 1.50 (s, 3H), 1.27-1.18 (m, 1H), 1.16 (s, 3H), 0.80 (d, J=7.1 Hz, 3H).

¹³C NMR (126 Hz, CDCl₃): δ212.1, 211.7, 159.1, 144.5, 130.9, 129.2 (2C), 119.6, 113.8 (2C), 112.4, 76.9, 73.0, 65.0, 63.9, 57.4, 55.4, 49.8, 45.5, 43.7, 40.0, 37.1, 35.2, 27.9, 24.9, 24.8, 24.3, 22.5, 21.4, 15.6.

HRMS-ESI (m/z): calculated for [C₃₀H₄₃O₆]⁺ 499.3060, found 499.3065.

### Synthesis of the Vinyl Triflate 76:

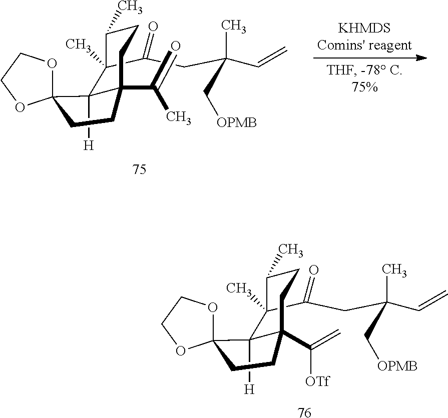

A solution of potassium bis(trimethylsilyl)amide in tetrahydrofuran (1 M, 508 μL, 508 μmol, 1.70 equiv) was added dropwise to a solution of the diketone 75 (149 mg, 299 μmol, 1 equiv) and N-(5-chloro-2-pyridyl)bis(trifluoromethanesulfonimide) (176 mg, 448 μmol, 1.50 equiv) in tetrahydrofuran at −78° C. The reaction mixture was stirred for 10 min at −78° C. and then was diluted with saturated aqueous sodium bicarbonate solution (2.0 mL). The diluted product mixture was warmed to 22° C. over 5 min. The warmed mixture was extracted with ether (3×1.5 mL). The organic layers were combined and the combined organic layers were dried over sodium sulfate. The dried solution was filtered and the filtrate was concentrated. The residue obtained was purified by flash-column chromatography (eluting with 5% ethyl acetate-hexanes initially, grading to 40% ethyl acetate-hexanes, linear gradient) to provide the vinyl triflate 76 as a colorless oil (189 mg, 75%).

R_{f}=0.35 (20% ethyl acetate-pentane; PAA, stains dark green).

¹H NMR (400 MHz, CDCl₃) δ 7.22 (d, J=8.6 Hz, 2H), 6.86 (d, J=8.6 Hz, 2H), 6.00 (dd, J=18.0, 10.5 Hz, 1H), 5.17 (d, J=4.9 Hz, 1H), 5.06 (d, J=4.9 Hz, 1H), 5.00 (d, J=11.8 Hz, 1H), 5.00 (d, J=16.0 Hz, 1H), 4.43 (d, J=12.4 Hz, 1H), 4.39 (d, J=11.7 Hz, 1H), 4.02-3.83 (m, 2H), 3.80 (s, 3H), 3.64-3.50 (m, 2H), 3.46 (d, J=8.5 Hz, 1H), 3.29 (d, J=8.6 Hz, 1H), 3.03 (s, 1H), 2.65 (d, J=18.1 Hz, 1H), 2.58 (d, J=17.9 Hz, 1H), 2.03-1.75 (m, 7H), 1.57-1.38 (m, 2H), 1.49 (s, 3H), 1.15 (s, 3H), 0.76 (d, J=6.9 Hz, 3H).

¹³C NMR (101 MHz, CDCl₃) δ 210.8, 162.4, 159.1, 144.5, 130.9, 129.1 (2C), 119.1, 118.5 (q, J=320 Hz),113.8 (2C), 112.4, 100.3, 76.7, 72.9, 64.6, 64.0, 55.4, 50.5, 49.4, 46.0, 43.4, 40.2, 36.9, 35.5, 31.1, 27.9, 24.6, 22.6, 21.8, 15.0.

¹⁹F NMR (376 MHz, CDCl₃) δ−74.88.

HRMS-ESI (m/z): calculated for [C₃₁H₄₁F₃O₈SNa]⁺ 653.2366, found 653.2365.

### Synthesis of the Alkyne 77:

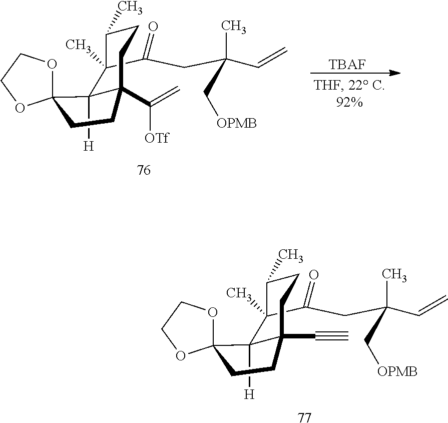

A solution of tetra-n-butylammonium fluoride in tetrahydrofuran (1.0 M, 560 μL, 558 μmol, 4.00 equiv) was added to a solution of the vinyl triflate 76 (88.0 mg, 140 μmol, 1 equiv) in tetrahydrofuran (1.4 mL) at 22° C. The reaction mixture was stirred for 30 min at 22° C. and then was diluted with saturated aqueous ammonium chloride solution (20 mL)/ The diluted product mixture was extracted with ether (3×20 mL). The organic layers were combined and the combined organic layers were washed with saturated aqueous ammonium chloride solution (50 mL). The washed organic layer was dried over sodium sulfate. The dried solution was filtered and the filtrate was concentrated to afford the alkyne 77 as a colorless oil (61.9 mg, 92%). The isolated sample contains minor amounts of the C12 diastereomer, which was inseparable.

R_{f}=0.59 (40% v/v ether-hexanes; PAA, stains blue).

¹H NMR (500 MHz, CDCl₃) δ 7.23 (d, J=8.6 Hz, 2H), 6.85 (d, J=8.7 Hz, 2H), 6.03 (dd, J=17.6, 10.9 Hz, 1H), 5.04-4.94 (m, 2H), 4.43 (d, J=12.0 Hz, 1H), 4.40 (d, J=11.8 Hz, 1H), 3.93-3.72 (m, 4H), 3.79 (s, 3H), 3.44 (d, J=8.5 Hz, 1H), 3.36 (d, J=8.6 Hz, 1H), 2.81 (d, J=17.3 Hz, 1H), 2.71 (s, 1H), 2.70 (d, J=17.2 Hz, 1H), 2.01 (s, 1H), 2.14-1.74 (m, 6H), 1.70-1.56 (m, 2H), 1.49-1.41 (m, 1H), 1.25 (s, 3H), 1.14 (s, 3H), 1.03 (d, J=6.9 Hz, 3H).

¹³C NMR (151 MHz, CDCl₃) δ 210.7, 159.0, 145.0, 131.1, 129.0, 119.0, 113.7, 112.0, 90.6, 76.8, 72.9, 70.7, 64.2, 62.5, 55.4, 52.5, 51.2, 44.0, 40.7, 40.3, 36.8, 36.5, 33.4, 30.4, 27.7, 22.3, 21.3, 16.0.

HRMS-ESI (m/z): calculated for [C₃₀H₄₁O₅]⁺ 481.2954, found 481.2956.

One-Step Synthesis of the Alkyne 77 from the Diketone 75:

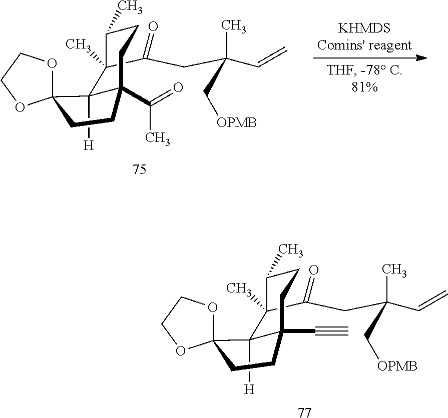

A solution of potassium bis(trimethylsilyl)amide in tetrahydrofuran (0.5 M, 860 μL, 430 μmol, 3.50 equiv) was added dropwise over 10 min to a solution of the diketone 75 (84.0 mg, 73% w/w purity, 123 μmol, 1 equiv) and N-(5-chloro-2-pyridyl)triflimide (Comins' reagent, 62.8 mg, 160 μmol, 1.30 equiv) tetrahydrofuran (2.4 mL) at −78° C. The resulting solution was stirred for 30 min at −78° C. and then methanol (1.2 mL) was added. The resulting mixture was warmed to 22° C. over 10 min. The warmed mixture was diluted with aqueous sodium hydroxide solution (1 M, 4.0 mL) and the diluted mixture Was extracted with ether (3×4.0 mL). The organic layers were combined and the combined organic layers were dried over magnesium sulfate. The dried solution was filtered and the filtrate was concentrated. The residue obtained was purified by flash-column chromatography (eluting with 5% ethyl acetate-hexanes initially, grading to 15% ethyl acetate-hexanes, linear gradient) to provide the alkyne 77 as a colorless oil (59.1 mg, 81%). In some instances, then trimethylsilyl-protected alkyne was formed in approximately 0-30% yield depending on the purity of diketone 75. In cases where this side product was formed, the aqueous sodium hydroxide solution was replaced with aqueous lithium hydroxide solution (4 M) and the resulting mixture was stirred at 22° C. for 0.5-4 h to quantitatively desilylate the alkyne. Spectroscopic data for the alkyne 77 obtained in this way were in agreement with those obtained above (76→77).

### Synthesis of Alcohol S12:

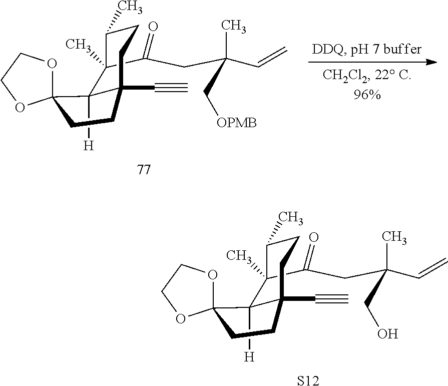

Aqueous potassium phosphate buffer solution (10 mM, pH 7, 130 μL) and 2,3-dichloro-5,6-dicyano-p-benzoquinone (DDQ, 117 mg, 515 μmol, 4.00 equiv) were added in sequence to a solution of the alkyne 77 (61.9 mg, 129 μmol, 1 equiv) in dichloromethane (430 μL) at 22° C. The resulting solution was stirred for 30 min at 22° C. open to air. The product mixture was diluted with saturated aqueous sodium bicarbonate solution (5.0 mL). The diluted product mixture was extracted with dichloromethane (3×5.0 mL). The organic layers were combined and the combined organic layers were dried over sodium sulfate. The dried solution was filtered and the filtrate was concentrated. The residue obtained was purified by flash-column chromatography (eluting with 10% ethyl acetate-pentane initially, grading to 30% ethyl acetate-pentane, four steps) to provide the alkynyl alcohol S12 as a colorless oil (45.4 mg, 98%). The isolated sample contains minor amounts of the C12 diastereomer, which was inseparable.

R_{f}=0.19 (40% ether-pentane; PAA, stains brown).

¹H NMR (500 MHz, CDCl₃) δ 5.84 (dd, J=18.0, 10.6 Hz, 1H), 5.03 (d, J=11.4 Hz, 1H), 5.02 (d, J=17.0 Hz, 1H), 3.99-3.76 (m, 4H), 3.55 (d, J=11.0 Hz, 1H), 3.49 (d, J=10.9 Hz, 1H), 3.09 (s, br, 1H), 2.82 (d, J=17.0 Hz, 1H), 2.75 (d, J=17.0 Hz, 1H), 2.67 (s, 1H), 2.13 (s, 1H), 2.17-1.72 (m, 6H), 1.72-1.54 (m, 2H), 1.49-1.38 (m, 1H), 1.27 (s, 3H), 1.06 (s, 3H), 1.05 (d, J=7.0 Hz, 3H).

¹³C NMR (101 Hz, CDCl₃), δ 212.8, 145.0, 118.9, 112.6, 90.2, 70.8, 70.0, 64.2, 62.4, 52.6, 51.4, 45.3, 42.0, 40.4, 36.63, 36.61, 36.4, 33.4, 27.9, 21.0, 20.7, 15.9.

HRMS-ESI (m/z): calculated for [C₂₂H₃₂O₄Na]⁺ 383.2198, found 383.2207.

### Synthesis of the Aldehyde 78:

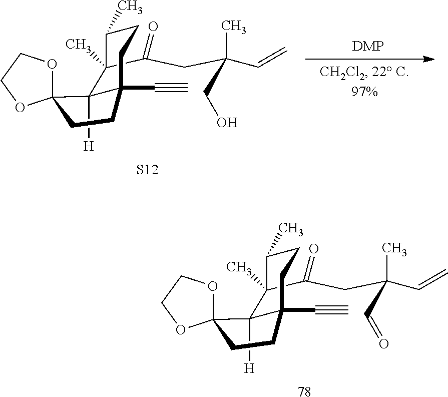

The Dess-Martin periodinane (419 mg, 988 μmol, 4.00 equiv) was added in one portion to a solution of the alkynyl alcohol S12 (89.2 mg, 247 μmol, 1 equiv) in dichloromethane (2.5 mL) at 22° C. The resulting mixture was stirred for 1 h at 22° C. open to air. The product mixture was diluted sequentially with ether (2.5 mL), aqueous sodium thiosulfate solution (20% w/v, 2.0 mL), and saturated aqueous sodium bicarbonate solution (2.0 mL). The resulting mixture was stirred until it became clear (approximately 15 min) and then extracted with ether (3×3.0 mL). The organic layers were combined and the combined organic layers were dried over magnesium sulfate. The dried solution was filtered and the filtrate was concentrated to provide the alkynyl aldehyde 78 as a colorless oil (88.5 mg, 97%). The product so obtained was judged to be of >95% purity (¹H NMR analysis) and was used without further purification.

R_{f}=0.54 (40% ether-pentane; PAA, stains purple).

¹H NMR (400 MHz, CDCl₃) δ 9.50 (s, 1H), 5.75 (dd, J=17.5, 10.7 Hz, 1H), 5.18 (d, J=10.7 Hz, 1H), 5.12 (d, J=17.5 Hz, 1H), 4.01-3.77 (m, 4H), 3.35 (d, J=17.1 Hz, 1H), 2.95 (d, J=17.1 Hz, 1H), 2.70 (s, 1H), 2.16 (s, 1H), 2.11-1.76 (m, 6H), 1.72-1.55 (m, 2H), 1.48-1.39 (m, 1H), 1.27 (s, 3H), 1.18 (s, 3H), 1.05 (d, J=6.5 Hz, 3H).

¹³C NMR (101 MHz, CDCl₃) δ 209.8, 201.2, 138.9, 118.8, 116.1, 90.2, 71.2, 64.2, 62.4, 53.1, 51.2, 50.8, 46.2, 40.5, 36.58, 36.56, 36.5, 33.5, 27.8, 21.0, 18.6, 15.8.

HRMS-ESI (m/z): calculated for [C₁₂H₃₁O₄]⁺ 359.2222, found 359.2217.

### Synthesis of the Allylic Alcohol 79:

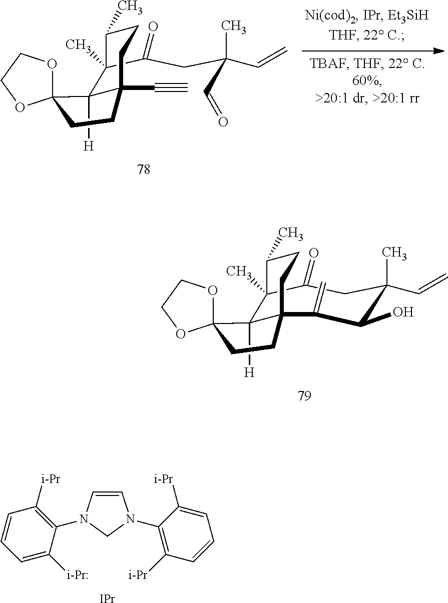

A solution of bis(1,5-cyclooctadiene)nickel(0) (46.0 mg, 167 μmol, 1.00 equiv) and 1,3-bis(2,6-diisopropylphenyl)imidazol-2-ylidene (IPr, 65.4 mg, 167 μmol, 1.00 equiv) in tetrahydrofuran (1.0 mL) was stirred for 30 min at 22° C. in. A portion of this solution (250 μL, 25 mol % of nickel and ligand) was added to a solution of the alkynyl aldehyde 78 (60.0 mg, 167 μmol, 1 equiv) and triethylsilane (80.0 μL, 502 μmol, 3.00 equiv) in tetrahydrofuran (3.0 mL) at 22° C. The resulting solution was stirred for 4 h at 22° C. A second portion of the metal-ligand stock solution (100 μL, 10 mol % of nickel and ligand) was added to the reaction mixture and the resulting solution was stirred for an additional 2 h. The resulting mixture was filtered through a short pad of silica gel (eluting with 50% ethyl acetate-hexanes). The filtrate was concentrated and the residue obtained was dissolved in tetrahydrofuran (840 μL). A solution of tetra-n-butylammonium fluoride in tetrahydrofuran (1.0 M, 837 μL, 837 μmol, 5.00 equiv) was added and the resulting solution was stirred for 15 min at 22° C. under air. The product mixture was diluted sequentially with saturated aqueous ammonium chloride solution (3.0 mL) and water (2.0 mL). The diluted solution was extracted with ether (3×4.0 mL). The organic layers were combined and the combined organic layers were dried over magnesium sulfate. The dried solution was filtered and the filtrate was concentrated. The residue obtained was purified by flash-column chromatography (eluting with 20% ethyl acetate-hexanes initially, grading to 45% ethyl acetate-hexanes, linear gradient) to provide the allylic alcohol 79 as a colorless oil (36.0 mg, 60%).

R_{f}=0.28 (40% v/v ethyl acetate-hexanes; PAA, stains pink).

¹H NMR (600 MHz, C₆D₆) δ 5.57-5.50 (m, 2H), 5.32 (s, 1H), 4.97 (d, J=17.4 Hz, 1H), 4.87 (d, J=10.7 Hz, 1H), 4.15 (s, 1H), 3.48-3.42 (m, 1H), 3.41-3.34 (m, 2H), 3.31-3.24 (m, 1H), 2.95 (s, 1H), 2.87 (d, J=12.2 Hz, 1H), 2.58-2.50 (m, 1H), 2.15-2.03 (m, 1H), 1.86-1.73 (m, 5H), 1.65 (d, J=12.2 Hz, 1H), 1.49 (d, J=7.1 Hz, 3H), 1.45-1.42 (m, 1H), 1.40 (s, 3H), 1.36 (s, 1H), 1.20 (s, 3H), 1.17-1.14 (m, 1H).

¹³C NMR (151 MHz, C₆D₆) δ 212.2, 148.9, 146.5, 119.6, 116.4, 114.8, 72.3, 64.5, 62.1, 51.8, 51.6, 49.0, 47.4, 45.5, 37.1, 35.5, 34.9, 29.1, 26.6, 20.1, 16.4, 14.4.

HRMS-ESI (m/z): calculated for [C₂₂H₃₃O₄]⁺ 361.2379, found 361.2383.

### Synthesis of the Alcohol S13:

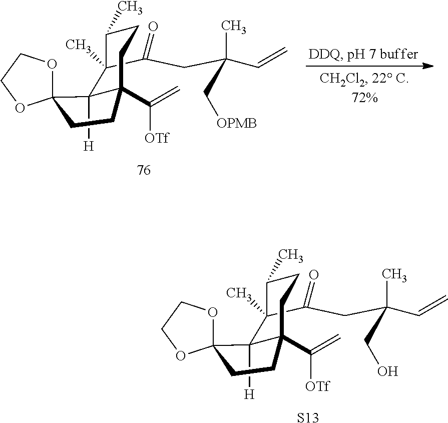

Aqueous potassium phosphate buffer solution (10 mM, pH 7, 100 μL) and 2,3-dichloro-5,6-dicyano-p-benzoquinone (DDQ, 14.4 mg, 63.4 μmol, 4.00 equiv) were added in sequence to a solution of the vinyl triflate 76 (10.0 mg, 15.9 μmol, 1 equiv) in dichloromethane (300 μL) at 22° C. The resulting green solution was stirred for 1 h at 22° C. open to air. The product mixture was diluted with saturated aqueous sodium bicarbonate solution (5.0 mL). The diluted product mixture was extracted with dichloromethane (3×5.0 mL). The organic layers were combined and the combined organic layers were dried over sodium sulfate. The dried solution was filtrated and the filtrate was concentrated. The residue obtained was purified by flash-column chromatography (eluting with 25% ethyl acetate-hexanes) to provide the alcohol S13 as a colorless oil (5.8 mg, 72%).

R_{f}=0.35 (25% ethyl acetate-pentane; PAA, stains brown).

¹H NMR (400 MHz, CDCl₃) δ 5.86 (dd, J=17.5, 10.9 Hz, 1H), 5.19 (d, J=4.9 Hz, 1H), 5.07 (d, J=4.6 Hz, 1H), 5.13-5.00 (m, 2H), 4.01-3.45 (m, 2H), 3.71-3.57 (m, 2H), 3.57-3.42 (m, 2H), 3.00 (s, 1H), 2.71-2.48 (m, 3H), 2.06-1.73 (m, 7H), 1.67-1.42 (m, 2H), 1.51 (s, 3H), 1.12 (s, 3H), 0.80 (d, J=7.0 Hz, 3H).

¹³C NMR (151 MHz, CDCl₃) δ 212.5, 162.0, 144.1, 119.0, 118.5 (q, J=320 Hz), 113.4, 100.6, 69.8, 64.7, 64.0, 50.8, 49.3, 46.1, 44.3, 41.6, 36.9, 35.5, 31.4, 28.1, 24.7, 21.7, 21.5, 15.1.

¹⁹F NMR (376 MHz, CDCl₃) δ−74.88.

HRMS-ESI (m/z): calculated for [C₂₃H₃₃F₃NaO₇S]⁺ 533.1791, found 533.1791.

### Synthesis of the Aldehyde 80:

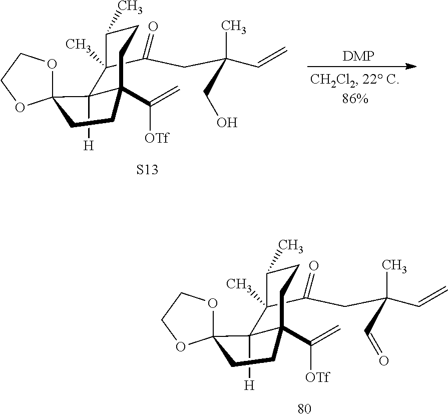

The Dess-Martin periodinane (DMP, 19.2 mg, 45.5 μmol, 4.00 equiv) was added in one portion to a solution of the alkynyl alcohol S13 (5.8 mg, 11.4 μmol, 1 equiv) in dichloromethane (200 μL) at 22° C. The resulting mixture was stirred for 90 min at 22° C. open to air. The product mixture was diluted sequentially with ether (500 μL), aqueous sodium thiosulfate solution (20% w/v, 200 μL), and saturated aqueous sodium bicarbonate solution (200 μL). The resulting mixture was stirred until it became clear (approximately 30 min) and then extracted with ether (3×3.0 mL). The organic layers were combined and the combined organic layers were dried over sodium sulfate. The dried solution was filtered and the filtrate was concentrated. The residue obtained was purified by flash-column chromatography (eluting with 10% ethyl acetate-pentane) to provide the aldehyde 80 as a colorless oil (5.0 mg, 86%). The isolated sample contains minor amounts of the C12 diastereomer, which was inseparable.

R_{f}=0.32 (20% ethyl acetate-pentane; PAA, stains purple).

¹H NMR (600 MHz, CDCl₃) δ 9.53 (s, 1H), 5.81 (dd, J=17.6, 10.7 Hz, 1H), 5.16 (m, 4H), 3.94-3.83 (m, 2H), 3.70-3.47 (m, 2H), 3.09 (d, J=18.0 Hz, 1H), 2.96 (s, 1H), 2.82 (d, J=18.1 Hz, 1H), 2.05-1.45 (m, 9H), 1.52 (s, 3H), 1.25 (s, 3H), 0.79 (d, J=7.1 Hz, 3H).

¹³C NMR (151 MHz, CDCl₃) δ 209.9, 202.2, 139.0, 118.9, 117.1 (q, J_{C—F}=322 Hz), 116.3, 116.2, 100.8, 64.7, 63.9, 50.2, 50.1, 49.2, 46.3, 45.8, 36.9, 35.5, 31.4, 28.3, 24.8, 11.8, 19.2, 14.9.

¹⁹F NMR (470 MHz, CDCl₃) δ−74.86.

HRMS-ESI (m/z): calculated for [C₂₃H₃₂F₃O₇S]⁺ 509.1815, found 509.1818.

### Attempted Nozaki-Hiyama-Kishi Cyclization of the Aldehyde 81:

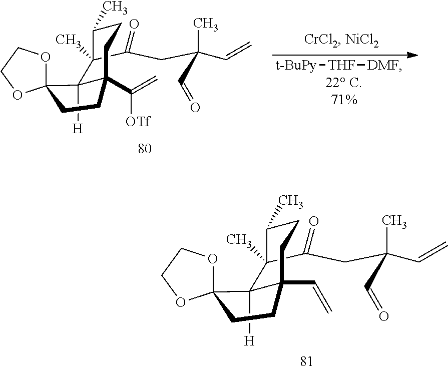

N,N-Dimethylformamide (150 μL) and 4-tert-butylpyridine (50 μL) were added to the solution of the aldehyde 80 (5.0 mg; 9.83 μmol, 1 equiv), nickel(II) chloride (1.3 mg, 9.83 μmol, 1.00 equiv), and chromium(II) chloride (8.5 mg, 68.8 μmol, 7.00 equiv) in tetrahydrofuran (300 μL) at 22° C. The resulting green solution was stirred for 22 h at 22° C. The product mixture was diluted with pentane (300 μL), ethyl acetate (300 μL), aqueous sodium bicarbonate solution (0.5 M, 300 μL), and aqueous DL-serine solution (0.5 M, 300 μL). The resulting purple solution was stirred for 1 h at 22° C. and then was extracted with ethyl acetate (3×3.0 mL). The organic layers were combined and the combined organic layers were dried over sodium sulfate. The dried solution was filtered and the filtrate was concentrated. The residue obtained was purified by flash-column chromatography (eluting with 10% ether-pentane) to provide the aldehyde 81 as a colorless oil (2.5 mg, 71%).

R_{f}=0.32 (20% ether-pentane PAA, stains purple).

¹H NMR (500 MHz, C₆D₆, 60° C.) δ 9.59 (s, 1H), 5.94 (dd, J=17.4, 10.9 Hz, 1H), 5.76 (dd, J=17.6, 10.8 Hz, 1H), 4.96 (m, 4H), 3.61-3.33 (m, 4H), 2.96 (d, J=17.7 Hz, 1H), 2.76 (d, J=17.6 Hz, 1H), 2.58 (s, 1H), 2.03-1.73 (m, 4H), 1.69-1.36 (m, 5H), 1.33 (s, 3H), 1.14 (s, 3H), 1.01 (d, J=7.1 Hz, 3H).

¹³C NMR (126 MHz, C₆D₆, 60° C.) 209.7, 200.5, 147.3, 140.3, 120.3, 115.2, 111.5, 64.2, 62.9, 54.1, 51.1, 50.5, 46.7, 46.6, 37.1, 36.8, 34.7, 30.1, 26.9, 21.8, 19.4, 15.9.

HRMS-ESI (m/z): calculated for [C₂₂H₃₃O₄]⁺ 361.2373, found 361.2369.

### Attempted Reductive Cyclization of the Alkenyl Aldehyde 82:

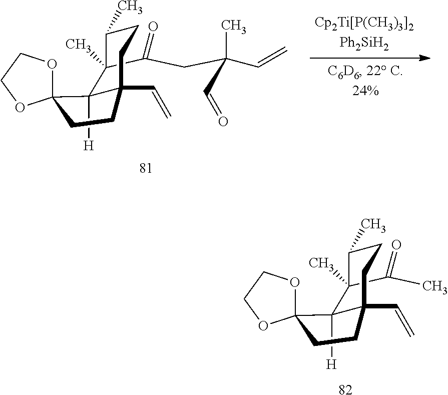

Bis(cyclopentadienyl)bis(trimethylphosphine)titanium (2.0 mg, 6.10 μmol, 1.00 equiv) and the aldehyde 81 (2.2 mg, 6.10 μmol, 1 equiv) were added in sequence to benzene-d₆ (600 μL) in a nitrogen-filled glovebox. The resulting mixture was transferred to a J-Young NMR tube. Diphenylsilane (1.00 μL, 6.10 μmol, 1.00 equiv) was added to the mixture and the tube was sealed with a Teflon-lined cap. The sealed tube was removed from the glovebox. The resulting mixture was left to age for 21 h at 22° C. The product mixture was concentrated and the residue obtained was purified by flash-column chromatography (eluting with 10% ether-pentane) to provide the methyl ketone 82 as a colorless oil (0.4 mg, 24%).

R_{f}=0.31 (20% ether-pentane; PAA, stains green).

¹H NMR (600 MHz, CDCl₃), δ 5.76 (dd, J=17.5, 10.8 Hz, 1H), 5.02 (d, J=17.5 Hz, 1H), 4.96 (d, J=10.8 Hz, 1H), 3.99-3.76 (m, 4H), 2.44 (s, 1H), 2.04 (s, 3H), 2.08-1.17 (m, 9H), 1.25 (s, 3H), 0.97 (d, J=7.0 Hz, 3H).

¹³C NMR (600 MHz, CDCl₃) δ 211.3, 129.3, 120.1, 112.2, 64.2, 62.6, 51.2, 46.7, 36.6, 36.1, 32.1, 29.9, 26.1, 24.9, 22.8, 21.2, 15.9.

HRMS-ESI (m/z): calculated for [C₁₇H₂₇O₃]⁺ 279.1955 found 279.1947.

### Synthesis of the Aldehyde 83:

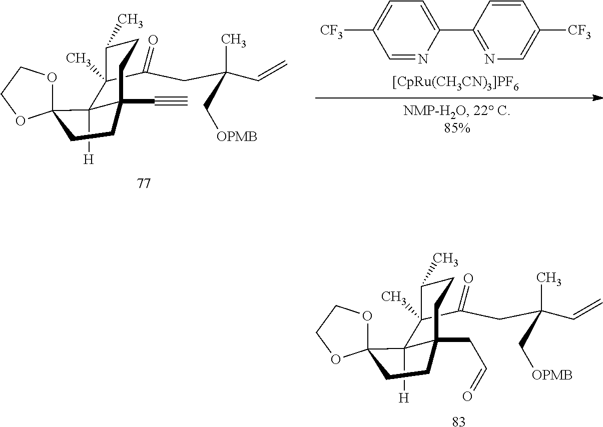

A solution of (η⁵-cyclopentadienyl)ruthenium tris(acetonitrile) hexafluorophosphate (5.7 mg, 13.1 μmol, 1.00 equiv) and 5,5′-bis(trifluoromethyl)-2,2′-bipyridine (3.8 mg, 13.1 μmol, 1.00 equiv) in N-methylpyrrolidinone (240 μL) was added to the alkyne 77 (6.3 mg, 13.1 μmol, 1 equiv) in a 1-dram vial in a nitrogen-filled glovebox. The vial was sealed with Teflon-lined cap. The sealed vial was removed from the glovebox and placed into a nitrogen-filled bag. The vial was opened and water (60 μL) was added to the vial. The vial was sealed with a Teflon-lined cap. The resulting mixture was stirred for 93 h at 22° C. The product mixture was diluted sequentially with saturated aqueous ammonium chloride solution (2.0 mL) and ethyl acetate (1.5 mL). The layers that formed were separated and the aqueous layer was extracted with ethyl acetate (3×1.5 mL). The organic layers were combined and the combined organic layers were dried over sodium sulfate. The dried solution was filtered and the filtrate was concentrated. The residue obtained was purified by flash-column chromatography (eluting with 5% ethyl acetate-hexanes) to provide the aldehyde 83 as a colorless oil (5.5 mg, 85%).

R_{f}=0.32 (20% ethyl acetate-pentane; PAA, stains pink).

¹H NMR (500 MHz, C₆D₆, 60° C.) δ 9.69 (t, J=2.4 Hz, 1H), 7.21 (d, J=8.6 Hz, 2H), 6.80 (d, J=8.5 Hz, 2H), 6.17 (dd, J=17.7, 11.0 Hz, 1H), 5.08 (dd, J=17.7, 1.3 Hz, 1H), 5.03 (dd, J=10.9, 1.2 Hz, 1H), 4.35 (s, 2H), 3.71 (q, J=7.3 Hz, 1H), 3.60 (d, J=8.4 Hz, 1H), 3.46 (d, J=8.1 Hz, 1H), 3.56-3.28 (m, 3H), 3.36 (s, 3H), 2.81 (s, 1H), 2.72-2.63 (m, 3H), 2.34 (dd, J=15.7, 2.5 Hz, 1H), 1.97-1.77 (m, 2H), 1.70-1.50 (m, 5H), 1.40-1.28 (m, 2H), 1.37 (m, 3H), 1.26 (m, 3H), 0.85 (d, J=7.0 Hz, 3H).

¹³C NMR (126 MHz, C₆D₆, 60° C.) δ 211.2, 201.6, 159.9, 145.4, 131.5, 129.4 (2C), 120.5, 114.3 (2C), 112.1, 77.0, 73.4, 64.5, 63.8, 55.3, 54.9, 51.4, 49.7, 43.7, 42.8, 40.6, 37.7, 36.6, 34.3, 29.6, 25.4, 22.8, 22.0, 15.5.

HRMS-ESI (m/z): calculated for [C₃₀H₄₂NaO₆]⁺ 521.2874, found 521.2875.

### Synthesis of the Alcohol S14:

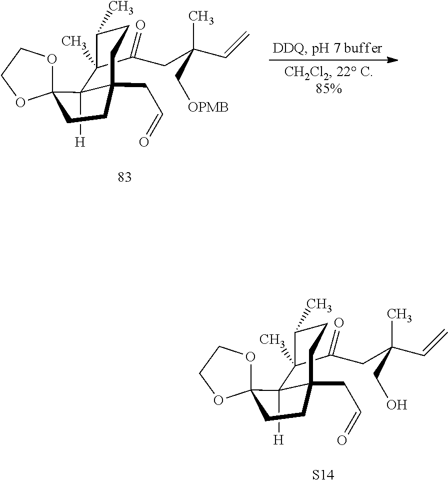

Aqueous potassium phosphate buffer solution (10 mM, pH 7, 100 μL) and 2,3-dichloro-5,6-dicyano-p-benzoquinone (DDQ, 12.0 mg, 43.3 μmol, 4.00 equiv) were in sequence to a solution of the aldehyde 83 (5.4 mg, 10.8 μmol, 1 equiv) its dichloromethane (300 μL) at 22° C. The resulting solution was stirred for 1 h at 22° C. open to air. The product mixture was diluted with saturated aqueous sodium bicarbonate solution (5.0 mL). The diluted product mixture was extracted with dichloromethane (3×5 mL). The organic layers were combined and the combined layers were dried over sodium sulfate. The dried solution was filtered and the filtrate was concentrated. The residue obtained was purified by flash-column chromatography (eluting with 20% ethyl acetate-pentane, grading to 30% ethyl acetate-pentane, two steps) to provide the alcohol S14 as a colorless oil (3.5 mg, 85%).

R_{f}=0.29 (50% ethyl acetate-pentane; PAA, stains purple).

¹H NMR (500 MHz, C₆D₆, 60° C.) δ 9.68 (t, 2.3 Hz, 1H), 5.90 (m, 1H), 4.99 (m, 2H), 3.68 (q, J=7.3 Hz, 1H), 3.56-3.35 (m, 4H), 3.31 (q, J=7.0 Hz, 1H), 2.80 (s, 1H), 2.63 (dd, J=15.6, 2.1 Hz, 1H), 2.56 (d, J=17.7 Hz 1H), 2.45 (d, J=17.7 Hz, 1H), 2.35 (dd, J=15.6, 2.6 Hz, 1H), 1.97-1.77 (m, 2H), 1.65-1.49 (m, 4H), 1.43-1.25 (m, 3H), 1.34 (s, 3H), 1.12 (s, 3H), 0.79 (d, J=7.0 Hz, 3H).

¹³C NMR (126 MHz, C₆D₆, 60° C.) 212.3, 201.6, 145.1, 120.5, 1.12.7, 69.6, 64.5, 63.8, 55.4, 51.5, 49.4, 44.1, 42.7, 41.7, 37.5, 36.6, 34.2, 29.4, 25.2, 21.90, 21.87, 15.5.

HRMS-ESI (m/z): calculated for [C₂₂H₃₄NaO₅]⁺ 401.2298, found 401.2289.

### Synthesis of Dialdehyde 84:

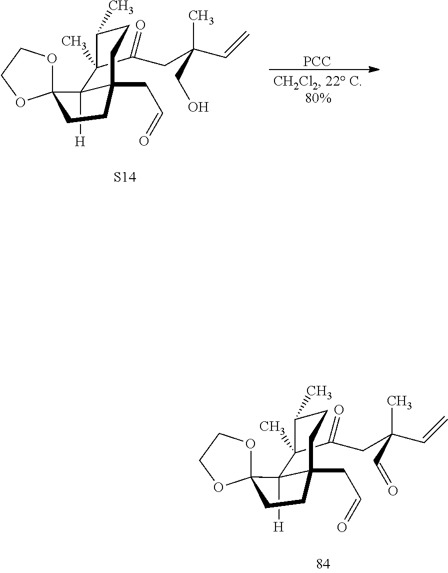

Pyridinium chlorochromate (5.7 mg, 26.4 μmol, 5.00 equiv) was added in one portion to a solution of the alcohol S14 (2.0 mg, 5.28 μmol, 1 equiv) in dichloromethane (200 μL) at 22° C. The resulting mixture was stirred for 1 h at 22° C. open to air. The product mixture was loaded directly onto a silica gel flash-column and purified by flash-column chromatography (eluting with 40% ether-pentane) to provide the dialdehyde 84 as a colorless oil (1.6 mg, 80%).

R_{f}=0.42 (20% ethyl acetate-pentane; PAA, stains purple).

¹H NMR (500 MHz, C₆D₆, 60° C.) δ 9.66 (t, J=2.2 Hz, 1H), 9.56 (s, 1H), 5.75 (dd, J=17.6, 10.8 Hz, 1H), 4.98 (d, J=10.7 Hz, 1H), 4.94 (d, J=17.6 Hz, 1H), 3.63 (q, J=7.3 Hz, 1H), 3.46-3.34 (m, 2H), 3.31 (q, J=7.0 Hz, 1H), 2.96 (d, J=17.1 Hz, 1H), 2.77 (s, 1H), 2.61-2.53 (m, 2H), 2.34 (dd, J=15.8, 2.5 Hz, 1H), 2.23 (s, 1H), 1.97-1.76 (m, 2H), 1.63-1.16 (m, 7H), 1.31 (s, 3H), 1.12 (s, 3H), 0.75 (d, J=7.3 Hz, 3H).

¹³C NMR (126 Hz, C₆D₆, 60° C.) δ 210.3, 201.5, 201.0, 140.3, 120.3, 115.3, 64.6, 63.9, 55.4, 50.8, 50.0, 49.4, 46.4, 42.7, 37.6, 36.6, 34.2, 29.4, 25.1, 21.9, 19.5, 15.3.

HRMS-ESI (m/z): calculated for [C₂₂H₃₃O₅]⁺ 377.2323, found 377.2330.

### Synthesis of the Alkene 86:

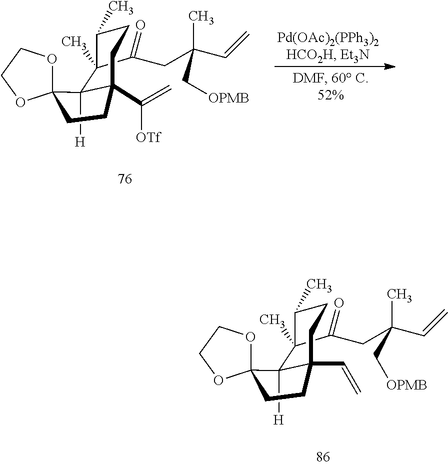

Triethylamine (44.2 μL, 317 μmol, 8.00 equiv) and formic acid (6.0 μL, 159 μmol, 4.00 equiv) were added in sequence to a solution of the vinyl triflate 76 (25.0 mg, 39.6 μmol, 1 equiv) and bis(triphenylphosphine) palladium(II) diacetate (5.9 mg, 7.93 μmol, 20.0 mol %) in N,N-dimethylformamide (400 μL) at 22° C. The resulting mixture was heated for 20 h at 60° C. The product mixture was cooled to 22° C. and the cooled product mixture was diluted sequentially with saturated aqueous ammonium chloride solution (1.5 mL) and ether (1.5 mL). The layers that formed were separated and the aqueous layer was extracted with ether (3×1.5 mL). The organic layers were combined and the combined organic layers were dried over magnesium sulfate. The dried solution was filtered and the filtrate was concentrated. The residue obtained was purified by preparative thin-layered chromatography (eluting with 20% ethyl acetate-pentane) to provide the alkene 86 as a colorless oil (10.0 mg, 52%).

R_{f}=0.62 (20% ethyl acetate-pentanel PAA, stains brown).

¹H NMR (500 MHz, C₆D₆, 60° C.) δ 7.22 (d, J=6.8 Hz, 2H), 6.80 (d, J=6.8 Hz, 2H), 6.21 (dd, J=17.7, 10.9 Hz, 1H), 6.04 (dd, J=17.5, 10.8 Hz, 1H), 5.13-4.91 (m, 4H), 4.36 (s, 2H), 3.66-3.36 (m, 6H), 3.35 (s, 3H), 2.83 (d, J=17.5 Hz, 1H), 2.69 (s, 1H), 2.69 (d, J=17.5 Hz, 1H), 2.10-1.92 (m, 2H), 1.90-1.76 (m, 2H), 1.69-1.52 (m, 4H), 1.46-1.37 (m, 1H), 1.43 (s, 3H), 1.28 (s, 3H), 1.11 (d, J=6.9 Hz, 3H).

¹³C NMR (126 MHz, C₆D₆, 60° C.) δ 210.6, 159.9, 147.6, 145.5, 131.6, 129.3 (2C), 120.5, 114.3 (2C), 112.0, 111.1, 77.3, 73.4, 64.2, 62.9, 54.9, 53.9, 51.7, 46.9, 44.0, 40.9, 37.3, 36.8, 34.8, 30.3, 27.1, 22.7, 22.0, 16.2.

HRMS-ESI (m/z): calculated for [C₃₀H₄₂NaO₅]⁺ 505.2924, found 505.2923.

### Synthesis of the Diol 89:

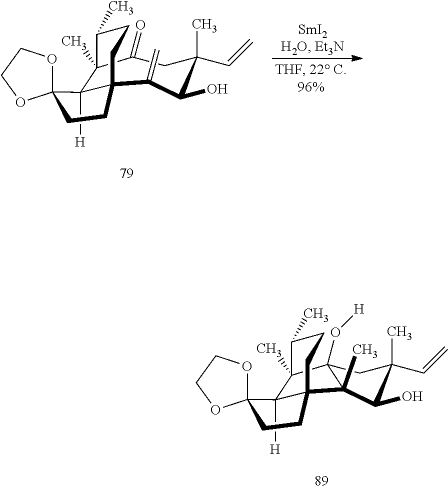

A solution of samarium (II) iodide in tetrahydrofuran (0.1 M, 581 μL, 58.1 μmol, 6.00 equiv) was added to the allylic alcohol 79 (2.8 mg, 7.77 μmol, 1 equiv) at 22° C., resulting in a dark blue solution. Triethylamine (26.0 μL, 186 μmol, 24.0 equiv) and water (5 μL, 280 μmol, 36.0 equiv) were then added in sequence, immediately inducing formation of a heterogeneous white solution. The resulting mixture was stirred for 5 min at 22° C. The product mixture was diluted with saturated aqueous ammonium chloride solution (1.5 mL) and the diluted product mixture was extracted with ether (3×1.5 mL). The organic layers were combined and the combined organic layers were dried over sodium sulfate. The dried solution was filtered and the filtrate was concentrated. The residue obtained was purified by flash-column chromatography (eluting with 20% ethyl acetate-pentane) to provide alcohol 89 as a white solid (2.7 mg, 96%).

R_{f}=0.30 (25% ethyl acetate-pentane; PAA, stains blue).

¹H NMR (600 MHz, CDCl₃) δ 5.83 (dd, J=17.4, 11.1 Hz, 1H), 5.04 (m, 2H), 3.95-3.86 (m, 2H), 3.82 (q, J=5.9 Hz, 1H), 3.75 (q, J=6.8 Hz, 1H), 3.59 (s, 1H), 2.44-2.33 (m, 1H), 2.28-2.20 (m, 1H), 2.19-2.11 (m, 1H), 1.98-1.88 (m, 1H), 1.81 (d, J=14.4 Hz, 1H), 1.69 (s, 1H), 1.73-1.63 (m, 1H), 1.55-1.46 (m, 3H), 1.44 (d, J=14.2 Hz, 1H), 1.20 (s, 3H), 1.23-1.17 (m, 1H), 1.11 (s, 3H), 1.04 (s, 3H), 1.04 (d, J=6.4 Hz, 3H).

¹³C NMR (151 MHz, CDCl₃) δ147.7, 115.4, 112.8, 97.5, 81.0, 65.1, 63.3, 58.3, 53.6, 53.1, 48.7, 46.6, 45.4, 42.5, 33.6, 30.5, 28.1, 26.8, 19.9, 17.9, 17.1, 11.7.

HRMS-ESI (m/z): calculated for [C₂₂H₃₃O₃]⁺ (—OH) 345.2424, found 345.2429.

### Synthesis of the Enone S15:

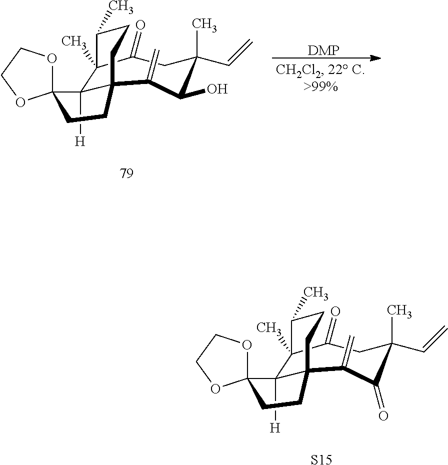

The Dess-Martin periodinane (61.2 mg, 144 μmol, 4.00 equiv) was added to a solution of the allylic alcohol 79 (13.0 mg, 36.1 μmol, 1 equiv) in dichloromethane (500 μL) at 22° C. The resulting mixture was stirred for 6 h at 22° C. open to air. The product mixture was diluted sequentially with ether (1.0 mL), aqueous sodium thiosulfate solution (20% w/v, 1.0 ML), and saturated aqueous sodium bicarbonate solution (1.0 mL). The resulting mixture was stirred until it became clear (approximately 15 min) and was then extracted with ether (3×2.0 mL). The organic layers were combined and the combined organic layers were dried over magnesium sulfate. The dried solution was filtered and the filtrate was concentrated to provide the enone S15 as a white solid (13.0 mg, >99%). The product so obtained was judged to be of >95% purity (¹H NMR analysis) and was used without further purification.

R_{f}=0.49 (15% ethyl acetate-hexanes; UV, PAA, stains yellow).

¹H NMR (400 MHz, C₆D₆) δ 6.33 (dd, J=17.5, 10.9 Hz, 1H), 5.06 (s, 1H), 4.99 (dd, J=10.9, 0.5 Hz, 1H), 4.84 (dd, J=17.5, 0.5 Hz, 1H), 4.76 (s, 1H), 3.45-3.24 (m, 3H), 3.23-3.15 (m, 1H), 3.04 (d, J=11.9 Hz, 1H), 2.72-2.59 (m, 2H), 2.52 (dqd, J=14.2, 7.1, 3.8 Hz, 1H), 2.08 (qd, J=12.9, 4.2 Hz, 1H), 1.79-1.59 (m, 5H), 1.47 (s, 3H), 1.46 (d, J=7.1 Hz, 3H), 1.37 (ddd, J=13.2, 7.0, 3.6 Hz, 1H), 1.27-1.22 (m, 1H), 1.21 (s, 3H).

¹³C NMR (101 MHz, C₆D₆) δ 210.5, 210.2, 152.3, 142.2, 119.3, 115.5, 112.3, 64.1, 62.0, 55.2, 53.4, 52.3, 49.2, 44.5, 36.8, 35.7, 35.6, 29.0, 26.9, 22.0, 20.1, 16.7.

HRMS-ESI (m/z): calculated for [C₂₂H₃₁O₄]⁺ 359.2222, found 359.2227.

### Synthesis of the Diketone 90:

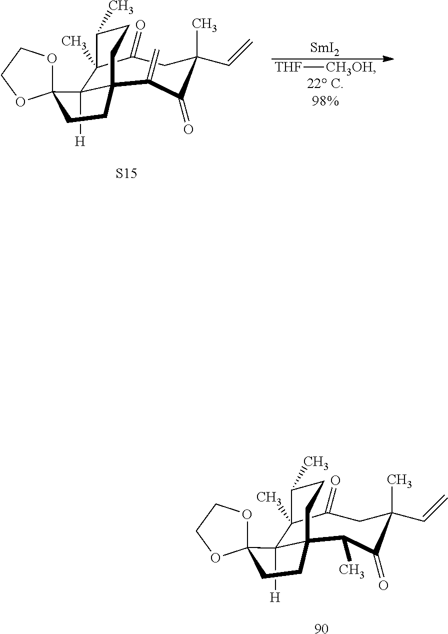

Methanol (1.0 mL) was added to a solution of samarium(II) iodide in tetrahydrofuran (0.1 M, 2.00 mL, 200 μmol, 4.00 equiv) at 22° C., resulting in a green solution. A solution of the enone S15 (17.7 mg, 49.4 μmol, 1 equiv) in tetrahydrofuran (1.0 mL) was then added. The resulting mixture was stirred for 5 min at 22° C. The product mixture was diluted sequentially with saturated aqueous ammonium chloride solution (2.0 mL) and water (2.0 mL). The diluted product mixture was extracted with ethyl acetate (3×2.0 mL). The organic layers were combined and the combined organic layers were washed with aqueous sodium thiosulfate solution (20% w/v, 2.0 mL). The washed organic layer was dried over magnesium sulfate. The dried solution was filtered and the filtrate was concentrated to provide the diketone 90 as a white solid (17.4 mg, 98%). The product so obtained was judged to be of >95% purity (¹H NMR analysis) and was used without further purification.

R_{f}=0.50 (15% ethyl acetate-hexanes; PAA, stains pink).

¹H NMR (500 MHz, CDCl₃) δ 6.20 (dd, J=17.6, 10.9 Hz, 1H), 5.13 (d, J=10.9 Hz, 1H), 5.00 (d, J=17.6 Hz, 1H), 3.98 (q, J=6.9 Hz, 1H), 3.89 (dd, J=13.9, 7.1 Hz, 1H), 3.84 (dd, J=13.5, 6.9 Hz, 1H), 3.74 (dd, J=13.8, 7.2 Hz, 1H), 3.09 (d, J=11.6 Hz, 1H), 3.07 (q, J=7.0 Hz, 1H), 2.36-2.25 (m, 1H), 2.17 (s, 1H), 2.11 (dd, J=23.3, 10.9 Hz, 1H), 1.93 (d, J=11.7 Hz, 1H), 1.81 (d, J=10.5 Hz, 2H), 1.79-1.69 (m, 1H), 1.54-1.48 (m, 2H), 1.42 (s, 3H), 1.41-1.34 (m, 1H), 1.31-1.27 (m, 1H), 1.22 (s, 3H), 1.21 (d, J=7.1 Hz, 3H), 0.95 (d, J=6.7 Hz, 3H).

¹³C NMR (126 MHz, CDCl₃) δ 216.3, 214.6, 141.9, 119.6, 112.5, 64.2, 62.0, 55.5, 53.6, 51.8, 46.8, 44.5, 41.3, 35.9, 35.1, 29.6, 27.4, 26.6, 20.6, 20.3, 16.4, 13.5.

HRMS-ESI (m/z): calculated for [C₂₂H₃₃O₄]⁺ 361.2379, found 361.2375.

Synthesis of the Diols 91 and 92 from the Diketone 90:

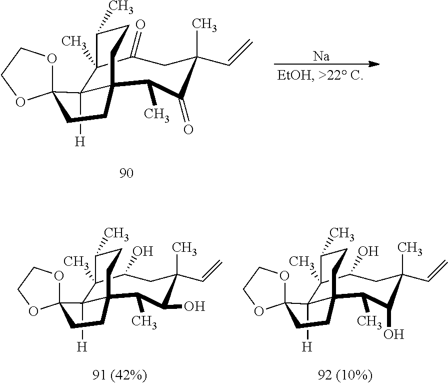

Freshly cut sodium metal (˜50 mg, excess) was added to a solution of the diketone 90 (5.0 mg, 13.9 μmol, 1 equiv) in ethanol (750 μL) at 22° C. CAUTION: THE ADDITION IS EXOTHERMIC. Additional freshly cut sodium metal (˜1.50 mg total) and ethanol (approx. 1.5 mL total) were added as needed until no further conversion of the substrate was observed by thin-layered chromatography (which occurred at approximately 50% conversion and in 20 min). The reaction mixture was diluted sequentially with aqueous saturated ammonium chloride solution (2.0 mL) and water (2.0 mL). The diluted mixture was extracted with ethyl acetate (3×5.0 mL). The organic layers were combined and the combined organic layers were dried over magnesium sulfate. The dried solution was filtered and the filtrate was concentrated. The residue obtained was dissolved in ethanol (750 μL) and resubjected to the above reaction conditions to achieve full conversion of the substrate. The diols 91 and 92 were formed in a 3:1 ratio based on ¹H NMR analysis of the unpurified product mixture. The mixture was purified by preparative thin-layered chromatography (eluting with 30% ethyl acetate-hexanes) to afford separately the diol 91 as a white solid (2.1 mg, 42%) and the diol 92 as a white solid (0.5 mg, 10%).

91

R_{f}=0.30 (30% ethyl acetate-hexanes; PAA, stains blue).

¹H NMR (500 MHz, C₆D₆) δ 5.31 (dd, J=17.5, 10.7 Hz, 1H), 4.94 (dd, J=17.5, 1.3 Hz, 1H), 4.83 (dd, J=10.7, 1.3 Hz, 1H), 4.31 (d, J=8.1 Hz, 1H), 3.62-3.53 (m, 1H), 3.50-3.45 (m, 1H), 3.44-3.39 (m, 1H), 3.35 (d, J=6.4 Hz, 1H), 3.33-3.27 (m, 1H), 2.61-2.48 (m, 1H), 2.26-2.11 (m, 3H), 1.81-1.66 (m, 3H), 1.65-1.42 (m, 3H), 1.41 (tt, J=14.2, 4.1 Hz, 1H), 1.25 (s, 3H), 1.29-1.21 (m, 3H), 1.18 (d, J=15.4 Hz, 1H), 1.12 (s, 3H), 1.08 (d, J=7.2 Hz, 3H), 1.07 (d, J=7.2 Hz, 3H).

¹³C NMR (126 MHz, C₆D₆) δ 148.4, 120.9, 113.6, 71.9, 67.4, 63.9, 61.6, 52.2, 47.2, 46.4, 45.8, 43.1, 36.4, 36.1, 34.4, 29.6, 28.9, 28.1, 19.0, 14.03, 13.97, 11.8.

HRMS-ESI (m/z): calculated for [C₂₂H₃₇O₄]⁺ 365.2694, found 365.2700.

92

R_{f}=0.49 (30% ethyl acetate-hexanes; PAA, stains purple).

¹H NMR (500 MHz, C₆D₆) δ 5.31 (dd, J=17.7, 11.0 Hz, 1H), 4.83-4.77 (m, 2H), 4.35 (d, J=6.0 Hz, 1H), 3.61 (dd, J=12.7, 6.9 Hz, 1H), 3.53 (dd, J=13.1, 6.8 Hz, 1H), 3.51-3.43 (m, 1H), 3.40-3.34 (m, 1H), 3.18 (s, 2H), 2.77-2.67 (m, 1H), 2.68-2.54 (m, 2H), 2.09 (q, J=7.0 Hz, 1H), 1.98-1.89 (m, 1H), 1.86-1.79 (m, 1H), 1.77-1.69 (m, 1H), 1.58-1.49 (m, 2H), 1.48-1.41 (m, 1H), 1.38 (s, 3H), 1.31 (s, 1H), 1.20 (ddd, J=12.7, 9.5, 3.4 Hz, 1H), 1.15 (d, J=7.3 Hz, 3H), 1.11 (d, J=15.0 Hz, 1H), 1.05 (d, J=7.0 Hz, 3H), 1.00 (s, 3H).

¹³C NMR (151 MHz, C₆D₆) δ 147.3, 121.8, 114.4, 84.4, 68.1, 63.6, 61.7, 51.3, 46.0, 44.4, 42.7, 40.3, 36.3, 35.7, 33.7, 33.2, 31.2, 28.8, 22.9, 20.7, 19.2, 14.2.

HRMS-ESI (m/z): calculated for [C₂₂H₃₇O₄]⁺ 365.2694, 365.2698.

Synthesis of (+)-12-epi-mutilin 94:

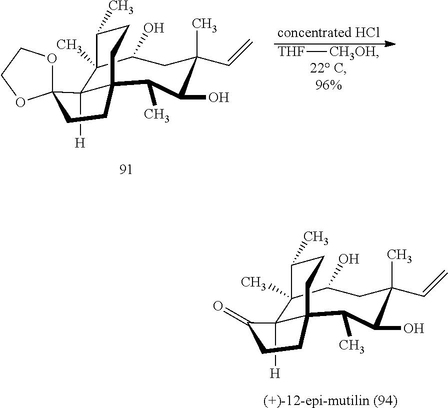

Concentrated aqueous hydrochloric acid solution (approximately 12 M, 50.0 μL) was added to a solution of 12-epi-mutilin-ketal 91 (2.5 mg, 6.86 μmol, 1 equiv) in tetrahydrofuran-methanol (1:1 v/v, 1.0 mL) at 22° C. The resulting mixture was stirred for 20 min at 22° C. open to air. The product mixture was diluted with water (3.0 mL). The diluted product mixture was extracted with ethyl acetate (3×3.0 mL). The organic layers were combined and the combined organic layers were dried over magnesium sulfate. The dried solution was filtered and the filtrate was concentrated to provide 12-epi-mutilin 94 as white solid (2.1 mg, 96%). The product so obtained was judged to be of >95% purity (¹H NMR analysis) and was used without further purification.

R_{f}=0.30 (30% ethyl acetate-hexanes; PAA, stains blue).

¹H NMR (600 MHz, CDCl₃) 5.76 (dd, J=17.7, 10.6 Hz, 1H), 5.21 (m, 2H), 4.37 (d, J=7.7 Hz, 1H), 3.41 (d, J=6.7 Hz, 1H), 2.31-2.13 (m, 3H), 2.05 (s, 1H), 1.99 (dd, J=15.6, 7.8 Hz, 1H) 1.79 (dq, J=15.4, 3.2 Hz, 1H), 1.73-1.38 (m, 5H), 1.35 (s, 3H), 1.20 (s, 3H), 1.23-1.10 (m, 2H), 0.98 (d, J=7.5 Hz, 3H), 0.95 (d, J=7.5 Hz, 3H).

¹³C NMR (151 Hz, CDCl₃) 217.8, 147.5, 115.0, 72.2, 66.7, 59.2, 46.2, 45.5, 45.3, 42.6, 37.0, 34.65, 34.61, 30.5, 27.4, 25.2, 18.4, 13.8, 13.6, 11.1.

HRMS-ESI (m/z): calculated for [C₂₀H₃₁O₂]⁺ 303.2319, found 303.2317. α_{D}²⁰=+20° (c=0.12, CHCl₃)

α_{D}²⁰=+34° (c=0.15, CHCl₃) (for 4 prepared by degradation of natural (+)-pleuromutilin)

Synthesis of (+)-11,12-di-epi-mutilin 95:

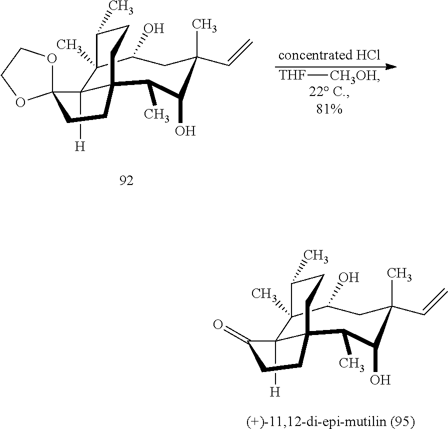

Concentrated aqueous hydrochloric acid solution (approximately 12 M, 50.0 μL) was added to a solution of 11,12-di-epi-mutilin-ketal 92 (2.1 mg, 5.76 μmol, 1 equiv) in tetrahydrofuran-methanol (1:1 v/v, 1.0 mL) at 22° C. The resulting mixture was stirred for 5 min at 22° C. open to air. The product mixture was diluted with water (3.0 mL). The diluted product mixture was extracted with ethyl acetate (3×3.0 mL). The organic layers were combined and the combined organic layers were dried over magnesium sulfate. The dried solution was filtered and the filtrate was concentrated. The residue obtained was purified by preparative thin-layered chromatography (eluting with 25% ethyl acetate-hexanes) to provide 11,12-di-epi-mutilin 95 as a white solid (1.5 mg, 81%). The isolated material contained small amounts of impurities. The yield is based on this material.

R_{f}=0.48 (25% ethyl acetate-hexanes; PAA, stains purple).

¹H NMR (600 MHz, CDCl₃) δ 5.71 (dd, J=17.7, 11.0 Hz, 1H), 5.20 (d, J=11.0 Hz, 1H), 5.13 (d, J=17.7 Hz, 1H), 4.39 (d, J=5.3 Hz, 1H), 3.45 (s, 1H), 2.98 (s, 1H), 2.44 (dd, J=22.5, 10.4 Hz, 1H), 2.36 (dd, J=15.2, 6.4 Hz, 1H), 2.25-2.08 (m, 4H), 2.06-1.97 (m, 1H), 1.71-1.55 (m, 3H), 1.42 (s, 3H), 1.39-1.24 (m, 2H), 1.20 (s, 3H), 1.13 (d, J=7.1 Hz, 3H), 1.13-1.06 (m, 2H), 1.00 (d, J=7.1 Hz, 3H).

¹³C NMR (151 MHz, CDCl₃) δ 220.3, 147.0, 115.2, 84.2, 67.5, 59.3, 45.2, 44.1, 42.5, 39.1, 37.5, 35.1, 33.7, 32.9, 27.9, 27.6, 22.5, 20.0, 18.6, 13.9.

HRMS-ESI (m/z): calculated for [C₂₀H₃₃O₃]⁺ 321.2430, found 321.24.22.

α_{D}²⁰=+14° (c=0.03, CHCl₃)

### Synthesis of the Ester S16:

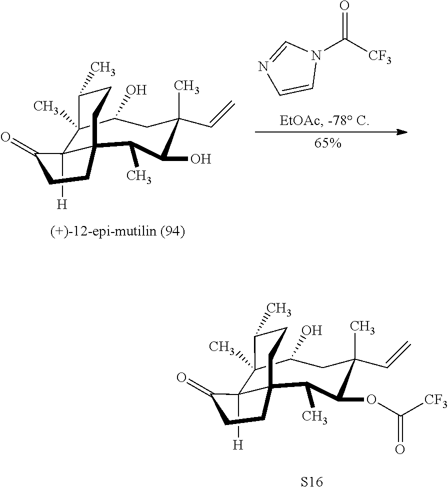

1-(Trifluoroacetyl)imidazole (37.7 μL, 331 μmol, 6.00 equiv) was added dropwise to a solution of 12-epi-mutilin 94 (17.7 mg, 55.2 μmol, 1 equiv) in ethyl acetate (1.0 mL) at −78° C. The resulting mixture was stirred for 50 min at −78° C. The product mixture was diluted with aqueous hydrochloric acid solution (1 M, 200 μL) and then was warmed to 22° C. over 1 h. The warmed product mixture was diluted with aqueous hydrochloric acid solution (1 M, 1 mL). The diluted product mixture was extracted with ethyl acetate (3×5 mL). The organic layers were combined and the combined organic layers were dried over sodium sulfate. The dried solution was filtered and the filtrate was concentrated. The residue obtained was purified by preparative thin-layered chromatography (eluting with 40% ether-pentane) to provide the ester S16 as a white solid (15.0 mg, 65%).

R_{f}=0.65 (40% ether-pentane, PAA stains purple)

¹H NMR (500 MHz, CDCl₃) 5.62 (dd, J=17.4, 10.8 Hz, 1H), 5.12-4.98 (m, 3H), 4.39-4.33 (m, 1H), 2.53 (p, J=7.2 Hz, 1H), 2.38-2.03 (m, 4H), 1.82-1.66 (m, 3H), 1.60-1.40 (m, 3H), 1.38 (s, 3H), 1.33 (s, 3H), 1.29-1.13 (m, 2H), 0.99 (d, J=7.1 Hz, 3H), 0.83 (d, J=7.1 Hz, 3H).

¹⁹F NMR (470 MHz, CDCl₃)−75.09 (s, 3F).

¹³C NMR (151 MHz, CDCl₃) 216.8, 156.8 (q, J=42.0 Hz), 145.0, 114.8 (q, J=286.1 Hz), 114.0, 80.1, 66.4, 59.2, 46.0, 45.2, 43.8, 42.7, 36.9, 34.9, 34.5, 30.4, 27.3, 25.3, 18.3, 15.0, 13.5, 11.6.

HRMS-ESI (m/z): calculated for [C₂₂H₃₁F₃O₃Na]⁺ 439.2067, found 439.2046.

Synthesis of (+)-12-epi-pleuromutilin (97):

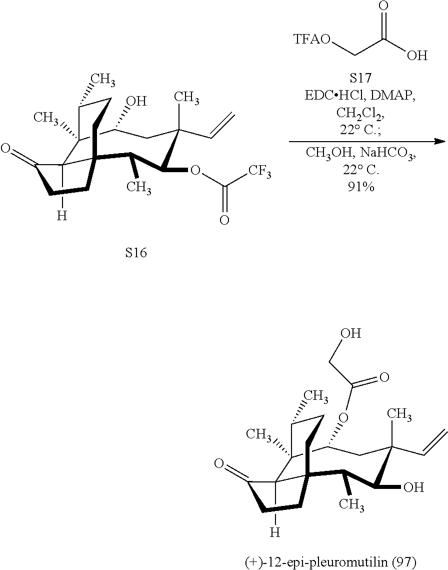

Trifluoroacetylglycolic acid (S17, 5.2 mg, 30.1 μmol, 3.30 equiv) was added dropwise to a solution of the ester S16 (3.8 mg, 9.12 μmol, 1 equiv) N-(3-dimethylaminopropyl)-N′-ethyl-carbodiimide hydrogen chloride (EDC.HCl, 4.7 mg, 30.1 μmol, 3.30 equiv), and 4-(dimethylamino)pyridine (3.7 mg, 30.1 μmol, 3.30 equiv) in dichloromethane (500 μL) at 22° C. under air. The resulting mixture was stirred for 30 min at 22° C. and then methanol (500 μL) and sodium bicarbonate (20.0 mg, 238 μmol, 26.1 equiv) were added in sequence. The resulting mixture was stirred at for 22 h at 22° C. The product mixture was diluted with saturated aqueous ammonium chloride solution (1.5 mL) and the diluted mixture was extracted with ethyl acetate (3×1.5 mL). The organic layers were combined and the combined organic layers were dried over sodium sulfate. The dried solution was filtered and the filtrate was concentrated. The residue obtained was dissolved in methanol (200 μL) and then sodium bicarbonate (12.0 mg, 143 μmol, 15.6 equiv) was added at 22° C. The resulting solution was stirred for 21 h at 22° C. The product mixture was diluted with saturated aqueous ammonium chloride solution (1.5 mL) and the diluted solution was extracted with ethyl acetate (3×1.5 mL). The organic layers were combined and the combined organic layers were dried over sodium sulfate. The dried solution was filtered and the filtrate was concentrated. The residue obtained was purified by preparative thin-layered chromatography (eluting with 40% ethyl acetate-pentane) to provide 12-epi-pleuromutilin 97 as a white solid (3.2 mg, 91%).

R_{f}=0.22 (40% ethyl acetate-pentane, PAA, stains puiple-blue).

¹H NMR (600 MHz, CDCl₃) 5.73 (m, 2H), 5.22 (m, 2H), 4.04 (dq, J=16.9, 5.3 Hz, 2H), 3.45 (d, J=6.4 Hz, 1H), 2.42-2.03 (m, 5H), 1.85-1.36 (m, 6H), 1.44 (s, 3H), 1.25 (s, 3H), 1.17-1.10 (m, 2H), 0.98 (d, J=7.1 Hz, 3H), 0.70 (d, J=7.0 Hz, 3H).

¹³C NMR (151 MHz, CDCl₃) 217.1, 172.3, 146.9, 115.5, 72.1, 70.2, 61.4, 58.3, 45.5, 45.4, 43.8, 42.0, 36.8, 34.6, 34.5, 30.3, 27.1, 25.1, 16.9, 15.0, 14.3, 11.0.

HRMS-ESI (m/z): calculated for [C₂₂H₃₄O₅Na]⁺ 401.2298, found 401.2297.

α_{D}²⁰=+36 (c=0.36, CHCl₃)

α_{D}²⁰=+37 (c=0.15, CHCl₃) [for 97 prepared by degradation of natural (+)-pleuromutilin (1)]

Synthesis of O-trityl-12-epi-pleuromutilin (96):

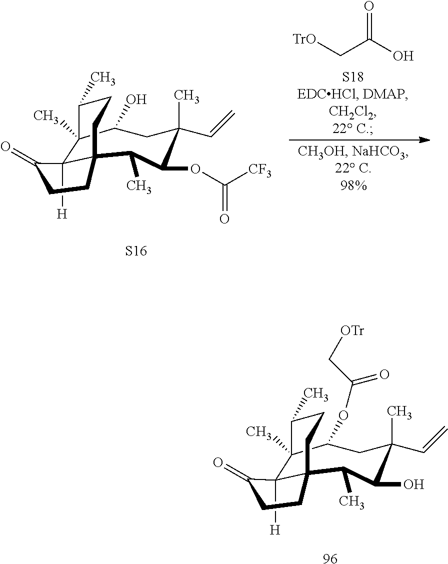

O-Tritylglycolic acid (S18, 10.3 mg, 32.5 μmol, 3.30 equiv) was added to a solution of the ester S16 (4.1 mg, 9.84 μmol, 1 equiv), N-(3-dimethylaminopropyl)-N′-ethyl-carbodiimide hydrogen chloride (EDC.HCl, 5.0 mg, 32.5 μmol, 3.30 equiv), and 4-(dimethylamino)pyridine (4.0 mg, 32.5 μmol, 3.30 equiv) in dichloromethane (500 μL) at 22° C. under air. The resulting mixture was stirred for 30 mm at 22° C. and then methanol (500 μL) and sodium bicarbonate (20.0 mg, 238 μmol, 24.2 equiv) were added in sequence. The resulting mixture was stirred for 46 h at 22° C. The product mixture was diluted with saturated aqueous ammonium chloride solution (1.5 mL) and the diluted product mixture was extracted with ethyl acetate (3×1.5 mL). The organic layers were combined and the combined organic layers were dried over sodium sulfate. The dried solution was filtered and the filtrate was concentrated. The residue obtained was purified by preparative thin-layered chromatography (eluting with 30% ethyl acetate-pentane) to provide O-trityl-12-epi-pleuromutilin 96 as a white solid (6.0 mg, 98%).

R_{f}=0.43 (40% ethyl acetate-pentane; PAA, stains green).

¹H NMR (600 MHz, CDCl₃) 7.49-7.43 (m, 6H), 7.33-7.21 (m, 9H), 5.72 (dd, J=17.4, 10.8 Hz, 1H), 5.67 (d, J=8.4 Hz, 1H), 5.25-5.17 (m, 2H), 3.75 (d, J=15.8 Hz, 1H), 3.65 (d, J=15.9 Hz, 1H), 3.42 (d, J=6.3 Hz, 1H), 2.40 (p, J=7.0 Hz, 1H), 2.29-2.14 (m, 2H), 2.09 (s, 1H), 1.98 (dd, J=15.9, 8.4 Hz, 1H), 1.80 (dd, J=14.6, 3.1 Hz, 1H), 1.68-1.35 (m, 5H), 1.42 (s, 3H), 1.23 (s, 3H), 1.12 (td, J=13.9, 4.8 Hz, 1H), 1.00-0.93 (m, 4H), 0.69 (d, J=6.8 Hz, 3H).

¹³C NMR (151 MHz, CDCl₃) 217.3, 169.0, 147.11, 143.4, 128.7, 128.1, 127.4, 115.3, 87.5, 72.1, 68.9, 63.3, 58.5, 45.6, 45.4, 43.7, 42.0, 36.9, 34.7, 34.5, 30.3, 27.1, 25.1, 17.0, 15.1, 14.3, 10.9.

HRMS-ESI (m/z): calculated for [C₄₁H₄₈O₅Na]⁺ 643.3394, found 643.3395.

Synthesis of (+)-pleuromutilin (1) and (+)-12-epi-pleuromutilin (97):

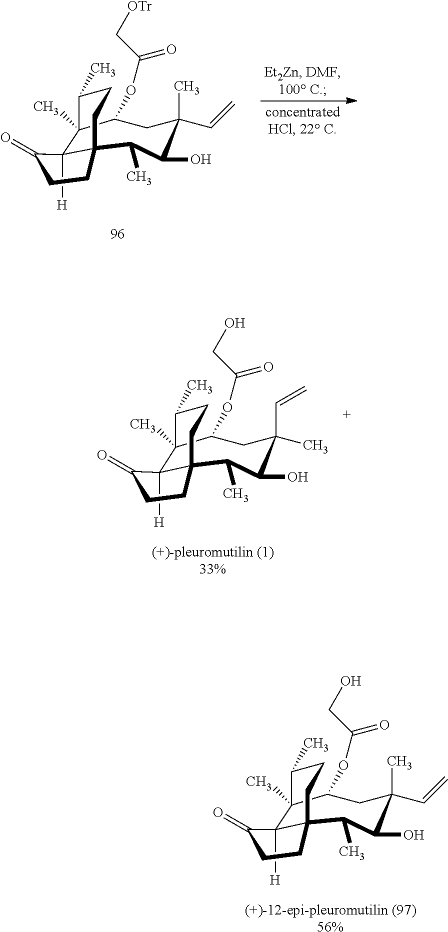

A solution of diethyl zinc in hexanes (1.0 M, 15.0 μL, 15.0 μmol, 1.03 equiv) was added to a solution of O-trityl-12-epi-pleuromutilin (96, 9.0 mg, 14.5 μmol, 1 equiv) in N,N-dimethylformamide (150 μL) at 22° C. The resulting mixture was heated at 100° C. for 2 h and then was cooled to 22° C. over 5 min. Concentrated aqueous hydrochloric acid solution (approximately 12 M, 50.0 μL) was added and the resulting mixture was stirred for 18 h at 22° C. The product mixture was diluted with saturated aqueous ammonium chloride solution (1.5 mL) and the diluted product mixture was extracted with ethyl acetate (3×1.5 mL). The organic layers were combined and the combined organic layers were dried over sodium sulfate. The dried solution was filtered and the filtrate was concentrated. The residue obtained was purified by preparative thin-layered chromatography (eluting with 25% ethyl acetate-dichloromethane, two steps) to provide separately (+)-pleuromutilin 1 (1.8 mg, 33%) and 12-epi-pleuromutilin 97 (3.1 mg, 56%) as white solids. The spectroscopic data for 1 were agreement with those obtained for a commercial sample.

1:

R_{f}=0.28 (25% ethyl acetate-dichloromethane, PAA, stains green-brown).

¹H NMR (400 MHz, CDCl₃) δ 6.50 (dd, J=17.4, 11.0 Hz, 1H), 5.85 (d, J=8.6 Hz, 1H), 5.37 (dd, J=11.0, 1.5 Hz, 1H), 5.22 (dd, J=17.4, 1.5 Hz, 1H), 4.04 (qd, J=17.1, 5.4 Hz, 2H), 3.37 (d, J=6.5 Hz, 1H), 2.29-2.41 (m, 1H), 2.17-2.19 (m, 2H), 2.11 (s, 1H), 2.06-2.16 (m, 1H), 1.78 (dd, J=14.4, 2.9 Hz, 1H), 1.63-1.74 (m, 2H), 1.51-1.61 (m, 1H), 1.45-1.55 (m, 1H), 1.44 (s, 3H), 1.35-1.43 (m, 1H), 1.32 (d, J=16.2 Hz, 1H), 1.18 (s, 3H), 1.08-1.18 (m, 1H), 0.90 (d, J=7.0 Hz, 3H), 0.71 (d, J=7.1 Hz, 3H).

¹³C NMR (151 MHz, CDCl₃) δ 217.0, 172.3, 139.0, 117.6, 74.7, 70.0, 61.5, 58.2, 45.6, 44.9, 44.2, 42.0, 36.8, 36.2, 34.6, 30.6, 27.0, 26.5, 25.0, 16.8, 14.9, 11.7.

α_{D}²³=+32+ (c=0.25, CHCl₃)

lit. α_{D}²⁰=+33° (c=0.2, CDCl₃)¹⁷

Synthesis of O-trityl-11,12-di-epi-pleuromutilin S19:

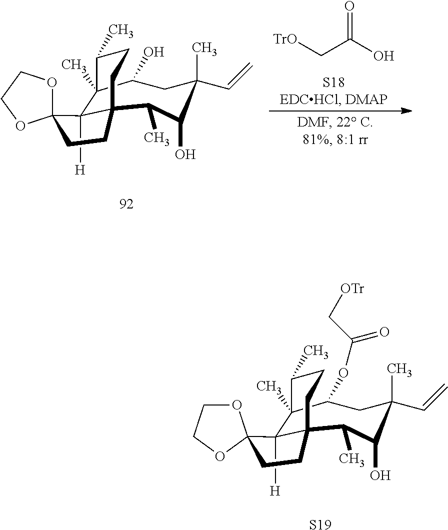

To a solid mixture of the diol 92 (5.4 mg, 14.8 μmol, 1 equiv), O-tritylglycolic acid (S18, 28.3 mg, 88.9 μmol, 6.00 equiv), 4-(dimethylamino)pyridine (25.3 mg, 207.4 μmol, 16.0 equiv), and N-(3-dimethylaminopropyl)-N′-ethyl-carbodiimide hydrogen chloride (EDC.HCl, 17.0 mg, 88.9 μmol, 6.00 equiv) was added N,N-dimethylformamide (450 μL) at 22° C. The resulting mixture was stirred for 18 h at 22° C. The product mixture was diluted with saturated aqueous sodium chloride solution (5.0 mL) and the diluted mixture was extracted with ethyl acetate (3×3.0 mL). The organic layers were combined and the combined layers were dried over magnesium sulfate. The dried solution was filtered and the filtrate was concentrated. The residue obtained was purified by preparative thin-layered chromatography (eluting with 25% ethyl acetate-hexanes) to provide O-trityl-11,12-di-epi-pleuromutilin (S19) as a white solid (7.9 mg, 81%, 8:1 rr, inseparable regioisomers). The mixture was used directly in the next step.

R_{f}=0.48 (25% ethyl acetate-hexanes; PAA, stains green).

¹H NMR (400 MHz, C₆D₆) δ 7.58 (d, J=7.6 Hz, 6H), 7.07 (t, J=7.6 Hz, 6H), 6.98 (t, J=7.3 Hz, 3H), 5.98 (d, J=7.7 Hz, 1H), 5.25 (dd, J=17.6, 11.1 Hz, 1H), 4.74 (d, J=11.1, 1H), 4.72 (d, J=17.6, 1H), 4.00 (d, J=15.4 Hz, 1H), 3.90 (d, J=15.4 Hz, 1H), 3.61-3.34 (m, 4H), 3.32 (s, 1H), 3.19 (s, 1H), 2.79-2.54 (m, 3H), 2.30 (q, J=7.1 Hz, 1H), 2.17-2.03 (m, 1H), 2.00-1.88 (m, 1H), 1.55 (s, 3H), 1.27 (s, 3H), 1.61-0.85 (m, 7H), 1.02 (d, J=6.8 Hz, 3H), 1.00 (d, J=6.8 Hz, 3H).

Synthesis of (+)-11,12-di-epi-pleuromutilin (93):

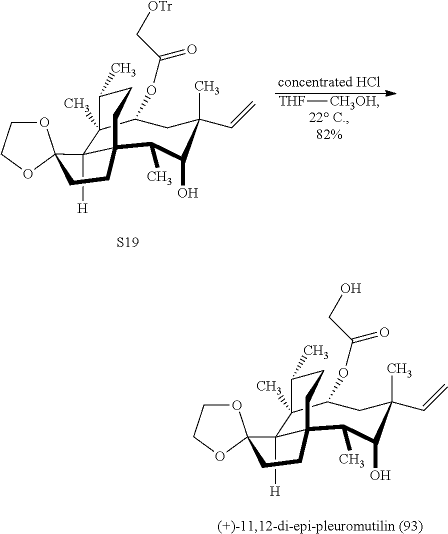

Concentrated hydrochloric acid (approximately 12 M, 50.0 μL) was added to a solution of O-trityl-11,12-di-epi-pleuromutilin (S19, 1.7 mg, 4.19 μmol, 1 equiv) in tetrahydrofuran-methanol (1:1 mixture, 1.0 mL) at 22° C. The resulting mixture was stirred for 1 h at 22° C. open to air and then was diluted with water (3.0 mL). The diluted product mixture was extracted with ethyl acetate (3×3.0 mL). The organic layers were combined and the combined organic layers were dried over magnesium sulfate. The dried solution was filtered and the filtrate was concentrated. The residue obtained was purified by preparative thin-layered chromatography (dining with 30% ethyl acetate-hexanes) to provide 11,12-di-epi-pleuromutilin (93) as a white solid (1.3 mg, 82%). The isolated material contained small amount of impurity. The yield is based on this material.

R_{f}=0.25 (30% ethyl acetate-hexanes; UV; PAA, stains black).

¹H NMR (600 MHz, CDCl₃) δ 5.80 (d, J=7.3 Hz, 1H), 5.69 (dd, J=17.7, 11.0 Hz, 1H), 5.19 (d, J=11.0 Hz, 1H), 5.09 (d, J=17.7 Hz, 1H), 4.09 (dd, J=17.0, 5.4 Hz, 1H), 4.02 (dd, J=17.0, 5.4 Hz, 1H), 3.47 (s, 1H), 3.10 (s, 1H), 2.53-2.41 (m, 2H), 2.34 (t, J=5.4 Hz, 1H), 2.31 (q, J=7.1 Hz, 1H), 2.25-2.08 (m, 3H), 1.70-1.57 (m, 3H), 1.50 (s, 3H), 1.39-1.32 (m, 2H), 1.22 (s, 3H), 1.15 (d, J=7.1 Hz, 1H), 1.12-1.07 (m, 1H), 1.08 (d, J=15.1 Hz, 2H), 0.70 (d, J=6.6 Hz, 3H).

¹³C NMR (151 MHz, CDCl₃) δ 219.6, 172.3, 146.7, 115.1, 84.2, 71.0, 61.6, 58.5, 45.1, 44.0, 42.3, 37.2, 37.1, 35.2, 33.4, 32.8, 27.5, 27.4, 216, 19.9, 16.8, 15.3.

HRMS-ESI (m/z): calculated for [C₂₂H₃₅O₅]⁺ 379.2485, found 379.2495.

α_{D}²⁰=+114° (c=0.04, CHCl₃)

### REFERENCES—FIRST SET DESCRIPTION

1. Newman, D. J.; Cragg, G. M. J. Nat. Prod. 2016, 79, 629.
2. (a) Kavanagh, F.; Hervey, A.; Robbins, W. J. Proc. Natl. Acad. Sci. U.S.A. 1951, 37, 570. (b) Kavanagh, F.; Hervey, A.; Robbins W. J. Proc. Natl. Acad. Sci. U.S.A. 1952, 38, 555. (c) Birch, A. J.; Holzapfel, C. W.; Rickards, R. W. Tetrahedron 1966, 22, 359. (d) Arigoni, D. Pure Appl. Chem. 1968, 17, 331.
3. Davidovich, C.; Bashan, A.; Auerbach-Neva, T.; Yaggie, R. D.; Gontarek, R. R.; Yonath, A. Proceedings of the National Academy of Sciences of the United States of America 2007, 104, 4291.
4. Fazakerley, N. J.; Procter, D. J. Tetrahedron 2014, 70, 6911.
5. Jones, R. N.; Fritsche, T. R.; Sader, H. S.; Ross, J. E. Antimicrob. Agents Chemother. 2006, 50, 2583.
6. Berner, H.; Vyplel, H.; Schulz, G.; Schneider, H. Monatshefte fur Chemie I Chemical Monthly 1986, 117, 1073.
7. Thirring, K.; Heilmayer, W.; Riedl, R.; Kollmann, H.; IvezicSchoenfeld, Z.; Wicha, W.; Paukner, S.; Strickmann, D. Preparation of 12-epi-pleuromutilin derivatives as antimicrobial agents. Patent W02015110481AI, 2015.
8. Blair, J. M. A.; Webber, M. A.; Baylay, A. J.; Ogbolu, D. 0.; Piddock, L. J. V. Nat Rev Micro 2015, 13, 42.
9. Liu, F.; Myers, A. G. Curr. Opin. Chem. Biol. 2016, 32, 48.
10. Seiple, I. B.; Zhang, Z.; Jakubec, P.; Langlois-Mercier, A.; Wright, P. M.; Hog, D. T.; Yabu, K.; Allu, S. R.; Fukuzaki, T.; Carlsen, P. N.; Kitamura, Y.; Zhou, X.; Condakes, M. L.; Szczypinski, F. T.; Green, W. D.; Myers, A. G. Nature 2016, 533, 338.
11. Wright, P. M.; Seiple, I. B.; Myers, A. G. Angew. Chem., Int. Ed. 2014, 53, 8840.
12. Murphy, S. K.; Zeng, M.; Herzon, S. B. Org. Lett. 2016, 18, 4880.
13. Crisp, G. T.; Scott, W. J.; Stile, J. K. J. Am. Chem. Soc. 1984, 106, 7500.
14. He, W.; Sun, X.; Frontier, A. J. J. Am. Chem. Soc. 2003, 125, 14278.
15. Nagata, W.; Yoshioka, M.; Terasawa, T. J. Am. Chem. Soc. 1972, 94, 4672.
16. Tsunoda, T.; Suzuki, M.; Noyori, R. Tetrahedron Lett. 1980, 21, 1357.
17. Comment on acylimine vs. enecarbamate.
18. Okutani, M.; Mori, Y. Chem. Pharm. Bull. 2015, 63, 393.
19. (a) Shareef, A.-R.; Sherman, D. H.; Montgomery, J. Chem. Sci. 2012, 3, 892. (b) Wang, H.; Negretti, S.; Knauff, A. R.; Montgomery, J. Org. Lett. 2015, 17, 1493.
20. Dess, D. B.; Martin, J. C. J. Am. Chem. Soc. 1991, 113, 7277.
21. Dahlen, A.; Hihnersson, G. Chem.—Eur. J. 2003, 9, 1123.

### REFERENCES—SECOND SET DESCRIPTION

1. Kavanagh, F.; Hervey, A.; Robbins, W. J. Proc. Natl. Acad. Sci. U.S.A. 1951, 37, 570.
2. Anchel, M. J. Biol. Chem. 1952, 199, 133.
3. Arigoni, D. Gazz. Chim. Ital. 1962, 92, 884.
4. Birch, A. J.; Holzapfel, C. W; Rickards, R. W. Tetrahedron 1966, 22, 359.
5. Dobler, M.; Duerr, B. G. Cryst. Struct. Comm. 1975, 4, 259.
6. Arigoni, D. Pure Appl. Chem. 1968, 17, 331.
7. (a) Schlunzen, F.; Pyetan, E.; Fucini, P.; Yonath, A.; Harms, J. M. Mol. Microbiol. 2004, 54, 1287. (b) Davidovich C.; Bashan, A.; Auerbach-Nevo, T.; Yaggie, R. D.; Gontarek, R. R.; Yonath, A. Proc. Natl. Acad. Sci. U.S.A. 2007, 104, 4291.
8. The positional numbering introduced by Arigoni (ref. 3) is used for all synthetic intermediates.
9. Egger, H.; Reinshagen, H. J. Antibiot. 1976, 29, 923.
10. For a review of synthetic and semisynthetic studies of (+)-pleuromutilin (1), see: Fazakerley, N. J.; Procter, D. J. Tetrahedron 2014, 70, 6911.
11. Daum, R. S.; Kar, S.; Kirkpatrick, P. Nat. Rev. Drug Discovery 2007, 6, 865.
12. Paukner, S.; Riedl, R. Cold Spring Harbor Perspect. Med. 2017, 7, a027110.
13. Walsh, C. T.; Wencewicz, T. A. J. Antibiot. 2014, 67, 7.
14. https://globenewswire.com/news-release/2017/09/18/1124000/0/en/Nabriva-Therapeutics-Announces-Positive-Topline-Results-from-Global-Phase-3-Clinical-Trial-Evaluating-IV-and-Oral-Lefamulin-for-the-Treatment-of-Community-Acquired-Bacterial-Pneumo.html accessed Oct. 9, 2017
15. Berner, H.; Vyplel, H.; Schulz, G; Schneider, H. Monatsh. Chem. 1986, 117, 1073.
16. (a) Paukner, S.; Gruss, A.; Fritsche, T. R.; Ivezic-Schoenfeld, Z.; Jones, R. N., In Vitro Activity of the Novel Pleuromutilin BC-3781 Tested Against Bacterial Pathogens Causing Sexually Transmitted Diseases (STD). In 53rd Interscience Conference on Antimicrobial Agents and Chemotherapy, Denver, Colo., 2013. (b) Paukner, S.; Strickmann, D. B.; Ivezic-Schoenfeld, Z., Extended Spectrum Pleuromutilins: Mode-of-action Studies. In 24th European Congress of Clinical Microbiology and Infectious Diseases (ECCMID), Barcelona, Spain, 2014. (c) Wicha, W. W.; Ivezic-Schoenfeld, Z., In Vivo Activity of Extended Spectrum Pleuromutilins in Murine Sepsis Model. In 24th European Congress of Clinical Microbiology and Infectious Diseases (ECCMID), Barcelona, Spain, 2014. (d) Thirring, K.; Heilmayer, W.; Riedl, R.; Kollmann, H.; Ivezic-Schoenfeld, Z.; Wicha, W.; Paukner, S.; Strickmann, D. Preparation of 12-epi-pleuromutilin derivatives as antimicrobial agents. Patent WO2015110481A1, 2015. (e) Paukner, S.; Kollmann, H.; Riedl, R.; Ivezic-Schoenfeld, Z., Kill curves of the Novel Extended-Spectrum Pleuromutilin Antibiotic BC-9529. In 25th European Congress of Clinical Microbiology and Infectious Diseases (ECCMID), Copenhagen, Denmark, 2015. (f) Paukner, S.; Wicha, W. W.; Heilmayer, W.; Thirring, K.; Riedl, R., Extended Spectrum Pleuromutilins: Potent Translation inhibitors with Broad-Spectrum Antibacterial Activity In Vitro an In Vivo. In Interscience Conference of Antimicrobial Agents and Chemotherapy/International Congress of Chemotherapy and Infection (ICAAC/ICC), San Diego, Calif., 2015. (g) Paukner, S.; Wicha, W. W.; Thirring, K.; Kollmann, H.; Ivezic-Schoenfeld, Z., In Vitro and In Vivo Efficacy of Novel Extended Spectrum Pleuromutilins Against S. aureus and S. pneumoniae. In 25th European Congress of Clinical Microbiology and Infectious Diseases (ECCMID), Copenhagen, Denmark, 2015. (h) Wicha, W. W.; Paukner, S.; Strickmann, D. B.; Thirring, K.; Kollmann, H.; Heilmayer, W.; Ivezic-Schoenfeld, Z., Efficacy of Novel Extended Spectrum Pleuromutilins Against E. coli In Vitro and In Vivo. In 25th European Congress of Clinical Microbiology and Infectious Diseases (ECCMID), Copenhagen, Denmark, 2015.
17. https://www.cdc.gov/drugresistance/biggest_threats.html, accessed May 13, 2017.
18. Post. K. W.; Songer, J. G. Anaerobe 2004, 10, 47.
19. Murphy, S. K.; Zeng, M.; Herzon, S. B. Org. Lett. 2016, 18, 4880.
20. Murphy, S. K.; Zeng, M.; Herzon, S. B. Science 2017, 356, 956.
21. Murphy, S. K.; Zeng, M.; Herzon, S. B. Org. Lett. 2017, 19, 4980.
22. Page, M. G. P. Beta-Lactam Antibiotics. In Antibiotic Discovery and Development; Dougherty, T. J., Pucci, M. J., Eds.; Springer: Amsterdam, 2012; pp 79-117.
23. Okano, A.; Isley, N. A.; Boger, D. L. Chem. Rev. 2017, 117, 11952.
24. Liu, F.; Myers, A. G. Curr. Opin. Chem. Biol. 2016, 32, 48.
25. Seiple, I. B.; Zhang, Z.; Jakubec, P.; Langlois-Mercier, A.; Wright, P. M.; Hog, D. T.; Yabu, K.; Allu, S. R.; Fukuzaki, T.; Carlsen, P. N.; Kitamura, Y.; Zhou, X.; Condakes, M. L.; Szczypiński, F. T.; Green, W. D.; Myers, A. G. Nature 2016, 533, 338.
26. For a review, see: Wright, P. M.; Seiple, I. B.; Myers, A. G. Angew Chem. Int. Ed. 2014, 53, 8840.
27. Gibbons, E. G. J. Am. Chem. Soc. 1982, 104, 1767.
28. Boeckman, R. K.; Springer, D. M.; Alessi, T. R. J. Am. Chem. Soc. 1989, 111, 8284.
29. Fazakerley, N. J.; Helm, M. D.; Procter, D. J. Chem.—Eur. J. 2013, 19, 6718.
30. (a) Kahn, M. Tetrahedron Lett. 1980, 21, 4547. (b) Paquette, L. A.; Wiedeman, P. E.; Page, P. C. B. J. Org. Chem. 1988, 53, 1441. (c) Paquette, L. A.; Page, P. C. B.; Pansegrau, P. D.; Wiedeman, P. E. J. Org. Chem. 1988, 53, 1450. (d) Paquette, L. A.; Pansegrau, P. D.; Wiedeman, P. E.; Springer, J. P. J. Org. Chem. 1988, 53, 1461. (e) Bacque, E.; Pautrat, F.; Zard, S. Z. Org. Lett. 2003, 5, 325. (f) Findley; T. J. K.; Sucunza, D.; Miller, L. C.; Helm, M. D.; Helliwell, M.; Davies, D. T.; Procter, D. J. Org. Biomol. Chem. 2011, 9, 2433. (g) Lotesta, S. D.; Liu, J.; Yates, E. V.; Krieger, I.; Sacchettini, J. C.; Freundlich, J. S.; Sorensen, E. J. Chem. Sci. 2011, 2, 1258. (h) Liu, J.; Lotesta, S. D.; Sorensen, E. J. Chem. Commun. 2011, 47, 1500.
31. For a discussion of step-economy, see: Wender, P. A.; Verma, V. A.; Paxton, T. J.; Pillow, T. H. Acc. Chem. Res. 2008, 41, 40.
32. (a) Takai, K.; Kimura, K.; Kuroda, T.; Hiyama, T.; Nozaki, H. Tetrahedron Lett. 1983, 24, 5281. (b) Jin, H.; Uenishi, J.; Christ, W. J.; Kishi, Y. J. Am. Chem. Soc. 1986, 108, 5644. For a review, see: (c) Takai, K. Addition of Organochromium Reagents to Carbonyl Compounds. In Organic Reactions; John Wiley & Sons, Inc.; Hoboken, 2004; pp 253-612.
33. For an early example, see: Kablaoui, N. M.; Buchwald, S. L. J. Am. Chem. Soc. 1995, 117, 6785.
34. For reviews of aldehyde-alkyne reductive coupling, see: (a) Moslin, R. M.; Miller-Moslin, K.; Jamison, T. F. Chem. Commun. 2007, 4441. (b) Jackson, E. P; Malik H. A.; Sormunen, G. J.; Baxter, R. D.; Liu, P.; Wang, H.; Shareef, A.-R.; Montgomery, J. Acc. Chem. Res. 2015, 48, 1736.
35. Carey, F. A.; Sundberg, R. J. 3.4 Carbocyclic Rings Other Than Six-Membered. In Advanced Organic Chemistry: 3rd ed.; Plenum: New York, 1990; Vol. A, pp 141-144.
36. Illuminati, G.; Mandolini, L.; Masci, B. J. Am. Chem. Soc. 1975, 97, 4960.
37. Morita, Y.; Suzuki, M.; Noyori, R. J. Org. Chem. 1989, 54, 1785.
38. Mander, L. N.; Sethi, S. P. Tetrahedron Lett. 1983, 24, 5425.
39. Heller, S. T.; Sarpong, R. Org. Lett. 2010, 12, 4572.
40. Crisp, G. T.; Scott, W. J.; Stille, J. K. J. Am. Chem. Soc. 1984, 106, 7500.
41. He, W; Sun, X.; Frontier, A. J. J. Am. Chem. Soc. 2003, 125, 14278.
42. (a) MacAlpine, G. A.; Raphael, R. A.; Shaw, A.; Taylor, A. W.; Wild, J.-J. J. Chem. Soc. Chem. Commun. 1974, 834. (b) Karpf, M.; Dreiding, A. S. Helv. Chim. Acta 1976, 59, 1226. (c) Hiyama, T.; Shinoda, M.; Saimoto, H.; Nozaki, H. Bull. Chem. Soc. Jpn. 1981, 54, 2747.
43. Hosokawa, S.; Sekiguchi, K.; Enemoto, M.; Kobayashi, S. Tetrahedron Lett. 2000, 41, 6419.
44. Zhana, Y.; Rovis, T. J. Am. Chem. Soc. 2004, 126, 15964.
45. (a) Wotal, A. C.; Weix, D. J. Org. Lett. 2012, 14, 1476. For a review of cross-electrophile coupling, see: (b) Weix, D. J. Acc. Chem. Res. 2015, 48, 1767.
46. (a) Katritzky, A. R.; Yao, J.; Qi, M. J. Org. Chem. 1997, 62, 8201. (b) Cacchi, S.; Morera, E.; Ortar, G. Synthesis 1986, 320.
47. (a) Genin, E.; Toullec, P. Y; Antoniotti, S.; Brancour; C.; Genêt, J.-P.; Michelet, V. J. Am. Chem. Soc. 2006, 128, 3112. For a review of gold-mediated alkyne activation, see: (b) Dorel, R.; Echavarren, A. M. Chem. Rev. 2015, 115, 9028.
48. Trost, B. M.; Jiang, C. J. Am. Chem. Soc. 2001, 123, 12907.
49. Williams, J. M.; Jobson, R. B.; Yasuda, N.; Marchesini, G.; Dolling, U.-H.; Grabowski, E. J. J. Tetrahedron Lett. 1995, 36, 5461.
50. Feringa, B. L.; Pineschi, M.; Arnold, L. A.; Imbos, R.; de Vries, A. H. M. Angew. Chem., Int. Ed. 1997, 36, 2620.
51. Tanino, K.; Onuki, K.; Asano, K.; Miyashita, M.; Nakamura, T.; Takahashi, Y.; Kuwajima, I. J. Am. Chem. Soc. 2003, 125, 1498.
52. Comins, D. L.; Dehghani, A. Tetrahedron Lett. 1992, 33, 6299.
53. Gagnier, S. V; Larock R. C. J. Am. Chem. Soc. 2003, 125, 4804.
54. Larionov, O. V.; Corey, E. J. Org. Lett. 2010, 12, 300.
55. (a) Boeckman, R. K.; Bruza, K. J. Org. Chem. 1979, 44, 4781. (b) Kramer, R.; Brückner, R. Eur. J. Org. Chem. 2013, 6563. For a review of the mechanisms of organocopper reactions, see: (c) Yoshikai, N.; Nakamura, E. Chem. Rev. 2012, 112, 2339.
56. Lipshutz, B. H.; Elsworth, E. L.; Siahaan, T. J. J. Am. Chem. Soc. 1989, 111, 1351.
57. For a review, see: Nagata, W; Yoshioka, M. Hydrocyanation of Conjugated Carbonyl Compounds. In Organic Reactions; John Wiley & Sons, Inc.: New York, 2004; pp 255-476.
58. Kraus, G. A.; Frazier, K. J. Org. Chem. 1980, 45, 4262.
59. Roth, G. J.; Liepold, B.; Müller, S. Bestmann, H. J. Synthesis 2004, 59.
60. For the exo-selective reductive cyclization of ynals, see: (a) Shareef, A.-R.; Sherman, D. H.; Montgomery, J. Chem. Sci. 2012, 3, 892. (b) Wang, H.; Negretti, S.; Knauff, A. R.; Montgomery, J. Org. Lett. 2015, 17, 1493.
61. Dess, D. B.; Martin, J. C. J. Am. Chem. Soc. 1991, 113, 7277.
62. Okutani, M.; Mori, Y. Chem. Pharm. Bull. 2015, 63, 393.
63. Li, L.; Zeng, J.; Herzon, S. B. Angew. Chem., Int. Ed. 2014, 53, 7892.
64. Pidathala, C.; Hoang, L.; Vignola, N.; List, B. Angew. Chem., Int. Ed. 2003, 42, 2785.
65. Cacchi, S.; Morera, E.; Ortar, G. Palladium-Catalyzed Reduction of Vinyl Trifluoromethanesulfonates to Alkenes: Cholesta-3,5-diene. In Organic Syntheses: John Wiley & Sons, Inc.: 2003.
66. Gradillas, A.; Pérez-Castells, J. Angew. Chem., Int. Ed. 2006, 45, 6086.
67. Hutton, T. K.; Muir, K.; Procter, D. J. Org. Lett. 2002, 4, 2345.

### REFERENCES—EXAMPLES—FIRST SET OF EXPERIMENTS

1. Still, W. C.; Kahn, M.; Mitra, A., J. Org. Chem. 1978, 43, 2923-2925.
2. Pangborn, A. B.; Giardello, M. A.; Grubbs., R. H.; Rosen, R. K.; Timmers, F. J., Organometallics 1996, 15, 1518-1520.
3.Krasovskiy, A.; Knochel, P., Synthesis 2006, 890-891.
4. Lin, H.-S.; Paquette, L. A., Synth. Commun. 1994, 24, 2503-2506.
5. Feringa B. L.; Pineschi, M.; Arnold, L. A.; Imbos, R.; de Vries, A. H. M., Angew Chem Int Ed. Engl. 1997, 36, 2620-2623.
6. Xu, Q.-Q.; Zhao, Q.; Shan, G.-S.; Yang, X.-C.; Shi, Q.-Y.; Lei, X., Tetrahedron 2013, 69, 10739-10746.
7. Nishikawa, K.; Nakahara, H.; Shirokura Y.; Nogata, Yoshimura, E.; Umezawa, T.; Okino, T.; Matsuda, F., Org. Lett. 2010, 12, 904-907.
8. Roth, G. J.; Liepold, B.; Müller, S. G.; Bestmann, H. J., Synthesis 2004, 2004, 59-62.
9. Frigerio, M.; Santagostino, M.; Sputore, S., J. Org. Chem. 1999, 64, 4537-4538.
10. Meyer, S. D.; Schreiber, S. L., J. Org. Chem. 1994, 59, 7549-7552.
11. Arduengo, A. J.; Krafczyk, R.; Schmutzler, R.; Craig, H. A.; Goerlich, J. R; Marshall, W. J.; Unverzagt, M., Tetrahedron 1999, 55, 14523-14534.
12. Blom, B.; Driess, M.; Gallego, D.; Inoue, S., Chem.—Eur. J. 2012, 18, 13355-13360.
13. Iorio, M. A.; Molinari, M.; Brossi. A., Can. J. Chem. 1981, 59, 283-284.
14. Rijkers, D. T. S.; Höppener, J. W. M.; Posthuma, G.; Lips, C. J. M.; Liskamp, R. M. J., Chem.—Eur. J. 2002, 8, 4285-4291.
15. Murphy, S. K.; Zeng, M.; Herzon, S. B., Org. Lett 2016, 18, 4880-4883.
16. Trost, B. M.; Jiang, C., J. Am. Chem. Soc. 2001, 123, 12907-12908.
17. Fazakerley, N. S.; Helm, M. D.; Procter, D. J., Chem.—Eur. J. 2013, 19, 6718-6723.

### REFERENCES—EXAMPLES—SECOND SET OF EXPERIMENTS

1. F. Kavanagh, A. Hervey, W. J. Robbins, Proc. Natl. Acad. Sci. U.S.A. 37, 570 (1951).
2. F. Kavanagh, A. Hervey, W. J. Robbins, Proc. Natl. Acad. Sci. U.S.A. 38, 555 (1952).
3. A. J. Birch, C. W. Holzapfel, R. W. Rickards, Tetrahedron 22, 359 (1966).
4. D. Arigoni, Pure Appl. Chem. 17, 331 (1968).
5. C. Davidovich et al., Proc. Natl. Acad. Sci. U.S.A. 104, 4291 (2007).
6. S. Paukner, R. Riedl, Cold Spring Harbor Perspect. Med. 7, (2017).
7. N. J. Fazakerley, D. J. Procter, Tetrahedron 70, 6911 (2014).
8. R. N. Jones, T. R. Fritsche, H. S. Sader, S. E. Ross, Antimicrob. Agents Chemother. 50, 2583 (2006).
9. H. Berner, H. Vyplel, G. Schulz, H. Schneider, Monatsh. Chem. 117, 1073 (1986).
10. K. Thirring et al., “Preparation of 12-epi-pleuromutilin derivatives as antimicrobial agents” WO2015110481A1, Jul. 30, 2015
11. J. M. A. Blair, M. A. Webber, A. J. Baylay, D. O. Ogbolu, L. J. V. Piddock, Nat. Rev. Microbiol. 13, 42 (201.5).
12. H. J. Morrill, J. M. Pogue, K. S. Kaye, K. L. LaPlante, Open Forum Infect. Dis. 2, ofv050 (2015).
13. E. G. Gibbons, J. Am. Chem. Soc. 104, 1767 (1982).
14. R. K. Boeckman, D. M. Springer, T. R. Alessi, J. Am. Chem. Soc. 111, 8284 (1989).
15. N. J. Fazakerley, M. D. Helm, D. J. Procter, Chem.—Eur. J. 19, 6718 (2013).
16. M. Kahn, Tetrahedron Lett. 21, 4547 (1980).
17. L. A. Paquette, P. C. B, Page, P. D. Pansegrau, P. E. Wiedeman, J. Org. Chem. 53, 1450 (1988).
18. L. A. Paquette, P. D. Pansegrau, P. E. Wiedeman, J. P. Springer, J. Org. Chem. 53, 1461 (1988).
19. L. A. Paquette, P. E. Wiedeman, P. C. B. Page, J. Org. Chem. 53, 1441 (1988).
20. E. Bacque, F. Pautrat, S. Z. Zard, Org. Lett. 5, 325 (2003).
21. T. J. K. Findley et al., Org. Biomol. Chem. 9, 2433 (2011).
22. S. D. Lotesta et al., Chem. Sci. 2, 1258 (2011).
23. J. Liu, T. A. Bedell, J. G. West, E. J. Sorensen, Tetrahedron 72, 3579 (2016).
24. S. K. Murphy, M. Zeng, S. B, Herzon, Org. Lett. 18, 4880 (2016).
25. R. A. Bunce, R. Harris, J. Org. Chem. 57, 6981 (1992).
26. G. T. Crisp, W. J. Scott, J. K. Stille, J. Am. Chem. Soc. 106, 7500 (1984).
27. W. He, X. Sun, A. J, Frontier, J. Am. Chem. Soc. 125, 14278 (2003).
28. W. Nagata, M. Yoshioka, T. Terasawa, J. Am. Chem. Soc. 94, 4672 (1972).
29. T. Tsunoda, M. Suzuki, R. Noyori, Tetrahedron Lett. 21, 1357 (1980).
30. S. Hosokawa, K. Sekiguchi, M. Enemoto, S. Kobayashi, Tetrahedron Lett. 41, 6429 (2000).
31. R. M. Moslin, K. Miller-Moslin, T. F. Jamison, Chem. Commun., 4441 (2007).
32. E. P. Jackson et al., Acc. Chem. Res. 48, 1736 (2015).
33. A.-R. Shareef, D. H. Sherman, J. Montgomery, Chem. Sci. 3, 892 (2012).
34.H. Wang, S. Negretti, A. R. Knauff, J. Montgomery, Org. Lett. 17, 1493 (2015).
35. G. Illuminati, L. Mandolini, B. Masci, J. Am. Chem. Soc. 97, 4960 (1975).
36. D. B. Dess, J. C. Martin, J. Am. Chem. Soc. 113, 7277 (1991).
37. A. Dahlén, G. Hilmersson, Chem.—Eur. J. 9, 1123 (2003).
38. F. Liu, A. G. Myers, Curr. Opin. Chem. Biol. 32, 48 (2016).
39. I. B. Seiple et al., Nature 533, 338 (2016).
40. P. M. Wright, I. B. Seiple, A. G. Myers, Angew. Chem., Int. Ed. 53, 8840 (2014).
41. W. C. Still, M. Kahn, A. Mitra, J. Org. Chem. 43, 2923 (1978).
42. A. B. Pangborn, M. A. Giardello, R. H. Grubbs, R. K. Rosen, F. J. Timmers, Organometallics 15, 1518 (1996).
43. A. Krasovskiy, P. Knochel, Synthesis 5, 890 (2006).
44. H.-S. Lin, L. A. Paquette, Synth. Commun. 24, 2503 (1994).
45. B. L. Feringa, M. Pineschi, L. A. Arnold, R. Imbox, A. H. M. de Vries, Angew. Chem. Int. Ed. 36, 2620 (1997).
46. Q.-Q. Xu, Q. Zhao, G.-S. Shan, X.-C. Yang, Q.-Y. Shi, X. Lei, Tetrahedron 69, 10739 (2013).
47. K. Nishikawa, H. Nakahara, Y. Shirokura, Y. Nogata, E. Yoshimura, T. Umezawa, T. Okino, F. Matsuda, Org. Lett. 12, 904 (2010).
48. M. A. Iorio, M. Molinari, A. Brossi, Can. J. Chem. 59, 283 (1981).
49. D. T. S. Rijkers, J. W. M. Höppener, G. Posthuma, C. J. M. Lips, R. M. J. Liskamp, Chem. Eur. J. 8, 4285 (2002).
50. H. Hasler, PhD Thesis, ETH Zurich, (1979).
51. G. Schulz, H. Berner, Tetrahedron 40, 905 (1984).

1. A compound according to the chemical structure: 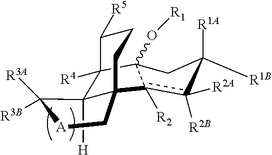
Where A is O, S, —N(R^{N})(C(R_{A})(R_{B}))_{g}— or —(C(R_{A})(R_{B}))ₕ—;
R^{N} is H or a C₁-C₃ alkyl group which is optionally substituted with from 1 to 3 hydroxyl groups or halogen groups (preferably fluoro groups);
R_{A} and R_{B} are each independently H, a halogen group (often F), a C₁-C₃ alkyl which is optionally substituted with from 1-3 halogen groups (often 1-3 fluoro groups) or 1-3 hydroxyl groups (often a single hydroxyl group) or together R_{A} and R_{B} form a cyclopropyl or cyclobutyl group on a single carbon;
R₁ is H, an optionally substituted C₁-C₇ alkyl group (preferably C₁-C₃ alkyl, preferably methyl) which is preferably substituted with from 1-5 halogens (F, Cl, Br or I), often from 1-3 fluoro groups or from 1-3 hydroxyl groups, a Sugar group wherein said sugar group is a monosaccharide or disaccharide sugar as otherwise described herein which forms a glycosidic linkage with the oxygen (preferably at the 1 or 4 carbon position of the sugar moiety bonded to the oxygen), an optionally substituted —(CH₂)ᵢ—C(O)—C₀-C₆ alkyl group (forming an ester) which is preferably substituted with from 1-5 halogens, often 1-3 fluoro groups and from 1-3 hydroxyl groups (preferably, R₁ forms a methyl ester group substituted with a single hydroxyl group) or a —(CH₂)ᵢ—C(O)—(CH₂)ᵢ—O-Sugar group;
R^{1A} and R^{1B} are each independently H, an optionally substituted C₁-C₆ alkyl or C₂-C₆ alkenyl group (preferably vinyl, often R^{1B} is a vinyl group wherein said alkyl group or said alkenyl group is preferably substituted with from 1-5 halogen groups and/or from 1-3 hydroxyl groups), an optionally substituted —(CH₂)ᵢNR^{NA}R^{NB} group, OH, an optionally substituted —(CH₂)ᵢO—C₁-C₆ alkyl group, an optionally substituted —(CH₂)ᵢC(O)—C₀-C₆ alkyl, an optionally substituted —(CH₂)ᵢC(O)O—C₁-C₆ alkyl or an optionally substituted —(CH₂)ᵢOC(O)—C₁-C₆ alkyl wherein each of the aforementioned alkyl groups is preferably substituted with from 1-5 halogen groups (often 1-3 fluoro groups) or from 1-3 hydroxyl groups, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar, an optionally substituted —(CH₂)ᵢ—C(O)—(CH₂)ᵢ—O-Sugar group, or R^{1A} or R^{1B} together with the carbon atom to which R₂ is attached form an optionally substituted 5-6 membered carbocyclic ring which link the carbon atoms which are bonded to R^{1A} or R^{1B} and R₂, respectively, wherein the alkylene group extends above or below the plane of the molecule;
R^{NA} and R^{NB} is each independently H, a C₁-C₆ alkyl which is optionally substituted with from 1-3 halo groups (preferably F) or 1-3 hydroxyl groups (often 1 hydroxyl group), an optionally substituted —(CH₂)ᵢO—C₁-C₆ alkyl, an optionally substituted —(CH₂)ᵢC(O)C₀-C₆ alkyl, an optionally substituted —(CH₂)ᵢC(O)OC₁-C₆ alkyl, an optionally substituted —(CH₂)ᵢOC(O)C₁-C₆ alkyl, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar or an optionally substituted —(CH₂)ᵢ—C(O)—(CH₂)ᵢ—O-Sugar group;
R₂ is H, an optionally substituted C₁-C₈ alkyl group which is preferably substituted with from 1-5 halo groups, often 1-3 fluoro groups or from 1-3 hydroxyl groups, OH, SH, an optionally substituted —(CH₂)ᵢNR^{NA}R^{NB} group, an optionally substituted —(CH₂)ᵢO—C₁-C₆ alkyl group, an optionally substituted —(CH₂)ᵢC(O)—C₁-C₆ alkyl, an optionally substituted —(CH₂)ᵢC(O)O—C₁-C₆ alkyl or an optionally substituted —(CH₂)ᵢOC(O)—C₁-C₆ alkyl wherein each of the aforementioned alkyl groups is preferably substituted with from 1-5 halogen groups (often 1-3 fluoro groups) or from 1-3 hydroxyl groups, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar or an optionally substituted —(CH₂)ᵢ—C(O)—(CH₂)ᵢ—O-Sugar group, or R₂ together with R^{1A} or R^{1B} forms a C₂-C₅ alkylene group optionally substituted with from 1 to 4 methyl groups which links the carbon atoms which are bonded to R₂ and R^{1A} or R^{1B}, respectively, wherein the alkylene group extends above or below the plane of the molecule;
R^{2A} and R^{2B} are each independently H, OH, an optionally substituted C₁-C₆ alkyl or C₂-C₆ alkenyl group (preferably vinyl) wherein said alkyl group or said alkenyl group is preferably substituted with from 1-5 halogen groups and from 1-3 hydroxyl groups), an optionally substituted —(CH₂)ᵢNR^{NA}R^{NB} group, an optionally substituted —(CH₂)ᵢO—C₁-C₆ alkyl, an optionally substituted —(CH₂)ᵢC(O)—C₀-C₆ alkyl (often C₁-C₆ alkyl), an optionally substituted —(CH₂)ᵢC(O)O—C₁-C₆ alkyl or an optionally substituted —(CH₂)ᵢOC(O)—C₁-C₆ alkyl wherein each of the aforementioned alkyl groups is preferably substituted with from 1-5 halogen groups (often 1-3 fluoro groups) or from 1-3 hydroxyl groups, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)ᵢSugar, an optionally substituted —(CH₂)ᵢO-Sugar or an optionally substituted —(CH₂)ᵢ—C(O)—(CH₂)ᵢ—O-Sugar group;
R^{3A} and R^{3B} are each independently H, OH, a C₁-C₆ optionally substituted alkyl group, an optionally substituted —(CH₂)ᵢO—C₁-C₆ alkyl group, or R^{3A} and R^{3B} together with the carbon atom to which they are attached form a C₂-C₆ diether group, often a C₃ or C₄ diether group (each of the two oxygens of the diether group being bonded to the carbon to which R^{3A} and R^{3B} are bonded) or a keto group (═O) with the carbon to which they are bonded;
R⁴ and R⁵ are each independently H or an optionally substituted C₁-C₈ alkyl group (preferably methyl) wherein said substitution is preferably from 1-5 halo groups (often F) or from 1-3 hydroxyl groups (often a single hydroxyl group);
g is 0, 1, 2 or 3;
h is 1, 2, 3 or 4;
i is 0, 1, 2, 3, 4, 5 or 6; and
the carbon atoms to which OR¹ and R₂ are attached optionally are bonded to each other; or a pharmaceutically acceptable salt, stereoisomer, solvate or polymorph thereof.

2. The compound according to claim 1 wherein A is CH₂, —N(R^{N})(C(R_{A})(R_{B}))_{g}— or —(C(R_{A})(R_{B}))ₕ— where RN is H or a C₁-C₃ alkyl group optionally substituted with from 1-3 fluoro groups or 1-3 hydroxyl groups) and R_{A} and R_{B} are each independently H, halogen (especially fluoro) or a C₁-C₃ alkyl group optionally substituted with from 1-3 fluoro groups (preferably 3 fluoro groups) or 1-3 hydroxyl groups (preferably 1 hydroxyl group);
R₁ is H, an optionally substituted C₁-C₇ alkyl group (preferably C₁-C₃ alkyl, preferably methyl) which is preferably substituted with from 1-5 halogens (F, Cl, Br or I), often from 1-3 fluoro groups or from 1-3 hydroxyl groups, preferably 1 hydroxyl group or a C(O)C₁-C₆ alkyl group optionally substituted with 1-3 fluoro groups or 1-3 hydroxyl groups or a —(CH₂)ᵢ—C(O)—(CH₂)ᵢ—O-Sugar group;
R^{1A} and R^{1B} are each H, a C₁-C₇ alkyl group or a C₂-C₆ alkenyl group, each of which is optionally substituted with 1-3 halogen (preferably fluoro) groups or 1-3 hydroxyl groups, a —(CH₂)ᵢ—O—C₁-C₆ alkyl group, a —(CH₂)ᵢ—C(O)C₁-C₆ alkyl group, a —(CH₂)ᵢ—O—C(O)C₁-C₆ alkyl group or a —(CH₂)ᵢ—C(O)O—C₁-C₆ alkyl group, each of which groups is optionally substituted with from 1-3 halogen (preferably fluoro) or from 1-3 hydroxyl groups, a —(CH₂)ᵢ-Sugar group, a —(CH₂)ᵢ—O-Sugar group, a —(CH₂)ᵢ—C(O)—(CH₂)ᵢ—O-Sugar group or a —(CH₂)ᵢ—NR^{NA}R^{NB} group, where R^{NA} and R^{NA} are each independently H, a C₁-C₆ alkyl group optionally substituted with 1-3 halogens (preferably fluoro) or 1-3 hydroxyl groups, a —(CH₂)ᵢ—O—C₁-C₆ alkyl group, a —(CH₂)ᵢ—C(O)C₁-C₆ alkyl group, a —(CH₂)ᵢ—O—C(O)C₁-C₆ alkyl group or a —(CH₂)ᵢ—C(O)O—C₁-C₆ alkyl group, each of which groups are optionally substituted with from 1-3 halogen (preferably fluoro) or from 1-3 hydroxyl groups, an optionally substituted —(CH₂)ᵢAryl, an optionally substituted —(CH₂)ᵢO-Aryl, an optionally substituted —(CH₂)ᵢHeteroaryl, an optionally substituted —(CH₂)ᵢO-Heteroaryl, an optionally substituted —(CH₂)ᵢSugar or an optionally substituted —(CH₂)ᵢO-Sugar group, or R^{1A} and the carbon to which R₂ is attached form a 5-6 membered carbocyclic ring, which is optionally substituted;
R₂ is H, a C₁-C₈ alkyl group optionally substituted with 1-3 halogens (preferably fluoro) or 1-3 hydroxyl groups, a —(CH₂)ᵢ—O—C₁-C₆ alkyl group which is optionally substituted with from 1-3 halogens (preferably fluoro) or from 1-3 hydroxyl groups, a —(CH₂)ᵢ—C(O)—(CH₂)ᵢ—O-Sugar group, or a —(CH₂)ᵢ—NR^{NA}R^{NB} group where R^{NA} and R^{NB} are the same as described above;
R^{2A} and R^{2B} are each independently H, a C₁-C₆ alkyl group or a C₂-C₆ alkenyl group each of which is optionally substituted with from 1-3 halogens (preferably fluoro) or from 1-3 hydroxyl groups, a —(CH₂)ᵢ—O—C₁-C₆ alkyl group, a —(CH₂)ᵢ—C(O)C₁-C₆ alkyl group, a —(CH₂)ᵢ—O—C(O)C₁-C₆ alkyl group or a —(CH₂)ᵢ—C(O)O—C₁-C₆ alkyl group, each of which groups is optionally substituted with from 1-3 halogen (preferably fluoro) or from 1-3 hydroxyl groups, a —(CH₂)ᵢ-Sugar group, a —(CH₂)ᵢ—O-Sugar group, a —(CH₂)ᵢ—C(O)—(CH₂)ᵢ—O-Sugar group, an optionally substituted —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl;
R^{3A} and R^{3B} are each independently H, OH, a C₁-C₆ alkyl group which is optionally substituted with from 1-3 halogens or from 1-3 hydroxyls, a keto group (C═O) or together with the carbon to which they are both attached, form a C₃ or C₄ diether group; and
R⁴ and R⁵ are each independently H or a C₁-C₃ alky group optionally substituted with from 1-3 halogens (preferably fluoro) or from 1-3 hydroxyl groups;
Each g is 0 or 1;
Each h is 1, 2 or 3; and
Each i is independently 0, 1, 2 or 3, or
a pharmaceutically acceptable salt or stereoisomer thereof.

3. A compound according to claim 1 wherein A is CH₂, NH or C(R_{A})(R_{B}) where R_{A} and R_{B} form an isopropyl group with C.

4. A compound according to claim 1 wherein R₁ is H, an optionally substituted C₁-C₃ alkyl group, a C(O)C₁-C₆ alkyl group optionally substituted with 1-3 fluoro groups or 1-3 hydroxyl groups or a —(CH₂)ᵢ—C(O)—(CH₂)ᵢ—O-Sugar group.

5. The compound according to claim 1 wherein R₁ is H, a C(O)CH₂OH group or a C(O)CH₂O-Sugar group.

6. The compound according to claim 1 wherein R^{1A} and R^{1B} are each independently H, an optionally substituted C₁-C₆ group or a C₂-C₆ alkenyl group, each of which is optionally substituted with 1-3 halogen groups or 1-3 hydroxyl groups, a —(CH₂)ᵢ——C₁-C₆ alkyl group, a —(CH₂)ᵢ—C(O)C₀-C₆ alkyl group, a —(CH₂)ᵢ—O—C(O)C₁-C₆ alkyl group, a —(CH₂)ᵢ—C(O)O—C₁-C₆ alkyl group, each of which groups is optionally substituted with from 1-3 halogen or from 1-3 hydroxyl groups, a —(CH₂)ᵢSugar, an optionally substituted —(CH₂)ᵢO-Sugar, an optionally substituted —(CH₂)ᵢ—C(O)—(CH₂)ᵢ—O-Sugar group, or a NH₂ group.

7. The compound according to claim 1 wherein R₂ is H or a C₁-C₈ optionally substituted alkyl group, (CH₂)ᵢNR^{NA}R^{NB}, an optionally substituted —(CH₂)ᵢAryl, an optionally substituted —(CH₂)ᵢO-Aryl, an optionally substituted —(CH₂)ᵢHeteroaryl, an optionally substituted —(CH₂)ᵢO-Heteroaryl, an optionally substituted —(CH₂)ᵢSugar, an optionally substituted —(CH₂)ᵢO-Sugar, or an optionally substituted —(CH₂)ᵢ—C(O)—(CH₂)ᵢ—O-Sugar group where R^{NA} and R^{NB} are each independently H, a C₁-C₆ alkyl which is optionally substituted with from 1-3 halo groups or 1-3 hydroxyl groups, an optionally substituted —(CH₂)ᵢO—C₁-C₆ alkyl (preferably OMe), an optionally substituted —(CH₂)ᵢC(O)C₁-C₆ alkyl, an optionally substituted —(CH₂)ᵢC(O)OC₁-C₆ alkyl, an optionally substituted —(CH₂)ᵢOC(O)C₁-C₆ alkyl, an optionally substituted —(CH₂)ᵢAryl, an optionally substituted —(CH₂)ᵢO-Aryl, an optionally substituted —(CH₂)ᵢHeteroaryl, an optionally substituted —(CH₂)ᵢO-Heteroaryl an optionally substituted —(CH₂)ᵢ₋Sugar, or an optionally substituted —(CH₂)ᵢ₋O-Sugar.

8. The compound according to claim 1 wherein R^{2A} and R^{2B} are each independently H, OH, an optionally substituted C₁-C₆ alkyl or C₂-C₆ alkenyl group wherein said alkyl group or said alkenyl group is preferably substituted with from 1-5 halogen groups and from 1-3 hydroxyl groups) or art optionally substituted —(CH₂)ᵢNR^{NA}R^{NB} group where R^{NA} and R^{NB} are each independently H, OMe, C₁-C₃ alkyl or —(CH₂)ᵢ₋O-Sugar.

9. The compound according to claim 7 wherein R^{NA} and R^{NB} are each independently H, methyl, OMe or —(CH₂)ᵢ₋Sugar.

10. The compound according to claim 1 wherein R^{1A} and the carbon to which R₂ is attached form a 5-6 membered carbocyclic ring.

11. The compound according to claim 1 wherein the carbon atoms to which OR¹ and R₂ are attached optionally are bonded to each other.

12. A compound having a chemical structure which is presented in any of FIGS. 4-20 hereof.

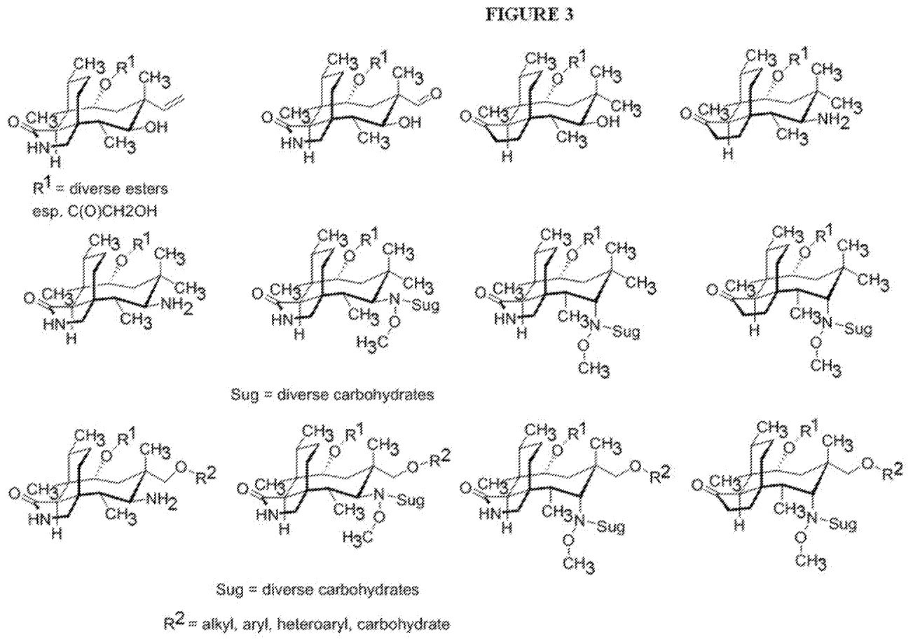

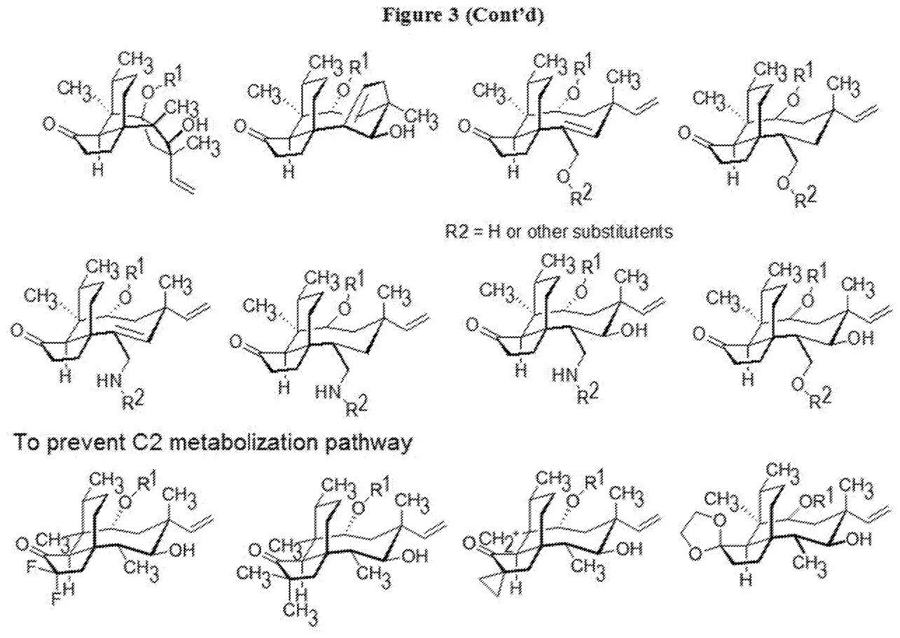

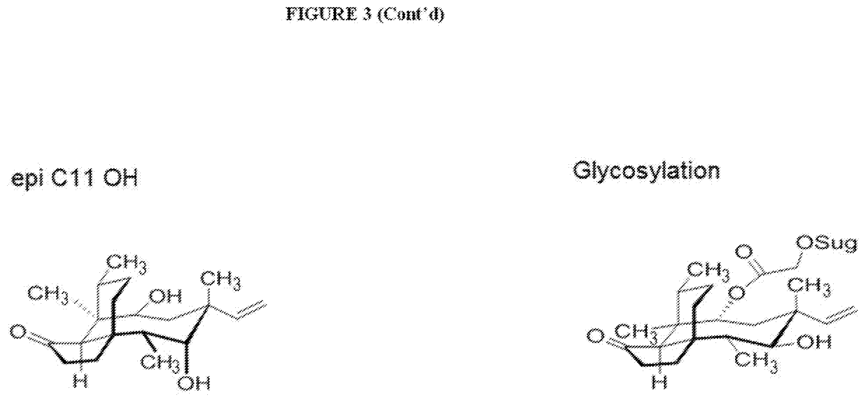

13. The compound according to claim 1 having a chemical structure which is presented in attached FIG. 3, where R¹ is H, a C₁-C₇ alkyl group which is optionally substituted with from 1-3 fluoro groups or 1-3 hydroxyl groups, a —C(O)—C₁-C₆ alkyl group which is optionally substituted with from 1-3 fluoro groups and 1-3 hydroxyl groups (more preferably a single hydroxyl group) or an optionally substituted —(CH₂)ᵢ—C(O)—(CH₂)ᵢ—O-Sugar group where each i is independently 0, 1 or 2; R² is H, a C₁-C₆ alkyl group which is optionally substituted with from 1-3 halo groups (preferably F) or 1-3 hydroxyl groups (often a single hydroxyl group), —C(O)C₁-C₆ alkyl which is optionally substituted with 1-3 halogens (preferably fluoride) and 1-3 hydroxyl groups (often a single hydroxyl group), —(CH₂)ᵢAryl, an optionally substituted —(CH₂)ᵢO-Aryl, an optionally substituted —(CH₂)ᵢHeteroaryl or an optionally substituted —(CH₂)ᵢO-Heteroaryl, an optionally substituted —(CH₂)₁Sugar, an optionally substituted —(CH₂)ᵢO-Sugar or an optionally substituted —(CH₂)ᵢ—C(O)—(CH₂)ᵢ—O-Sugar group where each i is independently 0, 1 or 2.

14. A pharmaceutical composition comprising an effective amount of a compound according to claim 1, further in combination with a pharmaceutically acceptable carrier, additive and/or excipient.

15. The composition according to claim 14 wherein said composition further comprises an additional antibiotic agent.

16. (canceled)

17. The composition according to claim 15 wherein said additional antibiotic agent is selected from the group consisting of Aminoglycosides including amikacin, gentamycin, kanamycin, neomycin, netilmicin, tobramycin, paromomycin, streptomycin, spectinomycin; Ansamycins, including geldanamycin, herbimycin and rifazimin; Carbacephems, including, loracarbef, ertapenem, doripenem, imipenem/cilastatin and meropenem; Cephalosporins, including cefadroxil, cefazolin, cefalothin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxxone, cefepime, ceftaroline fosamil and ceftobiprole; Glycopeptides, including teicoplanin, vancomycin, telavancin, dalbavancin and orivitavancin; Lincosamides, including clindamycin and lincomycin; Lipopeptides, including daptomycin; Macrolides, including azithromycin, clarithromycin, dirithromycin, erythromycin, roxithromycin, troleandomycin, telithromycin and spiramycin; Monobactams, including aztreonam; Nitrofurans, including furazolidone and nitrofurantoin; Oxazollidinones, including linezolid, posizolid, radezolid and torezolid; Penicillins, including amoxicillin, ampicillin, azlocillin, carbenicillin, cloxacillin, dicloxacillin, flucloxacillin, mezlicillin, methicillin, nafcillin, oxacillin, penicillin G, penicillin V, piperacillin, temocillin, ticarcillin, amoxicillin/clavulanate, ampicillin/sulbactam, piperacillin/tazobactam, ticarcillin/clavulanate; Polypeptides, including bacitracin, colistinand polymixin B; Quinolones/Fluoroquinolines, including ciprofloxacin, enoxacin, gatifloxacin, gemifloxacin, levofloxacin, lomefloxecin, moxifloxacin, naldixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfasalazine, sulfisoxazole; Trimethoprim-sulfamethoxazole and sulfonamidochysoidine; Tetracyclines, including demeclocycline, doxycycline, minocycline, oxytetracycline and tetracycline; Anti-Mycobacterial agents, including clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupiocin, platensimycin, quinupristin/dalfopristin, thiamphenicol, tigecycline, tinidazole and trimethoprim, and mixtures thereof.

18. (canceled)

19. (canceled)

20. A method of treating a bacterial infection comprising administering to a patient in need an effective amount of a composition according to claim 1.

21. The method according to claim 20 wherein said bacterial infection is a Staphylococcus aureus infection.

22. The method according to claim 21 wherein said infection is MRSA or MSSA infection.

23. A method of synthesizing a compound according to any of Schemes 1A, 1B, 2, 3, 4, 5, 6, 9, 10, 11, 12, 13, 14, 15, 16 or 17 according to the synthetic step(s) which are presented in those schemes.

24.-36. (canceled)
